(19) United States
(12) Patent Application Publication
(10) Pub. No.: US 2024/0140933 A1
(43) Pub. Date: May 2, 2024

(54) OXOINDOLINE COMPOUND FOR THE TREATMENT OF INFLAMMATORY DISEASES OR CANCER

(71) Applicant: Hoffmann-La Roche Inc., Little Falls, NJ (US)

(72) Inventors: Fabian DEY, Zürich (CH); Dong DING, Shanghai (CN); Xingchun HAN, Shanghai (CN); Chungen LIANG, Shanghai (CN); Hongtao XU, Shangahi (CN); Ge ZOU, Shanghai (CN)

(73) Assignee: Hoffmann-La Roche Inc., Little Falls, NJ (US)

(21) Appl. No.: 18/514,478

(22) Filed: Nov. 20, 2023

Related U.S. Application Data
(63) Continuation of application No. PCT/EP2022/063377, filed on May 18, 2022.

(30) Foreign Application Priority Data
May 21, 2021 (WO) PCT/CN2021/095205

(51) Int. Cl. C07D 401/14; C07D 401/04

(52) U.S. Cl. CPC C07D 401/14; C07D 401/04

(57) ABSTRACT

The present invention relates to compounds of formula (I),

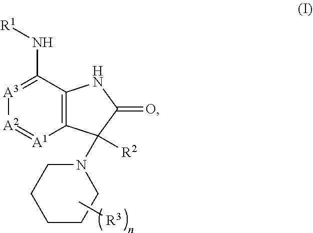

wherein R¹ to R³, A¹ to A³ and n are as described herein, and their pharmaceutically acceptable salt thereof, and compositions including the compounds and methods of using the compounds.

The present invention relates to organic compounds useful for the treatment and/or prevention of autoimmune and inflammatory disease or cancer; wherein autoimmune and inflammatory disease is selected from inflammatory bowel disease (IBD), chronic obstructive pulmonary disease (COPD), idiopathic pulmonary fibrosis (IPF), psoriasis, systemic lupus erythematosus (SLE); wherein cancer is selected from oropharyngeal squamous cell carcinoma, liver cancer, lung cancer, stomach cancer, and colon cancer. Specifically these molecules can inhibit Pyruvate dehydrogenase kinase (PDHK) and are useful for treating inflammatory bowel disease.

### BACKGROUND OF THE INVENTION

Chronic inflammation is the underlying cause of a broad spectrum of diseases, including rheumatoid arthritis (RA), chronic obstructive pulmonary disease (COPD), asthma, psoriasis, inflammatory bowel disease (IBD) and idiopathic pulmonary fibrosis (IPF), among others. Patients with inflammatory diseases suffer from chronic pain, poor sleep quality, obesity, physical impairment, and overall decreased quality of life. In the past decades, tremendous progress has been made to advance the treatment for inflammatory diseases, such as the discovery of cytokine blocking antibodies and Janus kinase (JAK) inhibitors. However, despite the increased treatment options, high unmet medical need remains due to the unsatisfied response rate and limited efficacy. Therefore, novel therapies with new mode of action are required to break the efficacy ceiling and provide sustained remission.

In recent years, there has been an increasing interest in immunometabolism as a promising field for the development of novel therapies. Metabolic changes are highly linked to cell activation and functionality during immune responses. Similar to Warburg effect, immune cells shift to aerobic glycolysis upon activation to meet their increased energy and structural requirements for cell proliferation and effector function. Pyruvate dehydrogenase kinase (PDHK), a member of the GHKL ATPase/kinase superfamily, is a key regulator in glycolysis and oxidative phosphorylation via controlling the activity of pyruvate dehydrogenase complex (PDC). PDC catalyzes the oxidative decarboxylation of pyruvate to acetyl-CoA, and represents a central node linking glycolysis and the tricarboxylic acid cycle (TCA) cycle. PDHK inhibits PDC activity by phosphorylating three serine residues on the E1 subunit (Korotchkina, L. G. et al. J Biol Chem 276, 37223-37229, 2001; Patel, M. S. et al. Biochem Soc Trans 34, 217-222, 2006). Inhibition of PDHK reverses the Warburg phenotype as evidenced by a decrease in lactate production and increased glycose oxidation (Sun, R. C. et al. Breast Cancer Res Treat 120, 253-260, 2010; Wong, J. Y. et al. Gynecol Oncol 109, 394-402, 2008).

CD4⁺ T cells play a critical role in inflammatory or autoimmune diseases. Antigen exposure initially leads to T cell activation, inducing rapid growth and proliferation. Depending on the cytokine environment during activation, CD4⁺ T cells then differentiate into effector (Teff) (Th1 and Th17) or Treg subsets. Each of these subsets plays a unique role in the adaptive immune system, with Teffs driving immunity and inflammation while Tregs play an opposing role, suppressing Teffs to limit excessive inflammatory responses. The balance between Teffs and Tregs is crucial to providing sufficient immune protection without promoting autoimmunity. Indeed, many autoimmune diseases, including multiple sclerosis (MS) and inflammatory bowel disease (IBD), involve an imbalance of Teffs to Tregs or decreased Treg function. Th17, in particular, plays a key proinflammatory role in many autoimmune diseases, including experimental autoimmune encephalomyelitis (EAE), IBD, and graft-versus-host disease. Accumulating evidence indicates PDHK plays an important role in the immune cell function. Inhibition of PDHK1 selectively suppressed Th17 cells, increased Treg, and protected animals against colitis and EAE (Gerriets, V. A. et al. J Cin Invest 125, 194-207, 2015). Knockdown of PDHK inhibited inflammatory macrophage (M1) polarization and inflammatory cytokine production while promoted the expression of regulatory macrophage (M2) signature genes (Tan, Z. et al. J Immunol 194, 6082-6089, 2015). In addition, inhibition of PDHK suppressed B cell proliferation and antibody secretion (Caro-Maldonado, A. et al. J Immunol 192, 3626-3636, 2014). Taken together, PDHK inhibition is expected to control the activation of immune cells, thus presents a promising therapeutic target for autoimmune and inflammatory diseases driven by T cells, B cells and macrophages. As such, we invented oral compounds that inhibit PDHK to improve the treatment outcome for the patients with autoimmune and inflammatory diseases, such as IBD, COPD, IPF, psoriasis and systemic lupus erythematosus (SLE). Besides inflammatory diseases, a considerable body of evidence points to an important role for PDHK in cancer with glycolytic phenotype (Lu, C-W. et al. Am J Pathol 179(3), 1405-1414, 2011; Chatterjee, N. et al. Cell Rep 28(9): 2317-2330, 2019; Golias, T. et al. Sci Rep 6:31146, 2016). Therefore, PDHK inhibitors that we have invented are also applicable for cancer treatment, including oropharyngeal squamous cell carcinoma (Golias, T. et al. Sci Rep 6:31146, 2016), liver cancer, lung cancer, stomach cancer (Shao et al. Cancer Commun, 39:54, 2019), and colon cancer (Lu, C-W. et al. Am J Pathol 179(3), 1405-1414).

### SUMMARY OF THE INVENTION

The present invention relates to novel compounds of formula (I),

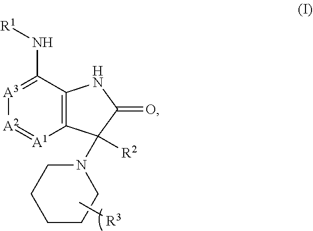

wherein
R¹ is C₁₋₆alkyl;
R² is C₁₋₆alkyl;
R³ is OR⁴, —(CH₂)ₙ—R⁵, NR⁶R⁷, COR⁸ or R⁹; wherein
   R⁴ is phenyl substituted once or twice by substituents independently selected from carboxy, halogen, hydroxy, formyl, halosulfonyl and C₁₋₆alkyl;
   R⁵ is phenyl substituted by carbamoyl, carboxy or cyano;
   R⁶ is H, C₁₋₆alkyl, C₁₋₆alkylcarbonyl or carbamoyl;
   R⁷ is C₂₋₆alkenylsulfonyl,
      benzoyl substituted once, twice or three times by substituents independently selected from halogen, hydroxy and halosulfonyl,
      COR^{a}, wherein R^{a} is 1,1-dioxothianyl, C₁₋₆alkyl, cyanoazaspiro[3.3]heptanyl, haloC₁₋₆alkylcarbonyl-azaspiro[3.3]heptanyl, C₂₋₆alkenylcarbonyl-azaspiro[3.3]heptanyl, cyanoazetidinyl or caynopiperidinyl,
      phenyl substituted once or twice by substituents independently selected from cyano, hydroxy, C₁₋₆alkoxy, hydroxyC₁₋₆alkylcarbonyl, halosulfonyl, halosulfonyloxy and C₂₋₆alkenylsulfonyl,
      pyrazinyl substituted by cyano,
      pyridinyl substituted once or twice by substituents independently selected from halogen, C₁₋₆alkyl, carboxy, carbamoyl, cyano, hydroxy, hydroxyC₁₋₆alkyl, C₁₋₆alkylsulfonyl, halosulfonyl and halosulfonyloxy, or pyrimidinyl substituted by cyano;
   R⁸ is amino, C₁₋₆alkylamino, phenylamino or benzylamino;
   R⁹ is C₁₋₆alkyl, halogen, hydroxy, phenyl, (carboxyphenoxy)phenyl, (carbamoylphenoxy)phenyl or (cyanophenoxy)phenyl;
A¹ is CH;
A² is CR^{b}, wherein R^{b} is halogen;
A³ is CH;
n is 1, 2, 3, 4, 5 or 6;
or a pharmaceutically acceptable salt thereof.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and propyl.

The term “oxy” denotes —O—.

The term “C₁₋₆alkoxy” denotes C₁₋₆alkyl-O—.

The term “C₂₋₆alkenyl” denotes an unsaturated, linear or branched chain alkenyl group containing 2 to 6, particularly 2 to 4 carbon atoms, for example vinyl, propenyl, allyl, butenyl and the like. Particular “C₂₋₆alkenyl” groups are allyl and vinyl.

The term “amino”, alone or in combination, signifies the primary amino group, the secondary amino group, or the tertiary amino group.

The term “aryl” denotes a monovalent aromatic carbocyclic mono- or bicyclic ring system comprising 6 to 10 carbon ring atoms. Examples of aryl moieties include phenyl and naphthyl.

The term “heteroaryl” denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl.

The term “halogen” and “halo” are used interchangeably herein and denote fluoro, chloro, bromo, or iodo.

The term “halosulfonyl” denotes —SO₂-halogen, particular “halosulfonyl” groups are fluorosulfonyl and chlorosulfonyl.

The term “halosulfonyloxy” denotes —O—SO₂-halogen, particular “halosulfonyloxy” groups are fluorosulfonyloxy and chlorosulfonyloxy.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example chloromethyl, 3,3,3-trifluoropropyl, 2-fluoroethyl, trifluoroethyl, fluoromethyl, difluoromethyl, difluoroethyl or trifluoromethyl.

The term “heterocyclyl” denotes a monovalent saturated or partly unsaturated mono- or bicyclic ring system of 3 to 9 ring atoms, comprising 1, 2, or 3 ring heteroatoms selected from N, O and S, the remaining ring atoms being carbon. In particular embodiments, heterocyclyl is a monovalent saturated monocyclic ring system of 4 to 7 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocyclyl are azaspiro[3.3]heptanyl, 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocyclyl are dihydrofuryl, imidazolinyl, dihydro-oxazolyl, tetrahydro-pyridinyl, or dihydropyranyl.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Inhibitor of PDHK1

The present invention relates to (i) a compound of formula (I),

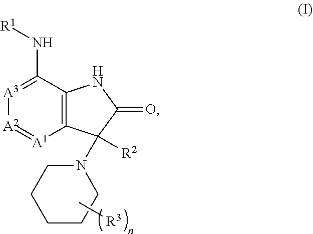

wherein
R¹ is C₁₋₆alkyl;
R² is C₁₋₆alkyl;
R³ is OR⁴, —(CH₂)ₙ—R⁵, NR⁶R⁷, COR⁸ or R⁹; wherein
   R⁴ is phenyl substituted once or twice by substituents independently selected from carboxy, halogen, hydroxy, formyl, halosulfonyl and C₁₋₆alkyl;
   R⁵ is phenyl substituted by carbamoyl, carboxy or cyano;
   R⁶ is H, C₁₋₆alkyl, C₁₋₆alkylcarbonyl or carbamoyl;
   R⁷ is C₂₋₆alkenylsulfonyl,
      benzoyl substituted once, twice or three times by substituents independently selected from halogen, hydroxy and halosulfonyl,
      COR^{a}, wherein R^{a} is 1,1-dioxothianyl, C₁₋₆alkyl, cyanoazaspiro[3.3]heptanyl, haloC₁₋₆alkylcarbonyl-azaspiro[3.3]heptanyl, C₂₋₆alkenylcarbonyl-azaspiro[3.3]heptanyl, cyanoazetidinyl or caynopiperidinyl,
      phenyl substituted once or twice by substituents independently selected from cyano, hydroxy, C₁₋₆alkoxy, hydroxyC₁₋₆alkylcarbonyl, halosulfonyl, halosulfonyloxy and C₂₋₆alkenylsulfonyl,
      pyrazinyl substituted by cyano,
      pyridinyl substituted once or twice by substituents independently selected from halogen, C₁₋₆alkyl, carboxy, carbamoyl, cyano, hydroxy, hydroxyC₁₋₆alkyl, C₁₋₆alkylsulfonyl, halosulfonyl and halosulfonyloxy, or pyrimidinyl substituted by cyano;
   R⁸ is amino, C₁₋₆alkylamino, phenylamino or benzylamino;
   R⁹ is C₁₋₆alkyl, halogen, hydroxy, phenyl, (carboxyphenoxy)phenyl, (carbamoylphenoxy)phenyl or (cyanophenoxy)phenyl;
A¹ is CH;
A² is CR^{b}, wherein R¹ is halogen;
A³ is CH;
n is 1, 2, 3, 4, 5 or 6;
or a pharmaceutically acceptable salt thereof.

Another embodiment of present invention is (ii) a compound of formula (II),

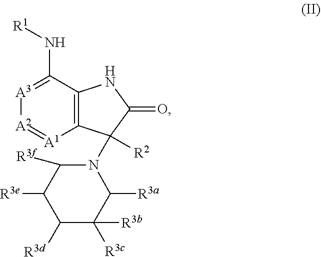

wherein
R¹ is C₁₋₆alkyl;
R² is C₁₋₆alkyl;
R^{3a} is H;
R^{3b} is OR⁴, —(CH₂)ₙ—R⁵, NR⁶R⁷ or COR⁸; wherein
   R⁴ is phenyl substituted once or twice by substituents independently selected from carboxy, halogen, hydroxy, formyl, halosulfonyl and C₁₋₆alkyl;
   R⁵ is phenyl substituted by carbamoyl, carboxy or cyano;
   R⁶ is H, C₁₋₆alkyl, C₁₋₆alkylcarbonyl or carbamoyl;
   R⁷ is C₂₋₆alkenylsulfonyl,
      benzoyl substituted once, twice or three times by substituents independently selected from halogen, hydroxy and halosulfonyl,
      COR^{a}, wherein R^{a} is 1,1-dioxothianyl, C₁₋₆alkyl, cyanoazaspiro[3.3]heptanyl, haloC₁₋₆alkylcarbonyl-azaspiro[3.3]heptanyl, C₂₋₆alkenylcarbonyl-azaspiro[3.3]heptanyl, cyanoazetidinyl or caynopiperidinyl,
      phenyl substituted once or twice by substituents independently selected from cyano, hydroxy, C₁₋₆alkoxy, hydroxyC₁₋₆alkylcarbonyl, halosulfonyl, halosulfonyloxy and C₂₋₆alkenylsulfonyl,
      pyrazinyl substituted by cyano,
      pyridinyl substituted once or twice by substituents independently selected from halogen, C₁₋₆alkyl, carboxy, carbamoyl, cyano, hydroxy, hydroxyC₁₋₆alkyl, C₁₋₆alkylsulfonyl, halosulfonyl and halosulfonyloxy, or
      pyrimidinyl substituted by cyano;
   R⁸ is amino, C₁₋₆alkylamino, phenylamino or benzylamino;
R^{3c} is H or C₁₋₆alkyl;
R^{3d} is H or R⁹; wherein R⁹ is C₁₋₆alkyl, halogen, hydroxy, phenyl, (carboxyphenoxy)phenyl, (carbamoylphenoxy)phenyl or (cyanophenoxy)phenyl;
R^{3e} is H or halogen;
R^{3f} is H;
A¹ is CH;
A² is CR^{b}, wherein R¹ is halogen;
A³ is CH;
n is 1, 2, 3, 4, 5 or 6;
or a pharmaceutically acceptable salt thereof.

A further embodiment of present invention is (iii) a compound of formula (I) or (II) according to (i) or (ii), or a pharmaceutically acceptable salt thereof, R⁴ is formyl(hydroxy)phenyl.

A further embodiment of present invention is (iv) a compound of formula (I) or (II), according to any one of (i) to (iii), or a pharmaceutically acceptable salt thereof, wherein R⁴ is 3-formyl-4-hydroxyphenyl.

A further embodiment of present invention is (v) a compound of formula (I) or (II) according to any one of (i) to (iv), wherein R⁶ is H.

A further embodiment of present invention is (vi) a compound of formula (I) or (II), or a pharmaceutically acceptable salt thereof, according to any one of (i) to (v), wherein
R⁷ is COR^{a}, wherein R^{a} is C₁₋₆alkyl, cyanoazaspiro[3.3]heptanyl, haloC₁₋₆alkylcarbonyl-azaspiro[3.3]heptanyl or C₂₋₆alkenylcarbonyl-azaspiro[3.3]heptanyl,
   phenyl substituted once or twice by substituents independently selected from C₁₋₆alkoxy, halosulfonyl and C₂₋₆alkenylsulfonyl, or
   pyridinyl substituted by cyano or halosulfonyloxy.

A further embodiment of present invention is (vii) a compound of formula (I) or (II), or a pharmaceutically acceptable salt thereof, according to any one of (i) to (vi), wherein
R⁷ is COR^{a}, wherein R^{a} is acetyl, cyanoazaspiro[3.3]heptanyl, (chloroacetyl)azaspiro[3.3]heptanyl or (propenoyl)azaspiro[3.3]heptanyl,
   phenyl substituted once or twice by substituents independently selected from methoxy, fluorosulfonyl and vinylsulfonyl, or
   pyridinyl substituted by cyano or fluorosulfonyloxy.

A further embodiment of present invention is (viii) a compound of formula (I) or (II), or a pharmaceutically acceptable salt thereof, according to any one of (i) to (vii), wherein R⁹ is C₁₋₆alkyl, phenyl or (cyanophenoxy)phenyl.

A further embodiment of present invention is (ix) a compound of formula (I) or (II), or a pharmaceutically acceptable salt thereof, according to any one of (i) to (viii), wherein R⁹ is methyl, phenyl or (cyanophenoxy)phenyl.

A further embodiment of present invention is (x) a compound of formula (I) or (II), or a pharmaceutically acceptable salt thereof, according to any one of (i) to (ix), wherein n is 1.

A further embodiment of present invention is (xi) a compound of formula (I), or a pharmaceutically acceptable salt thereof, according to any one of (i) to (x), wherein
R¹ is C₁₋₆alkyl;
R² is C₁₋₆alkyl;
R³ is OR⁴, NR⁶R⁷ or R⁹; wherein
   R⁴ is formyl(hydroxy)phenyl;
   R⁶ is H;
   R⁷ is COR^{a}, wherein R^{a} is C₁₋₆alkyl, cyanoazaspiro[3.3]heptanyl, haloC₁₋₆alkylcarbonyl-azaspiro[3.3]heptanyl or C₂₋₆alkenylcarbonyl-azaspiro[3.3]heptanyl,
      phenyl substituted once or twice by substituents independently selected from C₁₋₆alkoxy, halosulfonyl and C₂₋₆alkenylsulfonyl, or
      pyridinyl substituted by cyano or halosulfonyloxy;
   R⁹ is C₁₋₆alkyl, phenyl or (cyanophenoxy)phenyl;
A¹ is CH;
A² is CR^{b}, wherein R¹ is halogen;
A³ is CH;
or a pharmaceutically acceptable salt thereof.

A further embodiment of present invention is (xii) a compound of formula (I) or (II), or a pharmaceutically acceptable salt thereof, according to any one of (i) to (xi), wherein
R¹ is ethyl;
R² is methyl or ethyl;
R³ is OR⁴, NR⁶R⁷ or R⁹; wherein
   R⁴ is formyl(hydroxy)phenyl;
   R⁶ is H;
   R⁷ is COR^{a}, wherein R^{a} is methyl, cyanoazaspiro[3.3]heptanyl, (chloroacetyl)azaspiro[3.3]heptanyl or (propenoyl)azaspiro[3.3]heptanyl,
      phenyl substituted once or twice by substituents independently selected from methoxy, fluorosulfonyl and vinylsulfonyl, or
      pyridinyl substituted by cyano or fluorosulfonyloxy;
   R⁹ is methyl, phenyl or (cyanophenoxy)phenyl;
A¹ is CH;
A² is CR^{b}, wherein R^{b} is fluoro;
A³ is CH;
or a pharmaceutically acceptable salt thereof.

A further embodiment of present invention is (xiii) a compound of formula (I) or (II), or a pharmaceutically acceptable salt thereof, according to any one of (i) to (xii), wherein
R¹ is ethyl;
R² is methyl or ethyl;
R³ is OR⁴, NR⁶R⁷ or R⁹; wherein
   R⁴ is 3-formyl-4-hydroxyphenyl;
   R⁶ is H;
   R⁷ is acetyl, 2-cyano-2-azaspiro[3.3]heptane-6-carbonyl, 2-(2-chloroacetyl)-2-azaspiro[3.3]heptane-6-carbonyl or 2-prop-2-enoyl-2-azaspiro[3.3]heptane-6-carbonyl, 4-fluorosulfonyl-3-methoxyphenyl, 4-vinylsulfonylphenyl, 6-cyano-3-pyridinyl or 6-fluorosulfonyloxy-3-pyridinyl;
   R⁹ is methyl, phenyl or 2-(4-cyanophenoxy)phenyl;
A¹ is CH;
A² is CR^{b}, wherein R^{b} is fluoro;
A³ is CH;
or a pharmaceutically acceptable salt thereof.

A further embodiment of present invention is (xiv) a compound of formula (I) or (II), according to any one of (i) to (xiii), wherein
R¹ is C₁₋₆alkyl;
R² is C₁₋₆alkyl;
R^{3a} is H;
R^{3b} is OR⁴ or NR⁶R⁷; wherein
   R⁴ is formyl(hydroxy)phenyl;
   R⁶ is H;
   R⁷ is COR^{a}, wherein R^{a} is C₁₋₆alkyl, cyanoazaspiro[3.3]heptanyl, haloC₁₋₆alkylcarbonyl-azaspiro[3.3]heptanyl or C₂₋₆alkenylcarbonyl-azaspiro[3.3]heptanyl,
      phenyl substituted once or twice by substituents independently selected from C₁₋₆ alkoxy, halosulfonyl and C₂₋₆alkenylsulfonyl, or
   pyridinyl substituted by cyano or halosulfonyloxy;
R^{3c} is H or C₁₋₆alkyl;
R^{3d} is H or R⁹; wherein R⁹ is C₁₋₆alkyl, phenyl or (cyanophenoxy)phenyl;
R^{3e} is H;
R^{3f} is H;
A¹ is CH;
A² is CR^{b}, wherein R^{b} is halogen;
A³ is CH;
or a pharmaceutically acceptable salt thereof.

A further embodiment of present invention is (xv) a compound of formula (I) or (II), according to any one of (i) to (xiv), wherein
R¹ is ethyl;
R² is methyl or ethyl;
R^{3a} is H;
R^{3b} is OR⁴ or NR⁶R⁷; wherein
   R⁴ is formyl(hydroxy)phenyl;
   R⁶ is H;
   R⁷ is COR^{a}, wherein R^{a} is methyl, cyanoazaspiro[3.3]heptanyl, (chloroacetyl)azaspiro[3.3]heptanyl or (propenoyl)azaspiro[3.3]heptanyl,
      phenyl substituted once or twice by substituents independently selected from methoxy, fluorosulfonyl and vinylsulfonyl, or
   pyridinyl substituted by cyano or fluorosulfonyloxy;
R^{3c} is H or methyl;
R^{3d} is H or R⁹; wherein R⁹ is methyl, phenyl or (cyanophenoxy)phenyl;
R^{3e} is H;
R^{3f} is H;
A¹ is CH;
A² is CR^{b}, wherein R^{b} is fluoro;
A³ is CH;
or a pharmaceutically acceptable salt thereof.

A further embodiment of present invention is (xvi) a compound of formula (I) or (II), according to any one of (i) to (xv), wherein
R¹ is ethyl;
R² is methyl or ethyl;
R^{3a} is H;
R^{3b} is OR⁴ or NR⁶R⁷; wherein
   R⁴ is formyl(hydroxy)phenyl;
   R⁶ is H;
   R⁷ is acetyl, 2-cyano-2-azaspiro[3.3]heptane-6-carbonyl, 2-(2-chloroacetyl)-2-azaspiro[3.3]heptane-6-carbonyl or 2-prop-2-enoyl-2-azaspiro[3.3]heptane-6-carbonyl, 4-fluorosulfonyl-3-methoxyphenyl, 4-vinylsulfonylphenyl, 6-cyano-3-pyridinyl or 6-fluorosulfonyloxy-3-pyridinyl;
R^{3c} is H or methyl;
R^{3d} is H or R⁹; wherein R⁹ is methyl, phenyl or 2-(4-cyanophenoxy)phenyl yl;
R^{3e} is H;
R^{3f} is H;
A¹ is CH;
A² is CR^{b}, wherein R^{b} is fluoro;
A³ is CH;
or a pharmaceutically acceptable salt thereof

Another embodiment of present invention is a compound of formula (I) or (II) selected from the following:
2-chloro-4-[[(3R)-1-[(3R)-7-(ethylamino)-5-fluoro-3-methyl-2-oxo-indolin-3-yl]-3-piperidyl]oxy]benzoic acid;
2-chloro-4-[[(3R)-1-[(3S)-7-(ethylamino)-5-fluoro-3-methyl-2-oxo-indolin-3-yl]-3-piperidyl]oxy]benzoic acid;
5-[[(3R)-1-[7-(ethylamino)-5-fluoro-3-methyl-2-oxo-indolin-3-yl]-3-piperidyl]oxy]-2-hydroxy-benzaldehyde;
4-[[(3R)-1-[(3R)-7-(ethylamino)-5-fluoro-3-methyl-2-oxo-indolin-3-yl]-3-piperidyl]oxy]benzenesulfonyl fluoride;
4-[[(3R)-1-[(3S)-7-(ethylamino)-5-fluoro-3-methyl-2-oxo-indolin-3-yl]-3-piperidyl]oxy]benzenesulfonyl fluoride;
4-[[(3R)-1-[(3R)-7-(ethylamino)-5-fluoro-3-methyl-2-oxo-indolin-3-yl]-3-piperidyl]oxy]-2-methoxy-benzenesulfonyl fluoride;
4-[[(3R)-1-[(3S)-7-(ethylamino)-5-fluoro-3-methyl-2-oxo-indolin-3-yl]-3-piperidyl]oxy]-2-methoxy-benzenesulfonyl fluoride;
4-[[(3S)-1-[7-(ethylamino)-5-fluoro-3-methyl-2-oxo-indolin-3-yl]-3-piperidyl]methyl]benzoic acid;
4-[[(3S)-1-[7-(ethylamino)-5-fluoro-3-methyl-2-oxo-indolin-3-yl]-3-piperidyl]methyl]benzamide;
4-[[(3S)-1-[7-(ethylamino)-5-fluoro-3-methyl-2-oxo-indolin-3-yl]-3-piperidyl]methyl]benzonitrile;
4-[[(3R)-1-[7-(ethylamino)-5-fluoro-3-methyl-2-oxo-indolin-3-yl]-3-piperidyl]amino]benzonitrile;
4-[[(3R)-1-[7-(ethylamino)-5-fluoro-3-methyl-2-oxo-indolin-3-yl]-3-piperidyl]amino]-2-hydroxy-benzonitrile;
5-[[(3S)-1-[7-(ethylamino)-5-fluoro-3-methyl-2-oxo-indolin-3-yl]-3-piperidyl]amino]pyridine-2-carboxylic acid;
5-[[(3R)-1-[7-(ethylamino)-5-fluoro-3-methyl-2-oxo-indolin-3-yl]-3-piperidyl]amino]pyridine-2-carboxylic acid;
5-[[(3R)-1-[7-(ethylamino)-5-fluoro-3-methyl-2-oxo-indolin-3-yl]-3-piperidyl]amino]pyridine-2-carboxamide;
5-[[(3R)-1-[7-(ethylamino)-5-fluoro-3-methyl-2-oxo-indolin-3-yl]-3-piperidyl]amino]pyridine-2-carbonitrile;
6-[[(3R)-1-[7-(ethylamino)-5-fluoro-3-methyl-2-oxo-indolin-3-yl]-3-piperidyl]amino]pyridine-3-carbonitrile;
5-[[(3R)-1-[7-(ethylamino)-5-fluoro-3-methyl-2-oxo-indolin-3-yl]-3-piperidyl]amino]pyrazine-2-carbonitrile;
6-[[(3R)-1-[7-(ethylamino)-5-fluoro-3-methyl-2-oxo-indolin-3-yl]-3-piperidyl]amino]pyridazine-3-carbonitrile;
5-[[(3R)-1-[7-(ethylamino)-5-fluoro-3-methyl-2-oxo-indolin-3-yl]-3-piperidyl]amino]pyrimidine-2-carbonitrile;
7-(ethylamino)-5-fluoro-3-[(3R)-3-[[6-(hydroxymethyl)-3-pyridyl]amino]-1-piperidyl]-3-methyl-indolin-2-one;
7-(ethylamino)-5-fluoro-3-[(3R)-3-[4-(2-hydroxyacetyl)anilino]-1-piperidyl]-3-methyl-indolin-2-one;
7-(ethylamino)-5-fluoro-3-methyl-3-[(3R)-3-[(6-methylsulfonyl-3-pyridyl)amino]-1-piperidyl]indolin-2-one;
7-(ethylamino)-5-fluoro-3-[(3R)-3-[(6-hydroxy-3-pyridyl)amino]-1-piperidyl]-3-methyl-indolin-2-one;
3-chloro-5-[[(3R)-1-[7-(ethylamino)-5-fluoro-3-methyl-2-oxo-indolin-3-yl]-3-piperidyl]amino]pyridine-2-carbonitrile;
5-[[(3R)-1-[7-(ethylamino)-5-fluoro-3-methyl-2-oxo-indolin-3-yl]-3-methyl-3-piperidyl]amino]pyridine-2-carbonitrile;
5-[[(3R)-1-[7-(ethylamino)-5-fluoro-3-methyl-2-oxo-indolin-3-yl]-3-piperidyl]-methyl-amino]pyridine-2-carboxamide;
N-(6-cyano-3-pyridyl)-N-[(3R)-1-[3-ethyl-7-(ethylamino)-5-fluoro-2-oxo-indolin-3-yl]-3-piperidyl]acetamide;
1-(6-cyano-3-pyridyl)-1-[(3R)-1-[3-ethyl-7-(ethylamino)-5-fluoro-2-oxo-indolin-3-yl]-3-piperidyl]urea;
3-[[(3R)-1-[7-(ethylamino)-5-fluoro-3-methyl-2-oxo-indolin-3-yl]-3-piperidyl]amino]benzenesulfonyl fluoride;
4-[[(3R)-1-[7-(ethylamino)-5-fluoro-3-methyl-2-oxo-indolin-3-yl]-3-piperidyl]amino]-2-methoxy-benzenesulfonyl fluoride;
6-[[(3R)-1-[7-(ethylamino)-5-fluoro-3-methyl-2-oxo-indolin-3-yl]-3-piperidyl]amino]pyridine-3-sulfonyl fluoride;
4-methyl-6-[[(3R)-1-[7-(ethylamino)-5-fluoro-3-methyl-2-oxo-indolin-3-yl]-3-piperidyl]amino]pyridine-3-sulfonyl fluoride;
7-(ethylamino)-5-fluoro-3-methyl-2-oxo-3-[(3R)-3-(3-fluorosulfonyloxyanilino)-1-piperidyl]indoline;
7-(ethylamino)-5-fluoro-3-methyl-2-oxo-3-[(3R)-3-(4-fluorosulfonyloxyanilino)-1-piperidyl]indoline;
7-(ethylamino)-5-fluoro-3-methyl-2-oxo-3-[(3R)-3-[(6-fluorosulfonyloxy-3-pyridyl)amino]-1-piperidyl]indoline;
7-(ethylamino)-5-fluoro-3-methyl-3-[(3R)-3-(4-vinylsulfonylanilino)-1-piperidyl]indolin-2-one;
N-[(3R)-1-[7-(ethylamino)-5-fluoro-3-methyl-2-oxo-indolin-3-yl]-3-piperidyl]-3,5-difluoro-4-hydroxy-benzamide;
N-[(3R)-1-[7-(ethylamino)-5-fluoro-3-methyl-2-oxo-indolin-3-yl]-3-piperidyl]-1,1-dioxo-thiane-4-carboxamide;
2-cyano-N-[(3R)-1-[7-(ethylamino)-5-fluoro-3-methyl-2-oxo-indolin-3-yl]-3-piperidyl]-2-azaspiro[3.3]heptane-6-carboxamide;
1-cyano-N-[(3R)-1-[7-(ethylamino)-5-fluoro-3-methyl-2-oxo-indolin-3-yl]-3-piperidyl]piperidine-4-carboxamide;
2-cyano-N-[(3R)-1-[3-ethyl-7-(ethylamino)-5-fluoro-2-oxo-indolin-3-yl]-3-piperidyl]-2-azaspiro[3.3]heptane-6-carboxamide;
2-(2-chloroacetyl)-N-[(3R)-1-[7-(ethylamino)-5-fluoro-3-methyl-2-oxo-indolin-3-yl]-3-piperidyl]-2-azaspiro[3.3]heptane-6-carboxamide;
N-[(3R)-1-[7-(ethylamino)-5-fluoro-3-methyl-2-oxo-indolin-3-yl]-3-piperidyl]-2-prop-2-enoyl-2-azaspiro[3.3]heptane-6-carboxamide;
3-[[(3R)-1-[7-(ethylamino)-5-fluoro-3-methyl-2-oxo-indolin-3-yl]-3-piperidyl]carbamoyl]benzenesulfonyl fluoride;
4-[[(3R)-1-[7-(ethylamino)-5-fluoro-3-methyl-2-oxo-indolin-3-yl]-3-piperidyl]carbamoyl]benzenesulfonyl fluoride;
7-(ethylamino)-5-fluoro-3-[(3R)-3-[(3-fluorosulfonyloxybenzoyl)amino]-1-piperidyl]-3-methyl-2-oxo-indoline;
5-[[(3R,5R)-1-[7-(ethylamino)-5-fluoro-3-methyl-2-oxo-indolin-3-yl]-5-fluoro-3-piperidyl]amino]pyridine-2-carbonitrile;
5-[[(3R,5S)-1-[7-(ethylamino)-5-fluoro-3-methyl-2-oxo-indolin-3-yl]-5-fluoro-3-piperidyl]amino]pyridine-2-carbonitrile;
5-[[(3R,5S)-1-[7-(ethylamino)-5-fluoro-3-methyl-2-oxo-indolin-3-yl]-5-methyl-3-piperidyl]amino]pyridine-2-carbonitrile;
5-[[(3S,4S)-1-[7-(ethylamino)-5-fluoro-3-methyl-2-oxo-indolin-3-yl]-4-fluoro-3-piperidyl]amino]pyridine-2-carbonitrile;
5-[[(3S,4S)-1-[3-ethyl-7-(ethylamino)-5-fluoro-2-oxo-indolin-3-yl]-4-fluoro-3-piperidyl]amino]pyridine-2-carbonitrile;
5-[[(3S,4S)-1-[7-(ethylamino)-5-fluoro-3-methyl-2-oxo-indolin-3-yl]-4-hydroxy-3-piperidyl]amino]pyridine-2-carbonitrile;
5-[[(3S,4R)-1-[7-(ethylamino)-5-fluoro-3-methyl-2-oxo-indolin-3-yl]-4-hydroxy-3-piperidyl]amino]pyridine-2-carbonitrile;
N-[(3R,4R)-1-[7-(ethylamino)-5-fluoro-3-methyl-2-oxo-indolin-3-yl]-4-phenyl-3-piperidyl]acetamide;
N-[(3R,4R)-1-[7-(ethylamino)-5-fluoro-3-methyl-2-oxo-indolin-3-yl]-4-phenyl-3-piperidyl]ethenesulfonamide;
1-cyano-N-[(3R,4R)-1-[7-(ethylamino)-5-fluoro-3-methyl-2-oxo-indolin-3-yl]-4-phenyl-3-piperidyl]azetidine-3-carboxamide;
5-[[(3R,4R)-1-[7-(ethylamino)-5-fluoro-3-methyl-2-oxo-indolin-3-yl]-4-phenyl-3-piperidyl]amino]pyridine-2-carbonitrile;
4-[2-[(3R,4R)-3-acetamido-1-[7-(ethylamino)-5-fluoro-3-methyl-2-oxo-indolin-3-yl]-4-piperidyl]phenoxy]benzoic acid;
4-[2-[(3R,4R)-3-acetamido-1-[7-(ethylamino)-5-fluoro-3-methyl-2-oxo-indolin-3-yl]-4-piperidyl]phenoxy]benzamide;
N-[(3R,4R)-4-[2-(4-cyanophenoxy)phenyl]-1-[7-(ethylamino)-5-fluoro-3-methyl-2-oxo-indolin-3-yl]-3-piperidyl]acetamide;
(3R,4R)-1-[(3R)-7-(ethylamino)-5-fluoro-3-methyl-2-oxo-indolin-3-yl]-4-phenyl-piperidine-3-carboxamide;
(3R,4R)-1-[(3S)-7-(ethylamino)-5-fluoro-3-methyl-2-oxo-indolin-3-yl]-4-phenyl-piperidine-3-carboxamide;
(3R,4R)-1-[(3R)-7-(ethylamino)-5-fluoro-3-methyl-2-oxo-indolin-3-yl]-N-methyl-4-phenyl-piperidine-3-carboxamide;
(3R,4R)-1-[(3S)-7-(ethylamino)-5-fluoro-3-methyl-2-oxo-indolin-3-yl]-N-methyl-4-phenyl-piperidine-3-carboxamide;
(3R,4R)-1-[(3R)-7-(ethylamino)-5-fluoro-3-methyl-2-oxo-indolin-3-yl]-N,4-diphenyl-piperidine-3-carboxamide;
(3R,4R)-1-[(3S)-7-(ethylamino)-5-fluoro-3-methyl-2-oxo-indolin-3-yl]-N,4-diphenyl-piperidine-3-carboxamide;
(3R,4R)-N-benzyl-1-[(3R)-7-(ethylamino)-5-fluoro-3-methyl-2-oxo-indolin-3-yl]-4-phenyl-piperidine-3-carboxamide;
(3R,4R)-N-benzyl-1-[(3S)-7-(ethylamino)-5-fluoro-3-methyl-2-oxo-indolin-3-yl]-4-phenyl-piperidine-3-carboxamide;
(3S,4S)-1-[(3R)-7-(ethylamino)-5-fluoro-3-methyl-2-oxo-indolin-3-yl]-4-phenyl-piperidine-3-carboxamide; and
(3S,4S)-1-[(3S)-7-(ethylamino)-5-fluoro-3-methyl-2-oxo-indolin-3-yl]-4-phenyl-piperidine-3-carboxamide;
   or a pharmaceutically acceptable salt thereof.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of Formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of Formula (I) is formulated in an acetate buffer, at pH 5. In another embodiment, the compounds of Formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit the phosphorylation of the E1 subunit of PDC by PDHK1. For example, such amount may be below the amount that is toxic to normal cells, or the mammal as a whole.

In one example, the pharmaceutically effective amount of the compound of the invention administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, preferably contain from about 25-100 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 mg to 500 mg of the compound of the invention compounded with about 30 to 90 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I), or a stereoisomer or pharmaceutically acceptable salt thereof. In a further embodiment includes a pharmaceutical composition comprising a compound of Formula (I), or a stereoisomer or pharmaceutically acceptable salt thereof, together with a pharmaceutically acceptable carrier or excipient.

Another embodiment includes a pharmaceutical composition comprising a compound of Formula (I) for use in the treatment of an inflammatory diseases. Another embodiment includes a pharmaceutical composition comprising a compound of Formula (I) for use in the treatment of cancer.

The following example A and B illustrate typical compositions of the present invention, but serve merely as representative thereof.

Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention inhibit the kinase activity of PDHK1 and/or PDHK2 and/or PDHK3, and therefore enhance the activity of pyruvate dehydrogenase complex. Accordingly, the compounds of the invention are useful for reducing glycolysis and promoting oxidative phosphorylation in immune cells and cancer cells. Compounds of the invention are useful for treating IBD, COPD, IPF, psoriasis or systemic lupus erythematosus (SLE). Alternatively, compounds of the invention are useful for treating oropharyngeal squamous cell carcinoma, liver cancer, lung cancer, stomach cancer, and colon cancer in which hyper glycolysis are needed to support fast proliferation. More broadly, the compounds can be used for the treatment of autoimmune and inflammatory diseases driven by T cells, B cells and macrophages as well as all types of cancer with hyper glycolytic metabolism.

### Synthesis

The compounds of the present invention can be prepared by any conventional means. Suitable processes for synthesizing these compounds as well as their starting materials are provided in the schemes below and in the examples. Furthermore, and unless explicitly otherwise stated, all reactions, reaction conditions, abbreviations and symbols have the meanings well known to a person of ordinary skill in organic chemistry.

### Scheme 1

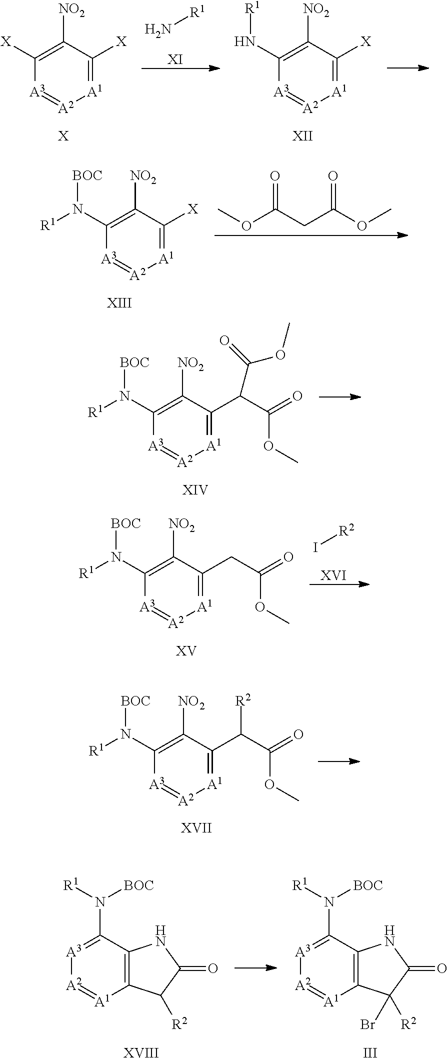

Wherein R¹ is C₁₋₆alkyl; R² is C₁₋₆alkyl; A¹ is CH; A² is CR^{b}, wherein R^{b} is halogen; A³ is CH; each X is independently F or Cl.

Compound of formula (III) can be prepared according to Scheme 1. Nucleophilic substitution of ortho-halo nitrobenzene (X) with amine R¹—NH₂ (XI) affords aniline (XII). The aniline (XII) can be protected with di-tert-butyl carbonate to give the protected aniline (XIII). The aromatic nucleophilic substitution of the protected aniline (XIII) with dimethyl malonate affords the nitro-malonate (XIV). Treatment of nitro-malonate (XIV) with LiCl resulted in decarboxylation to afford the nitro-phenyl acetate (XV). The nitro-phenyl acetate (XV) was deprotonated with a base, such as NaH and tBuOK, and then alkylated with compound of formula (XVI) to give compound of formula (XVII). Reduction of the nitro group yields the indolinone (XVIII), whose 3-position can be selectively brominated by pyridinium tribromide to afford compound of formula (III).

### Scheme 2

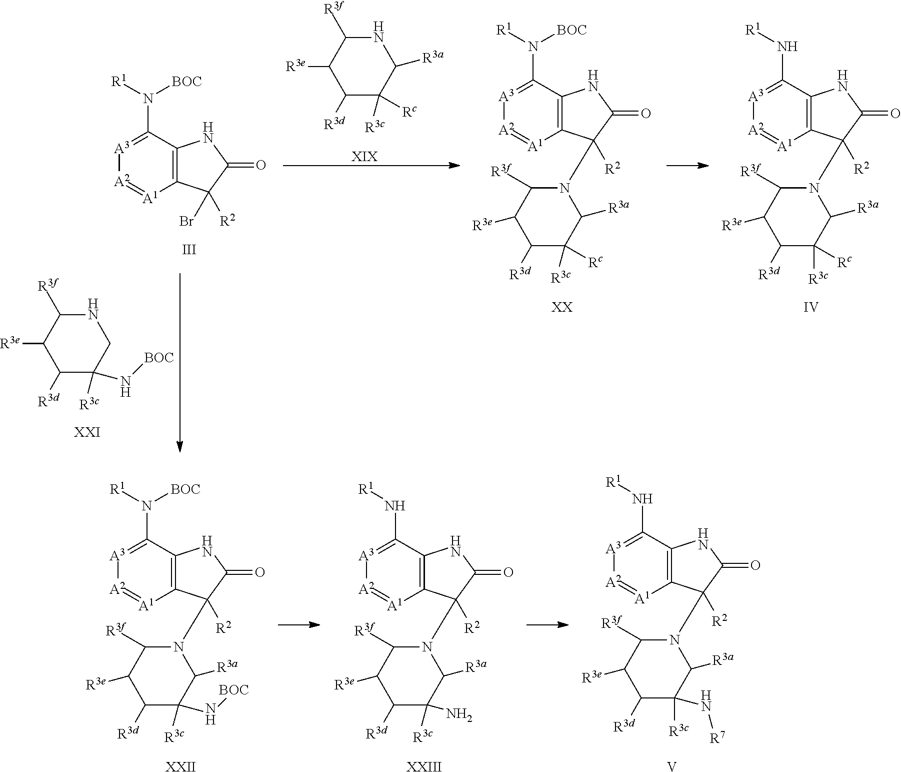

Wherein R^{c} is NR⁶R⁷, OR⁴ or —(CH₂)ₙ—R⁵; Q is halogen, such as F and Cl, or OH; n is 1, 2, 3, 4, 5 or 6.

Compound of formula (IV) and (V) can be prepared according to Scheme 2. Nucleophilic substitution of compound of formula (III) with substituted piperidine (XIX) in the presence of a base, such as DIPEA and K₂CO₃, affords compound of formula (XX). Deprotection of the Boc group to afford the final compound of formula (IV) using a suitable acid, such as trifluoroacetic acid.

Nucleophilic substitution of compound of formula (III) with substituted piperidine (XXI) in the presence of a base, such as DIPEA and K₂CO₃, affords compound of formula (XXII). Deprotection of the Boc group to afford the compound of formula (XXIII). The subsequent reaction between compound of formula (XXIV) and compound of formula (XXIII) can be achieved by aromatic substitution, acylation, sulfonation or coupling in the presence of a base, such as DIPEA and K₂CO₃, to provide the final compound of formula (V).

### Scheme 3

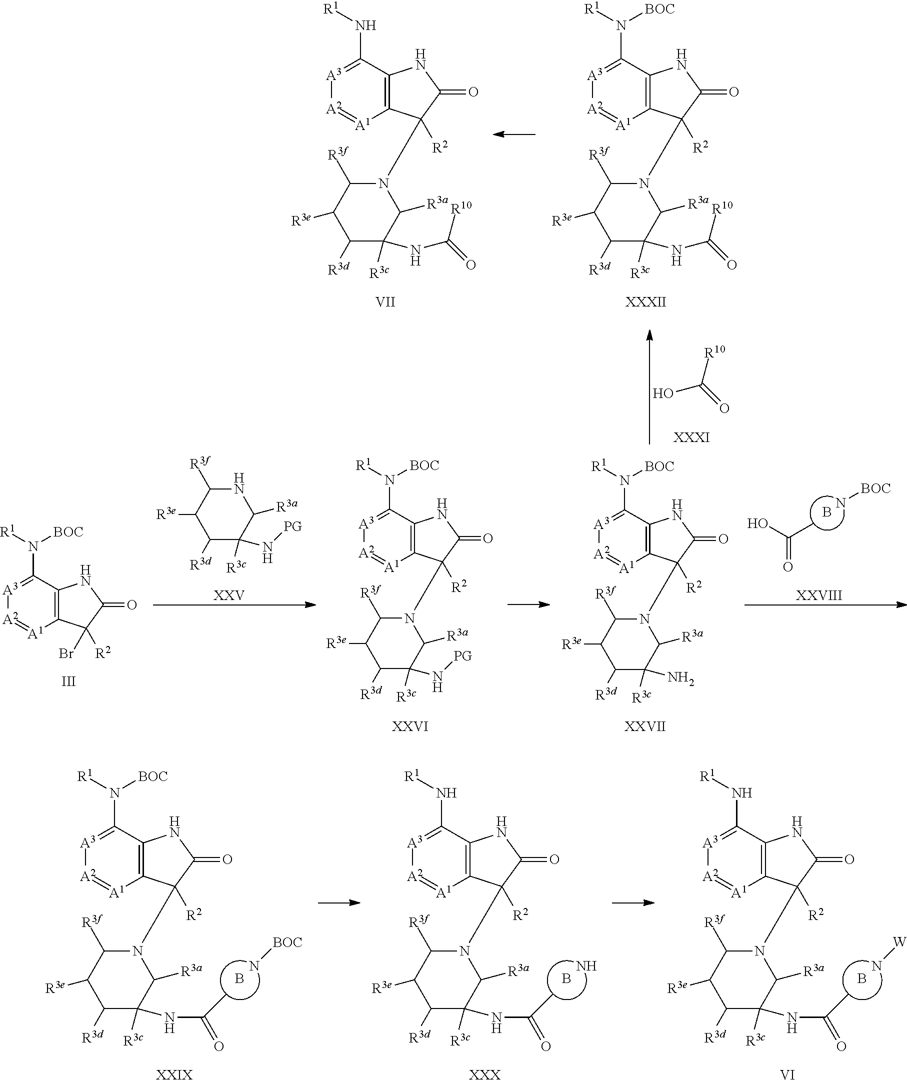

Wherein R¹, R², R^{3a}, R^{3c}, R^{3d}, R^{3e}, R^{3f}, A¹, A² and A³ are as defined above for the compound of formula (IV) and (V); R¹⁰ is 1,1-dioxothianyl, C₁₋₆alkyl, aryl, heteroaryl or heterocyclyl; PG is a nitrogen protecting group (such as Cbz, FMOC, or benzyl); ring B is unsubstituted or substituted heterocyclyl, such as azetidinyl, pyrrolidinyl, piperidinyl and azaspiro[3.3]heptanyl; W is cyano, haloC₁₋₆alkylcarbonyl, C₂₋₆alkenylcarbonyl or C₂₋₆alkenylsulfonyl.

Compound of formula (VI) and (VII) can be prepared according to Scheme 3. Nucleophilic substitution of compound of formula (III) with substituted piperidine (XXV) in the presence of a base, such as DIPEA and K₂CO₃ affords compound of formula (XXVI). Compound of formula (XXVI) undergoes selective deprotection of the nitrogen protecting group in piperidine ring to give a compound of formula (XXVII), which is condensed with acid (XXVIII) in the presence of a coupling reagent, such as HATU, to give the compound of formula (XXIX). Deprotection of the Boc groups and subsequent reaction with BrCN, chloroacetyl chloride, vinylformyl chloride, substituted vinylformyl chloride, vinylsulfonyl chloride or substituted vinylsulfonyl chloride, and the like in the presence of a base, such as DIPEA, TEA and KOAc, to afford the final compound of formula (VI).

The carboxylic acid (XXXI) can be condensed with amine (XXVII) in the presence of a coupling reagent, such as HATU, to give compound of formula (XXXII). Deprotection of the Boc group to afford the final compound of formula (VII) using a suitable acid, such as trifluoroacetic acid.

Compounds of this invention can be obtained as mixtures of diastereomers or enantiomers, which can be separated by methods well known in the art, e.g. (chiral) HPLC or SFC.

This invention also relates to a process for the preparation of a compound of formula (I) or (II) comprising any of the following steps:
a) deprotection of compound of formula (XX),

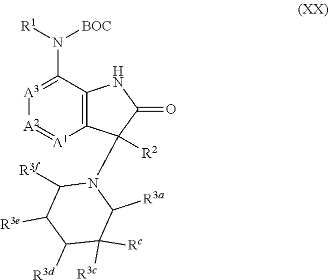

to form the compound of formula (IV),

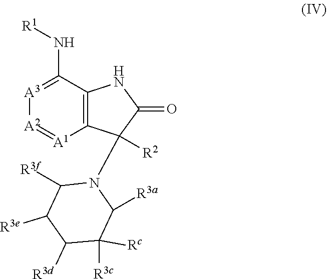

using a suitable acid, such as trifluoroacetic acid;
b) reaction between compound of formula (XXIV), R⁷-Q, with compound of formula (XXIII),

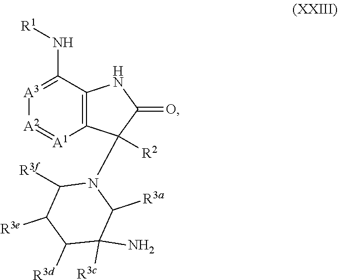

via aromatic substitution, acylation, sulfonation or coupling in the presence of a base, such as DIPEA and K₂CO₃, to provide the final compound of formula (V),

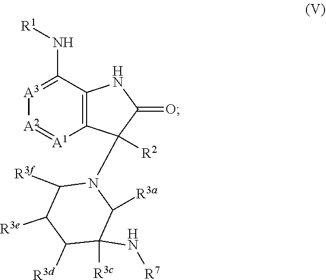

c) formation of compound of formula (VI),

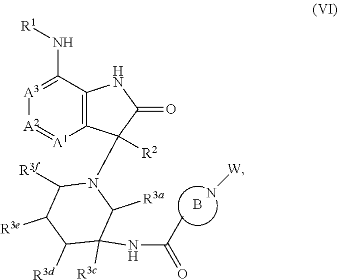

via reaction of compound of formula (XXX),

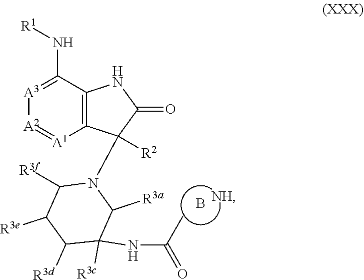

with BrCN, C₂₋₆alkenylcarbonyl chloride or C₂₋₆alkenylsulfonyl chloride in the presence of a base, such as DIPEA, TEA and KOAc;
d) deprotection of compound of formula (XXXII),

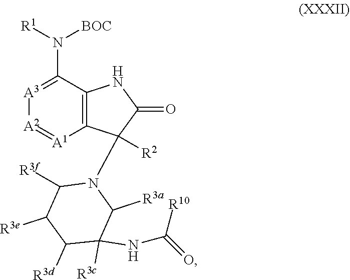

using trifluoroacetic acid, to afford compound of formula (VII),

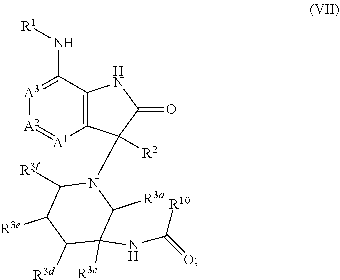

wherein R¹, R², R⁴, R⁵, R⁶, R⁷, R^{3a}, R^{3c}, R^{3d}, R^{3e}, R^{3f}, A¹, A², A³, n and X are as defined above, PG is a nitrogen protecting group (such as Cbz, FMOC, or benzyl); ring B is unsubstituted or substituted heterocyclyl containing 1-3 nitrogen atoms, such as azetidinyl, pyrrolidinyl, piperidinyl and azaspiro[3.3]heptanyl; W is cyano, haloC₁₋₆alkylcarbonyl, C₂₋₆alkenylcarbonyl or C₂₋₆alkenylsulfonyl; R^{c} is NR⁶R⁷, OR⁴ or —(CH₂)ₙ—R⁵; R¹⁰ is 1,1-dioxothianyl, C₁₋₆alkyl, aryl, heteroaryl or heterocyclyl.

A compound of formula (I) or (II) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN: acetonitrile
AISF: 4-(Acetylamino)phenyl]imidodisulfuryl difluoride
Boc₂O: di-tert-butyl dicarbonate
DABSO: 1,4-Diazabicyclo[2.2.2]octane bis(sulfur dioxide) adduct
DBU: 1,8-Diazabicyclo(5.4.0)undec-7-ene
DCE: dichloroethane
DCM: dichloromethane
DIPEA or DIEA: N,N-diisopropylethylamine
DMA: N,N-Dimethylacetamide
DMAP: 4-dimethylaminopyridine
EA or EtOAc: ethyl acetate
FA: formic acid
HATU: 1-[Bis(dimethylamino)methylene]-1H-1,2,3-triazolo[4,5-b]pyridinium 3-oxid hexafluorophosphate
IC₅₀: half inhibition concentration
LCMS liquid chromatography-mass spectrometry
MS: mass spectrometry
MTBE: methyl tert-butyl ether
NFSI: N-Fluorobenzenesulfonimide
PE: petroleum ether
PMB: p-methoxybenzyl or 4-methoxybenzyl
prep-HPLC: preparative high performance liquid chromatography
prep-TLC: preparative thin layer chromatography
rt: room temperature
RT: retention time
RPLC: reversed phase liquid chromatography
RuPhos Pd G2: chloro(2-dicyclohexylphosphino-2′,6′-diisopropoxy-1,1′-biphenyl)[2-(2′-amino-1,1′-biphenyl)]palladium(II) 2nd generation
SFC: supercritical fluid chromatography
SEMCl: 2-(Trimethylsilyl)ethoxymethyl chloride
TFA: trifluoroacetic acid
TFAA: trifluoroacetic anhydride
TLC: thin layer chromatography
TMP: 2,2,6,6-Tetramethylpiperidine
v/v volume ratio

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

Intermediates and final compounds were purified by RPLC (reversed phase liquid chromatography) on ISCO combi-flash chromatography instrument using SWPAFLASH® SW080 Bonded Spherical C18 (20-45 μm, 100 Å) column or Biotage® Sfär C18 (30 μm, 100 Å) column. Waters AutoP purification System (Sample Manager 2767, Pump 2525, Detector: Micromass ZQ and UV 2487, solvent system: acetonitrile and 0.1% ammonium hydroxide in water; acetonitrile and 0.10% FA in water or acetonitrile and 0.10% TFA in water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz or 500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate A

### tert-butyl N-(3-bromo-5-fluoro-3-methyl-2-oxo-indolin-7-yl)-N-ethyl-carbamate

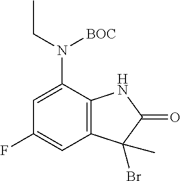 [SMILES: CCN(C(=O)OC(C)(C)C)c1cc(F)cc2c1NC(=O)C2(C)Br]

The titled compound was synthesized according to the following scheme:

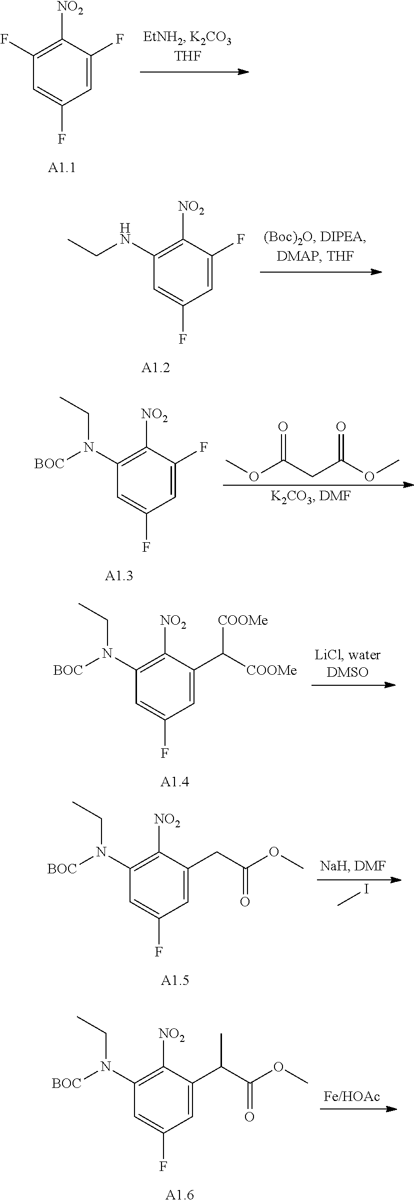

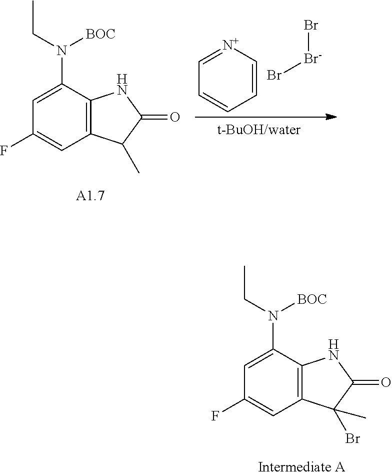

### Step (a): Preparation of N-ethyl-3,5-difluoro-2-nitro-aniline (Compound A1.2)

To a suspension of 2,4,6-trifluoronitrobenzene (compound A1.1, 200.0 g, 1129 mmol) and potassium carbonate (306.0 g, 2213 mmol) in THF (1000 mL) was added dropwise ethylamine (2 N in THF, 570.5 mL, 1141 mmol) at 0° C. within 15 min. The reaction mixture was stirred at room temperature for 3 hrs. The reaction mixture was filtered, and the filtrate was concentrated in vacuo. The residue was recrystallized in PE (500 mL) to afford compound A1.2 (200.0 g, 87.6% yield). MS: calc'd 203 [(M+H)⁺], measured 203 [(M+H)⁺].

### Step (b): Preparation of tert-butyl N-(3,5-difluoro-2-nitro-phenyl)-N-ethyl-carbamate (Compound A1.3)

To a solution of N-ethyl-3,5-difluoro-2-nitro-aniline (compound A1.2, 200.0 g, 791.5 mmol) in THF (2000 mL) was added di-t-butyldicarbonate (345.0 g, 1580 mmol), DMAP (9.6 g, 78.6 mmol) and DIEA (307.0 mL, 2375 mmol), the resultant reaction mixture was stirred at 65° C. for 3 hrs. After being cooled to room temperature, the reaction mixture was concentrated in vacuo to afford the crude product, which was purified by column chromatography (silica gel, 0% to 15% EA in PE) to afford compound A1.3 (215 g, 89.8% yield). MS: calc'd 203 [(M-Boc+H)⁺], measured 203 [(M-Boc+H)⁺].

### Step (c): Preparation of dimethyl 2-[3-[tert-butoxycarbonyl(ethyl)amino]-5-fluoro-2-nitro-phenyl]propanedioate (Compound A1.4)

To a solution of tert-butyl N-(3,5-difluoro-2-nitro-phenyl)-N-ethyl-carbamate (compound A1.3, 200.0 g, 463.16 mmol) and dimethyl malonate (78.7 mL, 688.8 mmol) in DMF (2000 mL) was added potassium carbonate (252.0 g, 1823 mmol), the resultant mixture was stirred at 60° C. overnight. After being cooled to room temperature, the reaction mixture was diluted with water (5000 mL), acidified by 1N HCl to PH about 6, and extracted with EA (2000 mL) twice. The combined organic layer was washed with brine, dried over Na₂SO₄, filtered and concentrated. The residue was purified by column chromatography (silica gel, 0% to 20% EA in PE) to afford compound A1.4 (85 g, 44.3% yield). MS: calc'd 315 [(M-Boc+H)⁺], measured 315 [(M-Boc+H)⁺].

### Step (d): Preparation of methyl 2-[3-[tert-butoxycarbonyl(ethyl)amino]-5-fluoro-2-nitro-phenyl]acetate (Compound A1.5)

To a solution of dimethyl 2-[3-[tert-butoxycarbonyl(ethyl)amino]-5-fluoro-2-nitro-phenyl]propanedioate (compound A1.4, 85.0 g, 205.1 mmol) in DMSO (500 mL) was added a solution of lithium chloride (4.8 g, 113.5 mmol) in water (8.0 mL, 445.1 mmol), the resultant mixture was stirred at 100° C. overnight. After being cooled to room temperature, the reaction mixture was poured into water (5000 mL), and extracted with EA (1000 mL) for three times. The combined organic layer was washed with brine, dried over Na₂SO₄, filtered and concentrated in vacuo. The residue was purified by column chromatography (silica gel, 10% to 50% EA in PE) to afford compound A1.5 (36 g, 49.3% yield). MS: calc'd 301 [(M−55)⁺], measured 301[(M−55)⁺].

### Step (e): Preparation of methyl 2-[3-[tert-butoxycarbonyl(ethyl)amino]-5-fluoro-2-nitro-phenyl]propanoate (Compound A1.6)

NaH (60% dispersion in mineral oil, 377.2 mg, 9.43 mmol) was added portion wise to a solution of 2-[3-[tert-butoxycarbonyl(ethyl)amino]-5-fluoro-2-nitro-phenyl]acetic acid methyl ester (compound A1.5, 5.0 g, 14.0 mmol) in DMF (30 mL) at 0° C., the reaction mixture was stirred at this temperature for 30 min, and then iodomethane (2.4 g, 1.1 mL, 16.8 mmol) was added at 0° C. The resultant mixture was warmed to room temperature slowly and stirred for 2 hrs. The reaction was quenched with saturated aqueous NH₄Cl solution and extracted with EA for three times. The combined organic layer was washed with water and brine, dried over Na₂SO₄, filtered and concentrated in vacuo. The residue was purified by flash chromatography (silica gel, 80 g, 0% to 20% EA in PE) to afford compound A1.6 (4.2 g, 80.8%). MS: calc'd 393 [(M+Na)⁺], measured 393 [(M+Na)⁺].

### Step (f): Preparation of tert-butyl N-ethyl-N-(5-fluoro-3-methyl-2-oxo-indolin-7-yl)carbamate (Compound A1.7)

To a stirred solution of methyl 2-[3-[tert-butoxycarbonyl(ethyl)amino]-5-fluoro-2-nitro-phenyl]propanoate (compound A1.6, 3.8 g, 10.3 mmol) in acetic acid (60 mL) was added iron (5.73 g, 103 mmol) at room temperature. The resultant mixture was stirred at 100° C. for 2 hrs. The mixture was filtered, while still warm, through a layer of celite and more acetic acid was used to wash off the residual product. The filtrate was concentrated under high vacuum. The residue was purified by flash chromatography (silica gel, 120 g, 0% to 80% EA in PE) to afford compound A1.7 (2.6 g, 82.5% yield). MS: calc'd 307 [(M−H)⁻], measured 307 [(M−H)⁻].

### Step (g): Preparation of tert-butyl N-(3-bromo-5-fluoro-3-methyl-2-oxo-indolin-7-yl)-N-ethyl-carbamate (Intermediate A)

To a solution of tert-butyl N-ethyl-N-(5-fluoro-3-methyl-2-oxo-indolin-7-yl)carbamate (compound A1.7, 3.6 g, 11.7 mmol) in t-BuOH/water (v:v=20:1, 63 mL) was added pyridinium tribromide (5.58 g, 17.55 mmol). The resultant mixture was stirred at room temperature overnight. The reaction mixture was diluted with water (300 mL), extracted with EA (100 mL) twice. The combined organic layer was washed with brine, dried over Na₂SO₄, filtered and concentrated. The residue was purified by flash chromatography (silica gel, 80 g, 0% to 70% EA in PE) to afford Intermediate A (3.9 g, 86.3%). MS: calc'd 387, 389 [(M−55)⁺], measured 331, 333[(M−55)⁺].

### Intermediate B

### tert-butyl N-(3-bromo-3-ethyl-5-fluoro-2-oxo-indolin-7-yl)-N-ethyl-carbamate

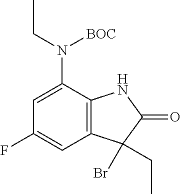 [SMILES: CCN(C(=O)OC(C)(C)C)c1cc(F)cc2c1NC(=O)C2(Br)CC]

tert-butyl N-(3-bromo-3-ethyl-5-fluoro-2-oxo-indolin-7-yl)-N-ethyl-carbamate (Intermediate B) was prepared in analogy to tert-butyl N-(3-bromo-5-fluoro-3-methyl-2-oxo-indolin-7-yl)-N-ethyl-carbamate (Intermediate A), by replacing iodomethane with iodoethane in step (e). MS: calc'd 346 [(M−55+H)⁺], measured 346 [(M-55+H)⁺].

### Intermediate C

### tert-butyl N-[3-[(3R)-3-amino-1-piperidyl]-5-fluoro-3-methyl-2-oxo-indolin-7-yl]-N-ethyl-carbamate

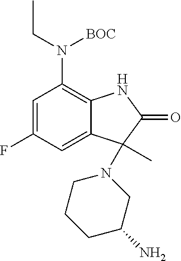 [SMILES: CCN(C(=O)OC(C)(C)C)c1cc(F)cc2c1NC(=O)C2(C)N1CCC[C@@H](N)C1]

The titled compound was synthesized according to the following scheme:

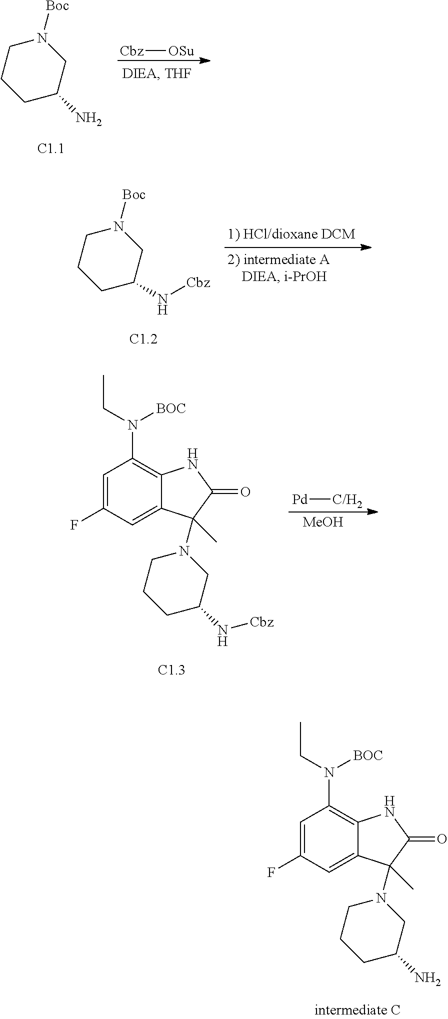

### Step (a): Preparation of tert-butyl (3R)-3-(benzyloxycarbonylamino)piperidine-1-carboxylate (Compound C1.2)

To a solution of tert-butyl (3R)-3-aminopiperidine-1-carboxylate (compound C1.1, 10.0 g, 49.9 mmol) in THF (200 mL) was added Cbz-OSu (14.9 g, 59.9 mmol) and DIEA (9661.5 mg, 74.9 mmol), the resultant mixture was stirred at 25° C. for 2 hrs. The reaction was quenched with 1N HCl (aq. 50 mL), and extracted with EA (50 mL) for three times. The combined organic layer was washed with brine, dried over Na₂SO₄, filtered and concentrated. The residue was purified by column chromatography (silica gel, 20% to 50% EA in PE) to afford compound C1.2 (13.0 g, 78.0% yield). MS: calc'd 335 [(M+H)⁺]; measured 357, [(M+Na)⁺].

### Step (b): Preparation of tert-butyl N-ethyl-N-[5-fluoro-3-methyl-2-oxo-3-[(3R)-3-(benzyloxycarbonylamino)-1-piperidyl]indolin-7-yl]carbamate (Compound C1.3)

To a solution of tert-butyl (3R)-3-(benzyloxycarbonylamino)piperidine-1-carboxylate (compound C1.2, 3.0 g, 8.98 mmol) in DCM (50 mL) was added HCl/dioxane (4N, 10 mL, 40 mmol). The resultant suspension was stirred at room temperature for 20 hrs., then concentrated to give a solid. To a solution of the residue and DIEA (5.79 g, 44.9 mmol) in isopropanol (60 mL) was added tert-butyl N-(3-bromo-5-fluoro-3-methyl-2-oxo-indolin-7-yl)-N-ethyl-carbamate (intermediate A, 3.47 g, 8.98 mmol). After being stirred at room temperature for 3 hrs., the reaction mixture was diluted with water (300 mL), and extracted with EA (80 mL) for three times. The combined organic layer was washed with brine, dried over Na₂SO₄, filtered and concentrated in vacuo. The residue was purified by flash chromatography (silica gel, 80 g, 20% to 100% EA in PE) to afford compound C1.3 (3.3 g, 68.1% yield). MS: calc'd 541 [(M+H)⁺]; measured 541 [(M+H)⁺].

### Step (c): Preparation of tert-butyl N-[3-[(3R)-3-amino-1-piperidyl]-5-fluoro-3-methyl-2-oxo-indolin-7-yl]-N-ethyl-carbamate (Intermediate C)

A mixture of tert-butyl N-ethyl-N-[5-fluoro-3-methyl-2-oxo-3-[(3R)-3-(benzyloxycarbonylamino)-1-piperidyl]indolin-7-yl]carbamate (compound C, 3.3 g, 6.1 mmol) and Pd—C (200 mg) in MeOH (150 mL) was hydrogenated by a hydrogen balloon at room temperature for 5 hrs. After the catalyst was filtered off, the filtrate was concentrated in vacuo. The residue was purified by flash chromatography (silica gel, 80 g, 0% to 10% MeOH in DCM) to afford intermediate C (2.0 g, 80.8% yield). MS: calc'd 407 [(M+H)⁺]; measured 407 [(M+H)⁺].

### Intermediate D

### tert-butyl N-[3-[(3R)-3-amino-1-piperidyl]-3-ethyl-5-fluoro-2-oxo-indolin-7-yl]-N-ethyl-carbamate

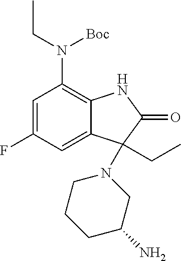 [SMILES: CCN(C(=O)OC(C)(C)C)c1cc(F)cc2c1NC(=O)C2(CC)N1CCC[C@@H](N)C1]

tert-butyl N-[3-[(3R)-3-amino-1-piperidyl]-3-ethyl-5-fluoro-2-oxo-indolin-7-yl]-N-ethyl-carbamate (intermediate D) was prepared in analogy to tert-butyl N-[3-[(3R)-3-amino-1-piperidyl]-5-fluoro-3-methyl-2-oxo-indolin-7-yl]-N-ethyl-carbamate (intermediate C), by replacing tert-butyl N-(3-bromo-5-fluoro-3-methyl-2-oxo-indolin-7-yl)-N-ethyl-carbamate (intermediate A) with tert-butyl N-(3-bromo-3-ethyl-5-fluoro-2-oxo-indolin-7-yl)-N-ethyl-carbamate (intermediate B) in step (b). MS: calc'd 421 [(M+H)⁺]; measured 421 [(M+H)⁺].

### Example 1A and 1B

### 2-chloro-4-[[(3R)-1-[(3R)-7-(ethylamino)-5-fluoro-3-methyl-2-oxo-indolin-3-yl]-3-piperidyl]oxy]benzoic acid and 2-chloro-4-[[(3R)-1-[(3S)-7-(ethylamino)-5-fluoro-3-methyl-2-oxo-indolin-3-yl]-3-piperidyl]oxy]benzoic acid

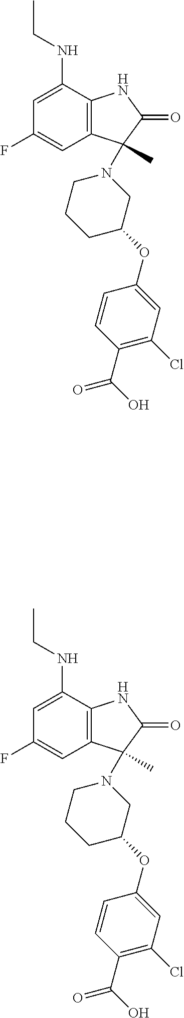 [SMILES: CCNc1cc(F)cc2c1NC(=O)[C@]2(C)N1CCC[C@@H](Oc2ccc(C(=O)O)c(Cl)c2)C1 ; CCNc1cc(F)cc2c1NC(=O)[C@@]2(C)N1CCC[C@@H](Oc2ccc(C(=O)O)c(Cl)c2)C1]

The titled compound was synthesized according to the following scheme:

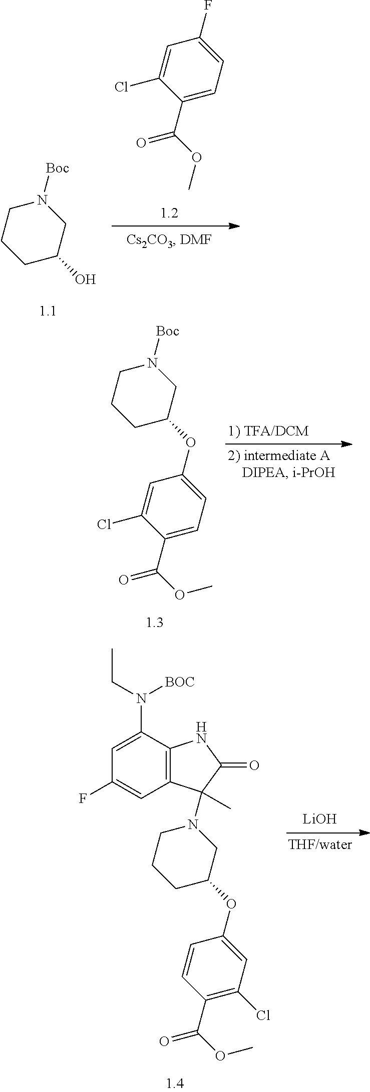 [SMILES: CC(C)(C)OC(=O)N1CCC[C@@H](O)C1 ; COC(=O)c1ccc(F)cc1Cl ; COC(=O)c1ccc(O[C@@H]2CCCN(C(=O)OC(C)(C)C)C2)cc1Cl ; CCN(C(=O)OC(C)(C)C)c1cc(F)cc2c1NC(=O)C2(C)N1CCC[C@@H](Oc2ccc(C(=O)OC)c(Cl)c2)C1 ; "LiOH"]

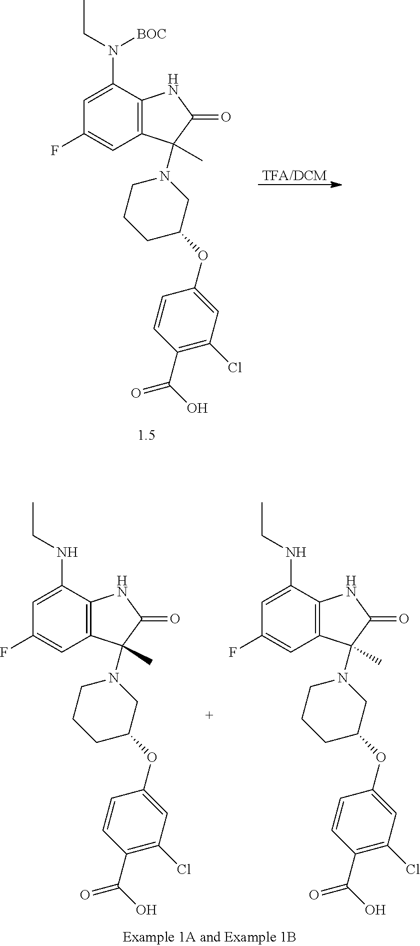 [SMILES: CCN(C(=O)OC(C)(C)C)c1cc(F)cc2c1NC(=O)C2(C)N1CCC[C@@H](Oc2ccc(C(=O)O)c(Cl)c2)C1 ; "TFA/DCM" ; CCNc1cc(F)cc2c1NC(=O)[C@]2(C)N1CCC[C@@H](Oc2ccc(C(=O)O)c(Cl)c2)C1 ; CCNc1cc(F)cc2c1NC(=O)[C@@]2(C)N1CCC[C@@H](Oc2ccc(C(=O)O)c(Cl)c2)C1]

### Step (a): Preparation of tert-butyl (3R)-3-(3-chloro-4-methoxycarbonyl-phenoxy)piperidine-1-carboxylate (compound 1.3)

To a solution of tert-butyl (3R)-3-hydroxypiperidine-1-carboxylate (compound 1.1, 450 mg, 2.24 mmol) in DMF (5 mL) was added Cs₂CO₃ (1457 mg, 4.47 mmol) and methyl 2-chloro-4-fluorobenzoate (compound 1.2, 506 mg, 2.68 mmol). The resultant mixture was heated to 80° C. and stirred for 16 hrs. After being cooled to room temperature, the reaction mixture was filtered and the filtrate was concentrated in vacuo. The residue was purified by RPLC to afford compound 1.3 (220 mg, 25.3% yield). MS: calc'd 370 [(M+H)⁺], measured 392 [(M+Na)⁺].

### Step (b): Preparation of methyl 4-[[(3R)-1-[7-[tert-butoxycarbonyl(ethyl)amino]-5-fluoro-3-methyl-2-oxo-indolin-3-yl]-3-piperidyl]oxy]-2-chloro-benzoate (Compound 1.4)

To a solution of tert-butyl (3R)-3-(3-chloro-4-methoxycarbonyl-phenoxy) piperidine-1-carboxylate (compound 1.3, 220 mg, 0.59 mmol) in DCM (5 mL) was added TFA (1.5 mL). The reaction mixture was stirred at room temperature for 2 hrs, then concentrated to afford an oil.

To a solution of the residue and DIEA (0.29 mL, 1.67 mmol) in isopropanol (5 mL) was added tert-butyl N-(3-bromo-5-fluoro-3-methyl-2-oxo-indolin-7-yl)-N-ethyl-carbamate (intermediate A, 259 mg, 0.67 mmol). After being stirred at room temperature for 2 hrs, the reaction mixture was diluted with water (50 mL), and extracted with EA (30 mL) twice. The combined organic layer was washed with brine, dried over Na₂SO₄, filtered and concentrated. The residue was purified by pre-HPLC to afford compound 1.4 (120 mg, 35.3% yield). MS: calc'd 576, 578 [(M+H)⁺], measured 576, 578 [(M+H)⁺].

### Step (c): Preparation of 4-[[(3R)-1-[7-[tert-butoxycarbonyl(ethyl)amino]-5-fluoro-3-methyl-2-oxo-indolin-3-yl]-3-piperidyl]oxy]-2-chloro-benzoic acid (Compound 1.5)

To a solution of methyl 4-[[(3R)-1-[7-[tert-butoxycarbonyl(ethyl)amino]-5-fluoro-3-methyl-2-oxo-indolin-3-yl]-3-piperidyl]oxy]-2-chloro-benzoate (compound 1.4, 100 mg, 0.17 mmol) in THF (5 mL) was added LiOH (21 mg, 0.88 mmol) in water (0.5 mL). The resultant mixture was heated to 70° C. and stirred for 3 hrs. After being cooled to room temperature, the mixture was diluted with water (20 mL), acidified with 1N HCl to pH about 5, and then extracted with EA (60 mL) twice. The combined organic layer was washed with brine, dried over Na₂SO₄, filtered and the filtrate was concentrated in vacuo to afford compound 1.5 (80 mg, 83.7% yield). MS: calc'd 562,564 [(M+H)⁺], measured 562, 564 [(M+H)⁺].

### Step (e): Preparation of 2-chloro-4-[[(3R)-1-[(3R)-7-(ethylamino)-5-fluoro-3-methyl-2-oxo-indolin-3-yl]-3-piperidyl]oxy]benzoic acid and 2-chloro-4-[[(3R)-1-[(3S)-7-(ethylamino)-5-fluoro-3-methyl-2-oxo-indolin-3-yl]-3-piperidyl]oxy]benzoic acid

To a solution of 4-[[(3R)-1-[7-[tert-butoxycarbonyl(ethyl)amino]-5-fluoro-3-methyl-2-oxo-indolin-3-yl]-3-piperidyl]oxy]-2-chloro-benzoic acid (compound 1.5, 80 mg, 0.14 mmol) in DCM (5 mL) was added TFA (2 mL). The reaction mixture was stirred at room temperature for 2 hrs, then concentrated to afford a crude product, which was purified by prep-HPLC to afford Example 1A (13.3 mg, 20.6% yield) and Example 1B (17.2 mg, 26.6% yield): 2-chloro-4-[[(3R)-1-[(3R)-7-(ethylamino)-5-fluoro-3-methyl-2-oxo-indolin-3-yl]-3-piperidyl]oxy]benzoic acid and 2-chloro-4-[[(3R)-1-[(3S)-7-(ethylamino)-5-fluoro-3-methyl-2-oxo-indolin-3-yl]-3-piperidyl]oxy]benzoic acid.

Example 1A MS: calc'd 462, 464 [(M+H)⁺], measured 462, 464 [(M+H)⁺]. ¹H NMR (400 MHz, DMSO-d₆) δ=10.05 (s, 1H), 7.81 (d, J=8.8 Hz, 1H), 7.06 (d, J=2.4 Hz, 1H), 6.98 (dd, J=8.8, 2.4 Hz, 1H), 6.37-6.24 (m, 2H), 4.52 (brs, 1H), 3.09-3.04 (m, 3H), 2.90-2.75 (m, 1H), 2.65-2.54 (m, 2H), 2.47-2.36 (m, 1H), 2.00-1.91 (m, 1H), 1.79-1.68 (m, 1H), 1.62-1.50 (m, 1H), 1.49-1.35 (m, 4H), 1.18 (t, J=7.2 Hz, 3H).

Example 1B MS: calc'd 462, 464 [(M+H)⁺], measured 462, 464 [(M+H)⁺]. ¹H NMR (400 MHz, DMSO-d₆) δ=10.08 (s, 1H), 7.79 (d, J=8.8 Hz, 1H), 7.03 (s, 1H), 6.95 (d, J=9.2 Hz, 1H), 6.49-6.45 (m, 1H), 6.36 (d, J=11.6 Hz, 1H), 4.51 (brs, 1H), 3.09-3.04 (m, 3H), 2.97-2.91 (m, 1H), 2.54-2.51 (m, 2H), 2.48-2.47 (m, 1H), 2.01-1.94 (m, 1H), 1.83-1.74 (m, 1H), 1.63-1.54 (m, 1H), 1.48-1.41 (m, 4H), 1.18 (t, J=7.2 Hz, 3H).

### Example 2

### 5-[[(3R)-1-[7-(ethylamino)-5-fluoro-3-methyl-2-oxo-indolin-3-yl]-3-piperidyl]oxy]-2-hydroxy-benzaldehyde

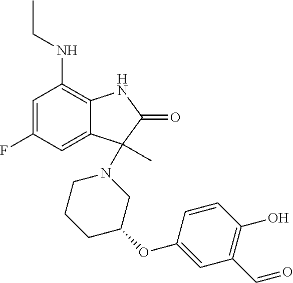 [SMILES: CCNc1cc(F)cc2c1NC(=O)C2(C)N1CCC[C@@H](Oc2ccc(O)c(C=O)c2)C1]

The titled compound was synthesized according to the following scheme:

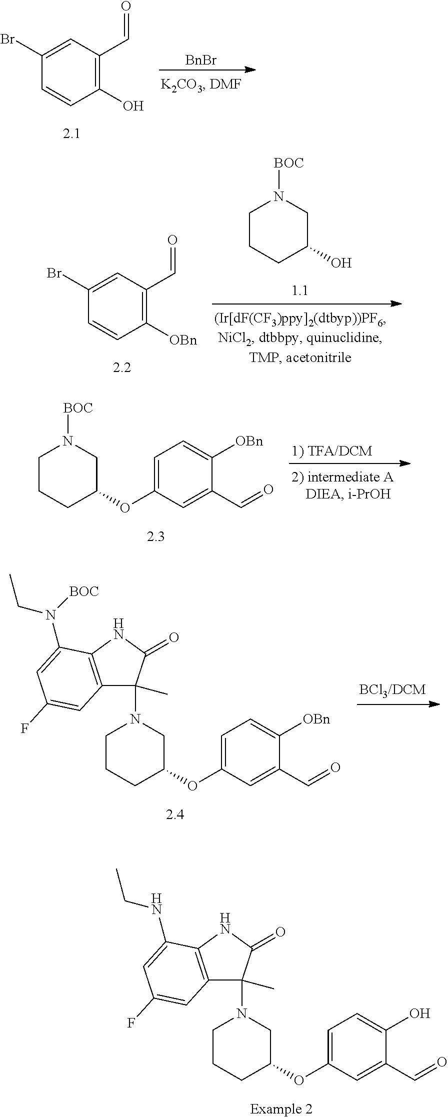

### Step (a): Preparation of 2-benzyloxy-5-bromo-benzaldehyde (Compound 2.2)

To a solution of 5-bromo-2-hydroxy-benzaldehyde (compound 2.1, 2.0 g, 9.95 mmol) in DMF (20 mL) was added K₂CO₃ (2750.1 mg, 19.9 mmol) and benzyl bromide (1.3 mL, 10.94 mmol). The resultant mixture was stirred at 75° C. for 4 hrs. After being cooled to room temperature, the reaction mixture was poured into water (200 mL), and extracted with EA (50 mL) for three times. The combined organic layer was washed with brine, dried over Na₂SO₄, filtered and concentrated in vacuo. The residue was triturated with MTBE (10 mL) twice, collected by filtration to afford compound 2.2 (2.0 g, 69.1% yield). ¹H NMR (400 MHz, CDCl₃) δ 10.47 (s, 1H), 7.96 (d, J=2.8 Hz, 1H), 7.61 (dd, J=2.8, 8.8 Hz, 1H), 7.45-7.35 (m, 5H), 6.96 (d, J=8.8 Hz, 1H), 5.19 (s, 2H).

### Step (b): Preparation of tert-butyl (3R)-3-(4-benzyloxy-3-formyl-phenoxy)piperidine-1-carboxylate (Compound 2.3)

To a solution of 2-benzyloxy-5-bromo-benzaldehyde (compound 2.2, 450.0 mg, 1.55 mmol) in acetonitrile (10 mL) was added tert-butyl (3R)-3-hydroxypiperidine-1-carboxylate (compound 1.1, 622.2 mg, 3.09 mmol), (Ir[dF(CF₃)ppy]₂(dtbpy))PF₆ (CAS: 870987-63-6, TCI, Catalog: D5817, 17.4 mg, 0.02 mmol), NiCl₂·dtbbpy (30.8 mg, 0.08 mmol), quinuclidine (17.2 mg, 0.15 mmol) and TMP (436.7 mg, 3.09 mmol), the resultant mixture was purged with N₂ for three times, and then stirred at room temperature under the exposure of blue LEDs for 14 hrs. The reaction was filtered through Celite and washed with DCM, and the filtrate was concentrated in vacuo. The residue was purified by RPLC to afford compound 2.3 (150 mg, 21.3% yield). MS: calc'd 412 [(M+H)⁺]; measured 312 [(M-Boc+H)⁺].

### Step (c): Preparation of tert-butyl N-ethyl-N-[5-fluoro-3-methyl-2-oxo-3-[(3R)-3-(4-benzyloxy-3-formyl-phenoxy)-1-piperidyl]indolin-7-yl]carbamate (Compound 2.4)

To a solution of tert-butyl (3R)-3-(4-benzyloxy-3-formyl-phenoxy)piperidine-1-carboxylate (compound 2.3, 115.0 mg, 0.28 mmol) in DCM (5 mL) was added TFA (2 mL). The reaction mixture was stirred at room temperature for 2 hrs, then concentrated to afford an oil. To a solution of the residue and DIEA (0.14 mL, 0.81 mmol) in isopropanol (5 mL) was added tert-butyl N-(3-bromo-5-fluoro-3-methyl-2-oxo-indolin-7-yl)-N-ethyl-carbamate (intermediate A, 104.68 mg, 0.27 mmol). The resultant mixture was stirred at room temperature for 1 hr. The reaction mixture was concentrated under vacuum to give a residue which was purified by RPLC to afford compound 2.4 (65 mg, 37.5% yield). MS: calc'd 618 [(M+H)⁺]; measured 618 [(M+H)⁺].

### Step (e): Preparation of 5-[[(3R)-1-[7-(ethylamino)-5-fluoro-3-methyl-2-oxo-indolin-3-yl]-3-piperidyl]oxy]-2-hydroxy-benzaldehyde (Example 2)

To a solution of tert-butyl N-ethyl-N-[5-fluoro-3-methyl-2-oxo-3-[(3R)-3-(4-benzyloxy-3-formyl-phenoxy)-1-piperidyl]indolin-7-yl]carbamate (compound 2.4, 50.0 mg, 0.08 mmol) in DCM (2 mL) was added dropwise BCl₃ (1 N in DCM, 0.5 mL, 0.50 mmol) at 0° C. The resultant mixture was stirred at room temperature for 1 hr. The reaction was quenched with sat.NaHCO₃ aqueous, and the reaction mixture was extracted with DCM (15 mL) for three times. The combined organic layer was washed with brine, dried over Na₂SO₄, filtered and concentrated in vacuo. The residue was purified by prep-HPLC to afford Example 2 (3.6 mg, 10.1% yield). MS: calc'd 428 [(M+H)⁺]; measured 428 [(M+H)⁺]. MS: calc'd 428 [(M+H)⁺]; measured 428 [(M+H)⁺]. ¹H NMR (400 MHz, METHANOL-d₄) δ=9.97 (s, 0.4H), 9.91 (s, 0.6H), 7.21-7.20 (m, 0.4H), 7.16-7.13 (m, 0.6H), 7.11-7.08 (m, 1H), 6.87-6.82 (m, 1H), 6.44-6.42 (m, 0.6H), 6.33-6.29 (m, 1.4H), 4.29-4.18 (m, 1H), 3.15-3.09 (m, 2H), 3.04-2.99 (m, 1H), 2.91-2.81 (m, 1.4H), 2.59-2.53 (m, 0.6H), 2.49-2.44 (m, 1H), 2.29-2.24 (m, 1H), 2.04-2.00 (m, 1H), 1.84-1.79 (m, 1H), 1.61-1.55 (m, 1H), 1.49-1.43 (m, 3H), 1.31-1.24 (m, 3H).

### Example 3A and 3B

### 4-[[(3R)-1-[(3R)-7-(ethylamino)-5-fluoro-3-methyl-2-oxo-indolin-3-yl]-3-piperidyl]oxy]benzenesulfonyl fluoride and 4-[[(3R)-1-[(3S)-7-(ethylamino)-5-fluoro-3-methyl-2-oxo-indolin-3-yl]-3-piperidyl]oxy]benzenesulfonyl fluoride

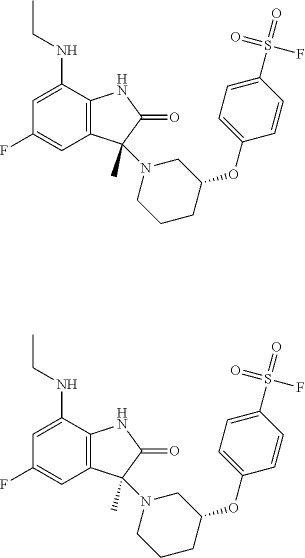 [SMILES: CCNc1cc(F)cc2c1NC(=O)[C@]2(C)N1CCC[C@@H](Oc2ccc(S(=O)(=O)F)cc2)C1 ; CCNc1cc(F)cc2c1NC(=O)[C@@]2(C)N1CCC[C@@H](Oc2ccc(S(=O)(=O)F)cc2)C1]

The titled compound was synthesized according to the following scheme:

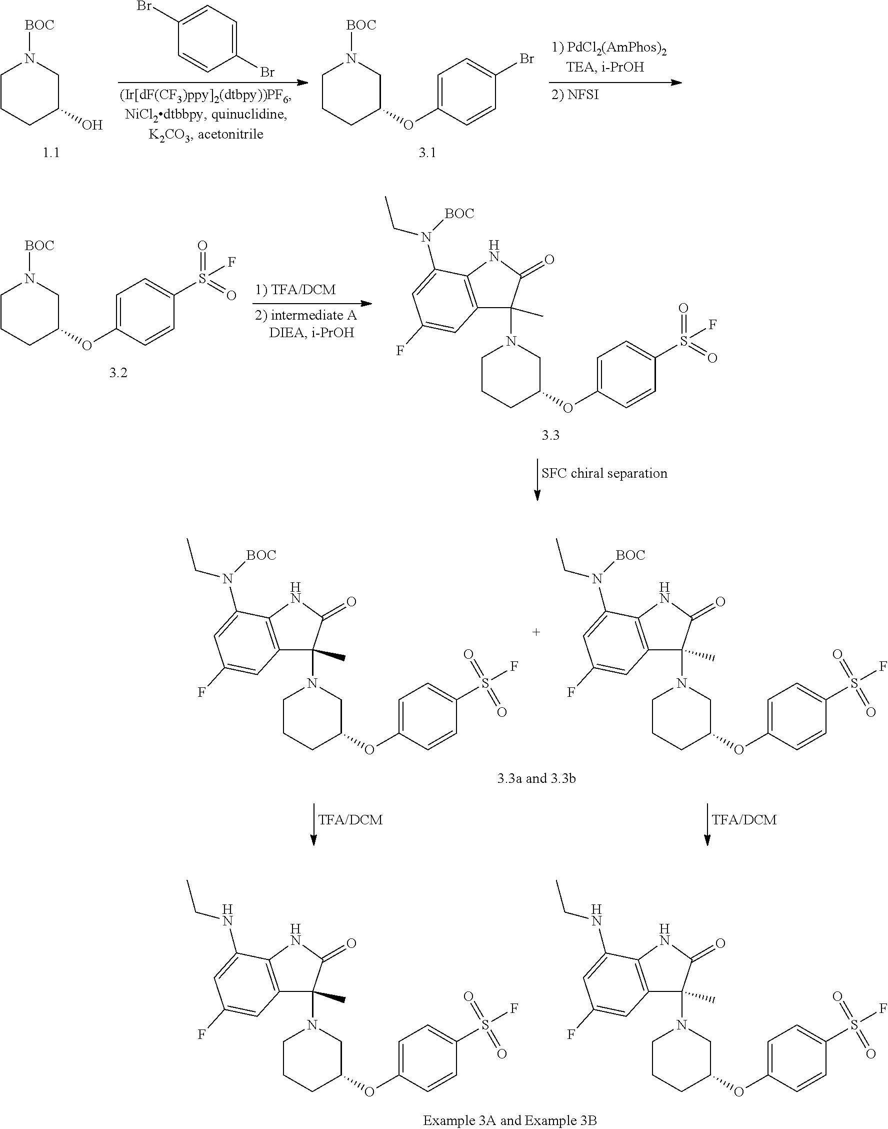

Step (a): Preparation of tert-butyl (3R)-3-(4-bromophenoxy)piperidine-1-carboxylate (Compound 3.1)

To a solution of 1,4-dibromobenzene (1.0 g, 4.24 mmol) in acetonitrile (20 mL) was added tert-butyl (3R)-3-hydroxypiperidine-1-carboxylate (compound 1.1, 1.71 g, 8.48 mmol), (Ir[dF(CF₃)ppy]₂(dtbpy))PF₆ (44.8 mg, 0.04 mmol), NiCl₂·dtbbpy (79.6 mg, 0.210 mmol), quinuclidine (47.13 mg, 0.42 mmol) and potassium carbonate (1172 mg, 8.48 mmol), the resultant mixture was purged with N₂ for three times, and then stirred at room temperature under the exposure of blue LEDs for 12 hrs. The reaction mixture was filtered through Celite and washed with DCM, and the filtrate was concentrated in vacuo. The residue was purified by flash chromatography (silica gel, 40 g, 5% to 30% EA in PE) to afford compound 3.1 (600 mg, 39.8% yield). MS: calc'd 356 [(M+H)⁺]; measured 300 [(M−55)⁺].

### Step (b): Preparation of tert-butyl N-ethyl-N-[5-fluoro-3-methyl-2-oxo-3-[(3R)-3-(4-fluorosulfonylphenoxy)-1-piperidyl]indolin-7-yl]carbamate (Compound 3.2)

To a solution of tert-butyl (3R)-3-(4-bromophenoxy)piperidine-1-carboxylate (compound 3.1, 500.0 mg, 1.40 mmol), DABSO (400.0 mg, 1.18 mmol) and triethylamine (0.60 mL, 4.30 mmol) in isopropanol (10 mL) was added PdCl₂(Amphos)₂ (50.0 mg, 0.070 mmol) under N₂, then the reaction mixture was stirred at 80° C. for 12 hrs under N₂. After being cooled to room temperature, NFSI (800.0 mg, 2.54 mmol) was added, and the resultant mixture was stirred at 20° C. for another 3 hrs. The reaction mixture was filtered through Celite and washed with DCM, and the filtrate was concentrated in vacuo. The residue was purified by flash chromatography (silica gel, 20 g, 10% to 20% EA in PE) to afford compound 3.2 (170 mg, 33.8% yield). MS: calc'd 360 [(M+H)⁺]; measured 304 [(M−55)⁺].

### Step (c): Preparation of tert-butyl N-ethyl-N-[5-fluoro-3-methyl-2-oxo-3-[(3R)-3-(4-fluorosulfonylphenoxy)-1-piperidyl]indolin-7-yl]carbamate (Compound 3.3)

To a solution of tert-butyl N-ethyl-N-[5-fluoro-3-methyl-2-oxo-3-[(3R)-3-(4-fluorosulfonylphenoxy)-1-piperidyl]indolin-7-yl]carbamate (compound 3.2, 150.0 mg, 0.42 mmol) in DCM (5 mL) was added TFA (2 mL). The reaction mixture was stirred at room temperature for 2 hrs, then concentrated to afford an oil. To a solution of the residue and DIEA (0.14 mL, 0.81 mmol) in isopropanol (5 mL) was added tert-butyl N-(3-bromo-5-fluoro-3-methyl-2-oxo-indolin-7-yl)-N-ethyl-carbamate (intermediate A, 105.0 mg, 0.27 mmol). The reaction mixture was stirred at room temperature for 2 hrs, then concentrated to afford a crude product, the residue was purified by pre-HPLC to afford compound 3.3 (120 mg, 78.6% yield). MS: calc'd 566 [(M+H)⁺]; measured 566 [(M+H)⁺].

### Step (d): Preparation of tert-butyl N-ethyl-N-[(3R)-5-fluoro-3-[(3R)-3-(4-fluorosulfonylphenoxy)-1-piperidyl]-3-methyl-2-oxo-indolin-7-yl]carbamate and tert-butyl N-ethyl-N-[(3S)-5-fluoro-3-[(3R)-3-(4-fluorosulfonylphenoxy)-1-piperidyl]-3-methyl-2-oxo-indolin-7-yl]carbamate (Compound 3.3a and 3.3b)

Compound 3.3 (120 mg) was resolved by SFC to give two single isomers: compound 3.3a (faster eluting, 50 mg, 41.6% yield). MS: calc'd 566 (M+H)⁺, measured 566 (M+H)⁺; and compound 3.3b (slower eluting, 55 mg, 45.8% yield). MS: calc'd 566 (M+H)⁺, measured 566 (M+H)⁺, with 40% isopropanol (0.05% DEA)/CO₂ on OJ (5 μm, 250×20 mm) column.

### Step (e): Preparation of 4-[[(3R)-1-[(3R)-7-(ethylamino)-5-fluoro-3-methyl-2-oxo-indolin-3-yl]-3-piperidyl]oxy]benzenesulfonyl fluoride

To a solution of tert-butyl N-ethyl-N-[(3R)-5-fluoro-3-[(3R)-3-(4-fluorosulfonylphenoxy)-1-piperidyl]-3-methyl-2-oxo-indolin-7-yl]carbamate (50 mg, 88.5 μmol) in DCM (5 mL) was added TFA (2 mL). The reaction mixture was stirred at room temperature for 2 hrs, then concentrated to afford a crude product, which was purified by prep-HPLC to afford 4-[[(3R)-1-[(3R)-7-(ethylamino)-5-fluoro-3-methyl-2-oxo-indolin-3-yl]-3-piperidyl]oxy]benzenesulfonyl fluoride (15.5 mg, 37.8% yield). MS: calc'd 466 [(M+H)⁺]; measured 466 [(M+H)⁺]. ¹H NMR (400 MHz, DMSO-d₆) δ=10.27 (s, 1H), 8.04 (d, J=8.8 Hz, 2H), 7.28 (d, J=8.8 Hz, 2H), 6.58-6.53 (m, 1H), 6.40 (d, J=12.0 Hz, 1H), 4.72 (brs, 1H), 3.21-3.17 (m, 1H), 3.08 (q, J=7.2 Hz, 2H), 2.99-2.93 (m, 1H), 2.86-2.63 (m, 1H), 2.58-2.54 (m, 1H), 2.46-2.42 (m, 1H), 2.04-1.97 (m, 1H), 1.88-1.82 (m, 1H), 1.66-1.51 (m, 5H), 1.19 (t, J=7.2 Hz, 3H).

### Step (e): Preparation of 4-[[(3R)-1-[(3S)-7-(ethylamino)-5-fluoro-3-methyl-2-oxo-indolin-3-yl]-3-piperidyl]oxy]benzenesulfonyl fluoride

To a solution of tert-butyl N-ethyl-N-[(3S)-5-fluoro-3-[(3R)-3-(4-fluorosulfonylphenoxy)-1-piperidyl]-3-methyl-2-oxo-indolin-7-yl]carbamate (55 mg, 97.3 μmol) in DCM (5 mL) was added TFA (2 mL). The reaction mixture was stirred at room temperature for 2 hrs, then concentrated to afford a crude product, which was purified by prep-HPLC to afford 4-[[(3R)-1-[(3S)-7-(ethylamino)-5-fluoro-3-methyl-2-oxo-indolin-3-yl]-3-piperidyl]oxy]benzenesulfonyl fluoride (15.2 mg, 33.6% yield). MS: calc'd 466 [(M+H)⁺]; measured 466 [(M+H)⁺]. ¹H NMR (400 MHz, DMSO-d₆) δ=10.13 (s, 1H), 8.01 (d, J=8.8 Hz, 2H), 7.26 (d, J=8.8 Hz, 2H), 6.30 (d, J=11.6 Hz, 2H), 4.68 (brs, 1H), 3.10-3.05 (m, 3H), 2.99-2.68 (m, 2H), 2.58-2.54 (m, 1H), 2.49-2.47 (m, 1H), 2.05-1.73 (m, 2H), 1.69-1.37 (m, 5H), 1.18 (t, J=7.2 Hz, 3H).

### Example 4A and 4B

### 4-[[(3R)-1-[(3R)-7-(ethylamino)-5-fluoro-3-methyl-2-oxo-indolin-3-yl]-3-piperidyl]oxy]-2-methoxy-benzenesulfonyl fluoride and 4-[[(3R)-1-[(3S)-7-(ethylamino)-5-fluoro-3-methyl-2-oxo-indolin-3-yl]-3-piperidyl]oxy]-2-methoxy-benzenesulfonyl fluoride

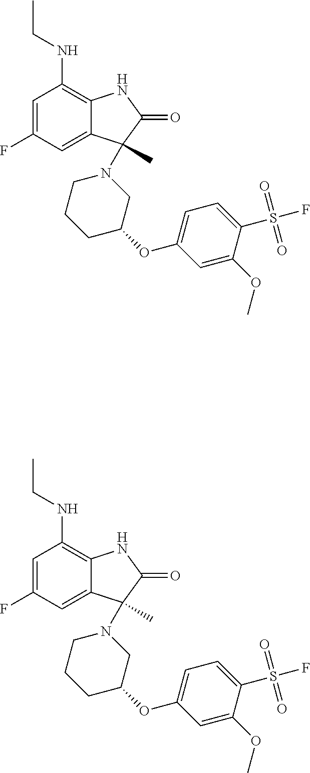 [SMILES: CCNc1cc(F)cc2c1NC(=O)[C@]2(C)N1CCC[C@@H](Oc2ccc(S(=O)(=O)F)c(OC)c2)C1 ; CCNc1cc(F)cc2c1NC(=O)[C@@]2(C)N1CCC[C@@H](Oc2ccc(S(=O)(=O)F)c(OC)c2)C1]

The titled compound was synthesized according to the following scheme:

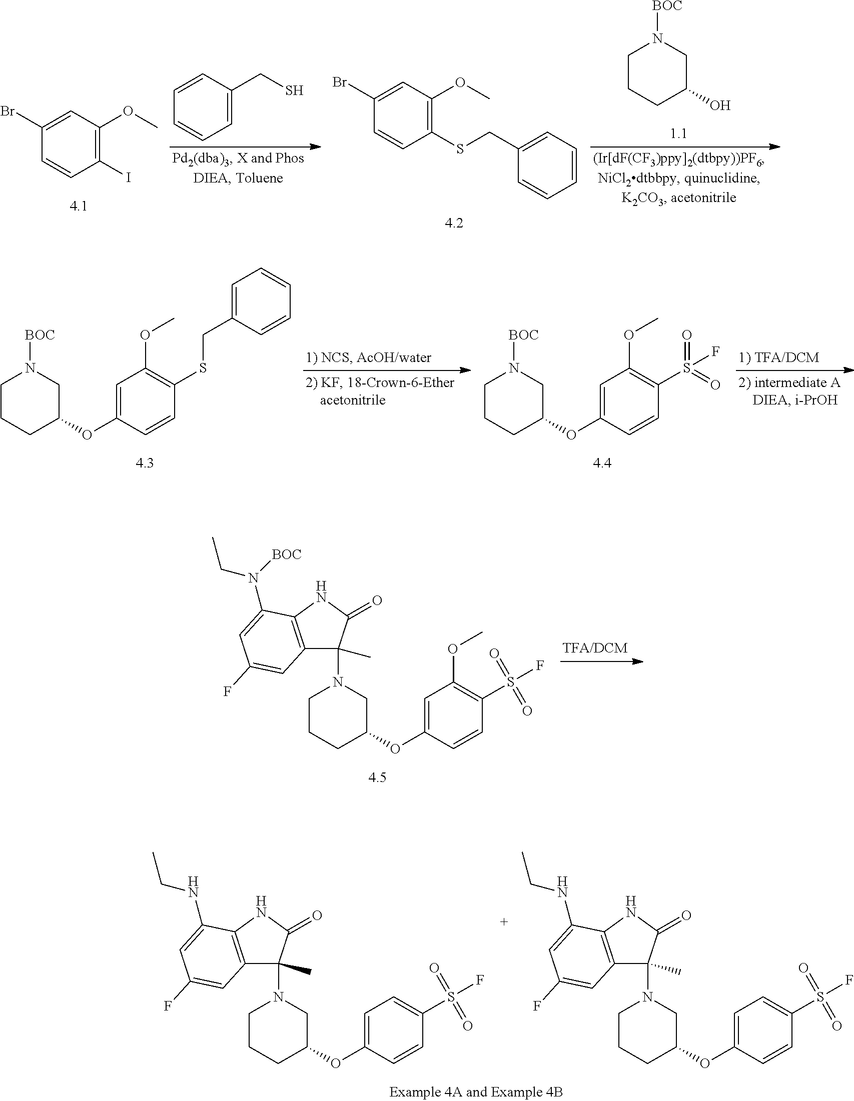

### Step (a): Preparation of 1-benzylsulfanyl-4-bromo-2-methoxy-benzene (Compound 4.2)

To a solution of phenylmethanethiol (600.0 mg, 4.83 mmol) in toluene (20 mL) was added 4-bromo-1-iodo-2-methoxy-benzene (compound 4.1, 1.50 g, 4.79 mmol), Xantphos (0.4 g, 0.69 mmol), DIEA (2.0 mL, 11.48 mmol) and Pd₂(dba)₃ (440.0 mg, 0.480 mmol), the resultant mixture was stirred at 100° C. for 12 hrs. After being cooled to room temperature, the reaction mixture was diluted with water (50 mL), and extracted with EA (50 mL) twice. The combined organic layer was washed with brine (50 mL), dried over Na₂SO₄, filtered and concentrated in vacuo. The residue was purified by flash chromatography (silica gel, 40 g, 4% to 10% EA in PE) to afford compound 4.2 (1400 mg, 94.9% yield).

### Step (b): Preparation of tert-butyl (3R)-3-(4-benzylsulfanyl-3-methoxy-phenoxy)piperidine-1-carboxylate (Compound 4.3)

To a solution of 1-benzylsulfanyl-4-bromo-2-methoxy-benzene (compound 4.2, 1.4 g, 4.53 mmol) in acetonitrile (5 mL) was added tert-butyl (3R)-3-hydroxypiperidine-1-carboxylate (compound 1.1, 1822 mg, 9.06 mmol), (Ir[dF(CF₃)ppy]₂(dtbpy))PF₆ (2.24 mg, 0.05 mmol), NiCl₂·dtbbpy (3.98 mg, 0.230 mmol), quinuclidine (50.34 mg, 0.450 mmol) and potassium carbonate (1251 mg, 9.06 mmol), the resultant mixture was purged with N₂ for three times, and then stirred at room temperature under the exposure of blue LEDs for 12 hrs. The reaction was filtered through Celite and washed with DCM, and the filtrate was concentrated in vacuo. The residue was purified by prep-TLC (50% EA in PE, R_{f}=0.3) to afford compound 4.3 (200 mg, 10.3% yield) as yellow solid. MS: calc'd 430 [(M+H)⁺], measured 430 [(M+H)⁺].

### Step (c): Preparation of tert-butyl (3R)-3-(4-fluorosulfonyl-3-methoxy-phenoxy)piperidine-1-carboxylate (Compound 4.4)

To a solution of tert-butyl (3R)-3-(4-benzylsulfanyl-3-methoxy-phenoxy)piperidine-1-carboxylate (compound 4.3, 150.0 mg, 0.35 mmol) in acetic acid (8 mL) and water (2 mL) was added N-chlorosuccinimide (200.0 mg, 1.5 mmol) at 0° C., the resultant mixture was stirred at room temperature for 2 hrs. The reaction mixture was diluted with water (50 mL), extracted with EA (50 mL). The organic layer was washed with brine (50 mL), dried over Na₂SO₄, filtered and the filtrate was concentrated to afford an oil. To a solution of the residue in acetonitrile (3 mL) was added potassium fluoride (52.0 mg, 0.90 mmol) and 18-crown-6 (80.0 mg, 0.30 mmol), and then the reaction mixture was stirred at room temperature for another 2 hrs. The reaction solution was diluted with water (30 mL) and extracted with EA (50 mL). The organic layer was washed with brine (50 mL), dried over Na₂SO₄, filtered and concentrated in vacuo to afford compound 4.4 (100 mg, 73.4% yield) which was used directly for the next step without further purification. MS: calc'd 390 [(M+H)⁺], measured 390 [(M+H)⁺].

### Step (d): Preparation of tert-butyl N-ethyl-N-[5-fluoro-3-methyl-2-oxo-3-[(3R)-3-(4-fluorosulfonyl-3-methoxy-phenoxy)-1-piperidyl]indolin-7-yl]carbamate (Compound 4.5)

To a solution of tert-butyl (3R)-3-(4-fluorosulfonyl-3-methoxy-phenoxy)piperidine-1-carboxylate (compound 4.4, 100.0 mg, 0.26 mmol) in DCM (5 mL) was added TFA (2 mL). The reaction mixture was stirred at room temperature for 2 hrs, then concentrated to afford an oil. To a solution of the residue and DIEA (0.35 mL, 2.0 mmol) in isopropanol (5 mL) was added tert-butyl N-(3-bromo-5-fluoro-3-methyl-2-oxo-indolin-7-yl)-N-ethyl-carbamate (intermediate A, 91.0 mg, 0.23 mmol). The reaction mixture was stirred at room temperature for 1 hr, then concentrated to afford a crude product, which was purified by prep-TLC (50% EA in PE, R_{f}=0.39) to afford compound 4.5 (100 mg, 64.6% yield). MS: calc'd 596 [(M+H)⁺], measured 596 [(M+H)⁺].

### Step (e): Preparation of 4-[[(3R)-1-[(3R)-7-(ethylamino)-5-fluoro-3-methyl-2-oxo-indolin-3-yl]-3-piperidyl]oxy]-2-methoxy-benzenesulfonyl fluoride and 4-[[(3R)-1-[(3S)-7-(ethylamino)-5-fluoro-3-methyl-2-oxo-indolin-3-yl]-3-piperidyl]oxy]-2-methoxy-benzenesulfonyl fluoride

To a solution of tert-butyl N-ethyl-N-[5-fluoro-3-methyl-2-oxo-3-[(3R)-3-(4-fluorosulfonyl-3-methoxy-phenoxy)-1-piperidyl]indolin-7-yl]carbamate (compound 4.5, 100.0 mg, 0.17 mmol) in DCM (5 mL) was added TFA (2 mL). The reaction mixture was stirred at room temperature for 2 hrs, then concentrated to afford a crude product, which was purified by prep-HPLC to afford Example 4A (11.0 mg, 13.1% yield) and Example 4B (10.2 mg, 12.1% yield): 4-[[(3R)-1-[(3R)-7-(ethylamino)-5-fluoro-3-methyl-2-oxo-indolin-3-yl]-3-piperidyl]oxy]-2-methoxy-benzenesulfonyl fluoride and 4-[[(3R)-1-[(3S)-7-(ethylamino)-5-fluoro-3-methyl-2-oxo-indolin-3-yl]-3-piperidyl]oxy]-2-methoxy-benzenesulfonyl fluoride.

Example 4A MS: calc'd 496 [(M+H)⁺], measured 496 [(M+H)⁺]. ¹H NMR (400 MHz, METHANOL-d₄) 8=7.84 (d, J=8.8 Hz, 1H), 6.84 (d, J=2.0 Hz, 1H), 6.73 (dd, J=8.8, 1.6 Hz, 1H), 6.67 (dd, J=8.0, 2.0 Hz, 1H), 6.49 (dd, J=11.6, 2.0 Hz, 1H), 3.98 (s, 3H), 3.38-3.50 (m, 1H), 3.30-3.24 (m, 2H), 3.19-3.12 (m, 3H), 2.16-1.85 (m, 5H), 1.74 (s, 3H), 1.28 (t, J=7.2 Hz, 3H).

Example 4B MS: calc'd 496 [(M+H)⁺], measured 496 [(M+H)⁺]. ¹H NMR (400 MHz, METHANOL-d₄) 8=7.84 (d, J=8.8 Hz, 1H), 6.83 (d, J=2.0 Hz, 1H), 6.73 (dd, J=8.8, 1.6 Hz, 1H), 6.67 (dd, J=8.0, 2.0 Hz, 1H), 6.49 (dd, J=11.6, 2.0 Hz, 1H), 3.98 (s, 3H), 3.50-3.41 (m, 1H), 3.30-3.25 (m, 2H), 3.19-3.12 (m, 3H), 2.15-1.88 (m, 5H), 1.74 (s, 3H), 1.27 (t, J=7.2 Hz, 3H).

### Example 5

### 4-[[(3S)-1-[7-(ethylamino)-5-fluoro-3-methyl-2-oxo-indolin-3-yl]-3-piperidyl]methyl]benzoic acid

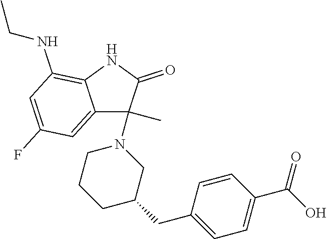 [SMILES: CCNc1cc(F)cc2c1NC(=O)C2(C)N1CCC[C@@H](Cc2ccc(C(=O)O)cc2)C1]

The titled compound was synthesized according to the following scheme:

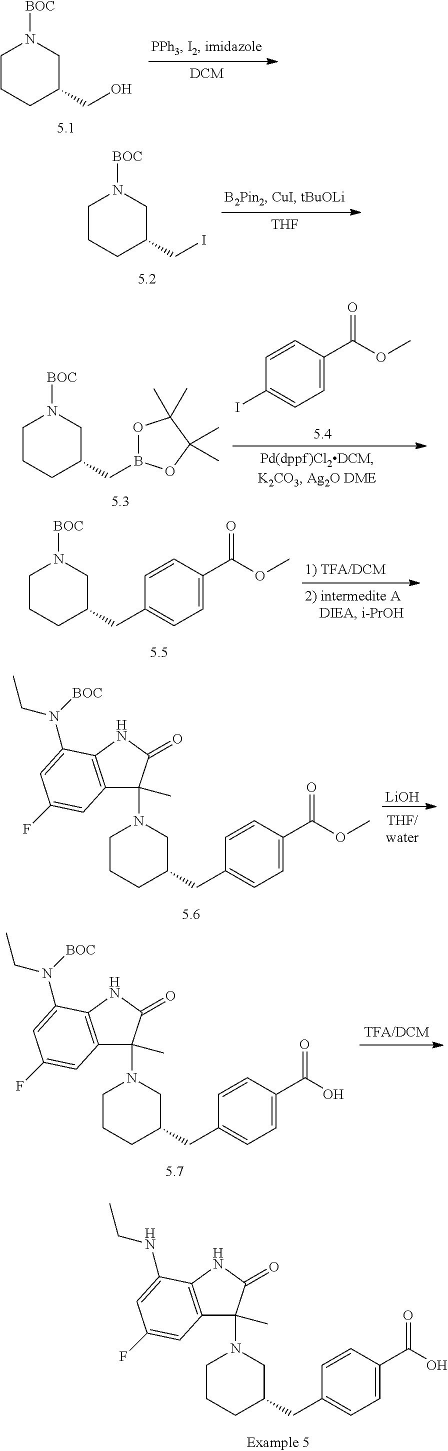

### Step (a): Preparation of tert-butyl (3R)-3-(iodomethyl)piperidine-1-carboxylate (Compound 5.2)

To a suspension of PPh₃ (3.65 g, 13.93 mmol), imidazole (0.95 g, 13.93 mmol) and iodine (3.54 g, 13.93 mmol) in DCM (40 mL) was added dropwise a solution of tert-butyl (3R)-3-(hydroxymethyl)piperidine-1-carboxylate (compound 5.1, 2.0 g, 9.29 mmol) in DCM (5 mL) at 0° C. The resultant mixture was stirred at room temperature for 16 hrs. The reaction was filtered through Celite and washed with DCM, and the filtrate was concentrated in vacuo. The residue was purified by RPLC to afford compound 5.2 (2.8 g, 92.7% yield) as a yellow oil. MS: calc'd 326 [(M+H)⁺], measured 270 [(M−55)⁺].

### Step (b): Preparation of tert-butyl (3R)-3-[(4,4,5,5-tetramethyl-1,3,2-dioxaborolan-2-yl)methyl]piperidine-1-carboxylate (Compound 5.3)

To a solution of tert-butyl (3R)-3-(iodomethyl)piperidine-1-carboxylate (compound 5.2, 2.8 g, 8.61 mmol) in THF (50 mL) was added bis(pinacolato)diboron (4.37 g, 17.22 mmol), CuI (328 mg, 1.72 mmol) and tBuOLi (1.38 g, 17.22 mmol), the resultant mixture was purged with N₂ for 3 times, and then the mixture was stirred at 50° C. for 5 hrs. After being cooled to room temperature, the reaction mixture was diluted with EA (300 mL), filtered and the filtrate was concentrated in vacuo. The residue was purified by flash chromatography (silica gel, 40 g, 0% to 20% EA in PE) to afford compound 5.3 (2 g, 71.4% yield). MS: calc'd 326 [(M+H)⁺], measured 326 [(M+H)⁺].

### Step (c): Preparation of tert-butyl (3S)-3-[(4-methoxycarbonylphenyl)methyl]piperidine-1-carboxylate (Compound 5.5)

To a solution of methyl 4-iodobenzoate (compound 5.4, 1332 mg, 5.08 mmol) in DME (20 mL) was added tert-butyl (3R)-3-[(4,4,5,5-tetramethyl-1,3,2-dioxaborolan-2-yl)methyl]piperidine-1-carboxylate (compound 5.3, 1500 mg, 4.61 mmol), Pd(dppf)Cl₂·CH₂Cl₂ (756 mg, 0.93 mmol), K₂CO₃ (1914 mg, 13.85 mmol) and Ag₂O (2697 mg, 11.54 mmol), the resultant mixture was stirred at 60° C. for 16 hrs. After being cooled to room temperature, the reaction mixture was diluted with water (50 mL) and extracted with EA (50 mL) for three times. The combined organic layer was washed with brine, dried over Na₂SO₄, filtered and concentrated in vacuo. The residue was purified by RPLC to afford compound 5.5 (850 mg, 55.3% yield). MS: calc'd 334 [(M+H)⁺], measured 234 [(M-BOC+H)⁺].

### Step (d): Preparation of methyl 4-[[(3S)-1-[7-[tert-butoxycarbonyl(ethyl)amino]-5-fluoro-3-methyl-2-oxo-indolin-3-yl]-3-piperidyl]methyl]benzoate (Compound 5.6)

To a solution of tert-butyl (3S)-3-[(4-methoxycarbonylphenyl)methyl]piperidine-1-carboxylate (compound 5.5, 400 mg, 1.2 mmol) in DCM (5 mL) was added TFA (2 mL). The reaction mixture was stirred at room temperature for 2 hrs, then concentrated to afford an oil. To a solution of the residue and DIEA (433 mg, 3.35 mmol) in isopropanol (5 mL) was added tert-butyl N-(3-bromo-5-fluoro-3-methyl-2-oxo-indolin-7-yl)-N-ethyl-carbamate (intermediate A, 432 mg, 1.12 mmol). The reaction mixture was stirred at room temperature for 1 hr., then concentrated to afford a crude product, which was purified by RPLC to afford compound 5.6 (350 mg, 54.1% yield). MS: calc'd 540 [(M+H)⁺], measured 540 [(M+H)⁺].

### Step (e): Preparation of 4-[[(3S)-1-[7-[tert-butoxycarbonyl(ethyl)amino]-5-fluoro-3-methyl-2-oxo-indolin-3-yl]-3-piperidyl]methyl]benzoic acid (Compound 5.7)

To a solution of 4-[[(3S)-1-[7-[tert-butoxycarbonyl(ethyl)amino]-5-fluoro-3-methyl-2-oxo-indolin-3-yl]-3-piperidyl]methyl]benzoate (compound 5.6, 350 mg, 0.65 mmol) in THF (10 mL) was added 0.5 N LiOH (5 mL). The resultant mixture was heated to 70° C. and stirred for 2 hrs. The mixture was concentrated, diluted with water (10 mL), and acidified with 1N HCl to pH about 5, and extracted with EA (50 mL) twice. The combined organic layer was washed with brine, dried over Na₂SO₄, filtered and concentrated to afford compound 5.7 (320 mg, 93.8% yield) which was used directly for the next step without further purification. MS: calc'd 526 [(M+H)⁺], measured 526 [(M+H)⁺].

### Step (f): Preparation of 4-[[(3S)-1-[7-(ethylamino)-5-fluoro-3-methyl-2-oxo-indolin-3-yl]-3-piperidyl]methyl]benzoic acid (Example 5)

To a solution of 4-[[(3S)-1-[7-[tert-butoxycarbonyl(ethyl)amino]-5-fluoro-3-methyl-2-oxo-indolin-3-yl]-3-piperidyl]methyl]benzoic acid (compound 5.7, 50 mg, 0.09 mmol) in DCM (5 mL) was added TFA (2 mL). The reaction mixture was stirred at room temperature for 2 hrs, then concentrated to afford a crude product, which was purified by pre-HPLC to afford Example 5 (6.6 mg, 17.3% yield). MS: calc'd 426 [(M+H)⁺], measured 426 [(M+H)⁺]. ¹H NMR (400 MHz, DMSO-d₆) δ=12.82 (s, 1H), 10.62 (s, 1H), 7.85 (d, J=8.0 Hz, 2H), 7.28 (d, J=8.0 Hz, 2H), 6.71-6.63 (m, 1H), 6.49-6.43 (m, 1H), 5.33 (brs, 1H), 3.27-3.21 (m, 2H), 3.10 (q, J=7.2 Hz, 2H), 2.76-2.69 (m, 1H), 2.58-2.55 (m, 2H), 2.45-2.41 (m, 1H), 2.08-2.00 (m, 1H), 1.79-1.50 (m, 6H), 1.20 (t, J=7.2 Hz, 3H), 1.12-1.02 (m, 1H).

### Example 6

### 4-[[(3S)-1-[7-(ethylamino)-5-fluoro-3-methyl-2-oxo-indolin-3-yl]-3-piperidyl]methyl]benzamide

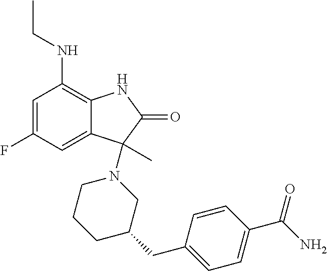 [SMILES: CCNc1cc(F)cc2c1NC(=O)C2(C)N1CCC[C@@H](Cc2ccc(C(N)=O)cc2)C1]

The titled compound was synthesized according to the following scheme:

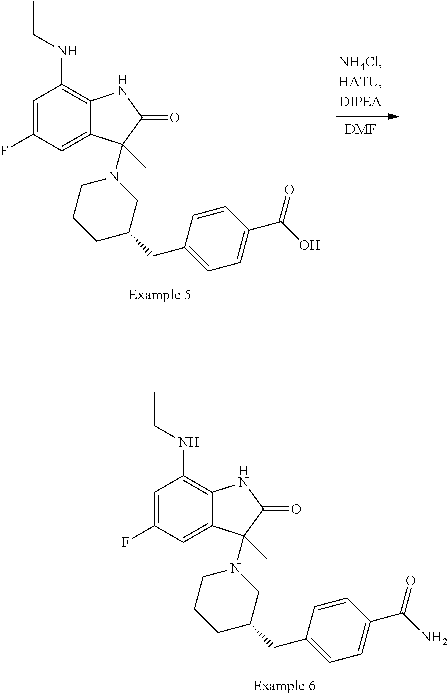

### Step (a): Preparation of 4-[[(3S)-1-[7-(ethylamino)-5-fluoro-3-methyl-2-oxo-indolin-3-yl]-3-piperidyl]methyl]benzamide (Example 6)

To a solution of 4-[[(3S)-1-[7-(ethylamino)-5-fluoro-3-methyl-2-oxo-indolin-3-yl]-3-piperidyl]methyl]benzoic acid (Example 5, 100 mg, 0.24 mmol) in DMF (5 mL) was added HATU (135 mg, 0.36 mmol) and DIEA (92 mg, 0.71 mmol). The resultant mixture was stirred at room temperature for 15 min, then NH₄Cl (19 mg, 0.36 mmol) was added, and then the reaction was stirred at this temperature for another 16 hrs. The reaction mixture was directly purified by pre-HPLC to afford Example 6 (13.8 mg, 13.8% yield). MS: calc'd 425 [(M+H)⁺], measured 425 [(M+H)⁺]. ¹H NMR (400 MHz, DMSO-d₆) δ=9.80 (s, 1H), 7.85 (s, 1H), 7.74 (d, J=8.0 Hz, 2H), 7.23 (s, 1H), 7.19 (d, J=8.0 Hz, 2H), 6.30-6.23 (m, 2H), 5.09 (t, J=4.0 Hz, 1H), 3.10-3.03 (m, 2H), 2.74-2.67 (m, 2H), 2.59-2.54 (m, 1H), 2.42-2.32 (m, 2H), 1.96 (t, J=10.0 Hz, 1H), 1.71-1.68 (m, 1H), 1.59-1.54 (m, 1H), 1.47-1.42 (m, 1H), 1.35-1.31 (m, 1H), 1.28 (s, 3H), 1.19 (t, J=7.2 Hz, 3H), 0.94-0.85 (m, 1H).

### Example 7

### 4-[[(3S)-1-[7-(ethylamino)-5-fluoro-3-methyl-2-oxo-indolin-3-yl]-3-piperidyl]methyl]benzonitrile

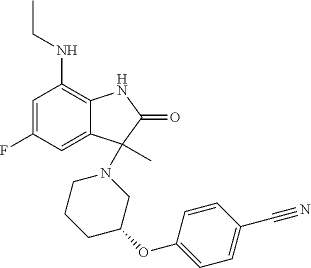 [SMILES: CCNc1cc(F)cc2c1NC(=O)C2(C)N1CCC[C@@H](Oc2ccc(C#N)cc2)C1]

4-[[(3S)-1-[7-(ethylamino)-5-fluoro-3-methyl-2-oxo-indolin-3-yl]-3-piperidyl]methyl]benzonitrile (Example 7) was prepared in analogy to Example 5, by replacing methyl 4-iodobenzoate (compound 5.4) with 4-iodobenzonitrile in step (c). MS: calc'd 407 [(M+H)⁺]; measured 407 [(M+H)⁺]. ¹H NMR (400 MHz, METHANOL-d₄) δ=7.68-7.62 (m, 2H), 7.38-7.35 (m, 2H), 6.65 (dd, J=8.0 Hz, 2.4 Hz, 0.5H), 6.60 (dd, J=, 8.0 Hz, 2.4 Hz, 0.5H), 6.54-6.48 (m, 1H), 3.64 (d, J=11.2 Hz, 0.5H), 3.53 (d, J=11.2 Hz, 0.5H), 3.37-3.34 (m, 1H), 3.22-3.14 (m, 2H), 3.12-3.03 (m, 1H), 2.85-2.61 (m, 3H), 2.21-2.13 (m, 1H), 2.02-1.95 (m, 1H), 1.81-1.72 (m, 5H), 1.30 (td, J=7.2 Hz, 3.2 Hz, 3H), 1.23-1.16 (m, 1H).

### Example 8

### 4-[[(3R)-1-[7-(ethylamino)-5-fluoro-3-methyl-2-oxo-indolin-3-yl]-3-piperidyl]amino]benzonitrile

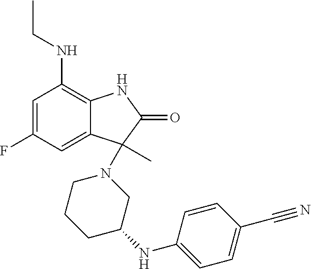 [SMILES: CCNc1cc(F)cc2c1NC(=O)C2(C)N1CCC[C@@H](Nc2ccc(C#N)cc2)C1]

The titled compound was synthesized according to the following scheme:

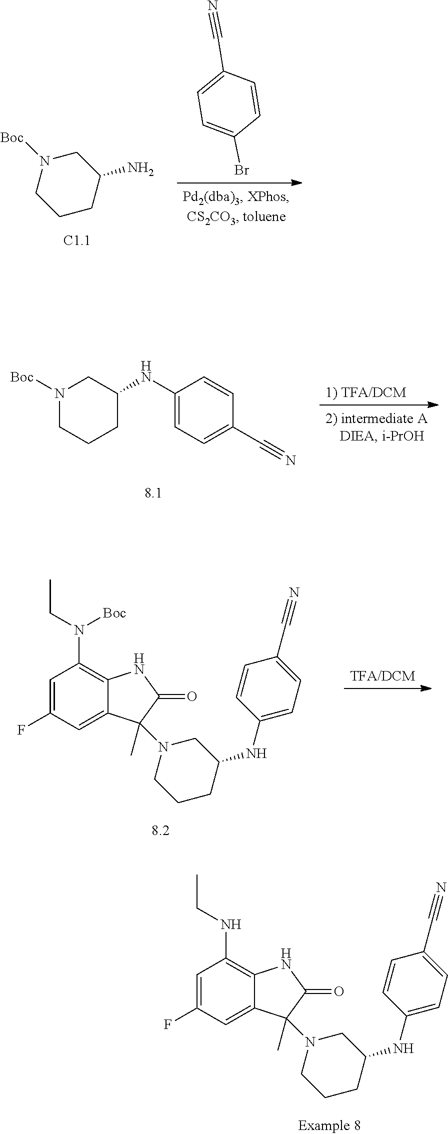 [SMILES: CC(C)(C)OC(=O)N1CCC[C@@H](N)C1 ; N#Cc1ccc(Br)cc1 ; CC(C)(C)OC(=O)N1CCC[C@@H](Nc2ccc(C#N)cc2)C1 ; CCN(C(=O)OC(C)(C)C)c1cc(F)cc2c1NC(=O)C2(C)N1CCC[C@@H](Nc2ccc(C#N)cc2)C1 ; CCNc1cc(F)cc2c1NC(=O)C2(C)N1CCC[C@@H](Nc2ccc(C#N)cc2)C1]

### Step (a): Preparation of tert-butyl (3R)-3-(4-cyanoanilino)piperidine-1-carboxylate (Compound 8.1)

To a solution of 4-bromobenzonitrile (450 mg, 2.5 mmol) in toluene (10 mL) was added tert-butyl (3R)-3-aminopiperidine-1-carboxylate (compound C1.1, 500 mg, 2.5 mmol), Pd₂(dba)₃ (450 mg, 0.5 mmol), XPhos (475 mg, 1 mmol) and cesium carbonate (2.5 g, 7.7 mmol), the resultant mixture was stirred at 110° C. for 16 hrs. After being cooled to room temperature, the reaction mixture was diluted with water (30 mL) and extracted with EA (20 mL) for three times. The combined organic layer was washed with brine, dried over Na₂SO₄, filtered and concentrated in vacuo. The residue was purified by flash chromatography (silica gel, 20 g, 15% to 25% EA in DCM) to afford compound 8.1 (600 mg, 79.7% yield). MS: calc'd 302 [(M+H)⁺], measured 246 [(M−55)⁺].

### Step (b): Preparation of tert-butyl N-[3-[(3R)-3-(4-cyanoanilino)-1-piperidyl]-5-fluoro-3-methyl-2-oxo-indolin-7-yl]-N-ethyl-carbamate (Compound 8.2)

To a solution of tert-butyl (3R)-3-(4-cyanoanilino)piperidine-1-carboxylate (compound 8.1, 60 mg, 0.20 mmol) in DCM (5 mL) was added TFA (2 mL). The reaction mixture was stirred at room temperature for 2 hrs., then concentrated to afford an oil. To a solution of the residue and DIEA (129 mg, 1.0 mmol) in isopropanol (5 mL) was added tert-butyl N-(3-bromo-5-fluoro-3-methyl-2-oxo-indolin-7-yl)-N-ethyl-carbamate (intermediate A, 77.2 mg, 0.2 mmol). The resultant mixture was stirred at room temperature for 1 hr. The reaction mixture was diluted with water (50 mL), and extracted with EA (20 mL) for three times. The combined organic layer was washed with brine, dried over Na₂SO₄, filtered and concentrated in vacuo. The residue was purified by flash chromatography (silica gel, 20 g, 20% to 100% EtOAc in PE) to afford compound 8.2 (60 mg, 59.2% yield). MS: calc'd 508 [(M+H)⁺], measured 508 [(M+H)⁺].

### Step (c): Preparation of 4-[[(3R)-1-[7-(ethylamino)-5-fluoro-3-methyl-2-oxo-indolin-3-yl]-3-piperidyl]amino]benzonitrile (Example 8)

To a solution of tert-butyl N-[3-[(3R)-3-(4-cyanoanilino)-1-piperidyl]-5-fluoro-3-methyl-2-oxo-indolin-7-yl]-N-ethyl-carbamate (compound 8.2, 60 mg, 0.12 mmol) in DCM (5 mL) was added TFA (2 mL). The reaction mixture was stirred at room temperature for 2 hrs, then concentrated to afford a crude product, which was purified by pre-HPLC to afford Example 8 (9.1 mg, 18.6% yield). MS: calc'd 408.3 [(M+H)⁺], measured 408.3 [(M+H)⁺]. ¹H NMR (400 MHz, DMSO-d₆) δ=10.27 (s, 1H) 7.47-7.41 (m, 2H), 6.63-6.51 (m, 4H), 6.43-6.36 (m, 1H), 3.10-3.00 (m, 4H), 2.64-2.56 (m, 1H), 2.42-2.37 (m, 1H), 1.95-1.72 (m, 2H), 1.68-1.45 (m, 4H), 1.37-1.27 (m, 1H), 1.20-1.13 (m, 4H).

### Example 9

### 4-[[(3R)-1-[7-(ethylamino)-5-fluoro-3-methyl-2-oxo-indolin-3-yl]-3-piperidyl]amino]-2-hydroxy-benzonitrile

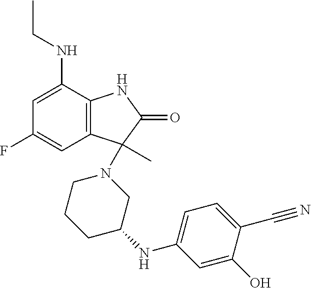 [SMILES: CCNc1cc(F)cc2c1NC(=O)C2(C)N1CCC[C@@H](Nc2ccc(C#N)c(O)c2)C1]

4-[[(3R)-1-[7-(ethylamino)-5-fluoro-3-methyl-2-oxo-indolin-3-yl]-3-piperidyl]amino]-2-hydroxy-benzonitrile (Example 9) was prepared in analogy to Example 8, by replacing 4-bromobenzonitrile with 4-bromo-2-[(4-methoxyphenyl)methoxy]benzonitrile in step (a). MS: calc'd 424 [M+H]⁺; measured 424 [M+H]⁺. ¹H NMR (400 MHz, DMSO-d₆) δ=7.22 (dt, J=8.3, 4.1 Hz, 1H), 6.62-6.54 (m, 1H), 6.44-6.40 (m, 1H), 6.11-6.05 (m, 2H), 3.69-3.49 (m, 1H), 3.30-3.11 (m, 2H), 3.10-2.99 (m, 2H), 2.83-2.70 (m, 1H), 2.46-2.43 (m, 2H), 1.97-1.80 (m, 2H), 1.72-1.62 (m, 1H), 1.57 (dd, J=8.0, 3.2 Hz, 3H), 1.36-1.25 (m, 1H), 1.17-1.12 (m, 3H).

### Example 10

### 5-[[(3S)-1-[7-(ethylamino)-5-fluoro-3-methyl-2-oxo-indolin-3-yl]-3-piperidyl]amino]pyridine-2-carboxylic acid

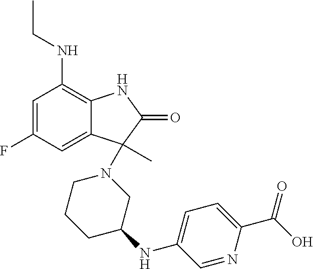 [SMILES: CCNc1cc(F)cc2c1NC(=O)C2(C)N1CCC[C@H](Nc2ccc(C(=O)O)nc2)C1]

The titled compound was synthesized according to the following scheme:

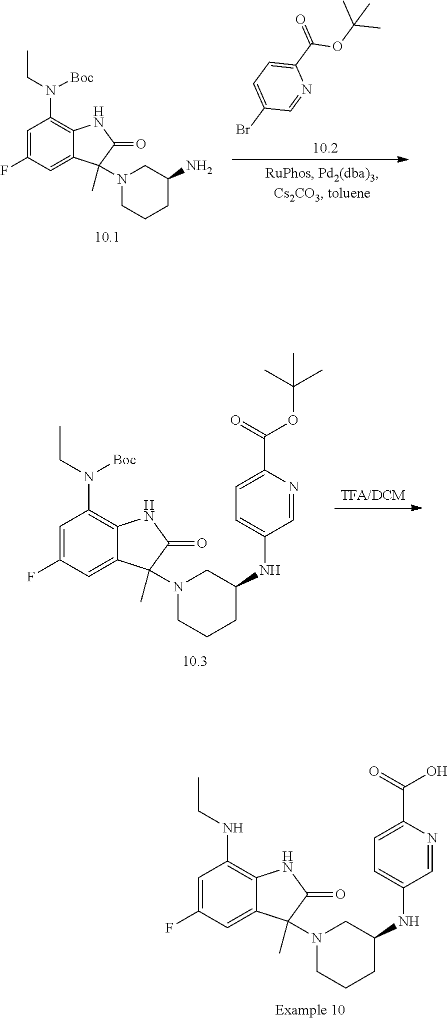 [SMILES: CCN(C(=O)OC(C)(C)C)c1cc(F)cc2c1NC(=O)C2(C)N1CCC[C@H](N)C1 ; CC(C)(C)OC(=O)c1ccc(Br)cn1 ; CCN(C(=O)OC(C)(C)C)c1cc(F)cc2c1NC(=O)C2(C)N1CCC[C@H](Nc2ccc(C(=O)OC(C)(C)C)nc2)C1 ; CCNc1cc(F)cc2c1NC(=O)C2(C)N1CCC[C@H](Nc2ccc(C(=O)O)nc2)C1]

### Step (a): Preparation of tert-butyl N-[3-[(3S)-3-amino-1-piperidyl]-5-fluoro-3-methyl-2-oxo-indolin-7-yl]-N-ethyl-carbamate (Compound 10.1)

tert-butyl N-[3-[(3S)-3-amino-1-piperidyl]-5-fluoro-3-methyl-2-oxo-indolin-7-yl]-N-ethyl-carbamate (compound 10.1) was prepared in analogy to intermediate C, by replacing tert-butyl (3R)-3-aminopiperidine-1-carboxylate (compound C1.1) with tert-butyl (3S)-3-aminopiperidine-1-carboxylate in step (a). MS: calc'd 407 [M+H]⁺; measured 407 [M+H]⁺.

### Step (b): Preparation of tert-butyl 5-[[(3S)-1-[7-[tert-butoxycarbonyl(ethyl)amino]-5-fluoro-3-methyl-2-oxo-indolin-3-yl]-3-piperidyl]amino]pyridine-2-carboxylate (Compound 10.3)

To a solution of tert-butyl N-[3-[(3S)-3-amino-1-piperidyl]-5-fluoro-3-methyl-2-oxo-indolin-7-yl]-N-ethyl-carbamate (compound 10.1, 400 mg, 0.98 mmol) in toluene (20 mL) was added tert-butyl 5-bromopyridine-2-carboxylate (compound 10.2, 254 mg, 0.98 mmol), Cs₂CO₃ (802 mg, 2.5 mmol), RuPhos (92 mg, 0.2 mmol) and Pd₂(dba)₃ (91.4 mg, 0.1 mmol), the resultant mixture was stirred at 110° C. for 16 hrs. After being cooled to room temperature, the reaction was diluted with water (30 mL) and extracted with EA (30 mL) twice. The combined organic layer was washed with brine, dried over Na₂SO₄, filtered and concentrated in vacuo. The residue was purified by pre-HPLC to afford compound 10.3 (280 mg, 48.9% yield). MS(ESI): calc'd 584 [(M+H)⁺]; measured 584 [(M+H)⁺].

### Step (c): Preparation of 5-[[(3S)-1-[7-(ethylamino)-5-fluoro-3-methyl-2-oxo-indolin-3-yl]-3-piperidyl]amino]pyridine-2-carboxylic acid (Example 10)

To a solution of tert-butyl 5-[[(3S)-1-[7-[tert-butoxycarbonyl(ethyl)amino]-5-fluoro-3-methyl-2-oxo-indolin-3-yl]-3-piperidyl]amino]pyridine-2-carboxylate (compound 10.3, 200.0 mg, 0.34 mmol) in DCM (10 mL) was added TFA (5 mL). The reaction mixture was stirred at room temperature for 2 hrs, then concentrated to afford a crude product, which was purified by prep-HPLC to afford Example 10 (23.4 mg, 16.8% yield). MS: calc'd 428 [(M+H)⁺]; measured 428 [(M+H)⁺]. ¹H NMR (400 MHz, DMSO-d₆) δ=10.03-9.79 (m, 1H), 8.07-7.85 (m, 1H), 7.79-7.58 (m, 1H), 6.99-6.76 (m, 1H), 6.48 (br d, J=7.2 Hz, 1H), 6.42-6.34 (m, 1H), 6.28 (dd, J=2.3, 12.0 Hz, 1H), 5.13 (br d, J=1.6 Hz, 1H), 3.05 (br dd, J=5.2, 7.0 Hz, 2H), 2.97-2.92 (m, 1H), 2.88-2.79 (m, 2H), 2.46 (br s, 1H), 2.28-2.17 (m, 1H), 2.27-2.17 (m, 1H), 2.12-2.00 (m, 1H), 1.91-1.78 (m, 1H), 1.71-1.58 (m, 1H), 1.56-1.43 (m, 1H), 1.36 (s, 3H), 1.18-1.14 (m, 3H).

### Example 11

### 5-[[(3R)-1-[7-(ethylamino)-5-fluoro-3-methyl-2-oxo-indolin-3-yl]-3-piperidyl]amino]pyridine-2-carboxylic acid

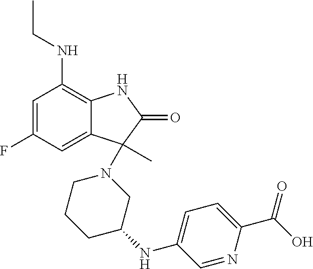 [SMILES: CCNc1cc(F)cc2c1NC(=O)C2(C)N1CCC[C@@H](Nc2ccc(C(=O)O)nc2)C1]

5-[[(3R)-1-[7-(ethylamino)-5-fluoro-3-methyl-2-oxo-indolin-3-yl]-3-piperidyl]amino]pyridine-2-carboxylic acid (Example 11) was prepared in analogy to Example 10, by replacing tert-butyl N-[3-[(3S)-3-amino-1-piperidyl]-5-fluoro-3-methyl-2-oxo-indolin-7-yl]-N-ethyl-carbamate (compound 10.1) with tert-butyl N-[3-[(3R)-3-amino-1-piperidyl]-5-fluoro-3-methyl-2-oxo-indolin-7-yl]-N-ethyl-carbamate (intermediate C) in step (b). MS: calc'd 428 [M+H]⁺; measured 428 [M+H]⁺. ¹H NMR (400 MHz, DMSO-d₆) δ=9.98-9.78 (m, 1H), 7.93 (d, J=1.5 Hz, 1H), 7.69 (d, J=8.9 Hz, 1H), 6.80 (dd, J=2.7, 8.8 Hz, 1H), 6.51-6.49 (m, 1H), 6.51-6.43 (m, 1H), 6.39 (dd, J=2.3, 8.3 Hz, 1H), 6.29 (d, J=2.6 Hz, 1H), 6.26 (d, J=2.4 Hz, 1H), 5.15-5.07 (m, 1H), 3.09-3.01 (m, 2H), 2.87-2.77 (m, 2H), 2.47-2.44 (m, 2H), 2.10-2.00 (m, 1H), 1.89-1.80 (m, 1H), 1.72-1.60 (m, 1H), 1.55-1.42 (m, 1H), 1.35 (s, 3H), 1.16 (t, J=7.0 Hz, 3H).

### Example 12

### 5-[[(3R)-1-[7-(ethylamino)-5-fluoro-3-methyl-2-oxo-indolin-3-yl]-3-piperidyl]amino]pyridine-2-carboxamide

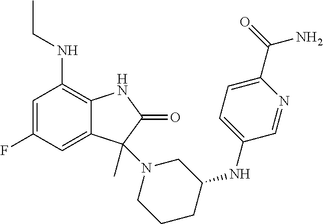 [SMILES: CCNc1cc(F)cc2c1NC(=O)C2(C)N1CCC[C@@H](Nc2ccc(C(N)=O)nc2)C1]

The titled compound was synthesized according to the following scheme:

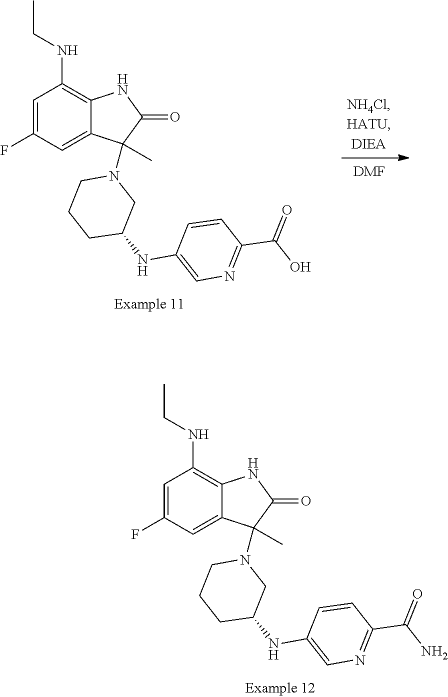

### Step (a): Preparation of 5-[[(3R)-1-[7-(ethylamino)-5-fluoro-3-methyl-2-oxo-indolin-3-yl]-3-piperidyl]amino]pyridine-2-carboxamide (Example 12)

To a solution of 5-[[(3R)-1-[7-(ethylamino)-5-fluoro-3-methyl-2-oxo-indolin-3-yl]-3-piperidyl]amino]pyridine-2-carboxylic acid (Example 11, 50.0 mg, 0.12 mmol) and DIEA (30.18 mg, 0.23 mmol) in DMF (2 mL) was added HATU (57.0 mg, 0.15 mmol), followed by NH₄Cl (64.2 mg, 1.2 mmol). The resultant mixture was stirred at room temperature for 2 hrs. The reaction mixture was directly purified by prep-HPLC to afford Example 12 (17.4 mg, 34.9% yield). MS: calc'd 427 [M+H]⁺; measured 427 [M+H]⁺. ¹H NMR (400 MHz, DMSO-d₆) (=10.03-9.69 (m, 1H), 7.92 (d, J=2.7 Hz, 1H), 7.73 (d, J=8.6 Hz, 1H), 7.69-7.63 (m, 1H), 7.15 (br d, J=2.0 Hz, 1H), 6.95 (dd, J=2.6, 8.7 Hz, 1H), 6.40-6.23 (m, 3H), 5.11 (br s, 1H), 3.11-3.02 (m, 2H), 3.00-2.92 (m, 1H), 2.82-2.65 (m, 2H), 2.46-2.40 (m, 1H), 2.27-2.15 (m, 1H), 1.92-1.82 (m, 1H), 1.72-1.60 (m, 1H), 1.52-1.40 (m, 1H), 1.35 (s, 3H), 1.23-1.14 (m, 4H).

### Example 13

### 5-[[(3R)-1-[7-(ethylamino)-5-fluoro-3-methyl-2-oxo-indolin-3-yl]-3-piperidyl]amino]pyridine-2-carbonitrile

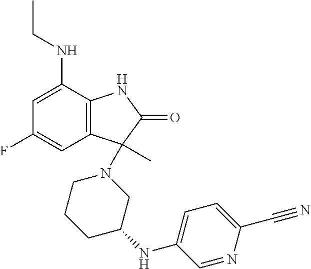 [SMILES: CCNc1cc(F)cc2c1NC(=O)C2(C)N1CCC[C@@H](Nc2ccc(C#N)nc2)C1]

The titled compound was synthesized according to the following scheme:

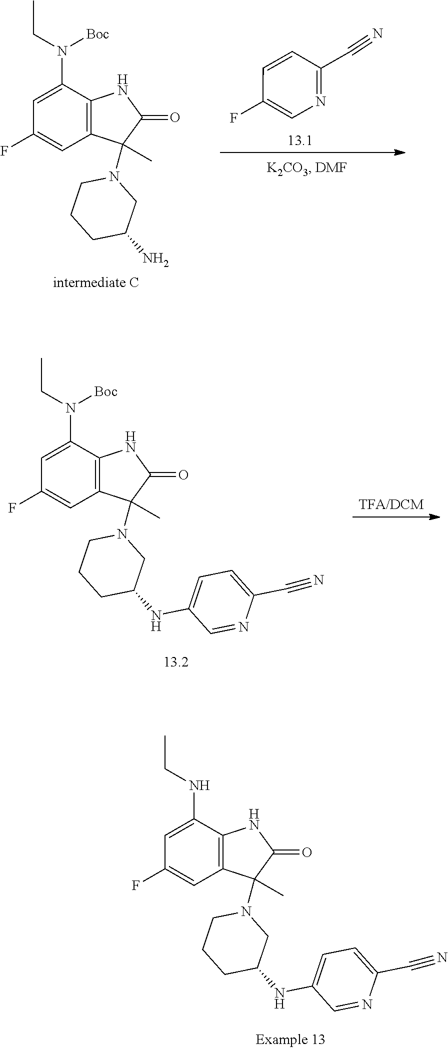

### Step (a): Preparation of tert-butyl N-ethyl-N-[5-fluoro-3-methyl-2-oxo-3-[(3R)-3-[(6-cyano-3-pyridyl)amino]-1-piperidyl]indolin-7-yl]carbamate (Compound 13.2)

To a solution of tert-butyl N-ethyl-N-[5-fluoro-3-methyl-2-oxo-3-[(3R)-3-amino-1-piperidyl]indolin-7-yl]carbamate (intermediate C, 100.0 mg, 0.25 mmol) in DMF (4 mL) was added 2-cyano-5-fluoropyridine (compound 13.1, 30.04 mg, 0.25 mmol) and K₂CO₃ (67.9 mg, 0.5 mmol), the resultant mixture was stirred at 80° C. for 12 hrs. After being cooled to room temperature, the reaction was diluted with water (60 mL) and extracted with EA (30 mL) twice. The combined organic layer was washed with brine, dried over Na₂SO₄, filtered and concentrated in vacuo. The residue was purified by prep-HPLC to afford compound 13.2 (50 mg, 39.4% yield). MS: calc'd 509 [(M+H)⁺]; measured 509 [(M+H)⁺].

### Step (b): Preparation of 5-[[(3R)-1-[7-(ethylamino)-5-fluoro-3-methyl-2-oxo-indolin-3-yl]-3-piperidyl]amino]pyridine-2-carbonitrile (Example 13)

To a solution of tert-butyl N-ethyl-N-[5-fluoro-3-methyl-2-oxo-3-[(3R)-3-[(6-cyano-3-pyridyl)amino]-1-piperidyl]indolin-7-yl]carbamate (compound 13.2, 50.0 mg, 0.10 mmol) in DCM (10 mL) was added TFA (2 mL). The reaction mixture was stirred at room temperature for 2 hrs, then concentrated to afford a crude product, which was purified by prep-HPLC to afford Example 13 (6.6 mg, 16.2% yield). MS: calc'd 409 [M+H]⁺; measured 409 [M+H]⁺. ¹H NMR (400 MHz, CHLOROFORM-d) 6=9.12-9.05 (m, 1H), 8.01-7.99 (m, 1H), 7.41-7.38 (m, 1H), 6.81-6.77 (m, 0.3H), 6.74-6.71 (m, 0.7H), 6.48-6.42 (m, 0.6H), 6.38-6.32 (m, 1.4H), 5.08 (s, 0.7H), 4.84 (s, 0.3H), 4.01-3.44 (m, 2H), 3.23-3.16 (m, 2H), 2.94-2.54 (m, 4H), 1.61-1.59 (m, 3H), 1.52 (s, 3H), 1.35 (t, J=7.2 Hz, 3H).

### Example 14

### 6-[[(3R)-1-[7-(ethylamino)-5-fluoro-3-methyl-2-oxo-indolin-3-yl]-3-piperidyl]amino]pyridine-3-carbonitrile

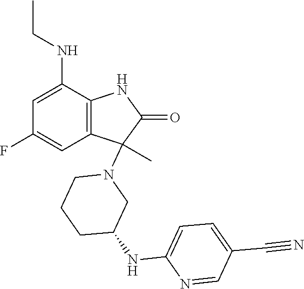 [SMILES: CCNc1cc(F)cc2c1NC(=O)C2(C)N1CCC[C@@H](Nc2ccc(C#N)cn2)C1]

6-[[(3R)-1-[7-(ethylamino)-5-fluoro-3-methyl-2-oxo-indolin-3-yl]-3-piperidyl]amino]pyridine-3-carbonitrile (Example 14) was prepared in analogy to Example 13, by replacing 2-cyano-5-fluoropyridine (compound 13.1) with 6-fluoropyridine-3-carbonitrile in step (a). MS: calc'd 409 [M+H]⁺; measured 409 [M+H]⁺. ¹H NMR (400 MHz, DMSO-d₆) δ=9.84-9.82 (m, 1H), 8.35 (d, J=2.4, 0.4H), 8.35 (d, J=2.4, 0.6H), 7.65-7.61 (m, 1H), 7.41-7.33 (m, 1H), 6.52 (dd, J=17.2, 8.8, Hz, 1H), 6.40-6.17 (m, 2H), 5.10 (t, J=4.2 Hz, 1H), 3.90 (brs, 1H), 3.09-3.02 (m, 2H), 2.93-2.91 (m, 0.4H), 2.81-2.74 (m, 1H), 2.63-2.60 (m, 0.6H), 2.45-2.31 (m, 2H), 2.26-2.11 (m, 1H), 1.81-1.77 (m, 1H), 1.66-1.61 (m, 1H), 1.46-1.32 (m, 4H), 1.24-1.16 (m, 3H).

### Example 15

### 5-[[(3R)-1-[7-(ethylamino)-5-fluoro-3-methyl-2-oxo-indolin-3-yl]-3-piperidyl]amino]pyrazine-2-carbonitrile

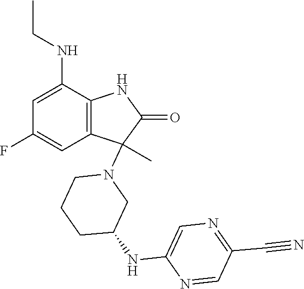 [SMILES: CCNc1cc(F)cc2c1NC(=O)C2(C)N1CCC[C@@H](Nc2cnc(C#N)cn2)C1]

5-[[(3R)-1-[7-(ethylamino)-5-fluoro-3-methyl-2-oxo-indolin-3-yl]-3-piperidyl]amino]pyrazine-2-carbonitrile (Example 15) was prepared in analogy to Example 13, by replacing 2-cyano-5-fluoropyridine (compound 13.1) with 5-chloropyrazine-2-carbonitrile in step (a). MS: calc'd 410 [(M+H)⁺], measured 410 [(M+H)⁺]. ¹H NMR (400 MHz, METHANOL-d₄) δ=8.27 (d, J=1.2, 1H), 7.94 (d, J=1.6, 1H), 6.37-6.34 (m, 1H), 6.32-6.29 (m, 1H), 4.16-4.09 (m, 1H), 3.15-3.12 (m, 2H), 2.95-2.90 (m, 1H), 2.77-2.72 (m, 1H), 2.53-2.46 (m, 2H), 1.77-1.72 (m, 2H), 1.56-1.50 (m, 2H), 1.45-1.43 (m, 3H), 1.31-1.26 (m, 3H).

### Example 16

### 6-[[(3R)-1-[7-(ethylamino)-5-fluoro-3-methyl-2-oxo-indolin-3-yl]-3-piperidyl]amino]pyridazine-3-carbonitrile

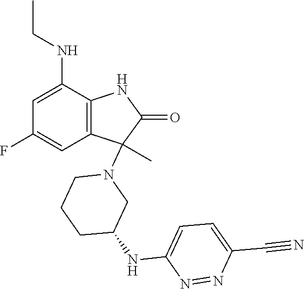 [SMILES: CCNc1cc(F)cc2c1NC(=O)C2(C)N1CCC[C@@H](Nc2ccc(C#N)nn2)C1]

5-[[(3R)-1-[7-(ethylamino)-5-fluoro-3-methyl-2-oxo-indolin-3-yl]-3-piperidyl]amino]pyrazine-2-carbonitrile (Example 16) was prepared in analogy to Example 13, by replacing 2-cyano-5-fluoropyridine (compound 13.1) with 6-chloropyridazine-3-carbonitrile in step (a). MS: calc'd 410 [(M+H)⁺], measured 410 [(M+H)⁺]. ¹H NMR (400 MHz, METHANOL-d₄) δ=7.65-7.60 (m, 1H), 6.96-6.90 (m, 1H), 6.57-6.43 (m, 2H), 4.51-4.41 (m, 1H), 3.62-3.51 (m, 1H), 3.40-3.33 (m, 1H), 3.19-3.14 (m, 2H), 3.09-2.93 (m, 2H), 2.09-2.01 (m, 2H), 1.92-1.81 (m, 1H), 1.73-1.59 (m, 4H), 1.28 (t, J=7.2 Hz, 3H).

### Example 17

### 5-[[(3R)-1-[7-(ethylamino)-5-fluoro-3-methyl-2-oxo-indolin-3-yl]-3-piperidyl]amino]pyrimidine-2-carbonitrile

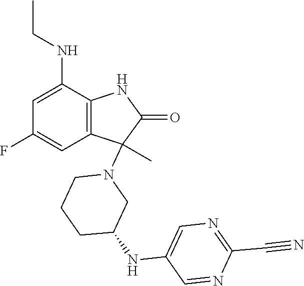 [SMILES: CCNc1cc(F)cc2c1NC(=O)C2(C)N1CCC[C@@H](Nc2cnc(C#N)nc2)C1]

5-[[(3R)-1-[7-(ethylamino)-5-fluoro-3-methyl-2-oxo-indolin-3-yl]-3-piperidyl]amino]pyrazine-2-carbonitrile (Example 17) was prepared in analogy to Example 13, by replacing 2-cyano-5-fluoropyridine (compound 13.1) with 5-fluoropyrimidine-2-carbonitrile in step (a). MS: calc'd 410 [(M+H)⁺], measured 410 [(M+H)⁺]. ¹H NMR (400 MHz, DMSO-d₆) δ=10.29-10.15 (m, 1H), 8.19 (s, 2H), 7.12 (d, J=7.2 Hz, 1H), 6.55-6.33 (m, 2H), 4.51-4.41 (m, 1H), 3.73-3.66 (m, 1H), 3.11-2.94 (m, 4H), 2.53-2.52 (m, 1H), 1.91-1.87 (m, 1H), 1.80-1.73 (m, 1H), 1.62-1.56 (m, 1H), 1.55-1.47 (m, 3H), 1.37-1.30 (m, 2H), 1.18 (t, J=7.2 Hz, 3H).

### Example 18

### 7-(ethylamino)-5-fluoro-3-[(3R)-3-[[6-(hydroxymethyl)-3-pyridyl]amino]-1-piperidyl]-3-methyl-indolin-2-one

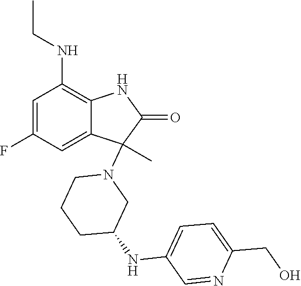 [SMILES: CCNc1cc(F)cc2c1NC(=O)C2(C)N1CCC[C@@H](Nc2ccc(CO)nc2)C1]

The titled compound was synthesized according to the following scheme:

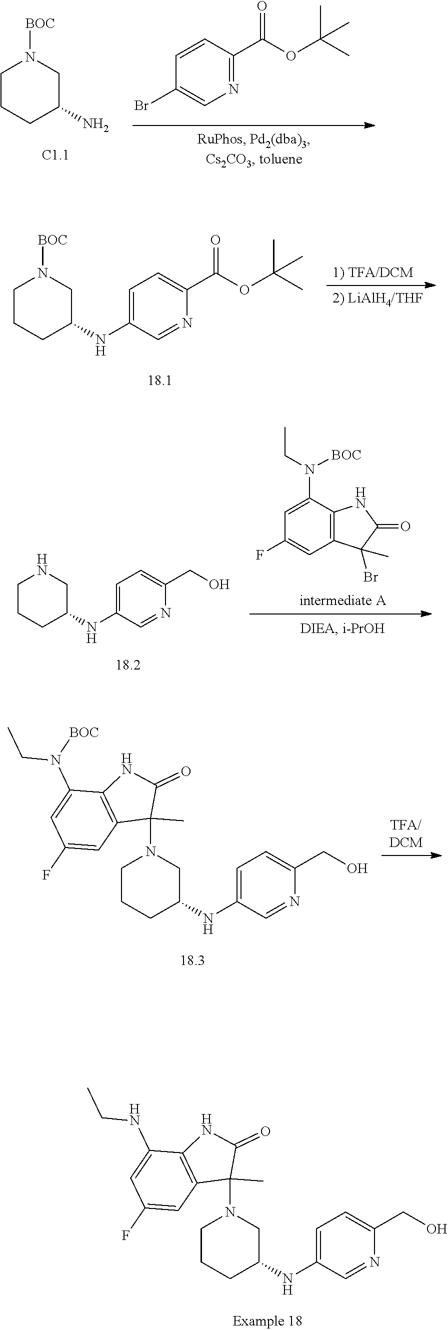

### Step (a): Preparation of tert-butyl 5-[[(3R)-1-tert-butoxycarbonyl-3-piperidyl]amino]pyridine-2-carboxylate (Compound 18.1)

To a solution of tert-butyl (3R)-3-aminopiperidine-1-carboxylate (compound C1.1, 300.0 mg, 1.5 mmol) in toluene (10 mL) was added tert-butyl 5-bromopyridine-2-carboxylate (385 mg, 1.5 mmol), Cs₂CO₃ (1220.1 mg, 3.7 mmol), RuPhos (139.8 mg, 0.3 mmol) and Pd₂(dba)₃ (180 mg, 0.2 mmol), the resultant mixture was stirred at 110° C. for 16 hrs. After being cooled to room temperature, the reaction mixture was diluted with water (50 mL), extracted with EA (50 mL) twice. The combined organic layer was washed with brine, dried over Na₂SO₄, filtered and concentrated. The residue was purified by pre-HPLC to afford compound 18.1 (150 mg, 26.5% yield). MS: calc'd 378 [(M+H)⁺], measured 378 [(M+H)⁺].

### Step (b): Preparation of [5-[[(3R)-3-piperidyl]amino]-2-pyridyl]methanol (Compound 18.2)

To a solution of tert-butyl 5-[[(3R)-1-tert-butoxycarbonyl-3-piperidyl]amino]pyridine-2-carboxylate (compound 18.1, 150.0 mg, 0.40 mmol) in DCM (10 mL) was added TFA (5 mL). The reaction mixture was stirred at room temperature for 2 hrs., then concentrated to afford an oil. To a solution of the residue in THF (5 mL) was added LiAlH₄ (68 mg, 1.81 mmol), the resultant mixture was stirred at room temperature for 2 hrs. 0.1 mL of water was added to the mixture, then 0.1 mL 15% NaOH (aq), and 0.3 mL water were added in order, and the mixture was stirred at room temperature for another 15 min. The mixture was filtered and the filtrate was concentrated in vacuo to afford compound 18.2 (50 mg, 60.1% yield) which was used directly for the next step without further purification. MS: calc'd 208 [(M+H)⁺], measured 208 [(M+H)⁺].

### Step (c): Preparation of tert-butyl N-ethyl-N-[5-fluoro-3-methyl-2-oxo-3-[(3R)-3-[[6-(hydroxymethyl)-3-pyridyl]amino]-1-piperidyl]indolin-7-yl]carbamate (Compound 18.3)

To a solution of [5-[[(3R)-3-piperidyl]amino]-2-pyridyl]methanol (compound 18.2, 50.0 mg, 0.24 mmol) and DIEA (62.2 mg, 0.50 mmol) in isopropanol (5 mL) was added tert-butyl N-(3-bromo-5-fluoro-3-methyl-2-oxo-indolin-7-yl)-N-ethyl-carbamate (intermediate A, 92.6 mg, 0.24 mmol). The resultant mixture was stirred at room temperature for 2 hrs., then concentrated to afford a crude product, the residue was purified by flash chromatography (silica gel, 20 g, 30% to 40% EA in PE) to afford compound 18.3 (50 mg, 40.6% yield). MS: calc'd 514 [(M+H)⁺], measured 514 [(M+H)⁺].

### Step (d): Preparation of 7-(ethylamino)-5-fluoro-3-[(3R)-3-[[6-(hydroxymethyl)-3-pyridyl]amino]-1-piperidyl]-3-methyl-indolin-2-one (Example 18)

To a solution of tert-butyl N-ethyl-N-[5-fluoro-3-methyl-2-oxo-3-[(3R)-3-[[6-(hydroxymethyl)-3-pyridyl]amino]-1-piperidyl]indolin-7-yl]carbamate (compound 18.3, 50.0 mg, 0.10 mmol) in DCM (5 mL) was added TFA (2 mL). The resultant mixture was stirred at room temperature for 2 hrs., then concentrated to afford a crude product, the residue was purified by pre-HPLC to afford Example 18 (20.2 mg, 50.3% yield). MS: calc'd 414 [(M+H)⁺], measured 414 [(M+H)⁺]. ¹H NMR (400 MHz, DMSO-d₆) δ=7.89 (s, 1H), 7.67-7.62 (m, 2H), 6.57-6.45 (m, 1H), 6.42-6.33 (m, 1H), 4.65 (d, J=4.0 Hz, 2H), 3.73-3.62 (m, 2H), 3.26-3.19 (m, 1H), 3.08 (dq, J=7.6, 3.2 Hz, 2H), 2.98-2.90 (m, 1H), 2.64-2.58 (m, 1H), 1.89-1.77 (m, 2H), 1.68-1.61 (m, 1H), 1.52 (s, 1.7H), 1.48 (s, 1.3H), 1.41-1.27 (m, 1H), 1.19 (t, J=6.8 Hz, 3H).

### Example 19

### 7-(ethylamino)-5-fluoro-3-[(3R)-3-[4-(2-hydroxyacetyl)anilino]-1-piperidyl]-3-methyl-indolin-2-one

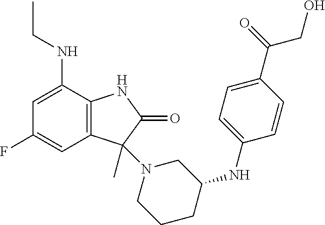 [SMILES: CCNc1cc(F)cc2c1NC(=O)C2(C)N1CCC[C@@H](Nc2ccc(C(=O)CO)cc2)C1]

The titled compound was synthesized according to the following scheme:

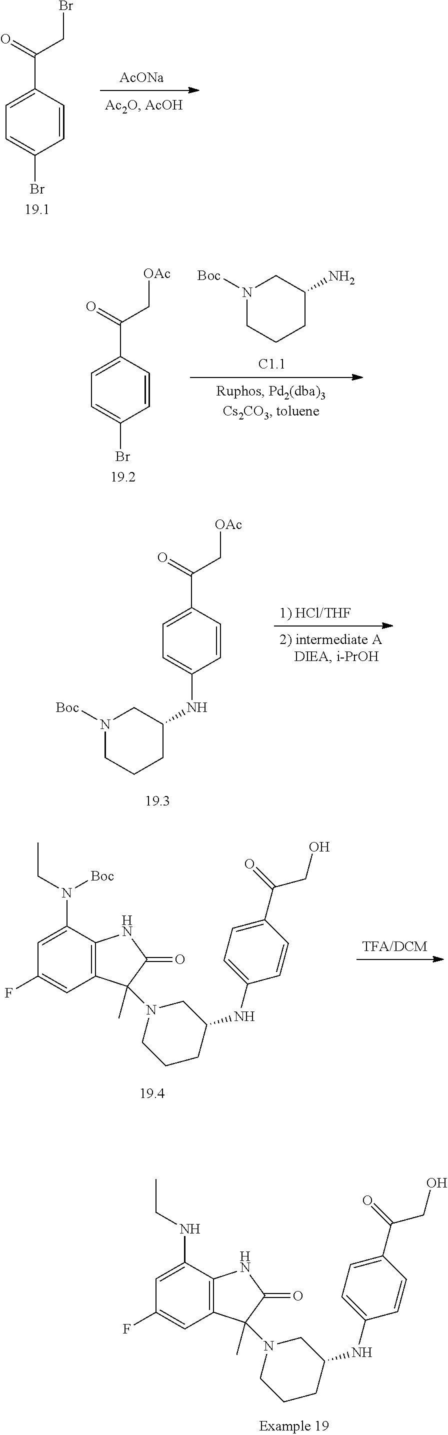

### Step (a): Preparation of [2-(4-bromophenyl)-2-oxo-ethyl] acetate (Compound 19.2)

To a solution of 2-bromo-1-(4-bromophenyl)ethanone (compound 19.1, 3.50 g, 12.60 mmol) in acetic acid (100 mL) was added NaOAc (1.0 g, 37.8 mmol) and Ac₂O (3.57 mL, 37.8 mmol), the resultant mixture was stirred at room temperature for 12 hrs. The reaction mixture was concentrated in vacuo to give a crude product which was further purified by flash chromatography on silica gel (silica gel, 80 g, 10% to 50% EtOAc in PE) to afford compound 19.2 (3.0 g, 93.0% yield). MS: calc'd 257 [(M+H)⁺], measured 214 [(M−42)⁺].

### Step (b): Preparation of tert-butyl (3R)-3-[4-(2-acetoxyacetyl)anilino]piperidine-1-carboxylate (Compound 19.3)

To a solution of [2-(4-bromophenyl)-2-oxo-ethyl] acetate (compound 19.2, 500.0 mg, 1.94 mmol) toluene (10 mL) was added tert-butyl (3R)-3-aminopiperidine-1-carboxylate (compound C1.1, 389 mg, 1.89 mmol), Pd₂(dba)₃ (90 mg, 0.1 mmol), Cs₂CO₃ (760 mg, 2.3 mmol) and RuPhos (90 mg, 0.19 mmol), the resultant mixture was stirred at 110° C. for 16 hrs. After being cooled to room temperature, the reaction was quenched with water (80 mL) and extracted with EA (50 mL) twice. The combined organic layer was washed with brine, dried over Na₂SO₄, filtered and the filtrate was concentrated in vacuo. The residue was purified by prep-HPLC to afford compound 19.3 (250 mg, 34.3% yield) as yellow solid. MS: calc'd 377 [(M+H)⁺]; measured 377 [(M+H)⁺].

### Step (c): Preparation of tert-butyl N-ethyl-N-[5-fluoro-3-[(3R)-3-[4-(2-hydroxyacetyl)anilino]-1-piperidyl]-3-methyl-2-oxo-indolin-7-yl]carbamate (Compound 19.4)

To a solution of tert-butyl (3R)-3-[4-(2-acetoxyacetyl)anilino]piperidine-1-carboxylate (compound 19.3, 300.0 mg, 0.8 mmol) in THF (5 mL) was added 6 N HCl (2.0 mL, 12 mmol), the resultant mixture was stirred at 70° C. for 3 hrs. The mixture was concentrated in vacuo to give a solid. To a solution of the residue and DIEA (88 mg, 0.70 mmol) in isopropanol (10 mL) was added tert-butyl N-(3-bromo-5-fluoro-3-methyl-2-oxo-indolin-7-yl)-N-ethyl-carbamate (intermediate A, 132 mg, 0.34 mmol). The reaction mixture was stirred at room temperature for 2 hrs, then concentrated to afford a crude product, which was purified by flash chromatography on silica gel (silica gel, 20 g, 30% to 50% EtOAc in PE) to afford compound 19.4 (100 mg, 54.5% yield). MS: calc'd 541 [(M+H)⁺]; measured 541 [(M+H)⁺].

### Step (e): Preparation of 7-(ethylamino)-5-fluoro-3-[(3R)-3-[4-(2-hydroxyacetyl)anilino]-1-piperidyl]-3-methyl-indolin-2-one (Example 19)

To a solution of tert-butyl N-ethyl-N-[5-fluoro-3-[(3R)-3-[4-(2-hydroxyacetyl)anilino]-1-piperidyl]-3-methyl-2-oxo-indolin-7-yl]carbamate (Example 19.4, 70.0 mg, 0.13 mmol) in DCM (2 mL) was added TFA (1 mL). The resultant mixture was stirred at room temperature for 2 hrs., then concentrated to afford a crude product, the residue was purified by pre-HPLC to afford Example 19 (16.8 mg, 29.3% yield). MS: calc'd 441 [(M+H)⁺]; measured 441 [(M+H)⁺]. ¹H NMR (400 MHz, DMSO-d₆) δ=7.64 (d, J=8.8 Hz, 1H), 7.58 (d, J=8.8 Hz, 1H), 6.5 (d, J=8.8 Hz, 1H), 6.49-6.37 (m, 2H), 6.27-6.24 (m, 1H), 4.58 (d, J=11.6 Hz, 2H), 3.45-3.26 (m, 1H), 3.02-2.95 (m, 2H), 2.88-2.83 (m, 1H), 2.73-2.58 (m, 2H), 2.22-2.16 (m, 1H), 1.90-1.85 (m, 1H), 1.79-1.72 (m, 1H), 1.66-1.60 (m, 1H), 1.51-1.39 (m, 1H), 1.32 (s, 1.8H), 1.31 (s, 1.2H), 1.15-1.09 (m, 3H).

### Example 20

### 7-(ethylamino)-5-fluoro-3-methyl-3-[(3R)-3-[(6-methylsulfonyl-3-pyridyl)amino]-1-piperidyl]indolin-2-one

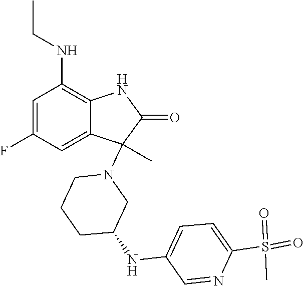 [SMILES: CCNc1cc(F)cc2c1NC(=O)C2(C)N1CCC[C@@H](Nc2ccc(S(C)(=O)=O)nc2)C1]

7-(ethylamino)-5-fluoro-3-methyl-3-[(3R)-3-[(6-methylsulfonyl-3-pyridyl)amino]-1-piperidyl]indolin-2-one (Example 20) was prepared in analogy to Example 10, by replacing tert-butyl 5-bromopyridine-2-carboxylate (compound 10.2) with 5-bromo-2-methylsulfonyl-pyridine in step (b). MS: calc'd 462 [M+H]⁺; measured 462 [M+H]⁺. ¹H NMR (400 MHz, METHANOL-d₄) δ=8.05 (d, J=2.4 Hz, 0.6H), 8.01 (d, J=2.4 Hz, 0.4H), 7.77 (d, J=8.8 Hz, 0.6H), 7.72 (d, J=8.8 Hz, 0.4H), 7.04 (dd, J=9.2, 2.8 Hz, 0.4H), 6.93 (dd, J=8.8, 2.4 Hz, 0.6H), 6.35-6.29 (m, 2H), 3.67-3.45 (m, 1H), 3.17-3.11 (m, 2H), 3.10-3.06 (m, 3H), 2.97-2.65 (m, 2H), 2.63-2.18 (m, 2H), 1.89-1.70 (m, 2H), 1.69-1.47 (m, 2H), 1.47 (s, 1.2H), 1.46 (s, 1.8H), 1.31-1.23 (m, 3H).

### Example 21

### 7-(ethylamino)-5-fluoro-3-[(3R)-3-[(6-hydroxy-3-pyridyl)amino]-1-piperidyl]-3-methyl-indolin-2-one

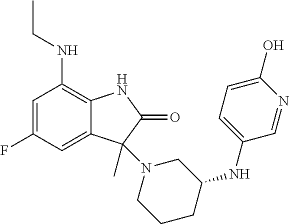 [SMILES: CCNc1cc(F)cc2c1NC(=O)C2(C)N1CCC[C@@H](Nc2ccc(O)nc2)C1]

The titled compound was synthesized according to the following scheme:

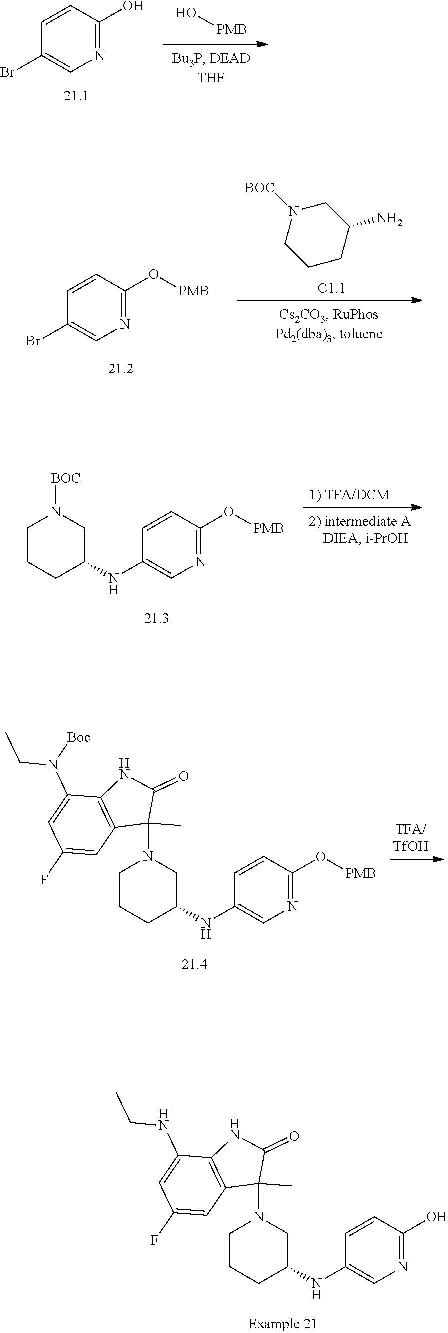

### Step (a): Preparation of 5-bromo-2-[(4-methoxyphenyl)methoxy]pyridine (Compound 21.2)

To a solution of 5-bromopyridin-2-ol (compound 21.1, 3.0 g, 17.24 mmol), (4-methoxyphenyl)methanol (3.60 g, 26.06 mmol) and Bu₃P (5.28 g, 26.1 mmol) in THF (60 mL) was slowly added DEAD (4.50 g, 25.85 mmol) at 20° C. under N₂, then the reaction mixture was stirred at 20° C. for another 2 hrs. The reaction mixture was concentrated to give the residue which was further purified by flash chromatography (silica gel, 120 g, 5% to 10% EA in PE) to afford compound 21.2 (4.0 g, 79.2% yield). MS: calc'd 294, 296 [(M+H)⁺]; measured 294, 296 [(M+H)⁺].

### Step (b): Preparation of tert-butyl (3R)-3-[[6-[(4-methoxyphenyl)methoxy]-3-pyridyl]amino]piperidine-1-carboxylate (Compound 21.3)

To a solution of 5-bromo-2-[(4-methoxyphenyl)methoxy]pyridine (compound 21.2, 1.50 g, 5.1 mmol) in toluene (20 mL) was added tert-butyl (3R)-3-aminopiperidine-1-carboxylate (compound C1.1, 1.50 g, 7.49 mmol), cesium carbonate (4.15 g, 12.75 mmol), RuPhos (416 mg, 0.51 mmol) and Pd₂(dba)₃ (228.5 mg, 0.25 mmol), then the resultant mixture was stirred at 110° C. for 12 hrs. After being cooled to room temperature, the reaction mixture was filtered through Celite and washed with DCM. The filtrate was concentrated to afford a crude product, which was purified by RPLC to afford compound 21.3 (600 mg, 28.5% yield) as yellow oil. MS: calc'd 414 [(M+H)⁺]; measured 414 [(M+H)⁺].

### Step (c): Preparation of tert-butyl N-ethyl-N-[5-fluoro-3-[(3R)-3-[[6-[(4-methoxyphenyl)methoxy]-3-pyridyl]amino]-1-piperidyl]-3-methyl-2-oxo-indolin-7-yl]carbamate (Compound 21.4)

To a solution of tert-butyl (3R)-3-[[6-[(4-methoxyphenyl)methoxy]-3-pyridyl]amino]piperidine-1-carboxylate (compound 21.3, 82.6 mg, 0.20 mmol) in DCM (10 mL) was added TFA (5 mL). The reaction mixture was stirred at room temperature for 2 hrs, then concentrated to afford an oil. To a solution of the residue and DIEA (129 mg, 1.0 mmol) in isopropanol (10 mL) was added tert-butyl N-(3-bromo-5-fluoro-3-methyl-2-oxo-indolin-7-yl)-N-ethyl-carbamate (intermediate A, 75.0 mg, 0.19 mmol). The reaction mixture was stirred at room temperature for 2 hrs, then concentrated to afford a crude product, which was purified by RPLC to afford compound 21.4 (95 mg, 76.7% yield). MS: calc'd 620 [(M+H)⁺]; measured 620 [(M+H)⁺].

### Step (d): Preparation of 7-(ethylamino)-5-fluoro-3-[(3R)-3-[(6-hydroxy-3-pyridyl)amino]-1-piperidyl]-3-methyl-indolin-2-one (Example 21)

A mixture of tert-butyl N-ethyl-N-[5-fluoro-3-[(3R)-3-[[6-[(4-methoxyphenyl)methoxy]-3-pyridyl]amino]-1-piperidyl]-3-methyl-2-oxo-indolin-7-yl]carbamate (compound 21.4, 95.0 mg, 0.15 mmol) in TFA (2.0 mL)/TfOH (0.5 mL) was stirred at room temperature for 1 hr. The reaction mixture was concentrated to afford a crude product, which was purified by prep-HPLC to afford Example 21 (11.2 mg, 18.7% yield). MS: calc'd 400 [(M+H)⁺]; measured 400 [(M+H)⁺]. ¹H NMR (400 MHz, METHANOL-d₄) δ=7.57-7.50 (m, 1H), 7.13-7.08 (m, 1H), 6.79-6.75 (m, 1H), 6.66 (dd, J=8.0, 2.0 Hz, 0.6H), 6.61-6.60 (m, 0.4H), 6.54-6.44 (m, 1H), 3.70-3.57 (m, 1H), 3.48-3.36 (m, 2H), 3.21-3.03 (m, 3H), 3.00-2.63 (m, 0.4H), 2.82-2.77 (m, 0.6H), 2.10-1.98 (m, 2H), 1.93-1.80 (m, 1H), 1.75 (s, 1.8H), 1.74 (s, 1.2H), 1.60-1.43 (m, 1H), 1.28 (t, J=7.2 Hz, 3H).

### Example 22

### 3-chloro-5-[[(3R)-1-[7-(ethylamino)-5-fluoro-3-methyl-2-oxo-indolin-3-yl]-3-piperidyl]amino]pyridine-2-carbonitrile

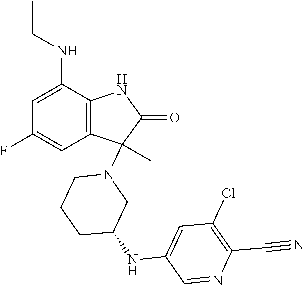 [SMILES: CCNc1cc(F)cc2c1NC(=O)C2(C)N1CCC[C@@H](Nc2cnc(C#N)c(Cl)c2)C1]

3-chloro-5-[[(3R)-1-[7-(ethylamino)-5-fluoro-3-methyl-2-oxo-indolin-3-yl]-3-piperidyl]amino]pyridine-2-carbonitrile (Example 22) was prepared in analogy to Example 8, by replacing 4-bromobenzonitrile with 5-bromo-3-chloro-pyridine-2-carbonitrile in step (a). MS: calc'd 443, 445 [M+H]⁺; measured 443, 445 [M+H]⁺. ¹H NMR (400 MHz, DMSO-d₆) δ=9.57 (s, 1H), 8.00 (d, J=2.4 Hz, 0.5H), 7.95 (d, J=2.4 Hz, 0.5H), 7.16-7.10 (m, 1.5H), 6.99-6.98 (m, 0.5H), 6.39 (dd, J=8.4, 2.0 Hz, 0.5H), 6.30-6.23 (m, 1.5H), 5.12 (t, J=4.0 Hz 1H), 3.55-3.39 (m, 1H), 3.09-3.03 (m, 2H), 2.83-2.76 (m, 1.5H), 2.70-2.66 (m, 0.5H), 2.46-2.27 (m, 1.5H), 2.14-2.10 (m, 0.5H), 1.85-1.77 (m 1H), 1.69-1.62 (m, 1H), 1.52-1.43 (m, 1H), 1.35 (s, 3H), 1.25-1.22 (m, 1H), 1.20-1.15 (m, 3H).

### Example 23

### 5-[[(3R)-1-[7-(ethylamino)-5-fluoro-3-methyl-2-oxo-indolin-3-yl]-3-methyl-3-piperidyl]amino]pyridine-2-carbonitrile

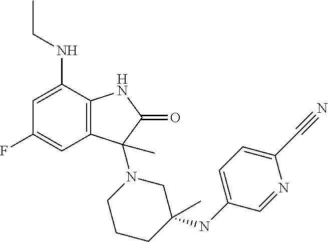 [SMILES: CCNc1cc(F)cc2c1NC(=O)C2(C)N1CCC[C@@](C)(Nc2ccc(C#N)nc2)C1]

The titled compound was synthesized according to the following scheme:

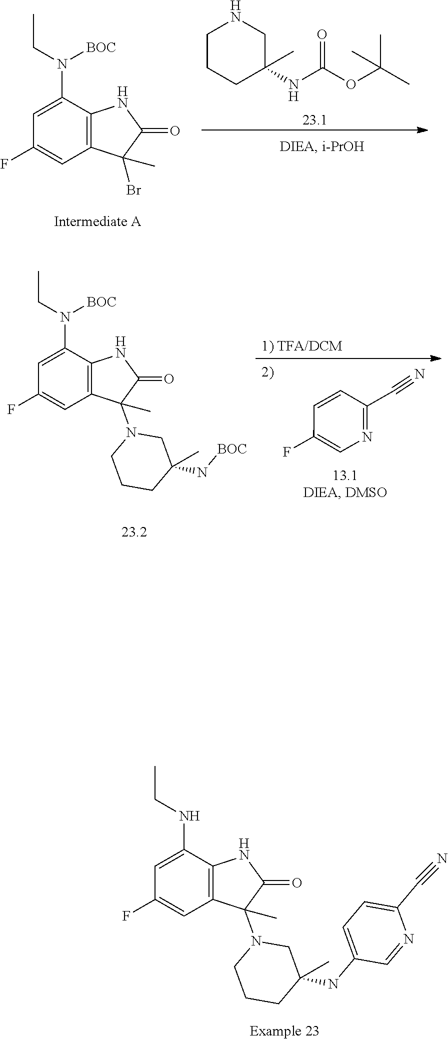 [SMILES: CCN(C(=O)OC(C)(C)C)c1cc(F)cc2c1NC(=O)C2(C)Br ; CC(C)(C)OC(=O)N[C@]1(C)CCCNC1 ; CCN(C(=O)OC(C)(C)C)c1cc(F)cc2c1NC(=O)C2(C)N1CCC[C@@](C)(NC(=O)OC(C)(C)C)C1 ; N#Cc1ccc(F)cn1 ; CCNc1cc(F)cc2c1NC(=O)C2(C)N1CCC[C@@](C)(Nc2ccc(C#N)nc2)C1]

### Step (a): Preparation of tert-butyl N-[3-[(3R)-3-(tert-butoxycarbonylamino)-3-methyl-1-piperidyl]-5-fluoro-3-methyl-2-oxo-indolin-7-yl]-N-ethyl-carbamate (Compound 23.2)

To a solution of tert-butyl N-[(3R)-3-methyl-3-piperidyl]carbamate (compound 23.1, CAS: 1169762-18-8, PharmaBlock, Catalog: PBN20120293, 100 mg, 0.5 mmol) and DIEA (301.5 mg, 2.3 mmol) in isopropanol (10 mL) was added tert-butyl N-(3-bromo-5-fluoro-3-methyl-2-oxo-indolin-7-yl)-N-ethyl-carbamate (Intermediate A, 180.7 mg, 468.1 μmol), the resultant mixture was stirred at room temperature for 2 hrs. The reaction mixture was diluted with water (50 mL), and extracted with EA (30 mL) twice. The combined organic layer was washed with brine, dried over Na₂SO₄, filtered and concentrated. The residue was purified by flash chromatography (silica gel, 20 g, 30% to 80% EA in PE) to afford compound 23.2 (165 mg, 67.9% yield). MS: calc'd 521 [(M+H)⁺], measured 521 [(M+H)⁺].

### Step (b): Preparation of 5-[[(3R)-1-[7-(ethylamino)-5-fluoro-3-methyl-2-oxo-indolin-3-yl]-3-methyl-3-piperidyl]amino]pyridine-2-carbonitrile (Example 23)

To a solution of tert-butyl N-[3-[(3R)-3-(tert-butoxycarbonylamino)-3-methyl-1-piperidyl]-5-fluoro-3-methyl-2-oxo-indolin-7-yl]-N-ethyl-carbamate (compound 23.2, 80 mg, 153.8 μmol) in DCM (10 mL) was added TFA (5 mL). The reaction mixture was stirred at room temperature for 2 hrs, then concentrated in vacuo to afford an oil. To a solution of the residue and DIEA (96.7 mg, 749.6 μmol) in DMSO (8 mL) was added 5-fluoropyridine-2-carbonitrile (compound 13.1, 22.0 mg, 180.3 μmol). The resultant mixture was stirred at 120° C. for 20 hrs. After being cooled to room temperature, the reaction mixture was diluted with water, and extracted with EA twice. The combined organic layer was washed with brine, dried over Na₂SO₄, filtered and concentrated. The residue was purified by flash chromatography (silica gel, 12 g, 0% to 10% MeOH in DCM) to afford Example 23 (32 mg, 50.5% yield). MS: calc'd 423 [(M+H)⁺], measured 423 [(M+H)⁺]. ¹H NMR (500 MHz, METHANOL-d₄) δ=8.18-8.14 (m, 1H), 7.52-7.45 (m, 1H), 7.19-7.05 (m, 1H), 6.46-6.27 (m, 2H), 3.18-3.11 (m, 2H), 3.02-2.87 (m, 1H), 2.83-2.74 (m, 0.5H), 2.65-2.58 (m, 0.5H), 2.49-2.27 (m, 2H), 2.22-1.94 (m, 2H), 1.67-1.49 (m, 2H), 1.47 (d, J=5.0 Hz, 3H), 1.37-1.31 (m, 3H), 1.31-1.27 (m, 3H).

### Example 24

### 5-[[(3R)-1-[7-(ethylamino)-5-fluoro-3-methyl-2-oxo-indolin-3-yl]-3-piperidyl]-methyl-amino]pyridine-2-carboxamide

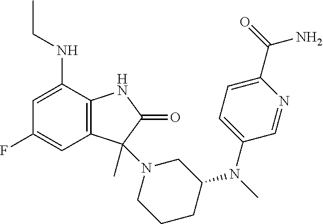 [SMILES: CCNc1cc(F)cc2c1NC(=O)C2(C)N1CCC[C@@H](N(C)c2ccc(C(N)=O)nc2)C1]

The titled compound was synthesized according to the following scheme:

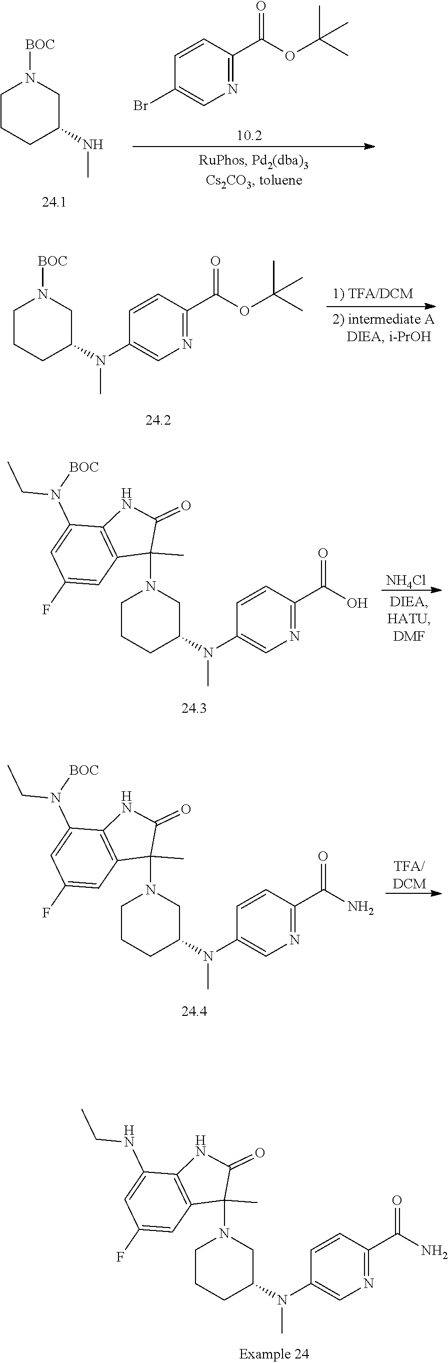

### Step (a): Preparation of tert-butyl 5-[[(3R)-1-tert-butoxycarbonyl-3-piperidyl]-methyl-amino]pyridine-2-carboxylate (Compound 24.2)

To a solution of tert-butyl (3R)-3-(methylamino)piperidine-1-carboxylate (compound 24.1, 130.0 mg, 0.61 mmol) in toluene (10 mL) was added Pd₂(dba)₃ (111.1 mg, 0.12 mmol), tert-butyl 5-bromopyridine-2-carboxylate (compound 10.2, 156.58 mg, 0.61 mmol), cesium carbonate (494.13 mg, 1.52 mmol) and RuPhos (56.57 mg, 0.120 mmol). The mixture was stirred at 100° C. for 16 hrs. After being cooled to room temperature, the reaction was diluted with water (30 mL) and extracted with EA (30 mL) twice. The combined organic layer was washed with brine, dried over Na₂SO₄, filtered and concentrated in vacuo. The residue was purified by RPLC to afford compound 24.2 (55 mg, 23.2% yield). MS: calc'd 392 [(M+H)⁺], measured 292 [(M-Boc+H)⁺].

### Step (b): Preparation of 5-[methyl-[(3R)-1-[7-[tert-butoxycarbonyl(ethyl)amino]-5-fluoro-3-methyl-2-oxo-indolin-3-yl]-3-piperidyl]amino]pyridine-2-carboxylic acid (Compound 24.3)

To a solution of tert-butyl 5-[[(3R)-1-tert-butoxycarbonyl-3-piperidyl]-methyl-amino]pyridine-2-carboxylate (compound 24.2, 55.0 mg, 0.14 mmol) in DCM (5 mL) was added trifluoroacetic acid (2 mL). The reaction mixture was stirred at room temperature for 3 hrs, then concentrated to afford an oil. To a solution of the residue and DIEA (0.07 mL, 0.43 mmol) in isopropanol (5 mL) was added tert-butyl N-(3-bromo-5-fluoro-3-methyl-2-oxo-indolin-7-yl)-N-ethyl-carbamate (intermediate A, 44.34 mg, 0.11 mmol). The reaction mixture was stirred at room temperature for 2 hrs., then concentrated to afford a crude product, which was purified by RPLC to afford compound 24.3 (40 mg, 52.7% yield). MS: calc'd 542 [(M+H)⁺], measured 542 [(M+H)⁺].

### Step (c): Preparation of tert-butyl N-ethyl-N-[5-fluoro-3-methyl-2-oxo-3-[(3R)-3-[(6-carbamoyl-3-pyridyl)-methyl-amino]-1-piperidyl]indolin-7-yl]carbamate (Compound 24.4)

To a solution of 5-[methyl-[(3R)-1-[7-[tert-butoxycarbonyl(ethyl)amino]-5-fluoro-3-methyl-2-oxo-indolin-3-yl]-3-piperidyl]amino]pyridine-2-carboxylic acid (compound 24.3, 40.0 mg, 0.07 mmol) in DMF (3 mL) was added NH₄Cl (19.75 mg, 0.35 mmol), HATU (33.7 mg, 0.09 mmol) and DIEA (0.04 mL, 0.22 mmol). The resultant mixture was stirred at room temperature for 2 hrs. The reaction mixture was diluted with water (30 mL), and extracted with EA (20 mL) twice. The combined organic layer was washed with brine, dried over Na₂SO₄, filtered and concentrated afford compound 24.4 (50 mg, crude) which was used directly for the next step without further purification. MS: calc'd 541 [(M+H)⁺], measured 541 [(M+H)⁺].

### Step (d): Preparation of 5-[[(3R)-1-[7-(ethylamino)-5-fluoro-3-methyl-2-oxo-indolin-3-yl]-3-piperidyl]-methyl-amino]pyridine-2-carboxamide (Example 24)

To a solution of tert-butyl N-ethyl-N-[5-fluoro-3-methyl-2-oxo-3-[(3R)-3-[(6-carbamoyl-3-pyridyl)-methyl-amino]-1-piperidyl]indolin-7-yl]carbamate (compound 24.5, 50.0 mg, 0.09 mmol) in DCM (5 mL) was added TFA (2 mL). The reaction mixture was stirred at room temperature for 2 hrs, then concentrated to afford a crude product, which was purified by prep-HPLC to afford Example 24 (15.8 mg, 39.8% yield). MS: calc'd 441 [(M+H)⁺], measured 441 [(M+H)⁺]. ¹H NMR (400 MHz, METHANOL-d₄) δ=8.23 (br s, 1H), 8.02 (br t, J=8.0 Hz, 1H), 7.37-7.36 (m, 1H), 6.73-6.60 (m, 1H), 6.50 (d, J=12.0 Hz, 1H), 4.31-4.19 (m, 1H), 3.55-3.47 (m, 1H), 3.43-3.36 (m, 1H), 3.22-3.14 (m, 2H), 3.13-3.02 (m, 1H), 3.02-2.88 (m, 4H), 2.14-2.02 (m, 1H), 2.01-1.87 (m, 3H), 1.74 (br d, J=4.0 Hz, 3H), 1.30 (t, J=7.2 Hz, 3H).

### Example 25

### N-(6-cyano-3-pyridyl)-N-[(3R)-1-[3-ethyl-7-(ethylamino)-5-fluoro-2-oxo-indolin-3-yl]-3-piperidyl]acetamide

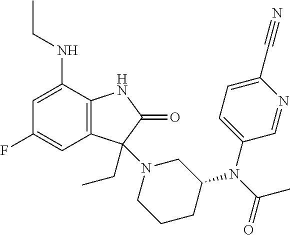 [SMILES: CCNc1cc(F)cc2c1NC(=O)C2(CC)N1CCC[C@@H](N(C(C)=O)c2ccc(C#N)nc2)C1]

The titled compound was synthesized according to the following scheme:

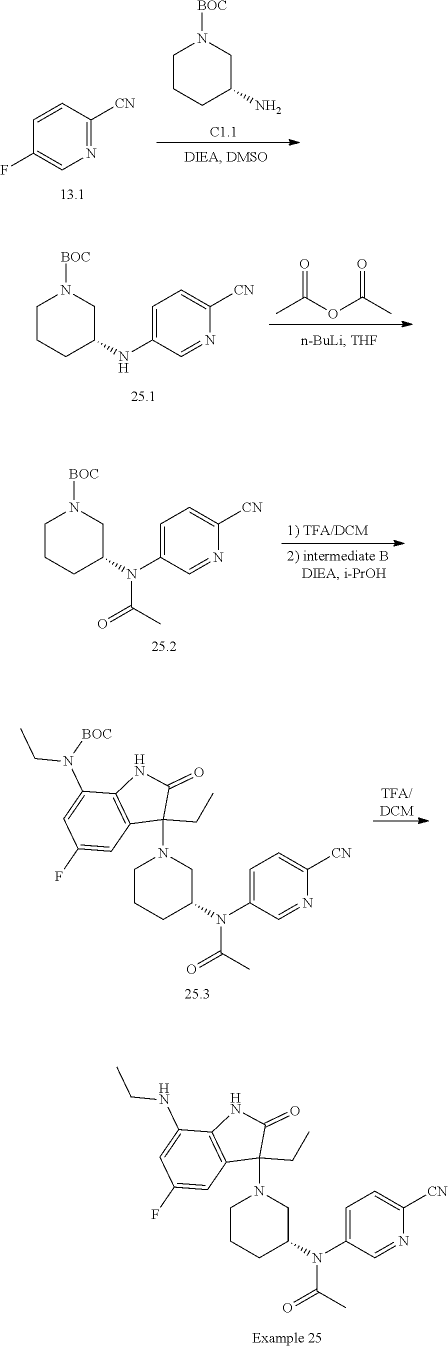

### Step (a): Preparation of tert-butyl (3R)-3-[(6-cyano-3-pyridyl)amino]piperidine-1-carboxylate (Compound 25.1)

To a solution of tert-butyl (3R)-3-aminopiperidine-1-carboxylate (compound C1.1, 984 mg, 4.91 mmol) in DMSO (10 mL) was added 5-fluoropyridine-2-carbonitrile (compound 13.1, 500 mg, 4.10 mmol) and DIEA (1.43 mL, 8.19 mmol). The resultant mixture was stirred at 100° C. for 1 h. After being cooled to room temperature, the reaction mixture was diluted with water (100 mL), and extracted with EA (50 mL) twice. The combined organic layer was washed with brine, dried over Na₂SO₄ and concentrated. The residue was purified by RPLC to afford compound 25.1 (800 mg, 64.6% yield). MS: calc'd 303 [(M+H)⁺], measured 303 [(M+H)⁺].

### Step (b): Preparation of tert-butyl (3R)-3-[acetyl-(6-cyano-3-pyridyl)amino]piperidine-1-carboxylate (Compound 25.2)

To a solution of tert-butyl (3R)-3-[(6-cyano-3-pyridyl)amino]piperidine-1-carboxylate (compound 25.1, 100.0 mg, 0.32 mmol) in THF (5 mL) was added dropwise n-BuLi (2.5 Min hexanes, 0.2 mL, 0.5 mmol) at −40° C. and stirred at same temperature for 0.5 h. Then, a solution of acetic anhydride (39.7 mg, 0.39 mmol) in THF (1 mL) was added, the reaction mixture was warmed to room temperature, and stirred at this temperature for another 2 hrs. The reaction was quenched with NH₄Cl (1N, 30 mL), and extracted with EA (30 mL) twice. The combined organic layer was washed with brine, dried over Na₂SO₄, filtered and concentrated. The residue was purified by flash chromatography (silica gel, 12 g, 10% to 40% EA in PE) to afford compound 25.3 (100 mg, 90.8% yield). MS: calc'd 345 [(M+H)⁺], measured 367 [(M+Na)⁺].

### Step (c): Preparation of N-(6-cyano-3-pyridyl)-N-[(3R)-1-[3-ethyl-7-(ethylamino)-5-fluoro-2-oxo-indolin-3-yl]-3-piperidyl]acetamide (Example 25)

N-(6-cyano-3-pyridyl)-N-[(3R)-1-[3-ethyl-7-(ethylamino)-5-fluoro-2-oxo-indolin-3-yl]-3-piperidyl]acetamide (Example 25) was prepared in analogy to Example 8, by replacing tert-butyl (3R)-3-(4-cyanoanilino)piperidine-1-carboxylate (compound 8.1) with tert-butyl (3R)-3-[acetyl-(6-cyano-3-pyridyl)amino]piperidine-1-carboxylate (compound 25.2), and tert-butyl N-(3-bromo-5-fluoro-3-methyl-2-oxo-indolin-7-yl)-N-ethyl-carbamate (intermediate A) with tert-butyl N-(3-bromo-3-ethyl-5-fluoro-2-oxo-indolin-7-yl)-N-ethyl-carbamate (Intermediate B) in step (b). MS: calc'd 465 [(M+H)⁺], measured 465 [(M+H)⁺]. ¹H NMR (400 MHz, METHANOL-d₄) δ=8.69 (s, 0.5H), 8.64 (s, 0.5H), 8.04-7.94 (m, 2H), 6.55-6.46 (m, 2H), 3.78-3.70 (m, 1H), 3.26-3.13 (m, 3H), 2.94-2.55 (m, 2H), 2.27-2.09 (m, 2H), 1.94-1.70 (m, 7H), 1.30 (t, J=7.2 Hz, 3H), 1.23-1.09 (m, 1H), 0.73-0.68 (m, 3H).

### Example 26

### 1-(6-cyano-3-pyridyl)-1-[(3R)-1-[3-ethyl-7-(ethylamino)-5-fluoro-2-oxo-indolin-3-yl]-3-piperidyl]urea

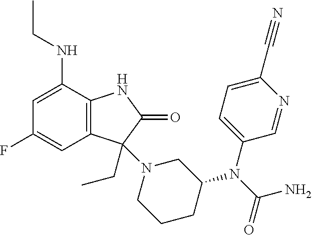 [SMILES: CCNc1cc(F)cc2c1NC(=O)C2(CC)N1CCC[C@@H](N(C(N)=O)c2ccc(C#N)nc2)C1]

The titled compound was synthesized according to the following scheme:

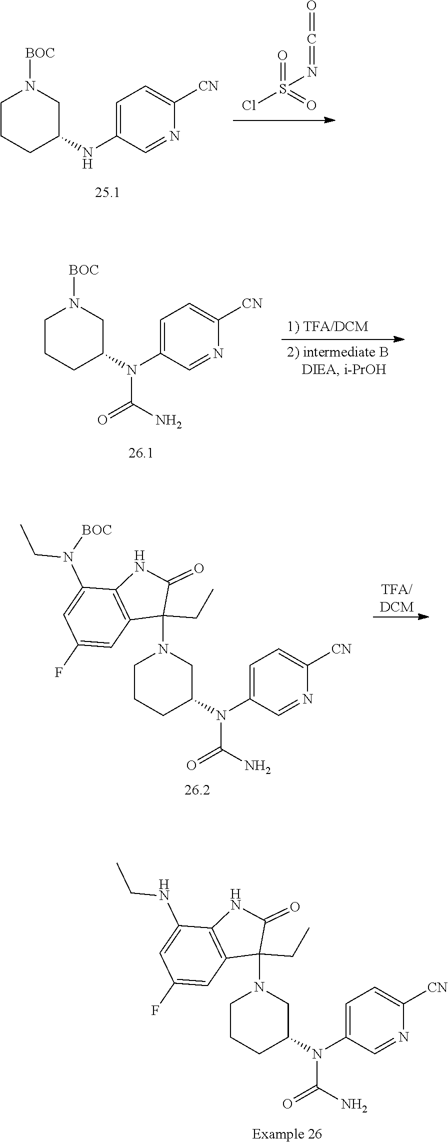 [SMILES: CC(C)(C)OC(=O)N1CCC[C@@H](Nc2ccc(C#N)nc2)C1 ; O=C=NS(=O)(=O)Cl ; CC(C)(C)OC(=O)N1CCC[C@@H](N(C(N)=O)c2ccc(C#N)nc2)C1 ; CCN(C(=O)OC(C)(C)C)c1cc(F)cc2c1NC(=O)C2(CC)N1CCC[C@@H](N(C(N)=O)c2ccc(C#N)nc2)C1 ; CCNc1cc(F)cc2c1NC(=O)C2(CC)N1CCC[C@@H](N(C(N)=O)c2ccc(C#N)nc2)C1]

### Step (a): Preparation of tert-butyl (3R)-3-[carbamoyl-(6-cyano-3-pyridyl)amino]piperidine-1-carboxylate (Compound 26.1)

To a solution of N-(oxomethylene)sulfamoyl chloride (458.71 mg, 3.24 mmol) in THF (4 mL) and EA (4 mL) was added tert-butyl (3R)-3-[(6-cyano-3-pyridyl)amino]piperidine-1-carboxylate (compound 25.1, 700.0 mg, 2.32 mmol) at −10° C. After being stirred for 10 min, water (4 mL) was added, the resultant mixture was warmed up to room temperature and stirred at this temperature for another 20 min. The reaction mixture was concentrated to afford a crude product, which was purified by RPLC to afford compound 26.1 (500 mg, 62.5% yield). MS: calc'd 346 [(M+H)⁺], measured 246 [(M-Boc+H)⁺].

### Step (b): Preparation of 1-(6-cyano-3-pyridyl)-1-[(3R)-1-[3-ethyl-7-(ethylamino)-5-fluoro-2-oxo-indolin-3-yl]-3-piperidyl]urea (Example 26)

1-(6-cyano-3-pyridyl)-1-[(3R)-1-[3-ethyl-7-(ethylamino)-5-fluoro-2-oxo-indolin-3-yl]-3-piperidyl]urea (Example 26) was prepared in analogy to Example 8, by replacing tert-butyl (3R)-3-(4-cyanoanilino)piperidine-1-carboxylate (compound 8.1) with tert-butyl (3R)-3-[carbamoyl-(6-cyano-3-pyridyl)amino]piperidine-1-carboxylate (compound 26.1) and tert-butyl N-(3-bromo-5-fluoro-3-methyl-2-oxo-indolin-7-yl)-N-ethyl-carbamate (intermediate A) with tert-butyl N-(3-bromo-3-ethyl-5-fluoro-2-oxo-indolin-7-yl)-N-ethyl-carbamate (intermediate B) in step (b). MS: calc'd 466 [(M+H)⁺], measured 466 [(M+H)⁺]. ¹H NMR (400 MHz, METHANOL-d₄) δ=8.67 (s, 0.5H), 8.60 (s, 0.5H), 8.02-7.91 (m, 2H), 6.59-6.50 (m, 2H), 4.70-4.59 (m, 1H), 3.90-3.86 (m, 0.5H), 3.79-3.72 (m, 0.5H), 3.26-3.13 (m, 3H), 3.13-2.89 (m, 1H), 2.72-2.62 (m, 1H), 2.35-2.14 (m, 2H), 1.98-1.74 (m, 3H), 1.32-1.21 (m, 4H), 0.73-0.69 (m, 3H).

### Example 27

### 3-[[(3R)-1-[7-(ethylamino)-5-fluoro-3-methyl-2-oxo-indolin-3-yl]-3-piperidyl]amino]benzenesulfonyl fluoride

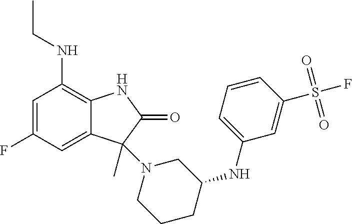 [SMILES: CCNc1cc(F)cc2c1NC(=O)C2(C)N1CCC[C@@H](Nc2cccc(S(=O)(=O)F)c2)C1]

The titled compound was synthesized according to the following scheme:

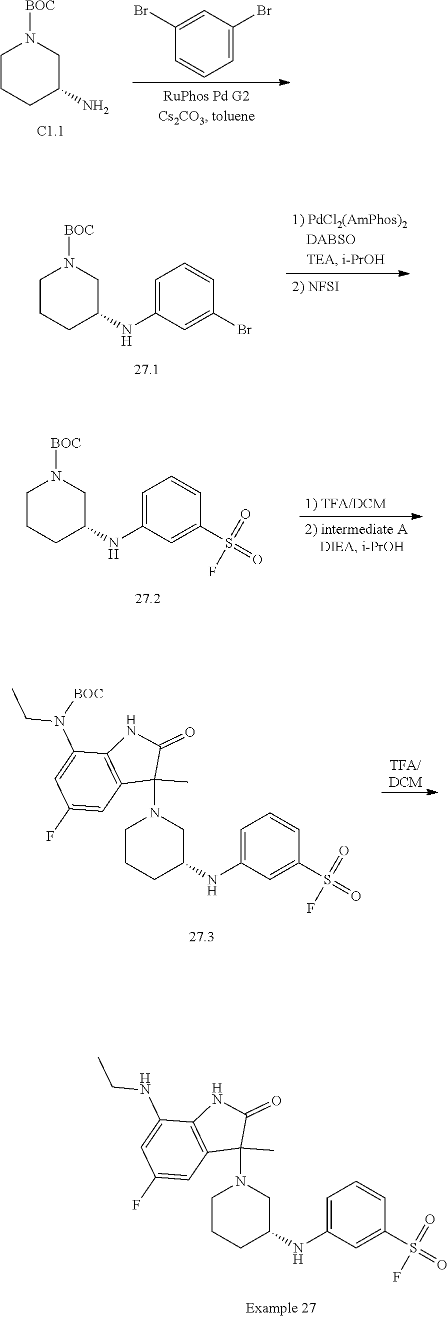 [SMILES: CC(C)(C)OC(=O)N1CCC[C@@H](N)C1 ; Brc1cccc(Br)c1 ; CC(C)(C)OC(=O)N1CCC[C@@H](Nc2cccc(Br)c2)C1 ; CC(C)(C)OC(=O)N1CCC[C@@H](Nc2cccc(S(=O)(=O)F)c2)C1 ; CCN(C(=O)OC(C)(C)C)c1cc(F)cc2c1NC(=O)C2(C)N1CCC[C@@H](Nc2cccc(S(=O)(=O)F)c2)C1 ; CCNc1cc(F)cc2c1NC(=O)C2(C)N1CCC[C@@H](Nc2cccc(S(=O)(=O)F)c2)C1]

### Step (a): Preparation of tert-butyl (3R)-3-(3-bromoanilino)piperidine-1-carboxylate (Compound 27.1)

To a solution of 1,3-dibromobenzene (3.54 g, 15.01 mmol) and tert-butyl (3R)-3-aminopiperidine-1-carboxylate (compound C1.1, 2.0 g, 9.99 mmol) in toluene (10 mL) was added cesium carbonate (6.60 g, 20.26 mmol) and Ruphs-Pd-G2 (1.54 g, 1.99 mmol), the resultant mixture was stirred at 110° C. for 12 hrs. After being cooled to room temperature, the reaction mixture was diluted with water (50 mL) and extracted with EA (50 mL) for three times. The combined organic layer was washed with brine, dried over Na₂SO₄, filtered and concentrated in vacuo. The residue was purified by RPLC to afford compound 27.1 (220 mg, 6.2% yield). MS: calc'd 355 [(M+H)⁺]; measured 355 [(M+H)⁺].

### Step (b): Preparation of tert-butyl (3R)-3-(3-fluorosulfonylanilino)piperidine-1-carboxylate (Compound 27.2)

To a solution of tert-butyl (3R)-3-(3-bromoanilino)piperidine-1-carboxylate (compound 27.1, 220.0 mg, 0.62 mmol) in isopropanol (2 mL) was added DABSO (90.0 mg, 0.37 mmol), trimethylamine (190.0 mg, 1.88 mmol) and PdCl₂(Amphos)₂ (44.0 mg, 0.060 mmol), the resultant mixture was stirred at 75° C. for 12 hrs. After being cooled to room temperature, NFSI (294.0 mg, 0.93 mmol) was added, and then the reaction mixture was stirred at room temperature for another 3 hrs. The reaction mixture was concentrated to afford a crude product, which was purified by RPLC to afford compound 27.2 (110 mg, 49.5% yield). MS: calc'd 359 [(M+H)⁺]; measured 303 [(M−55)⁺].

### Step (c): Preparation of 3-[[(3R)-1-[7-(ethylamino)-5-fluoro-3-methyl-2-oxo-indolin-3-yl]-3-piperidyl]amino]benzenesulfonyl fluoride (Example 27)

3-[[(3R)-1-[7-(ethylamino)-5-fluoro-3-methyl-2-oxo-indolin-3-yl]-3-piperidyl]amino]benzenesulfonyl fluoride (Example 27) was prepared in analogy to Example 8, by replacing tert-butyl (3R)-3-(4-cyanoanilino)piperidine-1-carboxylate (compound 8.1) with tert-butyl (3R)-3-(3-fluorosulfonylanilino)piperidine-1-carboxylate (compound 27.2) in step (b). MS: calc'd 465 [(M+H)⁺], measured 465 [(M+H)⁺]. ¹H NMR (400 MHz, DMSO-d₆) δ=10.47-10.37 (m, 1H), 7.46 (t, J=8.0 Hz, 1H), 7.22-7.12 (m, 2H), 7.06-7.04 (m, 1H), 6.66-6.64 (m, 1H), 6.56-6.54 (m, 1H), 6.45-6.38 (m, 1H), 3.74-3.64 (m, 1H), 3.42-2.99 (m, 4H), 2.74-2.59 (m, 2H), 1.92-1.83 (m, 2H), 1.76-1.64 (m, 1H), 1.59-1.55 (m, 3H), 1.44-1.30 (m, 1H), 1.18 (t, J=7.2 Hz, 3H).

### Example 28

### 4-[[(3R)-1-[7-(ethylamino)-5-fluoro-3-methyl-2-oxo-indolin-3-yl]-3-piperidyl]amino]-2-methoxy-benzenesulfonyl fluoride

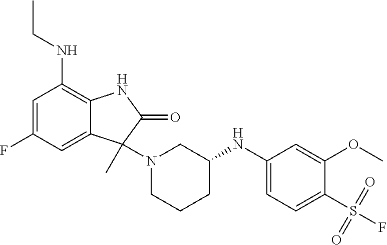 [SMILES: CCNc1cc(F)cc2c1NC(=O)C2(C)N1CCC[C@@H](Nc2ccc(S(=O)(=O)F)c(OC)c2)C1]

The titled compound was synthesized according to the following scheme:

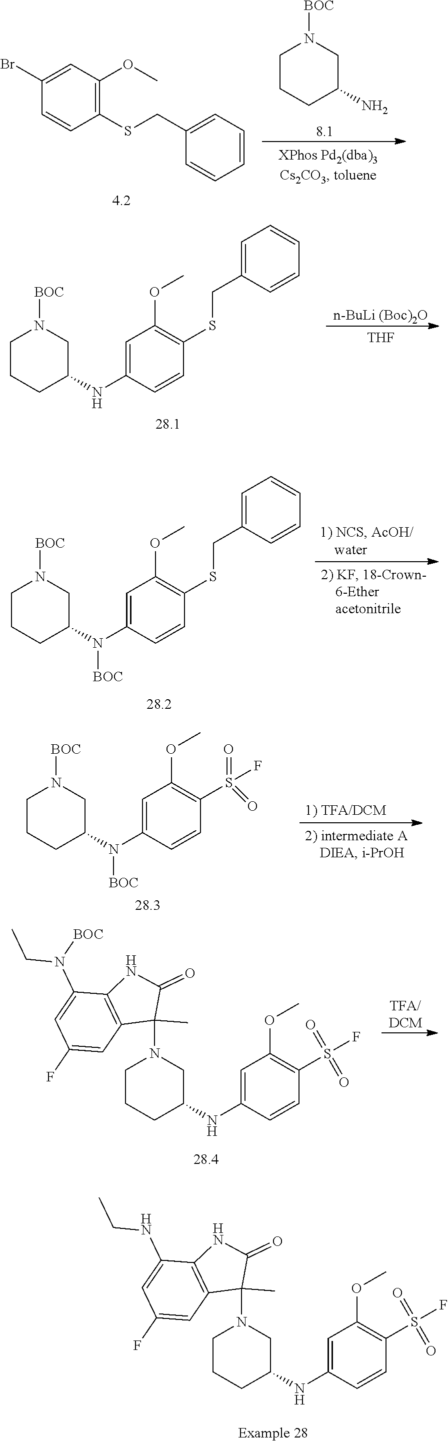

### Step (a): Preparation of tert-butyl (3R)-3-(4-benzylsulfanyl-3-methoxy-anilino)piperidine-1-carboxylate (Compound 28.1)

To a solution of tert-butyl (3R)-3-aminopiperidine-1-carboxylate (compound C1.1, 325 mg, 1.60 mmol) in toluene (25 mL) was added 1-benzylsulfanyl-4-bromo-2-methoxy-benzene (compound 4.2, 500 mg, 1.60 mmol), Pd₂(dba)₃ (297 mg, 0.3 mmol), XPhos (309 mg, 0.7 mmol) and cesium carbonate (1.6 g, 4.9 mmol). The resultant mixture was stirred at 110° C. for 16 hrs. After being cooled to room temperature, the reaction mixture was diluted with water (100 mL) and extracted with EA (50 mL) for three times. The combined organic layer was washed with brine, dried over Na₂SO₄, filtered and concentrated in vacuo. The residue was purified by flash chromatography (silica gel, 20 g, 10% to 20% EA in PE) to afford compound 28.1 (450 mg, 65.7% yield). MS: calculated 429 [(M+H)⁺], measured 429 [(M+H)⁺].

### Step (b): Preparation of tert-butyl (3R)-3-(4-benzylsulfanyl-N-tert-butoxycarbonyl-3-methoxy-anilino)piperidine-1-carboxylate (Compound 28.2)

To a solution of tert-butyl (3R)-3-(4-benzylsulfanyl-3-methoxy-anilino)piperidine-1-carboxylate (compound 28.1, 550 mg, 1.3 mmol) in THF (15 mL) was added n-BuLi (2.5 M in hexanes, 0.8 mL, 2 mmol) at −40° C. After the reaction mixture was stirred at 0° C. for 0.5 hrs., di-tert-butyl dicarbonate (370 mg, 1.7 mmol) was added, and the resultant mixture was stirred at 0° C. for another 3 hrs. The reaction was quenched with sat.NH₄Cl (aq, 20 mL), and extracted with EA (50 mL) for three times. The combined organic layer was washed with brine, dried over Na₂SO₄, filtered and concentrated in vacuo. The residue was purified by flash chromatography (silica gel, 20 g, 15% to 25% EA in PE) to afford compound 28.2 (374 mg, 52.3% yield). MS: calc'd 551 [(M+H)⁺], measured 551 [(M+H)⁺].

### Step (c): Preparation of tert-butyl (3R)-3-(N-tert-butoxycarbonyl-4-fluorosulfonyl-3-methoxy-anilino)piperidine-1-carboxylate (Compound 28.3)

To a solution of tert-butyl (3R)-3-(4-benzylsulfanyl-N-tert-butoxycarbonyl-3-methoxy-anilino)piperidine-1-carboxylate (compound 28.2, 200 mg, 0.40 mmol) in acetic acid (1.5 mL) and water (0.5 mL) was added NCS (152 mg, 1.1 mmol) at 0° C. The resultant mixture was stirred at this temperature for 7 hrs. The reaction mixture was diluted with EA (100 mL), washed with water and brine, dried over Na₂SO₄, filtered and concentrated in vacuo to afford an oil. To a solution of the residue in acetonitrile (5 mL) was added 18-crown-6 (105 mg, 0.40 mmol) and potassium fluoride (67 mg, 1.2 mmol). After being stirred at room temperature for 16 hrs., the reaction mixture was diluted with EA (100 mL), washed with water (50 mL) and brine (50 mL), dried over Na₂SO₄, filtered and concentrated to afford compound 28.3 (180 mg, 76.0% yield) which was used directly for the next step without further purification. MS: calc'd 489 [(M+H)⁺], measured 389 [(M-Boc+H)⁺].

### Step (d): Preparation of 4-[[(3R)-1-[7-(ethylamino)-5-fluoro-3-methyl-2-oxo-indolin-3-yl]-3-piperidyl]amino]-2-methoxy-benzenesulfonyl fluoride (Example 28)

4-[[(3R)-1-[7-(ethylamino)-5-fluoro-3-methyl-2-oxo-indolin-3-yl]-3-piperidyl]amino]-2-methoxy-benzenesulfonyl fluoride (Example 28) was prepared in analogy to Example 8, by replacing tert-butyl (3R)-3-(4-cyanoanilino)piperidine-1-carboxylate (compound 8.1) with tert-butyl (3R)-3-(N-tert-butoxycarbonyl-4-fluorosulfonyl-3-methoxy-anilino)piperidine-1-carboxylate (compound 28.3) in step (b). MS: calc'd 495 [(M+H)⁺], measured 495 [(M+H)⁺]. ¹H NMR (400 MHz, DMSO-d₆) δ=9.85 (s, 1H), 7.48 (d, J=9.2 Hz, 0.4H), 7.43 (d, J=9.2 Hz, 0.6H), 7.00-6.95 (m, 1H), 6.39-6.34 (m, 1H), 6.30-6.20 (m, 2H), 5.13-5.09 (m, 1H), 3.89 (s, 1.2H), 3.81 (s, 1.8H), 3.48-3.38 (m, 1H), 3.08-3.02 (m, 2H), 2.92-2.86 (m, 1H), 2.82-2.80 (m, 1H), 2.29-2.24 (m, 0.6H), 2.12-2.07 (m, 0.4H), 2.00-1.96 (m, 1H), 1.87-1.84 (m, 1H), 1.70-1.85 (m, 1H), 1.51-1.45 (m, 1H), 1.36 (s, 3H), 1.27-1.22 (m, 1H), 1.20-1.15 (m, 3H).

### Example 29

### 6-[[(3R)-1-[7-(ethylamino)-5-fluoro-3-methyl-2-oxo-indolin-3-yl]-3-piperidyl]amino]pyridine-3-sulfonyl fluoride

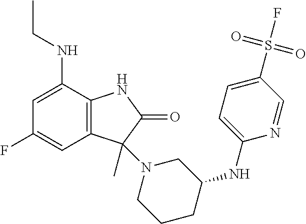 [SMILES: CCNc1cc(F)cc2c1NC(=O)C2(C)N1CCC[C@@H](Nc2ccc(S(=O)(=O)F)cn2)C1]

The titled compound was synthesized according to the following scheme:

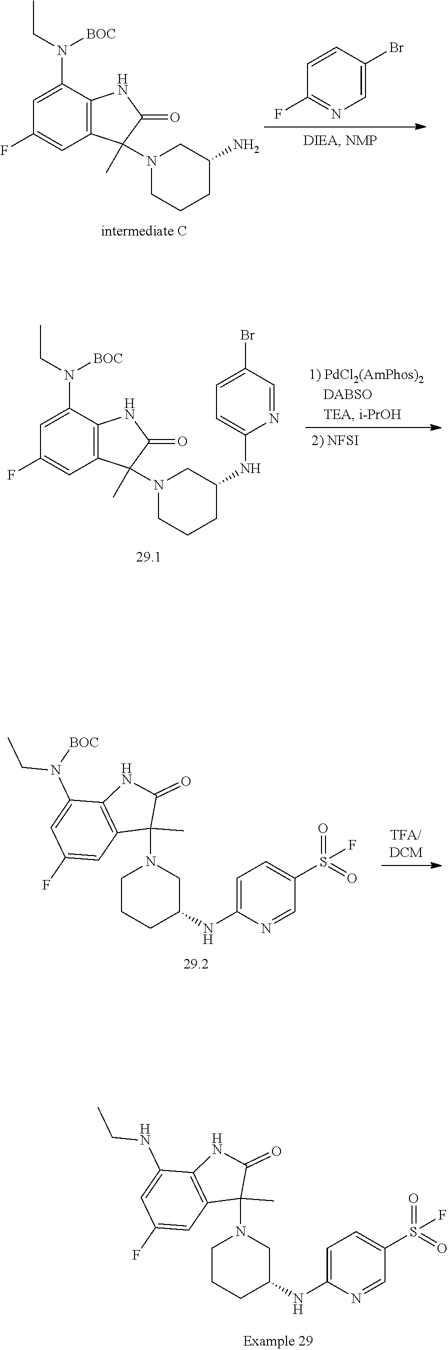 [SMILES: CCN(C(=O)OC(C)(C)C)c1cc(F)cc2c1NC(=O)C2(C)N1CCC[C@@H](N)C1 ; Fc1ccc(Br)cn1 ; CCN(C(=O)OC(C)(C)C)c1cc(F)cc2c1NC(=O)C2(C)N1CCC[C@@H](Nc2ccc(Br)cn2)C1 ; CCN(C(=O)OC(C)(C)C)c1cc(F)cc2c1NC(=O)C2(C)N1CCC[C@@H](Nc2ccc(S(=O)(=O)F)cn2)C1 ; CCNc1cc(F)cc2c1NC(=O)C2(C)N1CCC[C@@H](Nc2ccc(S(=O)(=O)F)cn2)C1]

### Step (a): Preparation of tert-butyl N-ethyl-N-[5-fluoro-3-methyl-2-oxo-3-[(3R)-3-[(5-bromo-2-pyridyl)amino]-1-piperidyl]indolin-7-yl]carbamate (Compound 29.1)

To a solution of tert-butyl N-ethyl-N-[5-fluoro-3-methyl-2-oxo-3-[(3R)-3-amino-1-piperidyl]indolin-7-yl]carbamate (intermediate C, 200.0 mg, 0.49 mmol) in NMP (3 mL) was added 5-bromo-2-fluoro-pyridine (150.0 mg, 0.85 mmol) and DIEA (0.3 mL, 1.72 mmol). The resultant mixture was heated at 155° C. for 6 hrs. After being cooled to room temperature, the reaction mixture was diluted with water (20 mL) and extracted with EA (50 mL) for three times. The combined organic layer was washed with brine, dried over Na₂SO₄, filtered and concentrated in vacuo. The residue was purified by prep-TLC (50% EA in PE, R_{f}=0.50) to afford compound 29.1 (100 mg, 36.2% yield). MS: calc'd 562, 564 [(M+H)⁺], measured 562, 564 [(M+H)⁺].

### Step (b): Preparation of tert-butyl N-ethyl-N-[5-fluoro-3-methyl-2-oxo-3-[(3R)-3-[(5-fluorosulfonyl-2-pyridyl)amino]-1-piperidyl]indolin-7-yl]carbamate (Compound 29.2)

To a solution of tert-butyl N-ethyl-N-[5-fluoro-3-methyl-2-oxo-3-[(3R)-3-[(5-bromo-2-pyridyl)amino]-1-piperidyl]indolin-7-yl]carbamate (compound 29.1, 100.0 mg, 0.18 mmol) in isopropanol (3 mL) was added PdCl₂(Amphos)₂ (7.0 mg, 0.010 mmol), DABSO (37.0 mg, 0.110 mmol) and triethylamine (0.1 mL, 0.72 mmol). The resultant mixture was stirred at 75° C. for 16 hrs. After being cooled to room temperature, NFSI (100.0 mg, 0.32 mmol) was added, and then the reaction mixture was stirred at room temperature for another 2 hrs. The reaction mixture was diluted with water (50 mL), extracted with EA (50 mL) twice. The combined organic layer was washed with brine, dried over Na₂SO₄, filtered and concentrated in vacuo. The residue was purified by prep-TLC (50% EA in PE, R_{f}=0.38) to afford compound 29.2 (60 mg, 58.9% yield). MS: calc'd 566 [(M+H)⁺], measured 566 [(M+H)⁺].

### Step (c): Preparation of 6-[[(3R)-1-[7-(ethylamino)-5-fluoro-3-methyl-2-oxo-indolin-3-yl]-3-piperidyl]amino]pyridine-3-sulfonyl fluoride (Example 29)

To a solution of tert-butyl N-ethyl-N-[5-fluoro-3-methyl-2-oxo-3-[(3R)-3-[(5-fluorosulfonyl-2-pyridyl)amino]-1-piperidyl]indolin-7-yl]carbamate (compound 29.2, 60.0 mg, 0.11 mmol) in DCM (10 mL) was added TFA (5 mL). The reaction mixture was stirred at room temperature for 2 hrs., then concentrated to afford a crude product, which was purified by pre-HPLC to afford Example 29 (35.6 mg, 69.6% yield). MS: calc'd 466 [(M+H)⁺], measured 466 [(M+H)⁺]. ¹H NMR (400 MHz, METHANOL-d₄) δ=8.57 (s, 1H), 7.89-7.85 (m, 1H), 6.69-6.58 (m, 2H), 6.53-6.47 (m, 1H), 4.42-4.41 (m, 1H), 3.70-3.69 (m, 1H), 3.49-3.43 (m, 1H), 3.19-3.13 (m, 3H), 2.96-2.95 (m, 1H), 2.12-2.04 (m, 2H), 1.96-1.92 (m, 1H), 1.77 (s, 3H), 1.64-1.62 (m, 1H), 1.28 (t, J=7.2 Hz, 3H).

### Example 30

### 4-methyl-6-[[(3R)-1-[7-(ethylamino)-5-fluoro-3-methyl-2-oxo-indolin-3-yl]-3-piperidyl]amino]pyridine-3-sulfonyl fluoride

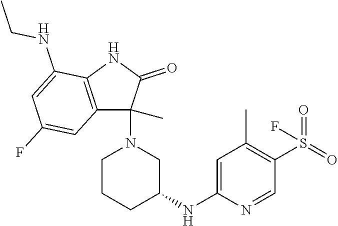 [SMILES: CCNc1cc(F)cc2c1NC(=O)C2(C)N1CCC[C@@H](Nc2cc(C)c(S(=O)(=O)F)cn2)C1]

The titled compound was synthesized according to the following scheme:

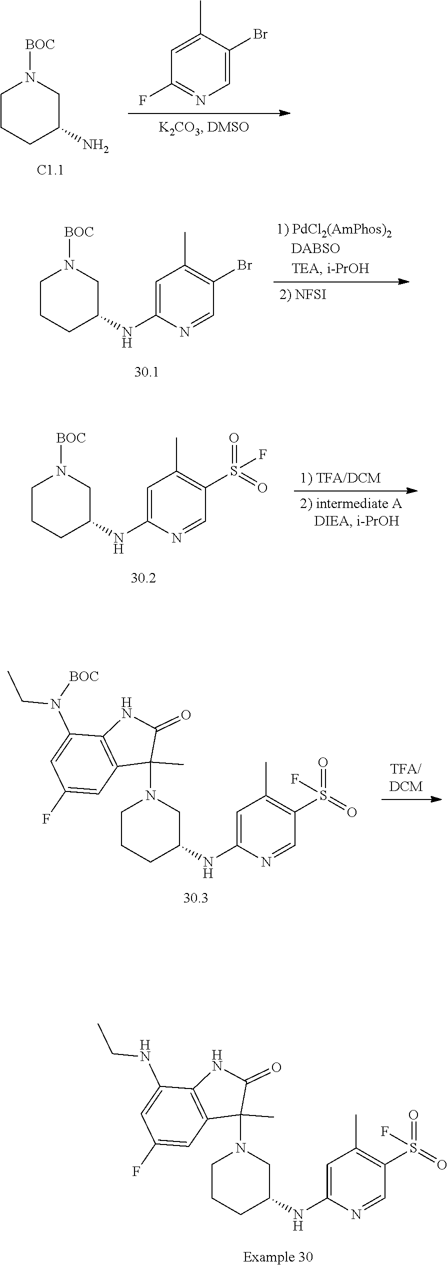

### Step (a): Preparation of tert-butyl (3R)-3-[(5-bromo-4-methyl-2-pyridyl)amino]piperidine-1-carboxylate (Compound 30.1)

A mixture of tert-butyl (3R)-3-aminopiperidine-1-carboxylate (compound C1.1, 6.0 g, 29.96 mmol), 5-bromo-2-fluoro-4-methyl-pyridine (4.0 g, 21.05 mmol) and potassium carbonate (8.80 g, 63.67 mmol) in DMSO (40 mL) was stirred at 120° C. for 16 hrs. After being cooled to room temperature, the reaction mixture was diluted with water (300 mL) and extracted with EA (50 mL) for three times. The combined organic layer was washed with brine, dried over Na₂SO₄, filtered and concentrated in vacuo. The residue was purified by flash chromatography (silica gel, 120 g, 5% to 10% EA in PE) to afford compound 30.1 (3.60 g, 46.3% yield). MS: calc'd 370, 372 [(M+H)⁺]; measured 370, 372 [(M+H)⁺].

### Step (b): Preparation of 4-methyl-6-[[(3R)-1-[7-(ethylamino)-5-fluoro-3-methyl-2-oxo-indolin-3-yl]-3-piperidyl]amino]pyridine-3-sulfonyl fluoride (Example 30)

4-methyl-6-[[(3R)-1-[7-(ethylamino)-5-fluoro-3-methyl-2-oxo-indolin-3-yl]-3-piperidyl]amino]pyridine-3-sulfonyl fluoride (Example 30) was prepared in analogy to Example 27, by replacing tert-butyl (3R)-3-(3-bromoanilino)piperidine-1-carboxylate (compound 27.1) with tert-butyl (3R)-3-[(5-bromo-4-methyl-2-pyridyl)amino]piperidine-1-carboxylate (compound 30.1) in step (b). MS: calc'd 480 [(M+H)⁺], measured 480 [(M+H)⁺]. ¹H NMR (400 MHz, DMSO-d₆) δ=10.64-10.29 (m, 1H), 8.48-8.47 (m, 1H), 8.00-7.94 (m, 1H), 6.62-6.60 (m, 2H), 6.52 (s, 1H), 6.39 (d, J=12.0 Hz, 1H), 4.42-4.13 (m, 1H), 3.37-3.19 (m, 2H), 3.08 (q, J=6.8 Hz, 2H), 2.84-2.57 (m, 2H), 2.40 (s, 3H), 1.92-1.81 (m, 1H), 1.74-1.65 (m, 1H), 1.58 (s, 1.5H), 1.57 (s, 1.5H), 1.45-1.39 (m, 1H), 1.27-1.23 (m, 1H), 1.20-1.17 (m, 3H).

### Example 31

### 7-(ethylamino)-5-fluoro-3-methyl-2-oxo-3-[(3R)-3-(3-fluorosulfonyloxyanilino)-1-piperidyl]indoline

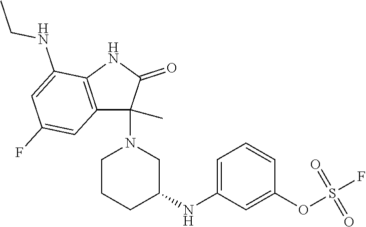 [SMILES: CCNc1cc(F)cc2c1NC(=O)C2(C)N1CCC[C@@H](Nc2cccc(OS(=O)(=O)F)c2)C1]

The titled compound was synthesized according to the following scheme:

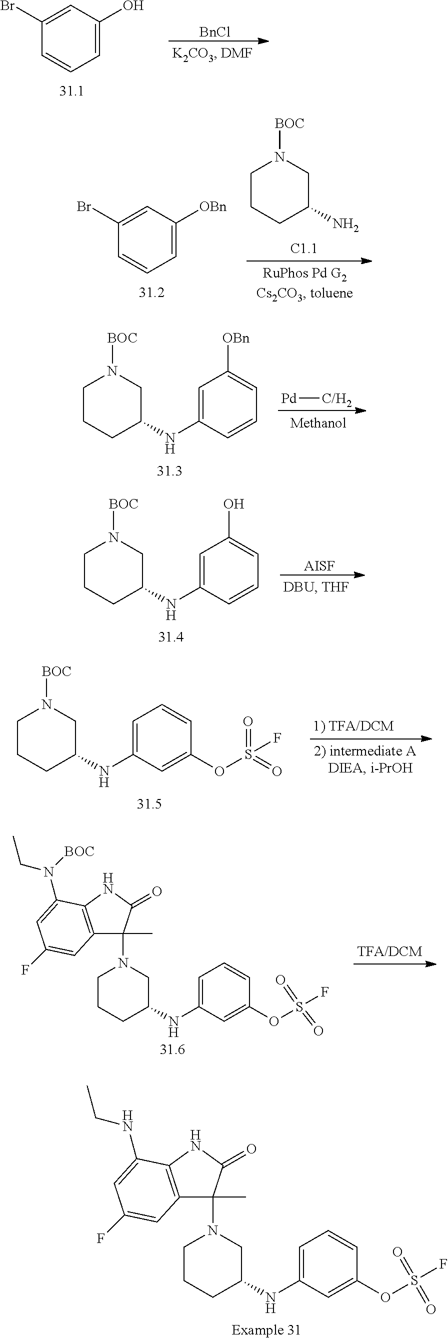

### Step (a): Preparation of 1-benzyloxy-3-bromo-benzene (Compound 31.2)

To a solution of 3-bromophenol (compound 31.1, 2.0 g, 11.56 mmol) in DMF (20 mL) was added K₂CO₃ (4.8 g, 34.68 mmol) and (chloromethyl)benzene (2.9 g, 23.12 mmol). The resultant mixture was stirred at 50° C. for 6 hrs. After being cooled to room temperature, the reaction mixture was diluted with water (100 mL) and extracted with EA (50 mL) for three times. The combined organic layer was washed with brine, dried over Na₂SO₄, filtered and concentrated in vacuo. The residue was recrystallized with PE to afford Example 31.2 (2.4 g, 9.12 mmol, 76.47% yield). ¹H NMR (400 MHz, CHLOROFORM-d) δ=7.47-7.31 (m, 5H), 7.19-7.07 (m, 3H), 6.93-6.91 (m, 1H), 5.06 (s, 2H).

### Step (b): Preparation of tert-butyl (3R)-3-(3-benzyloxyanilino)piperidine-1-carboxylate (Compound 31.3)

To a solution of 1-benzyloxy-3-bromo-benzene (compound 31.2, 1.5 g, 5.7 mmol) in toluene (30 mL) was added tert-butyl (3R)-3-aminopiperidine-1-carboxylate (compound C1.1, 1427 mg, 7.13 mmol), Cs₂CO₃ (5571 mg, 17.1 mmol) and RuPhos-Pd-G2 (886 mg, 1.14 mmol), the resultant mixture was stirred at 110° C. for 16 hrs. After being cooled to room temperature, the reaction mixture was diluted with water (50 mL), and extracted with EA (30 mL) for three times. The combined organic layer was washed with brine, dried over Na₂SO₄, filtered and concentrated in vacuo. The residue was purified by flash chromatography (silica gel, 120 g, 0% to 50% EA in PE) to afford compound 31.3 (1.7 g, 78.0% yield). MS: calc'd 383 [(M+H)⁺], measured 383 [(M+H)⁺].

### Step (c): Preparation of tert-butyl (3R)-3-(3-hydroxyanilino)piperidine-1-carboxylate (Compound 31.4)

A mixture of tert-butyl (3R)-3-(3-benzyloxyanilino)piperidine-1-carboxylate (compound 31.3, 430 mg, 1.12 mmol) and Pd—C (100 mg) in MeOH (50 mL) was hydrogenated by a hydrogen balloon at room temperature for 2 hrs. After the catalyst was filtered off, the filtrate was concentrated in vacuo to afford compound 31.4 (328 mg, crude). MS: calc'd 293 [(M+H)⁺], measured 293 [(M+H)⁺].

### Step (d): Preparation of tert-butyl (3R)-3-(3-fluorosulfonyloxyanilino)piperidine-1-carboxylate (Compound 31.5)

To a solution of tert-butyl (3R)-3-(3-hydroxyanilino)piperidine-1-carboxylate (compound 31.4, 328 mg, 1.12 mmol) and AISF (CAS: 2172794-56-6, Sigma-Aldrich, Catalog: 901243, 528 mg, 1.68 mmol) in THF (3 mL) was added DBU (682 mg, 4.49 mmol), the resultant mixture was stirred at room temperature for 10 min. The reaction was quenched with water (10 mL), and extracted with EA (10 mL) for three times. The combined organic layer was washed with brine, dried over Na₂SO₄, filtered and concentrated in vacuo. The residue was purified by flash chromatography (silica gel, 40 g, 10% to 50% EA in PE) to afford compound 31.5 (164 mg, 0.440 mmol, 39.0% yield). MS: calc'd 375 [(M+H)⁺], measured 375 [(M+H)⁺].

### Step (e): Preparation of tert-butyl N-ethyl-N-[5-fluoro-3-methyl-2-oxo-3-[(3R)-3-(3-fluorosulfonyloxyanilino)-1-piperidyl]indolin-7-yl]carbamate (Compound 31.6)

To a solution of tert-butyl (3R)-3-(3-fluorosulfonyloxyanilino)piperidine-1-carboxylate (compound 31.5, 80 mg, 0.210 mmol) in DCM (3 mL) was added TFA (1 mL). The reaction mixture was stirred at room temperature for 2 hrs, then concentrated to afford an oil. To a solution of the residue and DIEA (0.1 mL, 0.630 mmol) in isopropanol (5 mL) was added tert-butyl N-(3-bromo-5-fluoro-3-methyl-2-oxo-indolin-7-yl)-N-ethyl-carbamate (intermediate A, 82 mg, 0.21 mmol). The reaction mixture was stirred at room temperature for 2 hrs, then concentrated to afford a crude product, which was purified by flash chromatography (silica gel, 24 g, 10% to 100% EA in PE) to afford compound 31.6 (80 mg, 65.6% yield). MS: calc'd 581 [(M+H)⁺], measured 581 [(M+H)⁺].

### Step (f): Preparation of 7-(ethylamino)-5-fluoro-3-methyl-2-oxo-3-[(3R)-3-(3-fluorosulfonyloxyanilino)-1-piperidyl]indoline (Example 31)

To a solution of tert-butyl N-ethyl-N-[5-fluoro-3-methyl-2-oxo-3-[(3R)-3-(3-fluorosulfonyloxyanilino)-1-piperidyl]indolin-7-yl]carbamate (compound 31.6, 80 mg, 0.14 mmol) in DCM (10 mL) was added TFA (5 mL). The reaction mixture was stirred at room temperature for 2 hrs, then concentrated to afford a crude product, which was purified by prep-HPLC to afford Example 31 (33.7 mg, 50.1% yield). MS: calc'd 481 [(M+H)⁺], measured 481 [(M+H)⁺]. ¹H NMR (400 MHz, METHANOL-d₄) δ=7.24 (dt, J=8.4, 2 Hz, 1H), 6.70-6.59 (m, 4H), 6.52-6.47 (m, 1H), 3.79-3.72 (m, 1H), 3.63-3.53 (m, 1H), 3.47-3.39 (m, 1H), 3.19-3.13 (m, 2H), 3.11-2.96 (m, 1H), 2.84-2.79 (m, 1H), 2.13-2.03 (m, 2H), 1.96-1.86 (m, 1H), 1.76 (s, 3H), 1.55-1.44 (m, 1H), 1.28 (t, J=7.2 Hz, 1.5H), 1.27 (t, J=7.2 Hz, 1.5H).

### Example 32

### 7-(ethylamino)-5-fluoro-3-methyl-2-oxo-3-[(3R)-3-(4-fluorosulfonyloxyanilino)-1-piperidyl]indoline

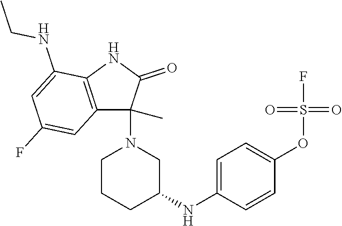 [SMILES: CCNc1cc(F)cc2c1NC(=O)C2(C)N1CCC[C@@H](Nc2ccc(OS(=O)(=O)F)cc2)C1]

7-(ethylamino)-5-fluoro-3-methyl-2-oxo-3-[(3R)-3-(4-fluorosulfonyloxyanilino)-1-piperidyl]indoline (Example 32) was prepared in analogy to Example 31, by replacing 3-bromophenol (compound 31.1) with 4-bromophenol in step (a). MS: calc'd 481 [(M+H)⁺], measured 481 [(M+H)⁺]. ¹H NMR (400 MHz, METHANOL-d₄) δ=7.11 (d, J=9.2 Hz, 1H), 7.04 (d, J=8.8 Hz, 1H), 6.66 (d, J=9.2 Hz, 1H), 6.56 (d, J=9.2 Hz, 1H), 6.45 (dd, J=8.0, 2.4 Hz, 0.5H), 6.40 (dd, J=8.0, 2.0 Hz, 0.5H), 6.33 (dd, J=4.4, 2.4 Hz, 0.5H), 6.30 (dd, J=4.4, 2.4 Hz, 0.5H), 3.52-3.47 (m, 0.5H), 3.40-3.35 (m, 0.5H), 3.16-3.09 (m, 2H), 2.99-2.91 (m, 1H), 2.84-2.81 (m, 0.5H), 2.70-2.60 (m, 1H), 2.47-2.39 (m, 1H), 2.12 (t, J=8.8 Hz, 0.5H), 1.85-1.70 (m, 2H), 1.64-1.50 (m, 1H), 1.46 (s, 1.5H), 1.45 (s, 1.5H), 1.42-1.37 (m, 1H), 1.29-1.24 (m, 3H).

### Example 33

### 7-(ethylamino)-5-fluoro-3-methyl-2-oxo-3-[(3R)-3-[(6-fluorosulfonyloxy-3-pyridyl)amino]-1-piperidyl]indoline

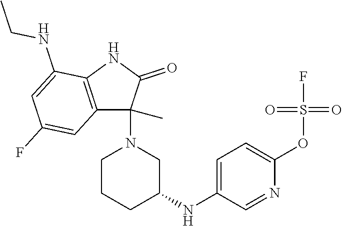 [SMILES: CCNc1cc(F)cc2c1NC(=O)C2(C)N1CCC[C@@H](Nc2ccc(OS(=O)(=O)F)nc2)C1]

7-(ethylamino)-5-fluoro-3-methyl-2-oxo-3-[(3R)-3-[(6-fluorosulfonyloxy-3-pyridyl)amino]-1-piperidyl]indoline (Example 33) was prepared in analogy to Example 31, by replacing 1-benzyloxy-3-bromo-benzene (compound 31.2) with 2-benzyloxy-5-iodo-pyridine in step (b). MS: calc'd 482 [(M+H)⁺], measured 482 [(M+H)⁺]. ¹H NMR (400 MHz, METHANOL-d₄) δ=7.69 (d, J=2.8 Hz, 0.3H), 7.67 (d, J=2.8 Hz, 0.7H), 7.20 (d, J=3.2 Hz, 0.3H), 7.17 (d, J=2.8 Hz, 0.7H), 7.15-7.13 (m, 1H), 6.63 (dd, J=8.0, 2.0 Hz, 0.3H), 6.57 (dd, J=8.0, 2.0 Hz, 0.7H), 6.50-6.44 (m, 1H), 3.77-3.70 (m, 1H), 3.48-3.35 (m, 2H), 3.19-3.13 (m, 2H), 3.04-2.96 (m, 1H), 2.82-2.76 (m, 1H), 2.07-1.99 (m, 2H), 1.89-1.79 (m, 1H), 1.72 (s, 1H), 1.71 (s, 2H), 1.54-1.47 (m, 1H), 1.30-1.26 (m, 3H).

### Example 34

### 7-(ethylamino)-5-fluoro-3-methyl-3-[(3R)-3-(4-vinylsulfonylanilino)-1-piperidyl]indolin-2-one

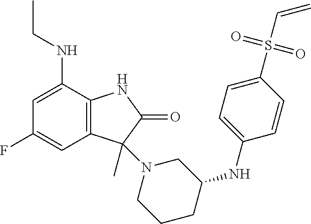 [SMILES: C=CS(=O)(=O)c1ccc(N[C@@H]2CCCN(C3(C)C(=O)Nc4c(NCC)cc(F)cc43)C2)cc1]

The titled compound was synthesized according to the following scheme:

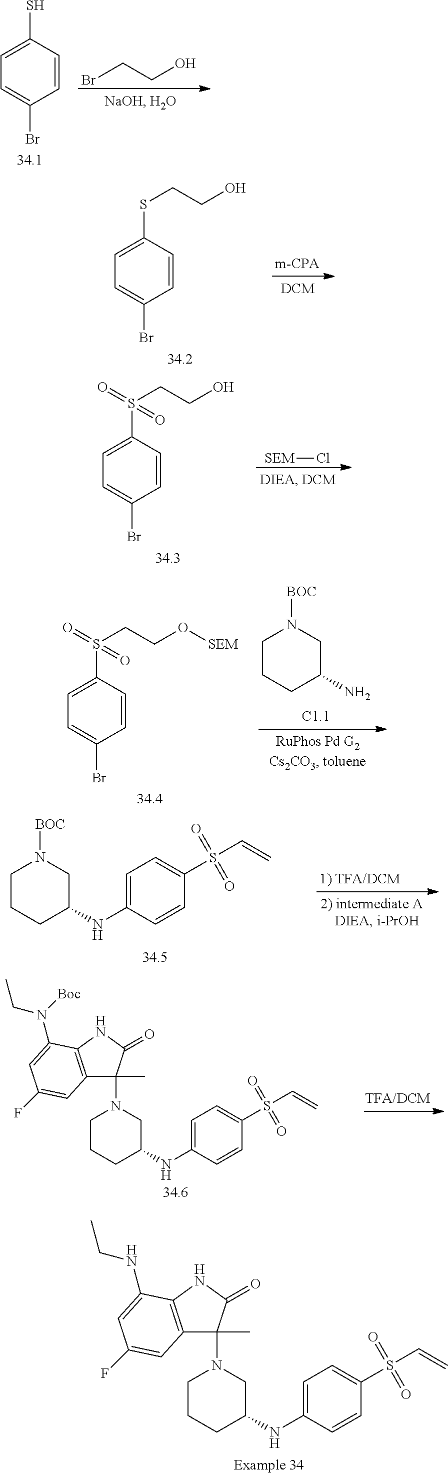

### Step (a): Preparation of 2-(4-bromophenyl)sulfanylethanol (Compound 34.2)

To a solution of 4-bromobenzenethiol (compound 34.1, 1.0 g, 5.29 mmol) and NaOH (1.06 g, 26.45 mmol) in water (15 mL) was added 2-bromoethanol (728 mg, 5.8 mmol) at 0° C., the resultant mixture was warmed to room temperature, and stirred at this temperature for 16 hrs. The reaction mixture was diluted with water (30 mL), extracted with EA (50 mL) twice. The combined organic layer was washed with brine, dried over Na₂SO₄, filtered and concentrated to afford compound 34.2 (1.2 g, 97.3% yield) which was used directly for the next. MS: calc'd 233, 235 [(M+H)⁺], measured 255, 257 [(M+Na)⁺].

### Step (b): Preparation of 2-(4-bromophenyl)sulfonylethanol (Compound 34.3)

To a solution of 2-(4-bromophenyl)sulfanylethanol (compound, 34.2, 1.2 g, 5.15 mmol) in DCM (30 mL) was added mCPBA (3.66 g, 18.02 mmol) at 0° C., the resultant mixture was warmed to room temperature, and stirred at this temperature for another 3 hrs. The reaction was quenched with sat.Na₂SO₃ (aq. 20 mL), and extracted with DCM (50 mL) twice. The combined organic layer was washed with brine, dried over Na₂SO₄, filtered and concentrated in vacuo. The residue was purified by flash chromatography (silica gel, 40 g, 20 to 40% EA in PE) to afford compound 34.3 (1.1 g, 80.2% yield). MS: calc'd 265, 267 [(M+H)⁺], measured 265, 267 [(M+H)⁺].

### Step (c): Preparation of 2-[2-(4-bromophenyl)sulfonylethoxymethoxy]ethyl-trimethyl-silane (Compound 34.4)

To a solution of 2-(4-bromophenyl)sulfonylethanol (compound 34.3, 500 mg, 1.89 mmol) and DIEA (488 mg, 3.78 mmol) in DCM (10 mL) was added dropwise SEMCl (472 mg, 2.83 mmol) at 0° C., the resultant mixture was stirred at room temperature for 2 hrs. The reaction mixture was diluted with DCM (100 mL), washed with brine (30 mL), dried over Na₂SO₄, filtered and concentrated in vacuo. The residue was purified by flash chromatography (silica gel, 40 g, 10% to 30% EA in PE) to give compound 34.4 (450 mg, 60.1% yield). MS: calc'd 395, 397 [(M+H)⁺], measured 417, 419 [(M+Na)⁺].

### Step (d): Preparation of tert-butyl (3R)-3-(4-allylsulfonylanilino)piperidine-1-carboxylate (Compound 34.5)

A mixture of 2-[2-(4-bromophenyl)sulfonylethoxymethoxy]ethyl-trimethyl-silane (compound 34.4, 300 mg, 0.76 mmol), tert-butyl (3R)-3-aminopiperidine-1-carboxylate (compound C1.1, 152 mg, 0.76 mmol), Cs₂CO₃ (742 mg, 2.28 mmol) and RuPhos Pd G2 (118 mg, 0.15 mmol) in toluene (5 mL) was stirred at 110° C. for 16 hrs. After being cooled to room temperature, the reaction was diluted with water (30 mL) and extracted with EA (30 mL) twice. The combined organic layer was washed with brine, dried over Na₂SO₄, filtered and concentrated in vacuo. The residue was purified by pre-HPLC to afford compound 34.5 (90 mg, 32.4% yield). MS: calc'd 367 [(M+H)⁺], measured 311 [(M−55)⁺].

### Step (e): Preparation of tert-butyl N-ethyl-N-[5-fluoro-3-methyl-2-oxo-3-[(3R)-3-(4-allylsulfonylanilino)-1-piperidyl]indolin-7-yl]carbamate (Compound 34.6)

To a solution of tert-butyl (3R)-3-(4-allylsulfonylanilino)piperidine-1-carboxylate (compound 34.5, 90 mg, 0.25 mmol) in DCM (5 mL) was added TFA (2 mL). The reaction mixture was stirred at room temperature for 2 hrs, then concentrated to afford an oil. To a solution of the residue and DIPEA (132 mg, 1.02 mmol) in isopropanol (5 mL) was added tert-butyl N-(3-bromo-5-fluoro-3-methyl-2-oxo-indolin-7-yl)-N-ethyl-carbamate (intermediate A, 96.5 mg, 0.25 mmol). The reaction mixture was stirred at room temperature for 2 hrs, then concentrated to afford a crude product, which was purified by pre-HPLC to afford compound 34.6 (75 mg, 52.4% yield). MS: calc'd 573 [(M+H)⁺], measured 573 [(M+H)⁺].

### Step (f): Preparation of 7-(ethylamino)-5-fluoro-3-methyl-3-[(3R)-3-(4-vinylsulfonylanilino)-1-piperidyl]indolin-2-one (Example 34)

To a solution of tert-butyl N-ethyl-N-[5-fluoro-3-methyl-2-oxo-3-[(3R)-3-(4-allylsulfonylanilino)-1-piperidyl]indolin-7-yl]carbamate (compound 34.6, 75 mg, 0.13 mmol) in DCM (5 mL) was added TFA (2 mL). The reaction mixture was stirred at room temperature for 2 hrs, then concentrated to afford a crude product, which was purified by prep-HPLC to afford Example 34 (39.6 mg, 64.5% yield). MS: calc'd 473 [(M+H)⁺], measured 473 [(M+H)⁺]. ¹H NMR (400 MHz, DMSO-d₆+D₂O) δ=7.52 (dd, J=8.8, 3.2 Hz, 2H), 6.89 (dd, J=9.6, 2.4 Hz, 0.5H), 6.85 (dd, J=9.6, 2.4 Hz, 0.5H), 6.68-6.60 (m, 3H), 6.47-6.43 (m, 1H), 6.15 (d, J=2.8 Hz, 0.5H), 6.11 (d, J=3.2 Hz, 0.5H), 6.01 (d, J=2.8 Hz, 0.5H), 5.99 (d, J=2.8 Hz, 0.5H), 3.77-3.70 (m, 1H), 3.36-3.30 (m, 1H), 3.24-3.18 (m, 1H), 3.04 (q, J=7.2 Hz, 2H), 2.87-2.75 (m, 1H), 2.62-2.57 (m, 1H), 1.94-1.87 (m, 2H), 1.77-1.71 (m, 1H), 1.63 (s, 1.5H), 1.61 (s, 1.5H), 1.41-1.30 (m, 1H), 1.17 (t, J=7.2 Hz, 3H).

### Example 35

### N-[(3R)-1-[7-(ethylamino)-5-fluoro-3-methyl-2-oxo-indolin-3-yl]-3-piperidyl]-3,5-difluoro-4-hydroxy-benzamide

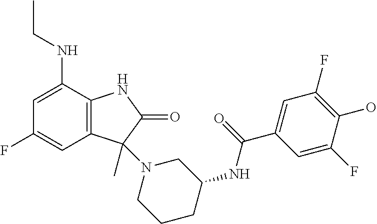 [SMILES: CCNc1cc(F)cc2c1NC(=O)C2(C)N1CCC[C@@H](NC(=O)c2cc(F)c(O)c(F)c2)C1]

The titled compound was synthesized according to the following scheme:

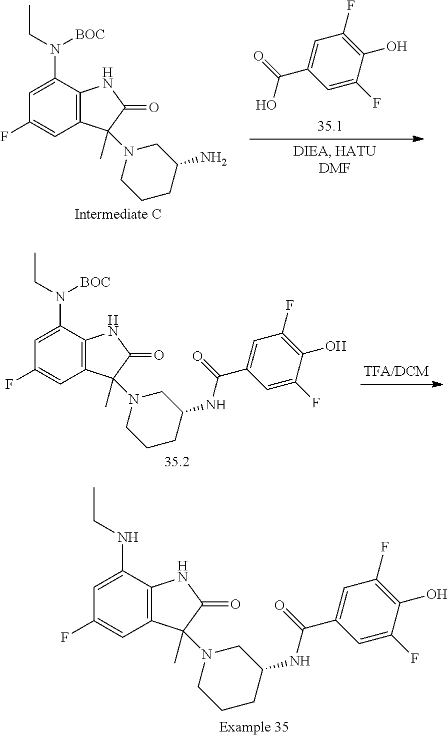 [SMILES: CCN(C(=O)OC(C)(C)C)c1cc(F)cc2c1NC(=O)C2(C)N1CCC[C@@H](N)C1 ; O=C(O)c1cc(F)c(O)c(F)c1 ; CCN(C(=O)OC(C)(C)C)c1cc(F)cc2c1NC(=O)C2(C)N1CCC[C@@H](NC(=O)c2cc(F)c(O)c(F)c2)C1 ; CCNc1cc(F)cc2c1NC(=O)C2(C)N1CCC[C@@H](NC(=O)c2cc(F)c(O)c(F)c2)C1]

### Step (a): Preparation of tert-butyl N-ethyl-N-[5-fluoro-3-methyl-2-oxo-3-[(3R)-3-[(3,5-difluoro-4-hydroxy-benzoyl)amino]-1-piperidyl]indolin-7-yl]carbamate (Compound 35.2)

To a solution of 3,5-difluoro-4-hydroxy-benzoic acid (compound 35.1, 37.69 mg, 0.20 mmol) in DMF (5 mL) was added DIEA (68.56 μL, 0.39 mmol) and HATU (82.32 mg, 0.20 mmol). After being stirred at room temperature for 0.5 hr, tert-butyl N-[3-[(3R)-3-amino-1-piperidyl]-5-fluoro-3-methyl-2-oxo-indolin-7-yl]-N-ethyl-carbamate (intermediate C, 80 mg, 0.2 mmol) was added, and the resultant mixture was stirred for additional 0.5 h. The reaction mixture was directly purified by prep-HPLC to afford compound 35.2 (60 mg, 53.3% yield). MS: calc'd 563 [(M+H)⁺], measured 563 [(M+H)⁺].

### Step (b): Preparation of N-[(3R)-1-[7-(ethylamino)-5-fluoro-3-methyl-2-oxo-indolin-3-yl]-3-piperidyl]-3,5-difluoro-4-hydroxy-benzamide (Example 35)

To a mixture of tert-butyl N-ethyl-N-[5-fluoro-3-methyl-2-oxo-3-[(3R)-3-[(3,5-difluoro-4-hydroxy-benzoyl)amino]-1-piperidyl]indolin-7-yl]carbamate (compound 35.2, 60.0 mg, 0.11 mmol) in DCM (4 mL) was added TFA (2 mL). The reaction mixture was stirred at room temperature for 2 hrs, then concentrated to afford a crude product, which was purified by prep-HPLC to afford Example 35 (15 mg, 29.5% yield). MS: calc'd 463 [(M+H)⁺], measured 463 [(M+H)⁺]. ¹H NMR (400 MHz, METHANOL-d₄) δ=7.45-7.37 (m, 2H), 6.45-6.40 (m, 1H), 6.36-6.29 (m, 1H), 4.15-3.96 (m, 1H), 3.34-3.11 (m, 2H), 3.03-2.96 (m, 0.5H), 2.83-2.68 (m, 2H), 2.53-2.35 (m, 1H), 2.28-2.22 (m, 0.5H), 1.81-1.68 (m, 2H), 1.64-1.49 (m, 2H), 1.47 (d, J=5.0 Hz, 3H), 1.31-1.25 (m, 3H).

### Example 36

### N-[(3R)-1-[7-(ethylamino)-5-fluoro-3-methyl-2-oxo-indolin-3-yl]-3-piperidyl]-1,1-dioxo-thiane-4-carboxamide

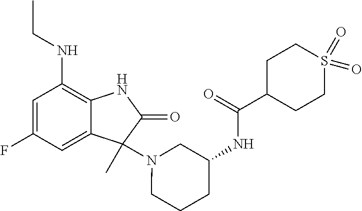 [SMILES: CCNc1cc(F)cc2c1NC(=O)C2(C)N1CCC[C@@H](NC(=O)C2CCS(=O)(=O)CC2)C1]

N-[(3R)-1-[7-(ethylamino)-5-fluoro-3-methyl-2-oxo-indolin-3-yl]-3-piperidyl]-1,1-dioxo-thiane-4-carboxamide (Example 36) was prepared in analogy to Example 35, by replacing 3,5-difluoro-4-hydroxy-benzoic acid (compound 35.1) with 1,1-dioxothiane-4-carboxylic acid in step (a). MS: calc'd 467 [(M+H)⁺], measured 467 [(M+H)⁺]. ¹H NMR (400 MHz, DMSO-d₆) δ=9.92-9.74 (m, 1H), 7.68 (d, J=8.0 Hz, 1H), 6.43-6.21 (m, 2H), 5.11 (s, 1H), 3.68-3.52 (m, 1H), 3.16-3.01 (m, 6H), 2.80-2.56 (m, 2H), 2.45-2.31 (m, 2H), 2.27-2.08 (m, 1H), 2.05-1.90 (m, 4H), 1.69-1.54 (m, 2H), 1.41-1.33 (m, 1H), 1.32 (s, 3H), 1.19 (t, J=7.2 Hz, 3H), 1.16-1.08 (m, 1H).

### Example 37

### 2-cyano-N-[(3R)-1-[7-(ethylamino)-5-fluoro-3-methyl-2-oxo-indolin-3-yl]-3-piperidyl]-2-azaspiro[3.3]heptane-6-carboxamide

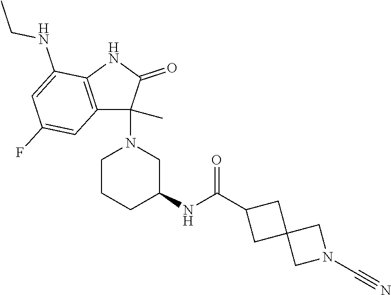 [SMILES: CCNc1cc(F)cc2c1NC(=O)C2(C)N1CCC[C@H](NC(=O)C2CC3(C2)CN(C#N)C3)C1]

The titled compound was synthesized according to the following scheme:

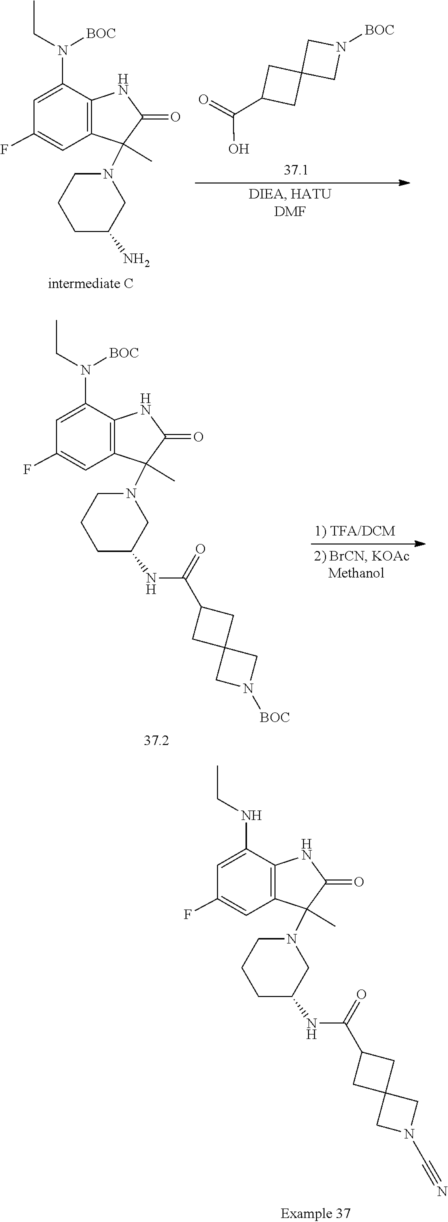

### Step (a): Preparation of tert-butyl 6-[[(3R)-1-[7-[tert-butoxycarbonyl(ethyl)amino]-5-fluoro-3-methyl-2-oxo-indolin-3-yl]-3-piperidyl]carbamoyl]-2-azaspiro[3.3]heptane-2-carboxylate (Compound 37.2)

To a solution of 2-tert-butoxycarbonyl-2-azaspiro[3.3]heptane-6-carboxylic acid (compound 37.1, CAS: 1211526-53-2, Bide Pharmatech, Catalog: BD227253, 41.55 mg, 172 μmol) and DIEA (66.77 mg, 517 μmol) in DMF (5 mL) was added HATU (72.03 mg, 189 μmol). After being stirred at room temperature for 30 min, tert-butyl N-[3-[(3R)-3-amino-1-piperidyl]-5-fluoro-3-methyl-2-oxo-indolin-7-yl]-N-ethyl-carbamate (intermediate C, 70 mg, 172 μmol) was added, and the resultant mixture was stirred at room temperature for another 2 hrs. The reaction mixture was diluted with water (30 mL), and extracted with EA (20 mL) twice. The combined organic layer was washed with brine, dried over Na₂SO₄, filtered and concentrated. The residue was purified by flash chromatography (silica gel, 20 g, 0% to 100% EA in PE) to compound 37.2 (80 mg, 73.8%). MS: calc'd 630 [(M+H)⁺], measured 630 [(M+H)⁺].

### Step (b): Preparation of 2-cyano-N-[(3R)-1-[7-(ethylamino)-5-fluoro-3-methyl-2-oxo-indolin-3-yl]-3-piperidyl]-2-azaspiro[3.3]heptane-6-carboxamide (Example 37)

To a solution of tert-butyl 6-[[(3R)-1-[7-[tert-butoxycarbonyl(ethyl)amino]-5-fluoro-3-methyl-2-oxo-indolin-3-yl]-3-piperidyl]carbamoyl]-2-azaspiro[3.3]heptane-2-carboxylate (compound 37.2, 80 mg, 127 μmol) in DCM (10 mL) was added TFA (5 mL). The reaction mixture was stirred at room temperature for 2 hrs, then concentrated to afford an oil. To a solution of the residue in methanol (5 mL) was added potassium acetate (37.3 mg, 381 μmol) and cyanogen bromide (16.2 mg, 152.4 μmol), the resultant mixture was stirred at room temperature for 5 hrs. The reaction mixture was quenched with water (0.5 mL), then concentrated, and the residue was purified by flash chromatography (silica gel, 12 g, 0% to 20% MeOH in DCM) to afford Example 37 (44 mg, 76.3% yield). MS: calc'd 455 [(M+H)⁺], measured 455 [(M+H)⁺]. ¹H NMR (500 MHz, METHANOL-d₄) δ=6.40-6.35 (m, 1H), 6.33-6.28 (m, 1H), 4.20-4.15 (m, 2H), 4.12-4.08 (m, 2H), 3.92-3.82 (m, 0.5H), 3.81-3.72 (m, 0.5H), 3.17-3.10 (m, 2H), 2.96-2.52 (m, 5H), 2.49-2.28 (m, 5H), 1.73-1.50 (m, 3H), 1.44-1.41 (m, 3H), 1.30-1.25 (m, 3H).

### Example 38

### 1-cyano-N-[(3R)-1-[7-(ethylamino)-5-fluoro-3-methyl-2-oxo-indolin-3-yl]-3-piperidyl]piperidine-4-carboxamide

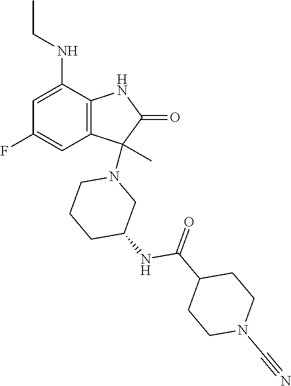 [SMILES: CCNc1cc(F)cc2c1NC(=O)C2(C)N1CCC[C@@H](NC(=O)C2CCN(C#N)CC2)C1]

1-cyano-N-[(3R)-1-[7-(ethylamino)-5-fluoro-3-methyl-2-oxo-indolin-3-yl]-3-piperidyl]piperidine-4-carboxamide (Example 38) was prepared in analogy to Example 37, by replacing 2-tert-butoxycarbonyl-2-azaspiro[3.3]heptane-6-carboxylic acid (compound 37.1) with 1-tert-butoxycarbonylpiperidine-4-carboxylic acid in step (a). MS: calc'd 443 [(M+H)⁺], measured 443 [(M+H)⁺]. ¹H NMR (500 MHz, METHANOL-d₄) δ=6.41-6.37 (m, 1H), 6.34-6.29 (m, 1H), 3.95-3.75 (m, 1H), 3.50-3.41 (m, 2H), 3.18-3.12 (m, 2H), 3.12-3.04 (m, 2H), 2.91-2.53 (m, 3H), 2.49-2.08 (m, 3H), 1.81-1.72 (m, 4H), 1.72-1.51 (m, 3H), 1.46-1.42 (m, 3H), 1.30-1.25 (m, 3H).

### Example 39

### 2-cyano-N-[(3R)-1-[3-ethyl-7-(ethylamino)-5-fluoro-2-oxo-indolin-3-yl]-3-piperidyl]-2-azaspiro[3.3]heptane-6-carboxamide

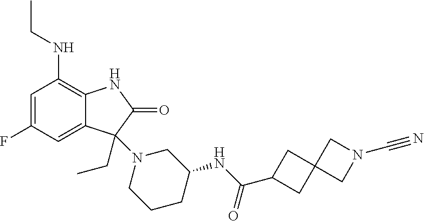 [SMILES: CCNc1cc(F)cc2c1NC(=O)C2(CC)N1CCC[C@@H](NC(=O)C2CC3(C2)CN(C#N)C3)C1]

2-cyano-N-[(3R)-1-[3-ethyl-7-(ethylamino)-5-fluoro-2-oxo-indolin-3-yl]-3-piperidyl]-2-azaspiro[3.3]heptane-6-carboxamide (Example 39) was prepared in analogy to Example 37, by replacing tert-butyl N-[3-[(3R)-3-amino-1-piperidyl]-5-fluoro-3-methyl-2-oxo-indolin-7-yl]-N-ethyl-carbamate (intermediate C) with tert-butyl N-[3-[(3R)-3-amino-1-piperidyl]-3-ethyl-5-fluoro-2-oxo-indolin-7-yl]-N-ethyl-carbamate (intermediate D) in step (a). MS: calc'd 469 [(M+H)⁺]; measured 469 [(M+H)⁺]. ¹H NMR (400 MHz, DMSO-d₆) δ=9.85 (s, 0.5H), 9.84 (s, 0.5H), 7.53 (d, J=8.8 Hz, 0.5H), 7.46 (d, J=8.8 Hz, 0.5H), 6.39-6.22 (m, 2H), 5.09 (br s, 1H), 4.13 (d, J=6.0 Hz, 2H), 4.04 (d, J=6.0 Hz, 2H), 3.65-3.52 (m, 1H), 3.13-3.01 (m, 2H), 2.82-2.69 (m, 3H), 2.30-2.10 (m, 6H), 1.83-1.70 (m, 2H), 1.69-1.51 (m, 2H), 1.42-1.29 (m, 1H), 1.19 (t, J=7.1 Hz, 3H), 1.14-1.02 (m, 1H), 0.68-0.52 (m, 3H).

### Example 40

### 2-(2-chloroacetyl)-N-[(3R)-1-[7-(ethylamino)-5-fluoro-3-methyl-2-oxo-indolin-3-yl]-3-piperidyl]-2-azaspiro[3.3]heptane-6-carboxamide

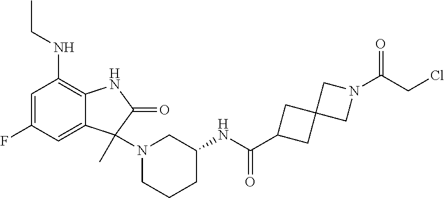 [SMILES: CCNc1cc(F)cc2c1NC(=O)C2(C)N1CCC[C@@H](NC(=O)C2CC3(C2)CN(C(=O)CCl)C3)C1]

The titled compound was synthesized according to the following scheme:

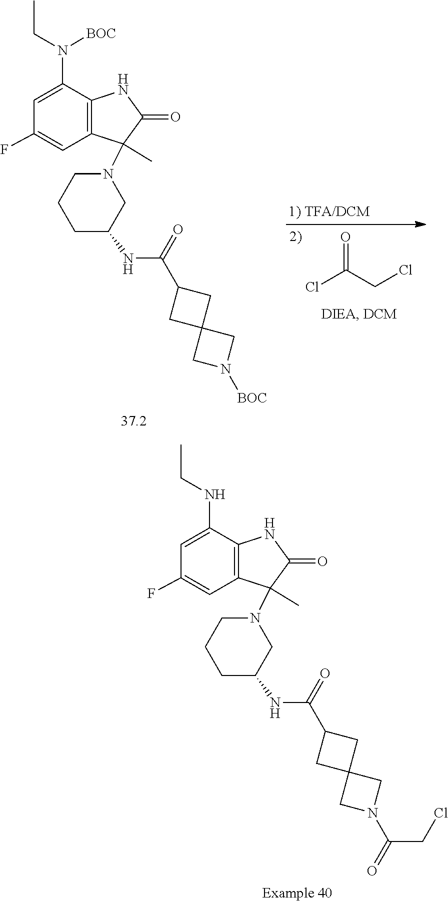 [SMILES: CCN(C(=O)OC(C)(C)C)c1cc(F)cc2c1NC(=O)C2(C)N1CCC[C@@H](NC(=O)C2CC3(C2)CN(C(=O)OC(C)(C)C)C3)C1 ; O=C(Cl)CCl ; CCNc1cc(F)cc2c1NC(=O)C2(C)N1CCC[C@@H](NC(=O)C2CC3(C2)CN(C(=O)CCl)C3)C1]

### Step (a): Preparation of 2-(2-chloroacetyl)-N-[(3R)-1-[7-(ethylamino)-5-fluoro-3-methyl-2-oxo-indolin-3-yl]-3-piperidyl]-2-azaspiro[3.3]heptane-6-carboxamide (Example 40)

To a solution of tert-butyl 6-[[(3R)-1-[7-[tert-butoxycarbonyl(ethyl)amino]-5-fluoro-3-methyl-2-oxo-indolin-3-yl]-3-piperidyl]carbamoyl]-2-azaspiro[3.3]heptane-2-carboxylate (compound 37.2, 70.0 mg, 110 μmol) in DCM (10 mL) was added TFA (5 mL). The reaction mixture was stirred at room temperature for 2 hrs, then concentrated to afford an oil. To a solution of the residue and DIEA (42.57 mg, 330 μmol) in DCM (5 mL) was added chloroacetyl chloride (12.32 mg, 110 μmol) at 0° C., the resultant mixture was stirred at room temperature for another 2 hrs. The reaction mixture was concentrated to afford a crude product, which was purified by prep-HPLC to afford Example 40 (4.6 mg, 8.3% yield). MS: calc'd 506 [(M+H)⁺], measured 506 [(M+H)⁺]. ¹H NMR (400 MHz, DMSO-d₆) δ=9.84 (s, 0.5H), 9.82 (s, 0.5H), 7.56-7.52 (m, 0.5H), 7.49-7.45 (m, 0.5H), 6.35-6.23 (m, 2H), 5.12 (s, 1H), 4.19 (d, J=6.0 Hz, 1H), 4.12-4.06 (m, 3H), 3.89 (d, J=5.6 Hz, 1H), 3.80 (d, J=5.6 Hz, 1H), 3.66-3.54 (m, 1H), 3.13-3.02 (m, 2H), 2.87-2.72 (m, 2H), 2.64-2.57 (m, 1H), 2.29-2.15 (m, 5H), 2.08-1.96 (m, 1H), 1.69-1.54 (m, 2H), 1.38-1.35 (m, 1H), 1.32 (s, 3H), 1.20 (t, J=7.1 Hz, 3H), 1.12-1.05 (m, 1H).

### Example 41

### N-[(3R)-1-[7-(ethylamino)-5-fluoro-3-methyl-2-oxo-indolin-3-yl]-3-piperidyl]-2-prop-2-enoyl-2-azaspiro[3.3]heptane-6-carboxamide

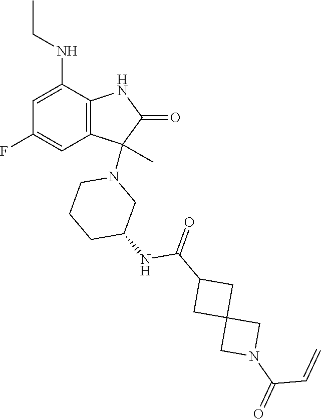 [SMILES: C=CC(=O)N1CC2(CC(C(=O)N[C@@H]3CCCN(C4(C)C(=O)Nc5c(NCC)cc(F)cc54)C3)C2)C1]

N-[(3R)-1-[7-(ethylamino)-5-fluoro-3-methyl-2-oxo-indolin-3-yl]-3-piperidyl]-2-prop-2-enoyl-2-azaspiro[3.3]heptane-6-carboxamide (Example 41) was prepared in analogy to Example 40, by replacing chloroacetyl chloride with prop-2-enoyl chloride in step (a). MS: calc'd 484 [(M+H)⁺], measured 484 [(M+H)⁺]. ¹H NMR (400 MHz, DMSO-d₆) δ=9.85 (s, 0.5H), 9.83 (s, 0.5H), 7.63-7.39 (m, 1H), 6.35-6.27 (m, 3H), 6.12-5.99 (m, 1H), 5.64 (dd, J=10.0, 2.4 Hz, 1H), 5.13 (br d, J=4.4 Hz, 1H), 4.21 (d, J=6.8 Hz, 1H), 4.10 (d, J=6.0 Hz, 1H), 3.90 (d, J=5.6 Hz, 1H), 3.80 (d, J=6.0 Hz, 1H), 3.69-3.51 (m, 1H), 3.13-3.03 (m, 2H), 2.87-2.73 (m, 3H), 2.28-2.16 (m, 6H), 1.70-1.52 (m, 2H), 1.35-1.26 (m, 4H), 1.19 (t, J=7.1 Hz, 3H), 1.13-1.04 (m, 1H).

### Example 42

### 3-[[(3R)-1-[7-(ethylamino)-5-fluoro-3-methyl-2-oxo-indolin-3-yl]-3-piperidyl]carbamoyl]benzenesulfonyl fluoride

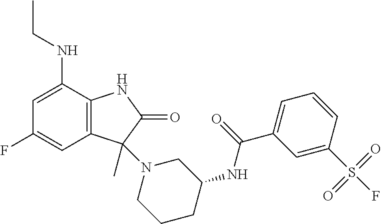 [SMILES: CCNc1cc(F)cc2c1NC(=O)C2(C)N1CCC[C@@H](NC(=O)c2cccc(S(=O)(=O)F)c2)C1]

The titled compound was synthesized according to the following scheme:

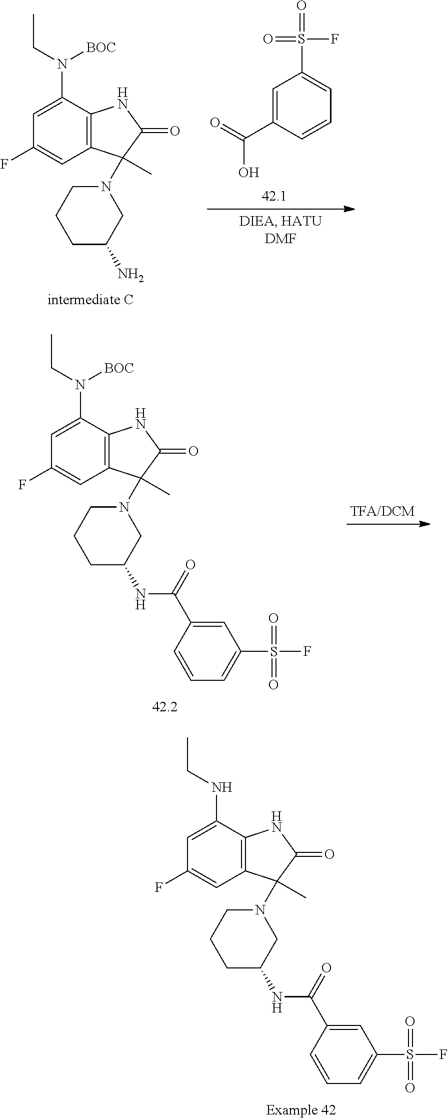

### Step (a): Preparation of tert-butyl N-ethyl-N-[5-fluoro-3-[(3R)-3-[(3-fluorosulfonylbenzoyl)amino]-1-piperidyl]-3-methyl-2-oxo-indolin-7-yl]carbamate (Compound 42.2)

To a solution of 3-fluorosulfonylbenzoic acid (compound 42.1, 60.0 mg, 294.1 μmol) and DIEA (150.9 mg, 1.17 mmol) in DMF (2 mL) was added HATU (111.7 mg, 294.1 μmol) at 0° C. After being stirred at 0° C. for 10 min, tert-butyl N-[3-[(3R)-3-amino-1-piperidyl]-5-fluoro-3-methyl-2-oxo-indolin-7-yl]-N-ethyl-carbamate (intermediate C, 101.5 mg, 250 μmol) was added, and the resultant mixture was stirred at room temperature for another 1 hr. The reaction mixture was poured into ice water (30 mL), and extracted with EA (30 mL) twice. The combined organic layer was washed with brine, dried over Na₂SO₄, filtered and concentrated to afford compound 42.2 (140 mg, crude) which was used directly for the next step without further purification. MS: calc'd 593 [(M+H)⁺]; measured 593 [(M+H)⁺].

### Step (b): Preparation of 3-[[(3R)-1-[7-(ethylamino)-5-fluoro-3-methyl-2-oxo-indolin-3-yl]-3-piperidyl]carbamoyl]benzenesulfonyl fluoride (Example 42)

To a solution of tert-butyl N-ethyl-N-[5-fluoro-3-[(3R)-3-[(3-fluorosulfonylbenzoyl)amino]-1-piperidyl]-3-methyl-2-oxo-indolin-7-yl]carbamate (compound 42.2, 140.0 mg, crude) in DCM (5 mL) was added TFA (2 mL). The reaction mixture was stirred at room temperature for 2 hrs, then concentrated to afford a crude product, which was purified by prep-HPLC to afford Example 42 (59.6 mg, two-step: 48.4% yield). MS: calc'd 493 [(M+H)⁺]; measured 493 [(M+H)⁺]. ¹H NMR (400 MHz, DMSO-d₆) δ=10.40 (s, 1H), 8.77 (d, J=7.6 Hz, 0.4H), 8.72 (d, J=7.6 Hz, 0.6H), 8.53 (s, 1H), 8.38 (d, J=8.0 Hz, 1H), 8.32 (d, J=8.0 Hz, 1H), 7.90 (t, J=8.0 Hz, 1H), 6.62-6.56 (m, 1H), 6.42 (d, J=12.4 Hz, 1H), 4.47-4.06 (m, 1H), 3.30-3.13 (m, 2H), 3.09 (q, J=7.2 Hz, 2H), 2.73-2.61 (m, 1H), 2.54-2.53 (m, 1H), 1.93-1.79 (m, 2H), 1.75-1.62 (m, 1H), 1.57 (s, 3H), 1.51-1.36 (m, 2H), 1.19 (t, J=7.2 Hz, 3H).

### Example 43

### 4-[[(3R)-1-[7-(ethylamino)-5-fluoro-3-methyl-2-oxo-indolin-3-yl]-3-piperidyl]carbamoyl]benzenesulfonyl fluoride

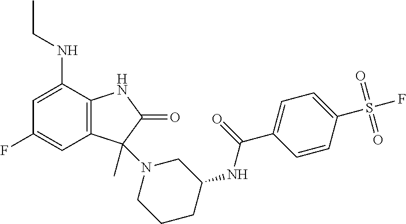 [SMILES: CCNc1cc(F)cc2c1NC(=O)C2(C)N1CCC[C@@H](NC(=O)c2ccc(S(=O)(=O)F)cc2)C1]

4-[[(3R)-1-[7-(ethylamino)-5-fluoro-3-methyl-2-oxo-indolin-3-yl]-3-piperidyl]carbamoyl]benzenesulfonyl fluoride (Example 43) was prepared in analogy to Example 42, by replacing 3-fluorosulfonylbenzoic acid (compound 42.1) with 4-fluorosulfonylbenzoic acid in step (a). MS: calc'd 493 [(M+H)⁺], measured 493 [(M+H)⁺]. ¹H NMR (400 MHz, DMSO-d₆) δ=10.40 (s, 1H), 8.73 (d, J=7.6 Hz, 0.4H), 8.68 (d, J=7.6 Hz, 0.6H), 8.27-8.24 (m, 2H), 8.15 (d, J=8.4 Hz, 2H), 6.59-6.56 (m, 1H), 6.42 (d, J=12.4 Hz, 1H), 4.14 (s, 1H), 3.27-3.13 (m, 1H), 3.09 (q, J=7.2 Hz, 2H), 2.74-2.55 (m, 1H), 2.49-2.31 (m, 2H), 1.92-1.77 (m, 2H), 1.75-1.60 (m, 1H), 1.56 (s, 3H), 1.48-1.33 (m, 2H), 1.19 (t, J=7.2 Hz, 3H).

### Example 44

### 7-(ethylamino)-5-fluoro-3-[(3R)-3-[(3-fluorosulfonyloxybenzoyl)amino]-1-piperidyl]-3-methyl-2-oxo-indoline

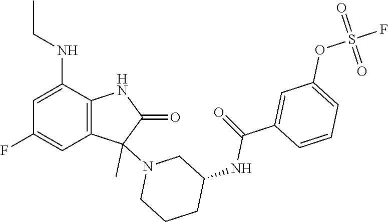 [SMILES: CCNc1cc(F)cc2c1NC(=O)C2(C)N1CCC[C@@H](NC(=O)c2cccc(OS(=O)(=O)F)c2)C1]

The titled compound was synthesized according to the following scheme:

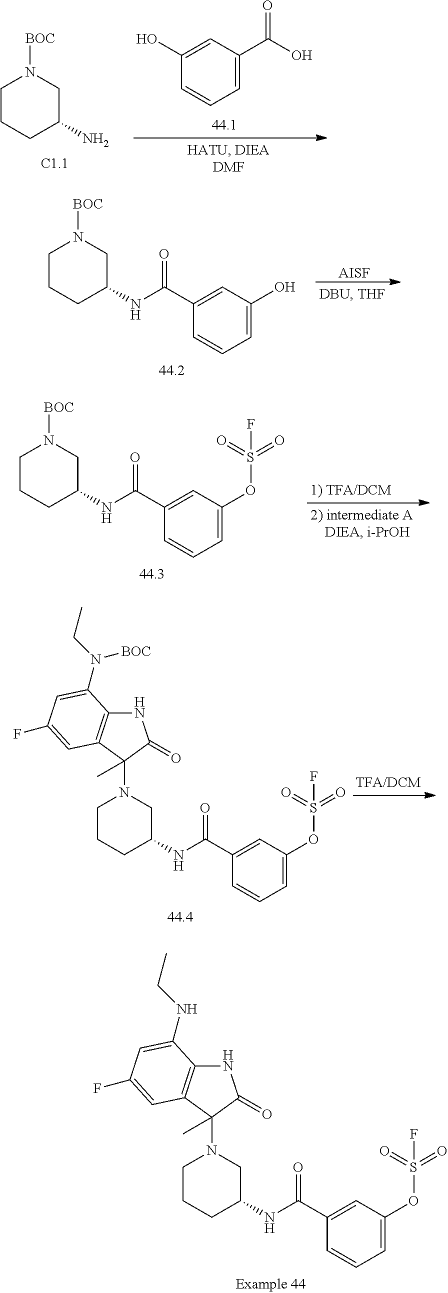

### Step (a): Preparation of tert-butyl (3R)-3-[(3-hydroxybenzoyl)amino]piperidine-1-carboxylate (Compound 44.2)

To a solution of 3-hydroxybenzoic acid (compound 44.1, 200.0 mg, 1.45 mmol) in DMF (5 mL) was added tert-butyl (3R)-3-aminopiperidine-1-carboxylate (compound 46.2, 348 mg, 1.74 mmol), DIEA (0.76 mL, 4.34 mmol) and HATU (340 mg, 1.45 mmol), the resultant mixture was stirred at room temperature for 1 h, then diluted with water (30 mL), and extracted with EA (30 mL) twice. The combined organic layer was washed with brine (60 mL), dried over Na₂SO₄, filtered and concentrated. The residue was purified by column chromatography (silica gel, 20 g, 20% to 50% EA in PE) to afford compound 44.2 (300 mg, 64.6% yield). MS: calc'd 321 [(M+H)⁺], measured 265 [(M−55)⁺].

### Step (b): Preparation of tert-butyl (3R)-3-[(3-fluorosulfonyloxybenzoyl)amino]piperidine-1-carboxylate (Compound 44.3)

To a solution of tert-butyl (3R)-3-[(3-hydroxybenzoyl)amino]piperidine-1-carboxylate (compound 44.2, 200.0 mg, 0.62 mmol), AISF (392 mg, 1.25 mmol) in THF (1 mL) was added a solution of DBU (399 mg, 2.62 mmol) in THF (0.5 mL), the mixture was stirred at 15° C. for 10 min. The reaction mixture was poured into water (20 mL), and extracted with EA (20 mL) for three times. The combined organic layer was washed with brine (30 mL), dried over Na₂SO₄, filtered and concentrated. The residue was purified by column chromatography (silica gel, 20 g, 30% to 50% EA in PE) to afford compound 44.3 (180 mg, 72.2% yield). MS: calc'd 403 [(M+H)⁺], measured 347 [(M−55)⁺].

### Step (c): Preparation of 7-(ethylamino)-5-fluoro-3-[(3R)-3-[(3-fluorosulfonyloxybenzoyl)amino]-1-piperidyl]-3-methyl-2-oxo-indoline (Example 44)

7-(ethylamino)-5-fluoro-3-[(3R)-3-[(3-fluorosulfonyloxybenzoyl)amino]-1-piperidyl]-3-methyl-2-oxo-indoline (Example 44) was prepared in analogy to Example 8, by replacing tert-butyl (3R)-3-(4-cyanoanilino)piperidine-1-carboxylate (compound 8.1) with tert-butyl (3R)-3-[(3-fluorosulfonyloxybenzoyl)amino]piperidine-1-carboxylate (compound 44.3) in step (b). MS: calc'd 509 [(M+H)⁺], measured 509 [(M+H)⁺]. ¹H NMR (400 MHz, DMSO-d₆) δ=10.44 (s, 1H), 8.57-8.51 (m, 1H), 8.00-7.90 (m, 2H), 7.71-7.65 (m, 2H), 6.69-6.63 (m, 1H), 6.54-6.49 (m, 1H), 4.19-4.12 (m, 1H), 3.40-3.15 (m, 2H), 3.10 (q, J=7.2 Hz, 2H), 2.81-2.59 (m, 2H), 1.94-1.85 (m, 2H), 1.73-1.64 (m, 1H), 1.58 (s, 3H), 1.50-1.26 (m, 2H), 1.20 (t, J=7.2 Hz, 3H).

### Example 45

### 5-[[(3R,5R)-1-[7-(ethylamino)-5-fluoro-3-methyl-2-oxo-indolin-3-yl]-5-fluoro-3-piperidyl]amino]pyridine-2-carbonitrile

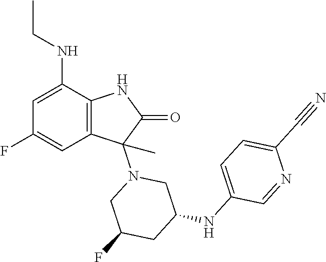 [SMILES: CCNc1cc(F)cc2c1NC(=O)C2(C)N1C[C@H](F)C[C@@H](Nc2ccc(C#N)nc2)C1]

5-[[(3R,5R)-1-[7-(ethylamino)-5-fluoro-3-methyl-2-oxo-indolin-3-yl]-5-fluoro-3-piperidyl]-amino]pyridine-2-carbonitrile (Example 45) was prepared in analogy to Example 23, by replacing tert-butyl N-[(3R)-3-methyl-3-piperidyl]carbamate (compound 23.1) with tert-butyl N-[(3R,5R)-5-fluoro-3-piperidyl]carbamate (CAS: 1363378-07-7, PharmaBlock, Catalog: PBZS2027) in step (a). MS: calc'd 427 [(M+H)⁺], measured 427 [(M+H)⁺]. ¹H NMR (500 MHz, METHANOL-d₄) δ=7.96-7.89 (m, 0.4H), 7.87-7.82 (m, 0.6H), 7.48-7.42 (m, 0.4H), 7.39-7.34 (m, 0.6H), 6.93-6.85 (m, 0.4H), 6.76-6.68 (m, 0.6H), 6.41-6.35 (m, 0.6H), 6.28-6.17 (m, 1.4H), 3.81-3.72 (m, 0.4H), 3.68-3.58 (m, 0.6H), 3.08-2.96 (m, 3H), 2.95-2.83 (m, 1H), 2.77-2.44 (m, 2H), 2.11-1.55 (m, 3H), 1.41-1.35 (m, 3H), 1.21-1.13 (m, 3H).

### Example 46

### 5-[[(3R,5S)-1-[7-(ethylamino)-5-fluoro-3-methyl-2-oxo-indolin-3-yl]-5-fluoro-3-piperidyl]amino]pyridine-2-carbonitrile

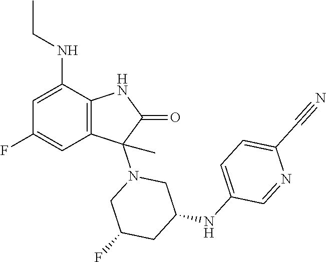 [SMILES: CCNc1cc(F)cc2c1NC(=O)C2(C)N1C[C@@H](F)C[C@@H](Nc2ccc(C#N)nc2)C1]

5-[[(3R,5S)-1-[7-(ethylamino)-5-fluoro-3-methyl-2-oxo-indolin-3-yl]-5-fluoro-3-piperidyl]-amino]pyridine-2-carbonitrile (Example 46) was prepared in analogy to Example 23, by replacing tert-butyl N-[(3R)-3-methyl-3-piperidyl]carbamate (compound 23.1) with tert-butyl N-[(3R,5S)-5-fluoro-3-piperidyl]carbamate (CAS: 1363378-08-8, PharmaBlock, Catalog: PBN20120299) in step (a). MS: calc'd 427 [(M+H)⁺], measured 427 [(M+H)⁺]. ¹H NMR (500 MHz, METHANOL-d₄) δ=7.92-7.89 (m, 0.5H), 7.88-7.84 (m, 0.5H), 7.44-7.40 (m, 0.5H), 7.39-7.35 (m, 0.5H), 6.89-6.84 (m, 0.5H), 6.80-6.73 (m, 0.5H), 6.36-6.30 (m, 0.5H), 6.27-6.22 (m, 0.5H), 6.22-6.16 (m, 1H), 4.67-4.59 (m, 0.5H), 4.57-4.48 (m, 0.5H), 3.61-3.54 (m, 0.5H), 3.50-3.44 (m, 0.5H), 3.07-2.99 (m, 2.5H), 2.93-2.85 (m, 0.5H), 2.77-2.60 (m, 2H), 2.50-2.38 (m, 0.5H), 2.21-1.98 (m, 1.5H), 1.72-1.50 (m, 1H), 1.40-1.33 (m, 3H), 1.20-1.12 (m, 3H).

### Example 47

### 5-[[(3R,5S)-1-[7-(ethylamino)-5-fluoro-3-methyl-2-oxo-indolin-3-yl]-5-methyl-3-piperidyl]amino]pyridine-2-carbonitrile

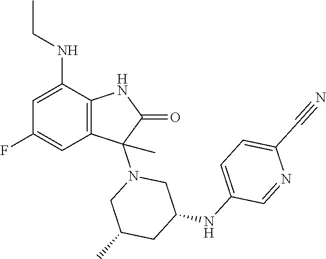 [SMILES: CCNc1cc(F)cc2c1NC(=O)C2(C)N1C[C@@H](C)C[C@@H](Nc2ccc(C#N)nc2)C1]

5-[[(3R,5S)-1-[7-(ethylamino)-5-fluoro-3-methyl-2-oxo-indolin-3-yl]-5-methyl-3-piperidyl]amino]pyridine-2-carbonitrile (Example 47) was prepared in analogy to Example 23, by replacing tert-butyl N-[(3R)-3-methyl-3-piperidyl]carbamate (compound 23.1) with tert-butyl N-[(3R,5S)-5-methylpiperidin-3-yl]carbamate (CAS: 1187055-56-6, PharmaBlock, Catalog: PB06173) in step (a). MS: calc'd 423 [(M+H)⁺], measured 423 [(M+H)⁺]. ¹H NMR (500 MHz, METHANOL-d₄) δ=7.89-7.84 (m, 0.5H), 7.82-7.74 (m, 0.5H), 7.46-7.42 (m, 0.5H), 7.36-7.32 (m, 0.5H), 6.90-6.83 (m, 0.5H), 6.74-6.69 (m, 0.5H), 6.33-6.26 (m, 1H), 6.26-6.19 (m, 1H), 3.45-3.36 (m, 0.5H), 3.36-3.27 (m, 0.5H), 3.16-3.30 (m, 1H), 3.08-2.99 (m, 2.5H), 2.95-2.82 (m, 1H), 2.77-2.70 (m, 0.5H), 2.06-1.95 (m, 1H), 1.95-1.88 (m, 1H), 1.82-1.72 (m, 1H), 1.71-1.56 (m, 1H), 1.41-1.33 (m, 3H), 1.19-1.13 (m, 3H), 0.83-0.77 (m, 3H).

### Example 48

### 5-[[(3S,4S)-1-[7-(ethylamino)-5-fluoro-3-methyl-2-oxo-indolin-3-yl]-4-fluoro-3-piperidyl]amino]pyridine-2-carbonitrile

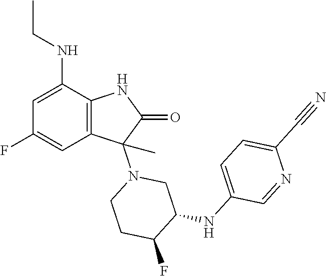 [SMILES: CCNc1cc(F)cc2c1NC(=O)C2(C)N1CC[C@H](F)[C@@H](Nc2ccc(C#N)nc2)C1]

5-[[(3S,4S)-1-[7-(ethylamino)-5-fluoro-3-methyl-2-oxo-indolin-3-yl]-4-fluoro-3-piperidyl]-amino]pyridine-2-carbonitrile (Example 48) was prepared in analogy to Example 23, by replacing tert-butyl N-[(3R)-3-methyl-3-piperidyl]carbamate (compound 23.1) with tert-butyl N-[(3S,4S)-4-fluoropiperidin-3-yl]carbamate (CAS: 1052713-48-0, PharmaBlock, Catalog: PBN20120291) in step (a). MS: calc'd 427 [(M+H)⁺], measured 427 [(M+H)⁺]. ¹H NMR (500 MHz, METHANOL-d₄) δ=8.10-8.02 (m, 1H), 7.58-7.49 (m, 1H), 7.10-6.97 (m, 1H), 6.54-6.41 (m, 1H), 6.41-6.30 (m, 1H), 4.55-4.46 (m, 0.5H), 4.45-4.35 (m, 0.5H), 3.83-3.65 (m, 1H), 3.19-2.98 (m, 3H), 2.91-2.67 (m, 2H), 2.60-2.39 (m, 1H), 2.21-2.02 (m, 1H), 1.96-1.73 (m, 1H), 1.56-1.48 (m, 3H), 1.30-1.20 (m, 3H).

### Example 49

### 5-[[(3S,4S)-1-[3-ethyl-7-(ethylamino)-5-fluoro-2-oxo-indolin-3-yl]-4-fluoro-3-piperidyl]amino]pyridine-2-carbonitrile

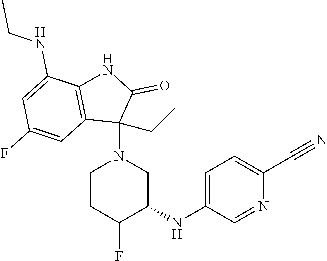 [SMILES: CCNc1cc(F)cc2c1NC(=O)C2(CC)N1CCC(F)[C@@H](Nc2ccc(C#N)nc2)C1]

5-[[(3S,4S)-1-[3-ethyl-7-(ethylamino)-5-fluoro-2-oxo-indolin-3-yl]-4-fluoro-3-piperidyl]-amino]pyridine-2-carbonitrile (Example 49) was prepared in analogy to Example 23, by replacing intermediate A with intermediate B and tert-butyl N-[(3R)-3-methyl-3-piperidyl]carbamate (compound 23.1) with tert-butyl N-[(3S,4S)-4-fluoropiperidin-3-yl]carbamate in step (a). calc'd 441 [(M+H)⁺], measured 441 [(M+H)⁺]. ¹H NMR (500 MHz, METHANOL-d₄) δ=8.08-8.05 (m, 0.5H), 8.04-8.01 (m, 0.5H), 7.57-7.54 (m, 0.5H), 7.53-7.50 (m, 0.5H), 7.09-7.04 (m, 0.5H), 7.01-6.94 (m, 0.5H), 6.52-6.46 (m, 0.5H), 6.44-6.31 (m, 1.5H), 4.54-4.47 (m, 0.5H), 4.45-4.36 (m, 0.5H), 3.82-3.66 (m, 1H), 3.18-3.12 (m, 2H), 3.11-3.02 (m, 2H), 2.94-2.71 (m, 1H), 2.62-2.36 (m, 1H), 2.22-1.73 (m, 4H), 1.31-1.23 (m, 3H), 0.73-0.63 (m, 3H).

### Example 50

### 5-[[(3S,4S)-1-[7-(ethylamino)-5-fluoro-3-methyl-2-oxo-indolin-3-yl]-4-hydroxy-3-piperidyl]amino]pyridine-2-carbonitrile

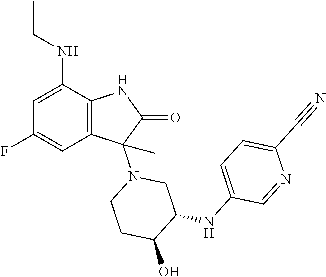 [SMILES: CCNc1cc(F)cc2c1NC(=O)C2(C)N1CC[C@H](O)[C@@H](Nc2ccc(C#N)nc2)C1]

5-[[(3S,4S)-1-[7-(ethylamino)-5-fluoro-3-methyl-2-oxo-indolin-3-yl]-4-fluoro-3-piperidyl]-amino]pyridine-2-carbonitrile (Example 50) was prepared in analogy to Example 23, by replacing tert-butyl N-[(3R)-3-methyl-3-piperidyl]carbamate (compound 23.1) with tert-butyl N-[(3S,4S)-4-hydroxypiperidin-3-yl]carbamate (CAS: 1932536-58-7, PharmaBlock, Catalog: PBS63423) in step (a). MS: calc'd 425 [(M+H)⁺], measured 425 [(M+H)⁺]. ¹H NMR (500 MHz, METHANOL-d₄) δ=8.06-8.02 (m, 0.4H), 8.00-7.94 (m, 0.6H), 7.56-7.50 (m, 0.4H), 7.48-7.43 (m, 0.6H), 7.05-6.99 (m, 0.4H), 6.93-6.86 (m, 0.6H), 6.47-6.43 (m, 0.6H), 6.42-6.37 (m, 0.4H), 6.35-6.29 (m, 1H), 3.52-3.39 (m, 1.6H), 3.36-3.32 (m, 0.4H), 3.18-3.05 (m, 2H), 3.01-2.86 (m, 1H), 2.81-2.73 (m, 1H), 2.39-2.30 (m, 1H), 2.19-2.09 (m, 1H), 2.01-1.90 (m, 1H), 1.71-1.53 (m, 1H), 1.49-1.44 (m, 3H), 1.29-1.21 (m, 3H).

### Example 51

### 5-[[(3S,4R)-1-[7-(ethylamino)-5-fluoro-3-methyl-2-oxo-indolin-3-yl]-4-hydroxy-3-piperidyl]amino]pyridine-2-carbonitrile

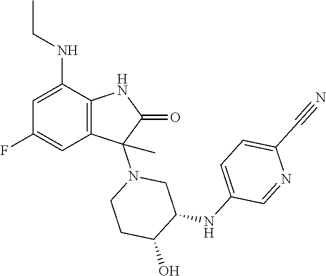 [SMILES: CCNc1cc(F)cc2c1NC(=O)C2(C)N1CC[C@@H](O)[C@@H](Nc2ccc(C#N)nc2)C1]

5-[[(3S,4R)-1-[7-(ethylamino)-5-fluoro-3-methyl-2-oxo-indolin-3-yl]-4-hydroxy-3-piperidyl]amino]pyridine-2-carbonitrile (Example 51) was prepared in analogy to Example 23, by replacing tert-butyl N-[(3R)-3-methyl-3-piperidyl]carbamate (compound 23.1) with tert-butyl N-[(3S,4R)-4-hydroxypiperidin-3-yl]carbamate (CAS: 1549812-73-8, PharmaBlock, Catalog: PBS63425) in step (a). MS: calc'd 425 [(M+H)⁺], measured 425 [(M+H)⁺]. ¹H NMR (500 MHz, METHANOL-d₄) δ=8.16-8.09 (m, 1H), 7.52-7.43 (m, 1H), 7.11-7.02 (m, 1H), 6.51-6.44 (m, 0.5H), 6.41-6.36 (m, 0.5H), 6.35-6.25 (m, 1H), 3.89-3.74 (m, 1.5H), 3.72-3.65 (m, 0.5H), 3.18-3.05 (m, 2H), 3.00-2.86 (m, 1H), 2.80-2.73 (m, 0.5H), 2.70-2.58 (m, 1.5H), 2.57-2.50 (m, 0.5H), 2.48-2.37 (m, 0.5H), 1.84-1.62 (m, 2H), 1.49-1.42 (m, 3H), 1.31-1.22 (m, 3H).

### Example 52

### N-[(3R,4R)-1-[7-(ethylamino)-5-fluoro-3-methyl-2-oxo-indolin-3-yl]-4-phenyl-3-piperidyl]acetamide

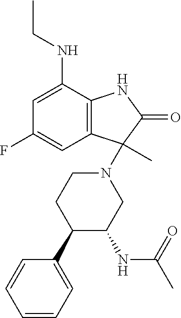 [SMILES: CCNc1cc(F)cc2c1NC(=O)C2(C)N1CC[C@H](c2ccccc2)[C@@H](NC(C)=O)C1]

The titled compound was synthesized according to the following scheme:

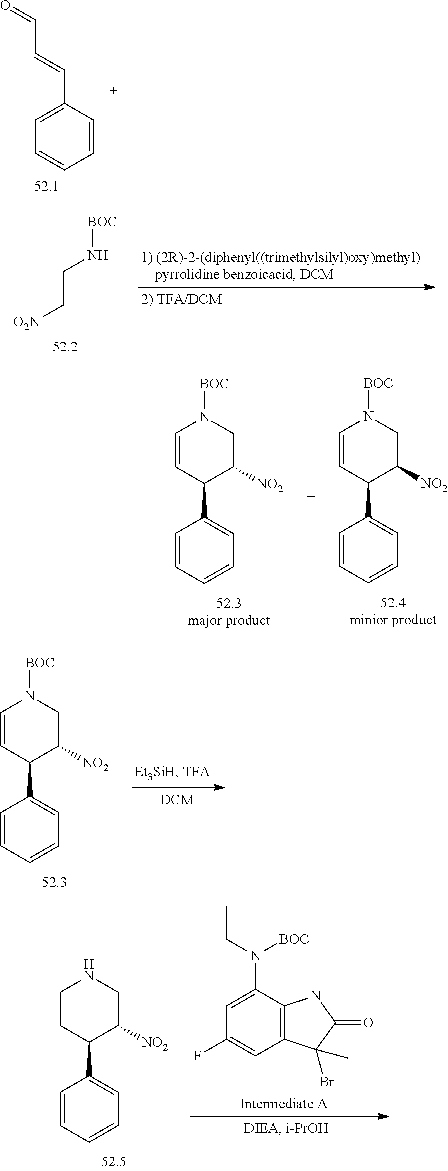

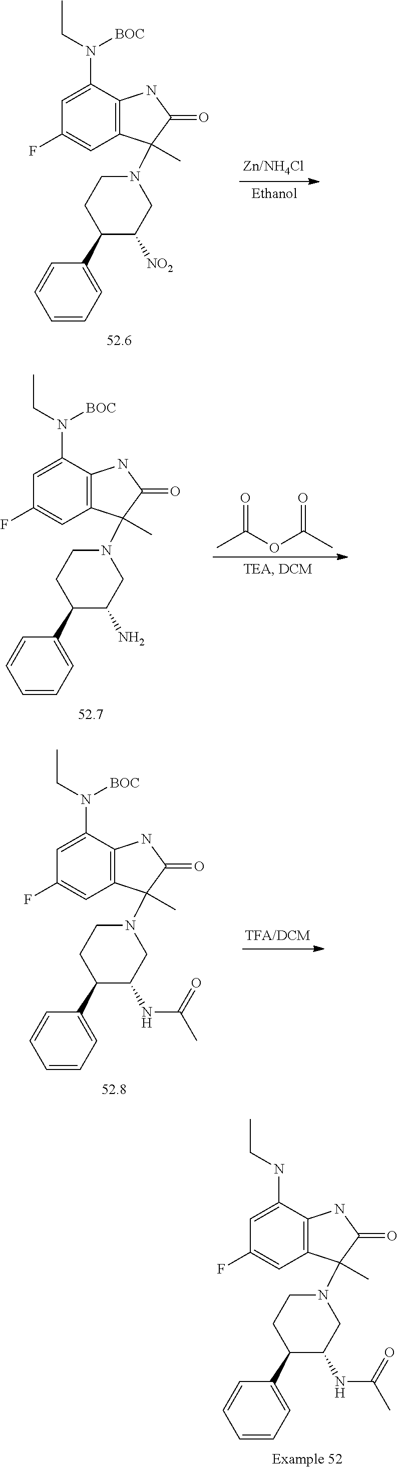

### Step (a): Preparation of tert-butyl (3R,4R)-3-nitro-4-phenyl-3,4-dihydro-2H-pyridine-1-carboxylate (Compound 52.3)

Compound 52.3 was prepared according to ref: Advanced Synthesis and Catalysis. 2012, 354, 991-994; Journal of Medicinal Chemistry. 2019, 62, 3268-3285; and references cited therein. To a solution of tert-butyl N-(2-nitroethyl)carbamate (compound 52.2, 993.0 mg, 5.2 mmol), (2R)-2-(diphenyl((trimethylsilyl)oxy)methyl) pyrrolidine (113.3 mg, 348.6 μmol) and benzoic acid (85.0 mg, 696.7 μmol) in dry DCM (10 mL) was slowly added (E)-3-phenylprop-2-enal (compound 52.1, 457.6 mg, 3.5 mmol). The resultant mixture was stirred at room temperature overnight. Then, TFA (793.7 mg, 7.0 mmol) was added dropwise, and the reaction mixture was stirred for another 2 hrs. The reaction was quenched by adding 1N aqueous sodium bicarbonate solution (20 mL) dropwise, stirred for another 10 min, and extracted with DCM (30 mL) for three times. The combined organic layer was washed with brine, dried over Na₂SO₄, filtered and concentrated in vacuo. The residue was purified by flash chromatography to afford the yellow oil, which was further purified by chiral-HPLC to afford compound 52.3 (300 mg, 28.32% yield) and compound 52.4 (55 mg, 5.2% yield). MS: calc'd 305 [(M+H)⁺], measured 305 [(M+H)⁺].

### Step (b): Preparation of (3R,4R)-3-nitro-4-phenyl-piperidine (Compound 52.5)

To a solution of tert-butyl (3R,4R)-3-nitro-4-phenyl-3,4-dihydro-2H-pyridine-1-carboxylate (compound 52.3, 300 mg, 1.0 mmol) and triethylsilane (229.2 mg, 2.0 mmol in DCM (10 mL) was added dropwise TFA (1.14 g, 10.0 mmol) at 0° C. After the addition was completed, the reaction was warmed up to room temperature and stirred overnight. Then the reaction was quenched by the dropping of saturated aqueous sodium bicarbonate (20 mL), stirred for another 10 min, and extracted with dichloromethane (20 mL) for three times. The combined organic layer was washed with saturated brine, dried over Na₂SO₄ and concentrated in vacuo to afford compound 52.5 (200 mg, 97.6% yield). MS: calc'd 207 [(M+H)⁺], measured 207 [(M+H)⁺].

### Step (c): Preparation of tert-butyl N-ethyl-N-[5-fluoro-3-methyl-3-[(3R,4R)-3-nitro-4-phenyl-1-piperidyl]-2-oxo-indolin-7-yl]carbamate (Compound 52.6)

To a solution of (3R,4R)-3-nitro-4-phenyl-piperidine (compound 52.5, 200 mg, 966.2 μmol) and DIEA (626.7 mg, 4.9 mmol) in isopropanol (10 mL) was added tert-butyl N-(3-bromo-5-fluoro-3-methyl-2-oxo-indolin-7-yl)-N-ethyl-carbamate (intermediate A, 375.5 mg, 972.8 μmol), the resultant mixture was stirred at room temperature for 2 hrs. The reaction mixture was diluted with water (50 mL), extracted with EA (30 mL) twice. The combined organic layer was washed with brine, dried over Na₂SO₄, filtered and concentrated. The residue was purified by flash chromatography (silica gel, 20 g, 30% to 80% EA in PE) to afford compound 52.6 (310 mg, 62.4% yield). MS: calc'd 513 [(M+H)⁺], measured 513 [(M+H)⁺].

### Step (d): Preparation of tert-butyl N-[3-[(3R,4R)-3-amino-4-phenyl-1-piperidyl]-5-fluoro-3-methyl-2-oxo-indolin-7-yl]-N-ethyl-carbamate (Compound 52.7)

To a suspension of tert-butyl N-ethyl-N-[5-fluoro-3-methyl-3-[(3R,4R)-3-nitro-4-phenyl-1-piperidyl]-2-oxo-indolin-7-yl]carbamate (compound 52.6, 310 mg, 0.6 mmol) in ethyl alcohol/water (v/v, 3:1, 20 mL) was added zinc (395.4 mg, 6.0 mmol) and ammonium chloride (323.5 mg, 6.0 mmol), then the mixture was stirred at room temperature overnight. After the reaction was completed, the mixture was filtered and filtrate was concentrated in vacuo. The residue was dissolved in ethyl acetate (80 mL), washed by saturated aqueous sodium bicarbonate and saturated brine, dried over Na₂SO₄, and concentrated. The residue was purified by flash chromatography (silica gel, 20 g, 0% to 10% MeOH in DCM) to afford compound 52.7 (240 mg, 82.23%). MS: calc'd 483 [(M+H)⁺], measured 483 [(M+H)⁺].

### Step (e): Preparation of tert-butyl N-[3-[(3R,4R)-3-acetamido-4-phenyl-1-piperidyl]-5-fluoro-3-methyl-2-oxo-indolin-7-yl]-N-ethyl-carbamate (Compound 52.8)

To a solution of tert-butyl N-[3-[(3R,4R)-3-amino-4-phenyl-1-piperidyl]-5-fluoro-3-methyl-2-oxo-indolin-7-yl]-N-ethyl-carbamate (compound 52.7, 40 mg, 82.8 μmol) and TEA (25.2 mg, 247 μmol) in DCM (5 mL) was added Ac₂O (10.2 mg, 0.1 mmol), the resultant mixture was stirred at room temperature for 30 min, then diluted with water, extracted with DCM. The organic layer was washed with brine, dried over Na₂SO₄, filtered and concentrated. The residue was purified by flash chromatography (silica gel, 20 g, 20% to 100% EA in PE) to afford compound 52.8 (35 mg, 80.5% yield). MS: calc'd 525 [(M+H)⁺], measured 525 [(M+H)⁺].

### Step (f): Preparation of N-[(3R,4R)-1-[7-(ethylamino)-5-fluoro-3-methyl-2-oxo-indolin-3-yl]-4-phenyl-3-piperidyl]acetamide (Example 52)

To a solution of tert-butyl N-[3-[(3R,4R)-3-acetamido-4-phenyl-1-piperidyl]-5-fluoro-3-methyl-2-oxo-indolin-7-yl]-N-ethyl-carbamate (compound 52.8, 30 mg, 57.2 μmol) in DCM (10 mL) was added TFA (5 mL). The reaction mixture was stirred at room temperature for 2 hrs, then concentrated to afford a crude product, which was purified by pre-HPLC to afford Example 52 (18 mg, 74.2% yield). MS: calc'd 425 [(M+H)⁺], measured 425 [(M+H)⁺]. ¹H NMR (500 MHz, METHANOL-d₄) δ=7.34-7.27 (m, 2H), 7.26-7.19 (m, 3H), 6.72-6.62 (m, 1H), 6.56-6.44 (m, 1H), 4.52-4.32 (m, 1H), 3.69-3.46 (m, 2H), 3.22-3.15 (m, 2H), 3.13-3.04 (m, 1H), 3.02-2.94 (m, 0.5H), 2.92-2.84 (m, 0.5H), 2.81-2.70 (m, 1H), 2.14-1.93 (m, 2H), 1.81-1.74 (m, 3H), 1.73-1.67 (m, 3H), 1.33-1.21 (m, 3H).

### Example 53

### N-[(3R,4R)-1-[7-(ethylamino)-5-fluoro-3-methyl-2-oxo-indolin-3-yl]-4-phenyl-3-piperidyl]ethenesulfonamide

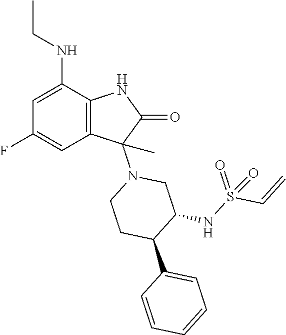 [SMILES: C=CS(=O)(=O)N[C@H]1CN(C2(C)C(=O)Nc3c(NCC)cc(F)cc32)CC[C@@H]1c1ccccc1]

The titled compound was synthesized according to the following scheme:

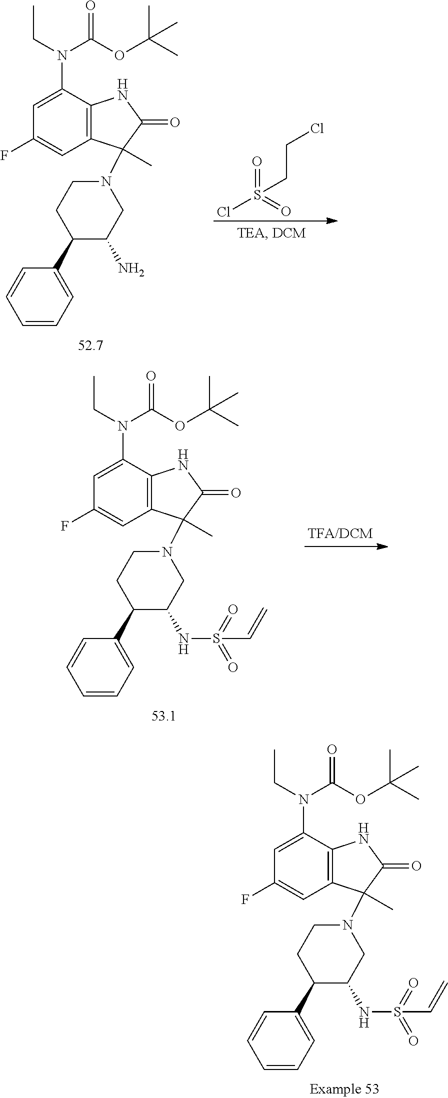 [SMILES: CCN(C(=O)OC(C)(C)C)c1cc(F)cc2c1NC(=O)C2(C)N1CC[C@H](c2ccccc2)[C@@H](N)C1 ; O=S(=O)(Cl)CCCl ; C=CS(=O)(=O)N[C@H]1CN(C2(C)C(=O)Nc3c(N(CC)C(=O)OC(C)(C)C)cc(F)cc32)CC[C@@H]1c1ccccc1 ; C=CS(=O)(=O)N[C@H]1CN(C2(C)C(=O)Nc3c(N(CC)C(=O)OC(C)(C)C)cc(F)cc32)CC[C@@H]1c1ccccc1]

### Step (a): Preparation of tert-butyl N-ethyl-N-[5-fluoro-3-methyl-2-oxo-3-[(3R,4R)-4-phenyl-3-(vinylsulfonylamino)-1-piperidyl]indolin-7-yl]carbamate (Compound 53.1)

To a solution of tert-butyl N-[3-[(3R,4R)-3-amino-4-phenyl-1-piperidyl]-5-fluoro-3-methyl-2-oxo-indolin-7-yl]-N-ethyl-carbamate (compound 52.7, 30 mg, 62.2 μmol) and TEA (18.9 mg, 185.2 μmol) in DCM (5 mL) was added dropwise 2-chloroethanesulfonyl chloride (12.2 mg, 75 μmol) in dichloromethane (1 mL) at 0° C., the resultant mixture was stirred at room temperature for 2 hrs. The reaction was quenched with aq.NaHCO₃, extracted with DCM. The organic layer was washed with brine, dried over Na₂SO₄, filtered and concentrated. The residue was purified by flash chromatography (silica gel, 12 g, 0% to 100% EA in PE) to afford compound 53.1 (20 mg, 56.2% yield). MS: calc'd 573 [(M+H)⁺], measured 573 [(M+H)⁺].

### Step (b): Preparation of N-[(3R,4R)-1-[7-(ethylamino)-5-fluoro-3-methyl-2-oxo-indolin-3-yl]-4-phenyl-3-piperidyl]ethenesulfonamide (Example 53)

To a solution of tert-butyl N-ethyl-N-[5-fluoro-3-methyl-2-oxo-3-[(3R,4R)-4-phenyl-3-(vinylsulfonylamino)-1-piperidyl]indolin-7-yl]carbamate (compound 53.1, 20 mg, 35 μmol) in DCM (10 mL) was added TFA (5 mL). The reaction mixture was stirred at room temperature for 2 hrs, then concentrated to afford a crude product, which was purified by pre-HPLC to afford Example 53 (9 mg, 54.5% yield). MS: calc'd 473 [(M+H)⁺], measured 473 [(M+H)⁺]. ¹H NMR (500 MHz, METHANOL-d₄) δ 7.36-7.30 (m, 2H), 7.29-7.20 (m, 3H), 6.65-6.55 (m, 1H), 6.52-6.44 (m, 1H), 5.81-5.70 (m, 1H), 5.56-5.48 (m, 1H), 5.47-5.41 (m, 0.5H), 5.40-5.34 (m, 0.5H), 3.65-3.52 (m, 2H), 3.47-3.34 (m, 1H), 3.21-3.14 (m, 2H), 2.96-2.68 (m, 2H), 2.61-2.48 (m, 1H), 2.08-1.90 (m, 2H), 1.74-1.65 (m, 3H), 1.34-1.25 (in, 3H).

### Example 54

### 1-cyano-N-[(3R,4R)-1-[7-(ethylamino)-5-fluoro-3-methyl-2-oxo-indolin-3-yl]-4-phenyl-3-piperidyl]azetidine-3-carboxamide

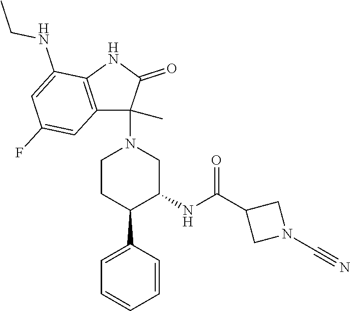 [SMILES: CCNc1cc(F)cc2c1NC(=O)C2(C)N1CC[C@H](c2ccccc2)[C@@H](NC(=O)C2CN(C#N)C2)C1]

The titled compound was synthesized according to the following scheme:

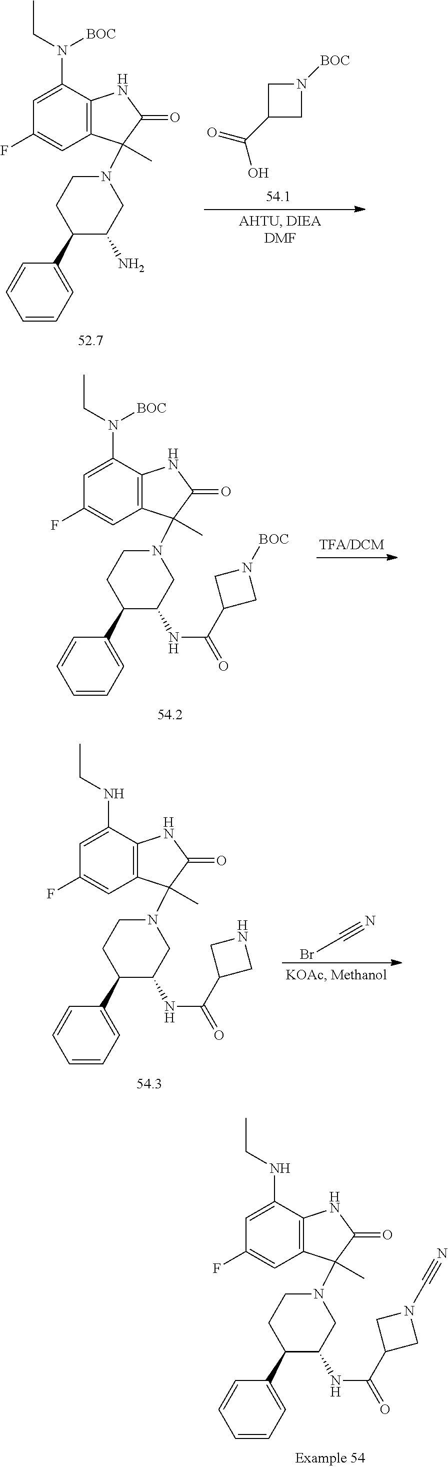

### Step (a): Preparation of tert-butyl 3-[[(3R,4R)-1-[7-[tert-butoxycarbonyl(ethyl)amino]-5-fluoro-3-methyl-2-oxo-indolin-3-yl]-4-phenyl-3-piperidyl]carbamoyl]azetidine-1-carboxylate (Compound 54.2)

To a solution of 1-tert-butoxycarbonylazetidine-3-carboxylic acid (compound 54.1, 25.0 mg, 124 μmol) and DIEA (48.2 mg, 373 μmol) in DMF (6 mL) was added HATU (52.0 mg, 137 μmol). After being stirred at room temperature for 30 min, tert-butyl N-[3-[(3R,4R)-3-amino-4-phenyl-1-piperidyl]-5-fluoro-3-methyl-2-oxo-indolin-7-yl]-N-ethyl-carbamate (compound 52.7, 60 mg, 124 μmol) was added to the reaction mixture, which was stirred at room temperature for another 2 hrs. The reaction mixture was diluted with water, extracted with EA. The organic layer was washed with brine, dried over Na₂SO₄, filtered and concentrated. The residue was purified by flash chromatography (silica gel, 20 g, 0% to 100% EA in PE) to afford compound 54.2 (70 mg, 84.6% yield) as colorless oil. MS: calc'd 666 [(M+H)⁺], measured 666 [(M+H)⁺].

### Step (b): Preparation of 1-cyano-N-[(3R,4R)-1-[7-(ethylamino)-5-fluoro-3-methyl-2-oxo-indolin-3-yl]-4-phenyl-3-piperidyl]azetidine-3-carboxamide (Example 54)

To a solution of tert-butyl 3-[[(3R,4R)-1-[7-[tert-butoxycarbonyl(ethyl)amino]-5-fluoro-3-methyl-2-oxo-indolin-3-yl]-4-phenyl-3-piperidyl]carbamoyl]azetidine-1-carboxylate (compound 54.2, 70 mg, 105 μmol) in DCM (10 mL) was added TFA (5 mL). The reaction mixture was stirred at room temperature for 2 hrs, then concentrated to afford an oil. To a solution of the residue in methanol (5 mL) was added potassium acetate (31.5 mg, 321 μmol) and cyanogen bromide (5.0 mg, 118 μmol), the resultant mixture was stirred at room temperature for 5 hrs. The reaction mixture was quenched with water (0.5 mL), then concentrated to afford a crude product, which was purified by flash chromatography (silica gel, 10 g, 0% to 20% MeOH in DCM) to afford Example 54 (23 mg, 44.7% yield). MS: calc'd 491 [(M+H)⁺], measured 491 [(M+H)⁺]. ¹H NMR (500 MHz, METHANOL-d₄) δ=7.27-7.23 (m, 2H), 7.22-7.15 (m, 3H), 6.45-6.40 (m, 1H), 6.35-6.29 (m, 1H), 4.26-4.01 (m, 3H), 4.00-3.91 (m, 1H), 3.54-3.49 (m, 0.5H), 3.43-3.39 (m, 0.5H), 3.24-3.10 (m, 4H), 2.98-2.90 (m, 0.5H), 2.84-2.76 (m, 0.5H), 2.71-2.62 (m, 0.5H), 2.59-2.50 (m, 0.5H), 2.48-2.38 (m, 1H), 2.34-2.23 (m, 0.5H), 2.20-2.05 (m, 0.5H), 1.89-1.67 (m, 2H), 1.52-1.47 (m, 3H), 1.30-1.26 (m, 3H).

### Example 55

### 5-[[(3R,4R)-1-[7-(ethylamino)-5-fluoro-3-methyl-2-oxo-indolin-3-yl]-4-phenyl-3-piperidyl]amino]pyridine-2-carbonitrile

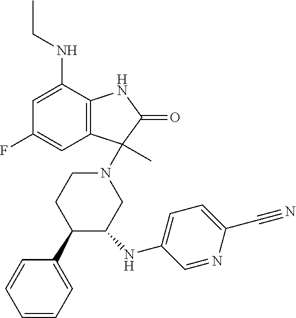 [SMILES: CCNc1cc(F)cc2c1NC(=O)C2(C)N1CC[C@H](c2ccccc2)[C@@H](Nc2ccc(C#N)nc2)C1]

The titled compound was synthesized according to the following scheme:

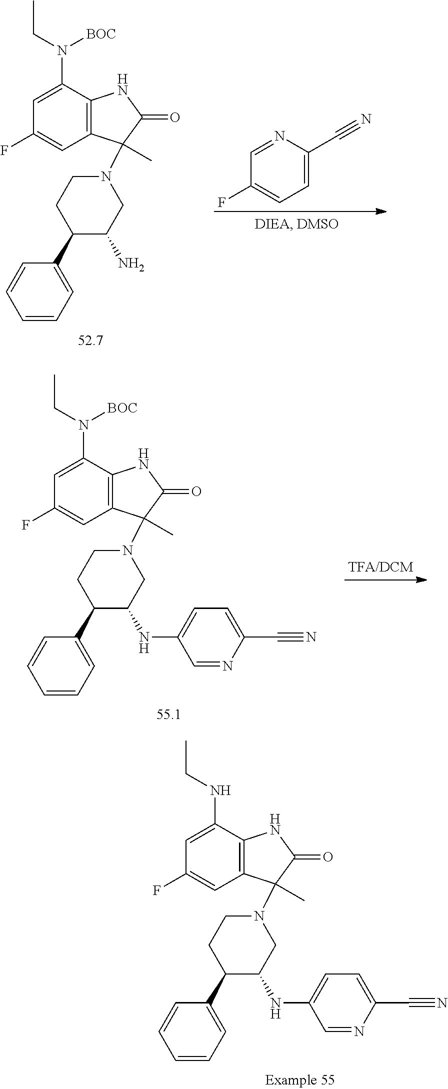 [SMILES: CCN(C(=O)OC(C)(C)C)c1cc(F)cc2c1NC(=O)C2(C)N1CC[C@H](c2ccccc2)[C@@H](N)C1 ; N#Cc1ccc(F)cn1 ; CCN(C(=O)OC(C)(C)C)c1cc(F)cc2c1NC(=O)C2(C)N1CC[C@H](c2ccccc2)[C@@H](Nc2ccc(C#N)nc2)C1 ; CCNc1cc(F)cc2c1NC(=O)C2(C)N1CC[C@H](c2ccccc2)[C@@H](Nc2ccc(C#N)nc2)C1]

### Step (a): Preparation of tert-butyl N-ethyl-N-[5-fluoro-3-methyl-2-oxo-3-[(3R,4R)-3-[(6-cyano-3-pyridyl)amino]-4-phenyl-1-piperidyl]indolin-7-yl]carbamate (Compound 55.1)

To a 5 mL microwave vial was added tert-butyl N-[3-[(3R,4R)-3-amino-4-phenyl-1-piperidyl]-5-fluoro-3-methyl-2-oxo-indolin-7-yl]-N-ethyl-carbamate (compound 52.7, 80 mg, 166 μmol), 5-fluoropyridine-2-carbonitrile (30.4 mg, 249 μmol) and DIEA (107.1 mg, 829 μmol) in DMSO (5 mL). The vial was capped and heated in the microwave at 120° C. for 5 hrs. After being cooled to room temperature, the reaction mixture was diluted with water, and extracted two times with EA. The combined organic layer was washed with water and brine, dried over Na₂SO₄ and concentrated to dryness. The residue was purified by flash chromatography (silica gel, 40 g, 0% to 50% EA in PE) to afford compound 55.1 (55 mg, 56.8% yield). MS: calc'd 585 [(M+H)⁺], measured 585 [(M+H)⁺].

### Step (b): Preparation of 5-[[(3R,4R)-1-[7-(ethylamino)-5-fluoro-3-methyl-2-oxo-indolin-3-yl]-4-phenyl-3-piperidyl]amino]pyridine-2-carbonitrile (Example 55)

To a solution of tert-butyl N-ethyl-N-[5-fluoro-3-methyl-2-oxo-3-[(3R,4R)-3-[(6-cyano-3-pyridyl)amino]-4-phenyl-1-piperidyl]indolin-7-yl]carbamate (compound 55.1, 55 mg, 94 μmol) in DCM (10 mL) was added TFA (5 mL). The reaction mixture was stirred at room temperature for 2 hrs, then concentrated to afford a crude product, which was purified by pre-HPLC to afford Example 55 (17 mg, 37.3% yield). MS: calc'd 485 [(M+H)⁺], measured 485 [(M+H)⁺]. ¹H NMR (500 MHz, METHANOL-d₄) δ=7.79-7.75 (m, 1H), 7.42-7.35 (m, 1H), 7.26-7.19 (m, 4H), 7.18-7.11 (m, 1H), 6.83-6.78 (m, 0.5H), 6.77-6.73 (m, 0.5H), 6.66-6.60 (m, 1H), 6.51-6.42 (m, 1H), 4.04-3.93 (m, 1H), 3.70-3.55 (m, 1H), 3.54-3.35 (m, 1H), 3.19-3.13 (m, 2H), 3.04-2.90 (m, 1H), 2.83-2.64 (m, 2H), 2.14-1.98 (m, 2H), 1.72 (s, 3H), 1.27 (t, J=7.1 Hz, 3H).

### Example 56

### 4-[2-[(3R,4R)-3-acetamido-1-[7-(ethylamino)-5-fluoro-3-methyl-2-oxo-indolin-3-yl]-4-piperidyl]phenoxy]benzoic acid

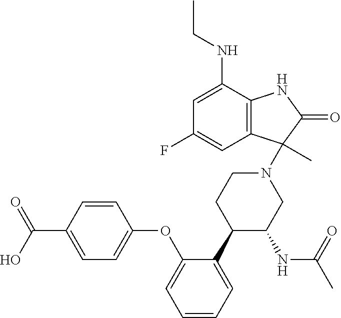 [SMILES: CCNc1cc(F)cc2c1NC(=O)C2(C)N1CC[C@H](c2ccccc2Oc2ccc(C(=O)O)cc2)[C@@H](NC(C)=O)C1]

The titled compound was synthesized according to the following scheme:

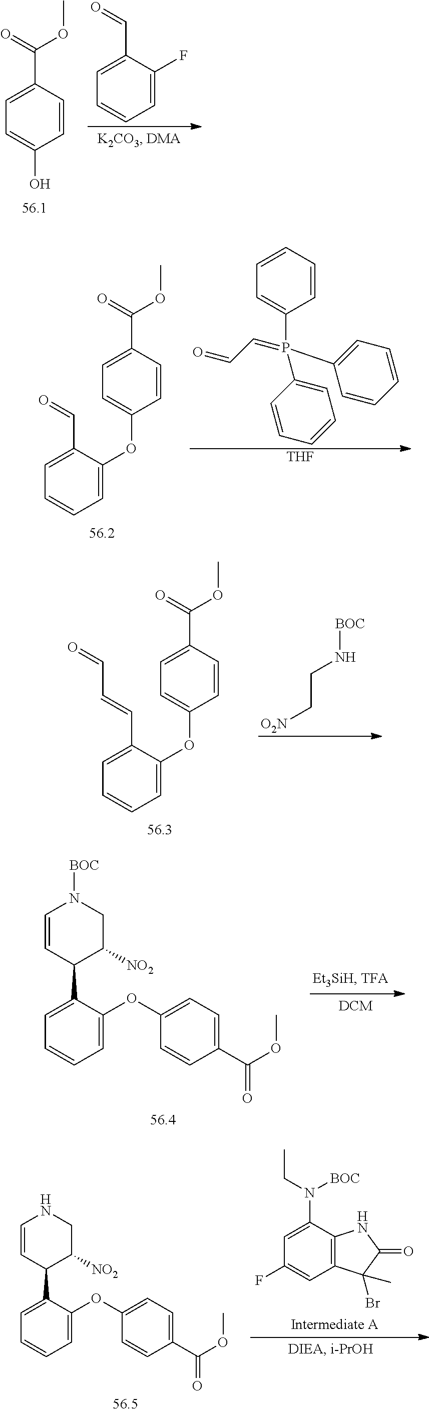

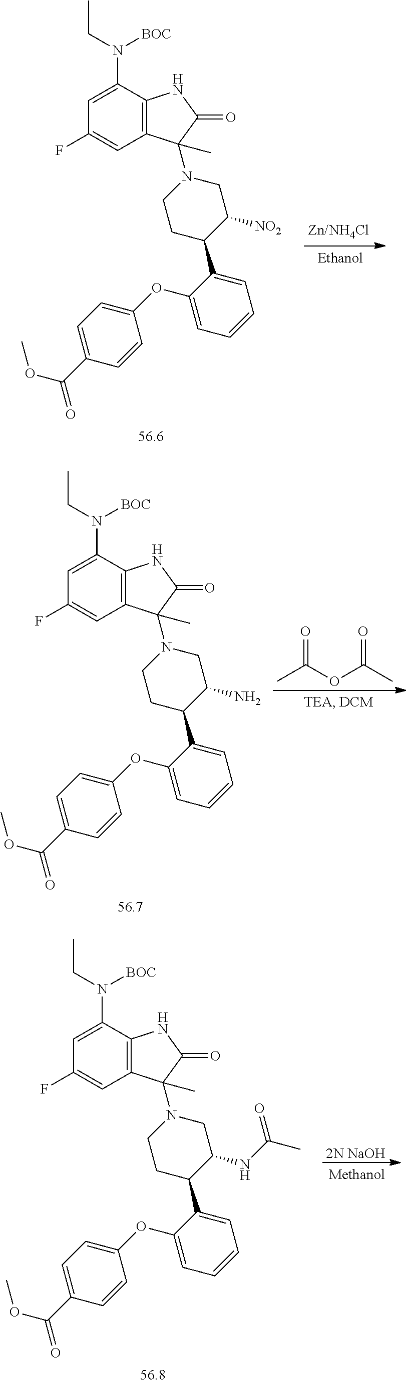

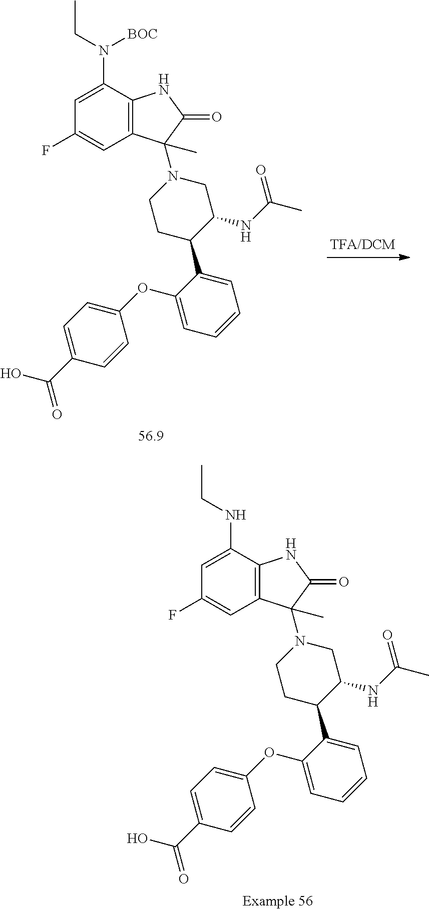 [SMILES: CCN(C(=O)OC(C)(C)C)c1cc(F)cc2c1NC(=O)C2(C)N1CC[C@H](c2ccccc2Oc2ccc(C(=O)O)cc2)[C@@H](NC(C)=O)C1 ; CCNc1cc(F)cc2c1NC(=O)C2(C)N1CC[C@H](c2ccccc2Oc2ccc(C(=O)O)cc2)[C@@H](NC(C)=O)C1]

### Step (a): Preparation of methyl 4-(2-formylphenoxy)benzoate (Compound 56.2)

To a solution of 2-fluorobenzaldehyde (815.7 mg, 6.6 mmol) in DMA (20 mL) was added methyl 4-hydroxybenzoate (compound 56.1, 1000 mg, 6.6 mmol) and K₂CO₃ (2.7 g, 19.7 mmol), the resultant mixture was stirred at 170° C. for 3 hrs. After being cooled to room temperature, the reaction mixture was diluted with water, extracted with EA. The organic layer was washed with brine, dried over Na₂SO₄, filtered and concentrated. The residue was purified by flash chromatography to afford compound 56.2 (1.1 g, 65.3% yield). MS: calc'd 257 [(M+H)⁺], measured 257 [(M+H)⁺].

### Step (b): Preparation of methyl 4-[2-[(E)-3-oxoprop-1-enyl]phenoxy]benzoate (Compound 56.3)

The mixture of methyl 4-(2-formylphenoxy)benzoate (compound 56.2, 1.1 g, 3.5 mmol) and (triphenylphosphoranylidene)acetaldehyde (1.1 g, 3.5 mmol) in THF (20 mL) was refluxed overnight. The reaction mixture was concentrated to afford a crude product, which was purified by flash chromatography (silica gel, 40 g, 20% to 100% EA in PE) to afford compound 56.3 (450 mg, 45.6% yield). MS: calc'd 283 [(M+H)⁺], measured 283 [(M+H)⁺].

### Step (c): Preparation of tert-butyl (3R,4R)-4-[2-(4-methoxycarbonylphenoxy)phenyl]-3-nitro-3,4-dihydro-2H-pyridine-1-carboxylate (Compound 56.4)

To a solution of tert-butyl N-(2-nitroethyl)carbamate (1374160-25-4, Bide Pharmatech, catalog: BD00810616; 464.9 mg, 2.4 mmol), (2R)-2-(diphenyl((trimethylsilyl)oxy)methyl) pyrrolidine (53.0 mg, 163.1 μmol) and benzoic acid (39.8 mg, 326.2 μmol) in dry dichloromethane (5 mL) was slowly added methyl 4-[2-[(E)-3-oxoprop-1-enyl]phenoxy]benzoate (compound 56.3, 464.9 mg, 2.4 mmol). The resultant mixture was stirred at room temperature overnight. Then TFA (547.2 mg, 4.8 mmol) was added dropwise, and the reaction mixture was stirred for another 2 hrs. The reaction was quenched by the dropping of 1 N aqueous sodium bicarbonate solution (20 mL) and stirred for another 10 min, extracted with DCM (50 mL) twice. The combined organic layer was washed with brine, dried over Na₂SO₄, filtered and concentrated in vacuo. The residue was purified by flash column chromatography to afford the yellow oil, which was purified by chiral-HPLC to afford compound 56.4 (260 mg, 35.1%). MS: calc'd 455 [(M+H)⁺], measured 455 [(M+H)⁺].

### Step (d): Preparation of methyl 4-[2-[(3R,4R)-3-nitro-4-piperidyl]phenoxy]benzoate (Compound 56.5)

To a solution of tert-butyl (3R,4R)-4-[2-(4-methoxycarbonylphenoxy)phenyl]-3-nitro-3,4-dihydro-2H-pyridine-1-carboxylate (compound 56.4, 240 mg, 0.5 mmol) and triethylsilane (122.8 mg, 1.1 mmol) in DCM (10 mL) was added dropwise TFA (602.1 mg, 5.2 mmol) at 0° C. After the addition was completed, the reaction was warmed up to room temperature and stirred overnight. Then the reaction was quenched by addition of saturated aqueous sodium bicarbonate (20 mL) dropwise, stirred for another 10 min, and extracted with dichloromethane (20 mL) for three times. The combined organic layer was washed with brine dried over Na₂SO₄, and concentrated in vacuo to afford compound 56.5 (170 mg, 95.5% yield). MS: calc'd 357 [(M+H)⁺], measured 357 [(M+H)⁺].

### Step (e): Preparation of methyl 4-[2-[(3R,4R)-1-[7-[tert-butoxycarbonyl(ethyl)amino]-5-fluoro-3-methyl-2-oxo-indolin-3-yl]-3-nitro-4-piperidyl]phenoxy]benzoate (Compound 56.6)

To a solution methyl 4-[2-[(3R,4R)-3-nitro-4-piperidyl]phenoxy]benzoate (compound 56.5, 100 mg, 280.8 μmol) and DIEA (181.3 mg, 1.4 mmol) in isopropanol (8 mL) was added tert-butyl N-(3-bromo-5-fluoro-3-methyl-2-oxo-indolin-7-yl)-N-ethyl-carbamate (intermediate A, 108.7 mg, 281.8 μmol), the resultant mixture was stirred at room temperature for 2 hrs, then diluted with water (50 mL), and extracted with EA (30 mL) twice. The combined organic layer was washed with brine, dried over Na₂SO₄, filtered and concentrated. The residue was purified by flash chromatography (silica gel, 20 g, 30% to 80% EA in PE) to afford compound 56.6 (160 mg, 86.0% yield). MS: calc'd 663 [(M+H)⁺], measured 663 [(M+H)⁺].

### Step (f): Preparation of methyl 4-[2-[(3R,4R)-3-amino-1-[7-[tert-butoxycarbonyl(ethyl)amino]-5-fluoro-3-methyl-2-oxo-indolin-3-yl]-4-piperidyl]phenoxy]benzoate (Compound 56.7)

To a suspension of methyl 4-[2-[(3R,4R)-1-[7-[tert-butoxycarbonyl(ethyl)amino]-5-fluoro-3-methyl-2-oxo-indolin-3-yl]-3-nitro-4-piperidyl]phenoxy]benzoate (compound 56.6, 200 mg, 302.1 μmol) in ethanol/water (v/v, 3:1, 20 mL) was added zinc (197.3 mg, 3.0 mmol) and ammonium chloride (161.4 mg, 3.0 mmol), then the resultant mixture was stirred at room temperature overnight. After the reaction is completed, the mixture was filtered and filtrate was concentrated in vacuo. The residue was dissolved in ethyl acetate (80 mL), washed by saturated aqueous sodium bicarbonate and saturated brine, and dried over Na₂SO₄. After solvent removal, the residue was purified by flash chromatography (silica gel, 20 g, 0% to 10% MeOH in DCM) to afford compound 56.7 (180 mg, 94.3% yield). MS: calc'd 633 [(M+H)⁺], measured 633 [(M+H)⁺].

### Step (f): Preparation of methyl 4-[2-[(3R,4R)-3-acetamido-1-[7-[tert-butoxycarbonyl(ethyl)amino]-5-fluoro-3-methyl-2-oxo-indolin-3-yl]-4-piperidyl]phenoxy]benzoate (Compound 56.8)

To a solution of methyl 4-[2-[(3R,4R)-3-amino-1-[7-[tert-butoxycarbonyl(ethyl)amino]-5-fluoro-3-methyl-2-oxo-indolin-3-yl]-4-piperidyl]phenoxy]benzoate (compound 56.7, 180 mg, 284.8 μmol) and Et₃N (86.4 mg, 847.0 μmol) in DCM (10 mL) was added Ac₂O (34.8 mg, 341.1 μmol), the resultant mixture was stirred at room temperature for 30 min. The reaction mixture was diluted with water, and extracted with DCM. The organic layer was washed with brine, dried over Na₂SO₄, filtered and concentrated in vacuo. The residue was purified by flash chromatography to afford compound 56.8 (160 mg, 83.3% yield). MS: calc'd 675 [(M+H)⁺], measured 675 [(M+H)⁺].

### Step (g): Preparation of 4-[2-[(3R,4R)-3-acetamido-1-[7-[tert butoxycarbonyl(ethyl)amino]-5-fluoro-3-methyl-2-oxo-indolin-3-yl]-4-piperidyl]phenoxy]benzoic acid (Compound 56.9)

To a solution of methyl 4-[2-[(3R,4R)-3-acetamido-1-[7-[tert-butoxycarbonyl(ethyl)amino]-5-fluoro-3-methyl-2-oxo-indolin-3-yl]-4-piperidyl]phenoxy]benzoate (compound 56.8, 170 mg, 251.9 μmol) in methanol (10 mL) was added aqueous sodium hydroxide (2 N, 5 mL). The resultant mixture was stirred at room temperature overnight. The reaction mixture was concentrated, diluted with water (10 mL), added 2 N HCl to acidify the mixture to pH about 5, and then extracted with EA (30 mL) twice. The combined organic layer was washed with brine, dried over Na₂SO₄, filtered and concentrated to afford compound 56.9 (160 mg, 96.1% yield). MS: calc'd 661 [(M+H)⁺], measured 661 [(M+H)⁺].

### Step (h): Preparation of 4-[2-[(3R,4R)-3-acetamido-1-[7-(ethylamino)-5-fluoro-3-methyl-2-oxo-indolin-3-yl]-4-piperidyl]phenoxy]benzoic acid (Example 56)

To a solution of 4-[2-[(3R,4R)-3-acetamido-1-[7-[tert-butoxycarbonyl(ethyl)amino]-5-fluoro-3-methyl-2-oxo-indolin-3-yl]-4-piperidyl]phenoxy]benzoic acid (compound 56.9, 50 mg, 75.8 μmol) in DCM (10 mL) was added TFA (5 mL). The reaction mixture was stirred at room temperature for 2 hrs, then concentrated to afford a crude product which was purified by pre-HPLC to afford Example 56 (40 mg, 94.2% yield). MS: calc'd 561 [(M+H)⁺], measured 561 [(M+H)⁺]. ¹H NMR (500 MHz, METHANOL-d₄) δ=8.09-7.95 (m, 2H), 7.44-7.32 (m, 1H), 7.30-7.22 (m, 1H), 7.22-7.13 (m, 1H), 7.06-6.97 (m, 2H), 6.94-6.84 (m, 1H), 6.63-6.52 (m, 1H), 6.51-6.39 (m, 1H), 4.66-4.44 (m, 1H), 3.56-3.34 (m, 2H), 3.19-3.13 (m, 2H), 3.12-2.71 (m, 3H), 2.09-1.95 (m, 2H), 1.75 (s, 3H), 1.72-1.67 (m, 3H), 1.31-1.23 (m, 3H)

### Example 57

### 4-[2-[(3R,4R)-3-acetamido-1-[7-(ethylamino)-5-fluoro-3-methyl-2-oxo-indolin-3-yl]-4-piperidyl]phenoxy]benzamide

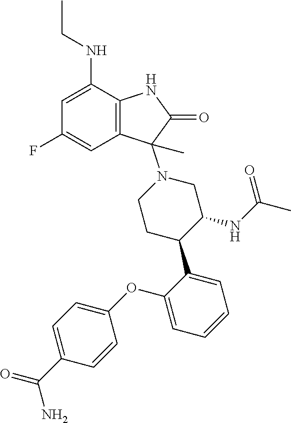 [SMILES: CCNc1cc(F)cc2c1NC(=O)C2(C)N1CC[C@H](c2ccccc2Oc2ccc(C(N)=O)cc2)[C@@H](NC(C)=O)C1]

The titled compound was synthesized according to the following scheme:

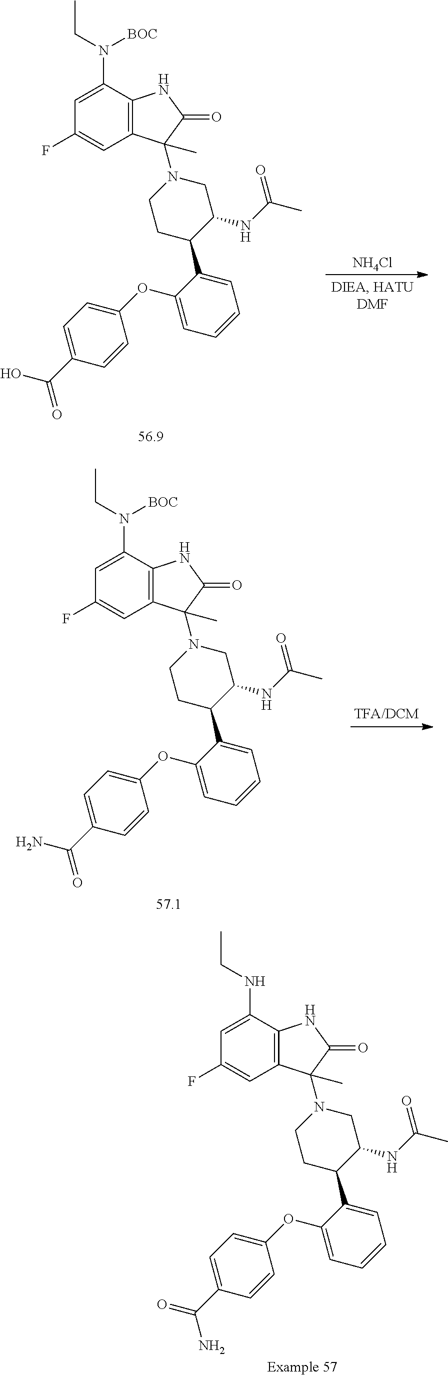 [SMILES: CCN(C(=O)OC(C)(C)C)c1cc(F)cc2c1NC(=O)C2(C)N1CC[C@H](c2ccccc2Oc2ccc(C(=O)O)cc2)[C@@H](NC(C)=O)C1 ; "NH4Cl" ; CCN(C(=O)OC(C)(C)C)c1cc(F)cc2c1NC(=O)C2(C)N1CC[C@H](c2ccccc2Oc2ccc(C(N)=O)cc2)[C@@H](NC(C)=O)C1 ; CCNc1cc(F)cc2c1NC(=O)C2(C)N1CC[C@H](c2ccccc2Oc2ccc(C(N)=O)cc2)[C@@H](NC(C)=O)C1]

### Step (a): Preparation of tert-butyl N-ethyl-N-[5-fluoro-3-methyl-2-oxo-3-[(3R,4R)-3-acetamido-4-[2-(4-carbamoylphenoxy)phenyl]-1-piperidyl]indolin-7-yl]carbamate (Compound 57.1)

To a solution of 4-[2-[(3R,4R)-3-acetamido-1-[7-[tert-butoxycarbonyl(ethyl)amino]-5-fluoro-3-methyl-2-oxo-indolin-3-yl]-4-piperidyl]phenoxy]benzoic acid (compound 56.9, 110 mg, 166.7 μmol) in DMF (5 mL) was added ammonium chloride (89.0 mg, 1.66 mmol), HATU (75.9 mg, 0.2 mmol) and DIEA (107.6 mg, 834.1 μmol), the resultant mixture was stirred at room temperature for 2 hrs. The reaction mixture was quenched with water and extracted twice with EA. The organic layer was washed with brine, dried over Na₂SO₄, filtered and concentrated in vacuo. The residue was purified by flash column chromatography (silica gel, 20 g, 0% to 15% MeOH in DCM) to afford compound 57.1 (82 mg, 74.7% yield). MS: calc'd 660 [(M+H)⁺], measured 660 [(M+H)⁺].

### Step (b): Preparation of 4-[2-[(3R,4R)-3-acetamido-1-[7-(ethylamino)-5-fluoro-3-methyl-2-oxo-indolin-3-yl]-4-piperidyl]phenoxy]benzamide (Example 57)

To a solution of tert-butyl N-ethyl-N-[5-fluoro-3-methyl-2-oxo-3-[(3R,4R)-3-acetamido-4-[2-(4-carbamoylphenoxy)phenyl]-1-piperidyl]indolin-7-yl]carbamate (compound 57.1, 25 mg, 37.9 μmol) in DCM (10 mL) was added TFA (5 mL). The reaction mixture was stirred at room temperature for 2 hrs, then concentrated to afford a crude product, which was purified by pre-HPLC to afford Example 57 (15 mg, 52.9% yield). MS: calc'd 560 [(M+H)⁺], measured 560 [(M+H)⁺]. ¹H NMR (500 MHz, METHANOL-d₄) δ=7.89 (d, J=8.7 Hz, 2H), 7.36 (br d, J=7.6 Hz, 1H), 7.29-7.22 (m, 1H), 7.19-7.13 (m, 1H), 7.06-6.98 (m, 2H), 6.90-6.84 (m, 1H), 6.67-6.57 (m, 1H), 6.54-6.44 (m, 1H), 4.67 (br s, 1H), 3.65-3.44 (m, 2H), 3.18-3.11 (m, 3H), 3.05-2.86 (m, 2H), 2.09-1.98 (m, 2H), 1.77-1.72 (m, 6H), 1.30-1.25 (m, 3H).

### Example 58

### N-[(3R,4R)-4-[2-(4-cyanophenoxy)phenyl]-1-[7-(ethylamino)-5-fluoro-3-methyl-2-oxo-indolin-3-yl]-3-piperidyl]acetamide

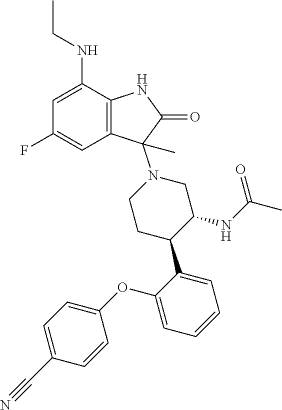 [SMILES: CCNc1cc(F)cc2c1NC(=O)C2(C)N1CC[C@H](c2ccccc2Oc2ccc(C#N)cc2)[C@@H](NC(C)=O)C1]

The titled compound was synthesized according to the following scheme:

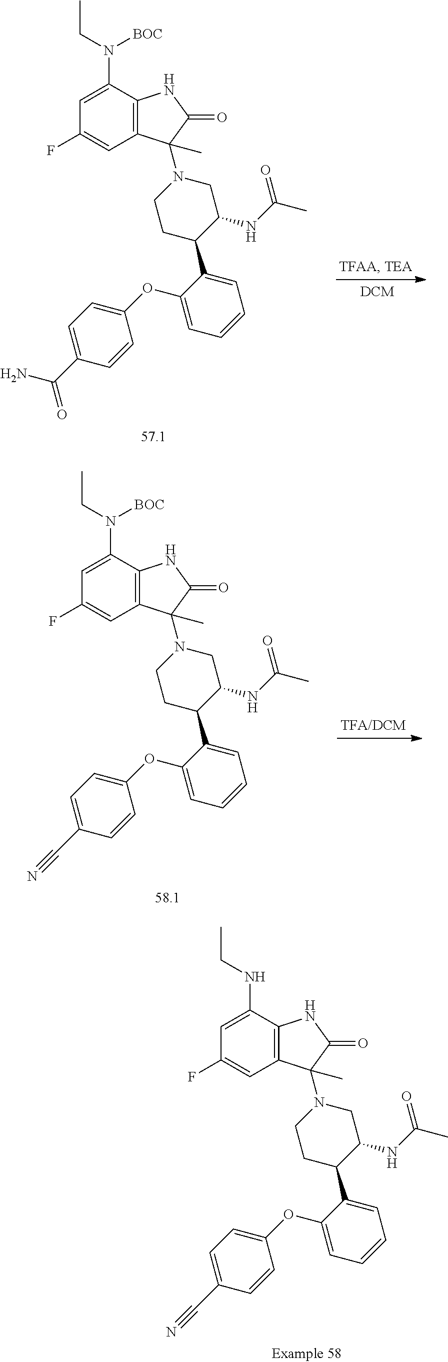 [SMILES: CCN(C(=O)OC(C)(C)C)c1cc(F)cc2c1NC(=O)C2(C)N1CC[C@H](c2ccccc2Oc2ccc(C(N)=O)cc2)[C@@H](NC(C)=O)C1 ; CCN(C(=O)OC(C)(C)C)c1cc(F)cc2c1NC(=O)C2(C)N1CC[C@H](c2ccccc2Oc2ccc(C#N)cc2)[C@@H](NC(C)=O)C1 ; CCNc1cc(F)cc2c1NC(=O)C2(C)N1CC[C@H](c2ccccc2Oc2ccc(C#N)cc2)[C@@H](NC(C)=O)C1]

### Step (a): Preparation of tert-butyl N-ethyl-N-[5-fluoro-3-methyl-2-oxo-3-[(3R,4R)-3-acetamido-4-[2-(4-cyanophenoxy)phenyl]-1-piperidyl]indolin-7-yl]carbamate (Compound 58.1)

To a solution of tert-butyl N-ethyl-N-[5-fluoro-3-methyl-2-oxo-3-[(3R,4R)-3-acetamido-4-[2-(4-carbamoylphenoxy)phenyl]-1-piperidyl]indolin-7-yl]carbamate (compound 57.1, 50 mg, 75.8 μmol) in DCM (10 mL) was added Et₃N (38.3 mg, 37.5 μmol), followed by TFAA (31.8 mg, 145.8 μmol). The resultant mixture was stirred at room temperature for 2 hrs. The reaction mixture was quenched with water and extracted twice with DCM. The combined organic layer was washed with brine, dried over Na₂SO₄, filtered and concentrated in vacuo. The residue was purified by flash column chromatography (silica gel, 10 g, 0% to 100% EA in PE) to afford compound 58.1 (35 mg, 72.0% yield). MS: calc'd 642 [(M+H)⁺], measured 642 [(M+H)⁺].

### Step (b): Preparation of N-[(3R,4R)-4-[2-(4-cyanophenoxy)phenyl]-1-[7-(ethylamino)-5-fluoro-3-methyl-2-oxo-indolin-3-yl]-3-piperidyl]acetamide (Example 58)

To a solution of tert-butyl N-ethyl-N-[5-fluoro-3-methyl-2-oxo-3-[(3R,4R)-3-acetamido-4-[2-(4-cyanophenoxy)phenyl]-1-piperidyl]indolin-7-yl]carbamate (compound 58.1, 35 mg, 54.6 μmol) in DCM (10 mL) was added TFA (5 mL). The reaction mixture was stirred at room temperature for 2 hrs, then concentrated to afford a crude product which was purified by pre-HPLC to afford Example 58 (15 mg, 50.7% yield). MS: calc'd 542 [(M+H)⁺], measured 542 [(M+H)⁺]. ¹H NMR (500 MHz, METHANOL-d₄) δ=7.72 (d, J=8.9 Hz, 2H), 7.38 (br d, J=7.8 Hz, 1H), 7.31-7.25 (m, 1H), 7.22 (d, J=7.6 Hz, 1H), 7.08 (d, J=8.4 Hz, 2H), 6.92 (br d, J=7.9 Hz, 1H), 6.61-6.56 (m, 1H), 6.50-6.42 (m, 1H), 4.63-4.47 (m, 1H), 3.54-3.35 (m, 2H), 3.19-3.13 (m, 2H), 3.09-2.99 (m, 1H), 2.96-2.76 (m, 2H), 2.09-1.94 (m, 2H), 1.74 (d, J=2.1 Hz, 3H), 1.70 (d, J=5.6 Hz, 3H), 1.32-1.24 (m, 3H).

### Example 59A and 59B

### (3R,4R)-1-[(3R)-7-(ethylamino)-5-fluoro-3-methyl-2-oxo-indolin-3-yl]-4-phenyl-piperidine-3-carboxamide and (3R,4R)-1-[(3S)-7-(ethylamino)-5-fluoro-3-methyl-2-oxo-indolin-3-yl]-4-phenyl-piperidine-3-carboxamide

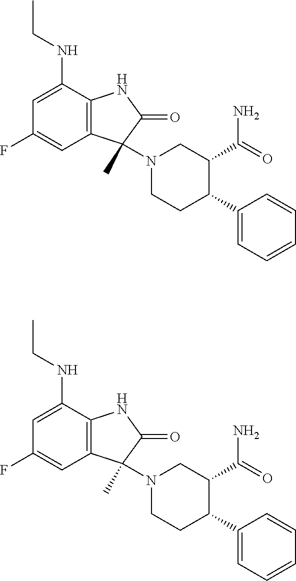 [SMILES: CCNc1cc(F)cc2c1NC(=O)[C@]2(C)N1CC[C@@H](c2ccccc2)[C@@H](C(N)=O)C1 ; CCNc1cc(F)cc2c1NC(=O)[C@@]2(C)N1CC[C@@H](c2ccccc2)[C@@H](C(N)=O)C1]

The titled compound was synthesized according to the following scheme:

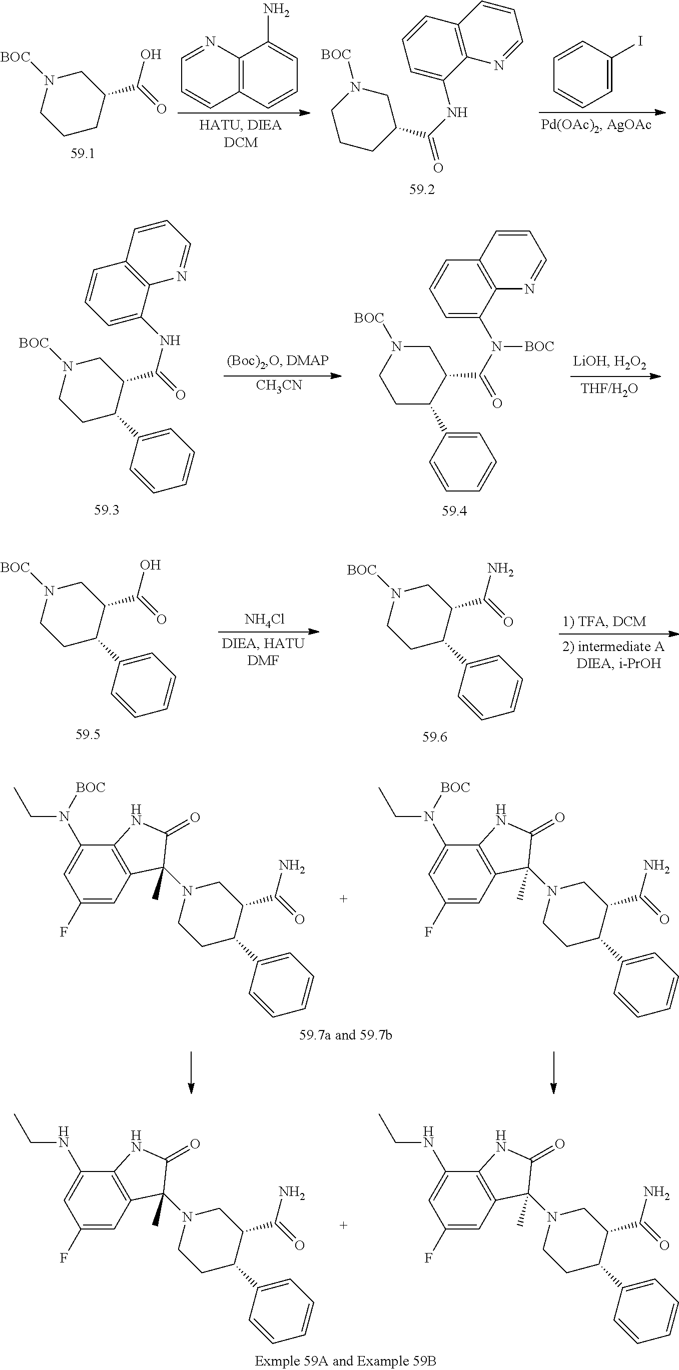

### Step (a): Preparation of tert-butyl (3R)-3-(8-quinolylcarbamoyl)piperidine-1-carboxylate (Compound 59.2)

To a solution of (3R)-1-tert-butoxycarbonylpiperidine-3-carboxylic acid (compound 59.1, 20 g, 87.23 mmol) and DIEA (30.39 mL, 174.47 mmol) in DCM (250 mL) was added HATU (39.8 g, 104.68 mmol) at 0° C., the resultant mixture was stirred at this temperature for 30 min, and then quinolin-8-amine (13.83 g, 95.96 mmol) was added. The reaction mixture was slowly warmed to 20° C. and stirred at 20° C. for another 16 hrs. The mixture was diluted with DCM (500 mL), washed with water (200 mL), brine (300 mL), dried over Na₂SO₄ and concentrated in vacuo. The residue was purified by flash chromatography (silica gel, 330 g 0 to 40% EA in PE) to afford compound 59.2 (18 g, 58.1% yield) as a white solid. MS: calc'd 356 [(M+H)⁺], measured 356 [(M+H)⁺].

### Step (b): Preparation of tert-butyl (3R,4R)-4-phenyl-3-(8-quinolylcarbamoyl)piperidine-1-carboxylate (Compound 59.3)

A mixture of tert-butyl (3R)-3-(8-quinolylcarbamoyl)piperidine-1-carboxylate (compound 59.2, 9 g, 25.32 mmol), iodobenzene (25.83 g, 126.61 mmol), Pd(OAc)₂ (1.14 g, 5.06 mmol) and AgOAc (2.59 mL, 50.64 mmol) was degassed and purged with N₂ for 3 times, and then the resultant mixture was stirred at 110° C. for 18 hrs. under N₂ atmosphere. After being cooled to room temperature, the reaction mixture was diluted with EA (500 mL) and water (200 mL), then filtered, and the filter cake was washed with EA (100 mL) twice. The combined organic layer was washed with brine (200 mL), dried over Na₂SO₄, filtered and concentrated in vacuo. The residue was purified by flash chromatography (silica gel, 120 g, 0 to 20% EA in PE) to afford compound 59.3 (4.25 g, 38.9% yield). MS: calc'd 432 [(M+H)⁺], measured 432 [(M+H)⁺].

### Step (c): Preparation of tert-butyl (3R,4R)-3-[tert-butoxycarbonyl(8-quinolyl)carbamoyl]-4-phenyl-piperidine-1-carboxylate (Compound 59.4)

To a solution of tert-butyl (3R,4R)-4-phenyl-3-(8-quinolylcarbamoyl)piperidine-1-carboxylate (compound 59.3, 3 g, 6.95 mmol) in acetonitrile (50 mL) was added (Boc)₂O (15.17 g, 69.52 mmol) and DMAP (2.27 g, 13.9 mmol), the resultant mixture was heated to 90° C. and stirred for 16 hrs. After being cooled to room temperature, the reaction mixture was diluted with EA (500 mL), washed with water (100 mL) and brine (100 mL), dried over Na₂SO₄ and concentrated in vacuo. The residue was purified by flash chromatography (silica gel, 40 g, 0% to 20% EA in PE) to afford compound 59.4 (3.2 g, 86.7% yield). MS: calc'd 532 [(M+H)⁺], measured 532 [(M+H)⁺].

### Step (d): Preparation of (3R,4R)-1-tert-butoxycarbonyl-4-phenyl-piperidine-3-carboxylic acid (Compound 59.5)

To a solution of tert-butyl (3R,4R)-3-[tert-butoxycarbonyl(8-quinolyl)carbamoyl]-4-phenyl-piperidine-1-carboxylate (compound 59.4, 3.2 g, 6.02 mmol) in THF (30 mL) and water (10 mL) was added LiOH (506 mg, 12.05 mmol) and H₂O₂ (10.23 g, 90.29 mmol) at 0° C. The resultant mixture was warmed to 20° C. and stirred at this temperature for 16 hrs. The reaction was quenched with aq. Na₂SO₃ (50 mL), and extracted with EA (300 mL). The organic layer was washed with water (100 mL) and brine (100 mL), dried over Na₂SO₄, filtered and concentrated in vacuo. The residue was purified by prep-HPLC to afford compound 59.5 (1.0 g, 51.7% yield). MS: calc'd 306 [(M+H)⁺], measured 328 [(M+Na)⁺].

### Step (e): Preparation of tert-butyl (3R,4R)-3-carbamoyl-4-phenyl-piperidine-1-carboxylate (Compound 59.6)

To a solution of (3R,4R)-1-tert-butoxycarbonyl-4-phenyl-piperidine-3-carboxylic acid (compound 59.5, 200 mg, 0.65 mmol) and DIEA (0.23 mL, 1.32 mmol) in DMF (5 mL) was added HATU (232 mg, 0.99 mmol) at 0° C., the resultant mixture was stirred at this temperature for 15 min, and then NH₄Cl (53.0 mg, 0.99 mmol) was added. The reaction mixture was slowly warmed to 20° C. and stirred at 20° C. for 16 hrs. The mixture was purified by pre-HPLC to afford compound 59.6 (150 mg, 75.9% yield). MS: calc'd 305 [(M+H)⁺], measured 327 [(M+Na)⁺].

### Step (f): Preparation of tert-butyl N-[(3R)-3-[(3R,4R)-3-carbamoyl-4-phenyl-1-piperidyl]-5-fluoro-3-methyl-2-oxo-indolin-7-yl]-N-ethyl-carbamate and tert-butyl N-[(3S)-3-[(3R,4R)-3-carbamoyl-4-phenyl-1-piperidyl]-5-fluoro-3-methyl-2-oxo-indolin-7-yl]-N-ethyl-carbamate (Compound 59.7a and 59.7b)

To a solution of tert-butyl (3R,4R)-3-carbamoyl-4-phenyl-piperidine-1-carboxylate (compound 59.6, 150 mg, 0.49 mmol) in DCM (5 mL) was added TFA (1.5 mL). The reaction mixture was stirred at room temperature for 2 hrs, then concentrated to afford an oil. To a solution of the residue and DIEA (0.26 mL, 1.47 mmol) in isopropanol (5 mL) was added tert-butyl N-(3-bromo-5-fluoro-3-methyl-2-oxo-indolin-7-yl)-N-ethyl-carbamate (intermediate A, 150 mg, 0.39 mmol). After being stirred at room temperature for 1 hrs., the reaction mixture was concentrated. The residue was purified by prep-HPLC to afford compound 59.7a (faster eluting, 90 mg, 36.0% yield) and compound 59.7b (slower eluting, 100 mg, 40.0% yield). MS: calc'd 511 [(M+H)⁺], measured 511 [(M+H)⁺].

### Step (g): Preparation of Example 59A and Example 59B

To a solution of compound 59.7a (90 mg, 176.1 μmol) in DCM (5 mL) was added TFA (1.5 mL). The reaction mixture was stirred at room temperature for 2 hrs, then concentrated to afford a crude product, which was purified by prep-HPLC to afford Example 59A (35.7 mg, 49.4% yield). MS: calc'd 411 [(M+H)⁺], measured 411 [(M+H)⁺]. ¹H NMR (400 MHz, DMSO-d₆) δ=9.99 (s, 1H), 7.77 (s, 1H), 7.23-7.12 (m, 5H), 6.92 (s, 1H), 6.37 (d, J=6.8 Hz, 1H), 6.31 (d, J=12.4 Hz, 1H), 5.17 (brs, 1H), 3.13-2.96 (m, 5H), 2.87-2.78 (m, 1H), 2.64-2.56 (m, 1H), 2.34-2.18 (m, 2H), 1.70-1.61 (m, 1H), 1.41 (s, 3H), 1.20 (t, J=7.2 Hz, 3H).

To a solution of compound 59.7b (100 mg, 195.6 μmol) in DCM (5 mL) was added TFA (1.5 mL). The reaction mixture was stirred at room temperature for 2 hrs, then concentrated to afford a crude product, which was purified by prep-HPLC to afford Example 59B (46.9 mg, 58.5% yield) as a white solid. MS: calc'd 411 [(M+H)⁺], measured 411 [(M+H)⁺]. ¹H NMR (400 MHz, DMSO-d₆) δ=9.96 (s, 1H), 7.77 (s, 1H), 7.21-7.11 (m, 5H), 6.83 (s, 1H), 6.39-6.30 (m, 2H), 5.17 (t, J=4.0 Hz, 1H), 3.13-3.06 (m, 2H), 3.06-3.00 (m, 2H), 2.86-2.81 (m, 1H), 2.65-2.61 (m, 1H), 2.54-2.52 (m, 1H), 2.39 (t, J=10.4 Hz, 1H), 2.24-2.13 (m, 1H), 1.64 (d, J=10.8 Hz, 1H), 1.40 (s, 3H), 1.20 (t, J=7.2 Hz, 3H).

### Example 60A and 60B

### (3R,4R)-1-[(3R)-7-(ethylamino)-5-fluoro-3-methyl-2-oxo-indolin-3-yl]-N-methyl-4-phenyl-piperidine-3-carboxamide and (3R,4R)-1-[(3S)-7-(ethylamino)-5-fluoro-3-methyl-2-oxo-indolin-3-yl]-N-methyl-4-phenyl-piperidine-3-carboxamide

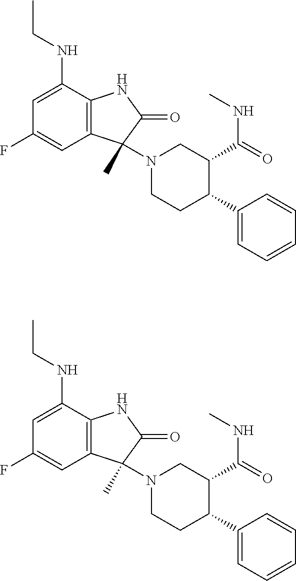 [SMILES: CCNc1cc(F)cc2c1NC(=O)[C@]2(C)N1CC[C@@H](c2ccccc2)[C@@H](C(=O)NC)C1 ; CCNc1cc(F)cc2c1NC(=O)[C@@]2(C)N1CC[C@@H](c2ccccc2)[C@@H](C(=O)NC)C1]

Example 60A and Example 60B were prepared in analogy to Example 59A and Example 59B, by replacing NH₄Cl with methylamine in step (e).

Example 60A MS: calc'd 425 [(M+H)⁺], measured 425 [(M+H)⁺]. ¹H NMR (400 MHz, DMSO-d₆) δ=10.69 (s, 1H), 7.90, (s, 1H), 7.34-7.27 (m, 2H), 7.26-7.20 (m, 1H), 7.12 (d, J=7.2 Hz, 2H), 6.66 (d, J=7.2 Hz, 1H), 6.49 (d, J=12.0 Hz, 1H), 3.48-3.24 (m, 4H), 3.13-3.08 (m, 3H), 2.89-2.87 (m, 1H), 2.43-2.42 (m, 4H), 1.89 (d, J=13.2 Hz, 1H), 1.68 (s, 3H), 1.20 (t, J=7.2 Hz, 3H).

Example 60B MS: calc'd 425 [(M+H)⁺], measured 425 [(M+H)⁺]. ¹H NMR (400 MHz, DMSO-d₆) δ=10.43 (s, 1H), 7.98 (s, 1H), 7.30-7.24 (m, 2H), 7.22-7.18 (m, 1H), 7.12-7.10 (m, 2H), 6.75-6.54 (m, 1H), 6.46 (d, J=12.4 Hz, 1H), 3.31-3.17 (m, 3H), 3.12 (q, J=7.2 Hz, 2H), 3.05-2.83 (m, 2H), 2.79-2.67 (m, 1H), 2.49-2.39 (m, 4H), 1.90-1.74 (m, 1H), 1.63 (s, 3H), 1.20 (t, J=7.2 Hz, 3H).

### Example 61A and 61B

### (3R,4R)-1-[(3R)-7-(ethylamino)-5-fluoro-3-methyl-2-oxo-indolin-3-yl]-N,4-diphenyl-piperidine-3-carboxamide and (3R,4R)-1-[(3S)-7-(ethylamino)-5-fluoro-3-methyl-2-oxo-indolin-3-yl]-N,4-diphenyl-piperidine-3-carboxamide

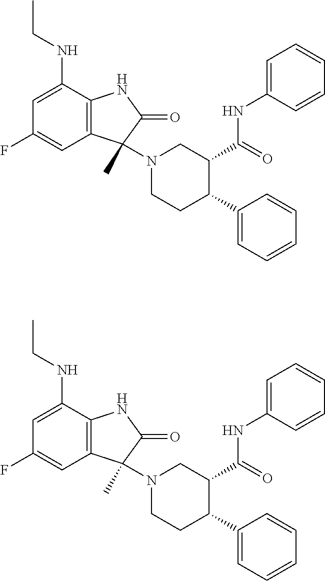 [SMILES: CCNc1cc(F)cc2c1NC(=O)[C@]2(C)N1CC[C@@H](c2ccccc2)[C@@H](C(=O)Nc2ccccc2)C1 ; CCNc1cc(F)cc2c1NC(=O)[C@@]2(C)N1CC[C@@H](c2ccccc2)[C@@H](C(=O)Nc2ccccc2)C1]

Example 61A and Example 61B were prepared in analogy to Example 59A and Example 59B, by replacing NH₄Cl with aniline in step (e).

Example 61A MS: calc'd 487 [(M+H)⁺], measured 487 [(M+H)⁺]. ¹H NMR (400 MHz, DMSO-d₆) δ=10.49 (s, 1H), 7.56-7.41 (m, 2H), 7.31-7.21 (m, 5H), 7.20-7.15 (m, 3H), 7.08-7.01 (m, 1H), 6.64-6.47 (m, 1H), 6.41 (d, J=10.2 Hz, 1H), 3.44-3.15 (m, 3H), 3.10 (q, J=7.2 Hz, 2H), 3.04-2.79 (m, 3H), 2.45-2.36 (m, 1H), 1.93-1.76 (m, 1H), 1.73-1.48 (m, 3H), 1.20 (t, J=7.2 Hz, 3H).

Example 61B MS: calc'd 487 [(M+H)⁺], measured 487 [(M+H)⁺]. ¹H NMR (400 MHz, DMSO-d₆) δ=10.39 (s, 1H), 7.57-7.40 (m, 2H), 7.31-7.20 (m, 5H), 7.19-7.14 (m, 3H), 7.07-7.01 (m, 1H), 6.74-6.51 (m, 1H), 6.49-6.37 (m, 1H), 3.43-3.18 (m, 3H), 3.11 (q, J=6.8 Hz, 2H), 2.99-2.77 (m, 3H), 2.47-2.37 (m, 1H), 1.90-1.80 (m, 1H), 1.73-1.49 (m, 3H), 1.21 (t, J=7.2 Hz, 3H).

### Example 62A and 62B

### (3R,4R)-N-benzyl-1-[(3R)-7-(ethylamino)-5-fluoro-3-methyl-2-oxo-indolin-3-yl]-4-phenyl-piperidine-3-carboxamide and (3R,4R)-N-benzyl-1-[(3S)-7-(ethylamino)-5-fluoro-3-methyl-2-oxo-indolin-3-yl]-4-phenyl-piperidine-3-carboxamide

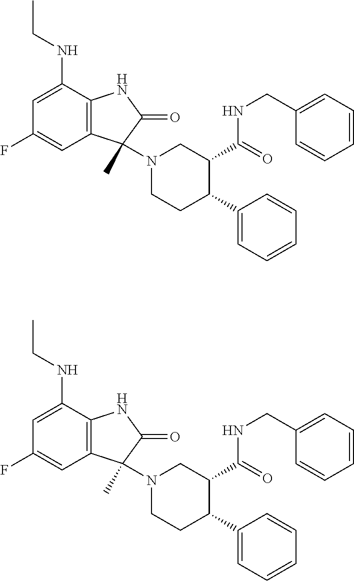 [SMILES: CCNc1cc(F)cc2c1NC(=O)[C@]2(C)N1CC[C@@H](c2ccccc2)[C@@H](C(=O)NCc2ccccc2)C1 ; CCNc1cc(F)cc2c1NC(=O)[C@@]2(C)N1CC[C@@H](c2ccccc2)[C@@H](C(=O)NCc2ccccc2)C1]

Example 62A and Example 62B were prepared in analogy to Example 59A and Example 59B, by replacing NH₄Cl with phenylmethanamine in step (e).

Example 62A MS: calc'd 501 [(M+H)⁺], measured 501 [(M+H)⁺]. ¹H NMR (400 MHz, DMSO-d₆) δ=9.97 (s, 1H), 8.75 (t, J=6.0 Hz, 1H), 7.32-7.26 (m, 2H), 7.26-7.22 (m, 1H), 7.21-7.17 (m, 5H), 7.12-7.07 (m, 2H), 6.33-6.25 (m, 2H), 5.18 (t, J=4.4 Hz, 1H), 4.27-4.15 (m, 2H), 3.12-3.01 (m, 4H), 2.87-2.81 (m, 1H), 2.65 (d, J=1.6 Hz, 1H), 2.57 (dd, J=12.0, 3.6 Hz, 2H), 2.34-2.22 (m, 1H), 1.65 (dd, J=13.2, 2.0 Hz, 1H), 1.35 (s, 3H), 1.19 (t, J=7.2 Hz, 3H).

Example 62B MS: calc'd 501 [(M+H)⁺], measured 501 [(M+H)⁺]. ¹H NMR (400 MHz, DMSO-d₆) δ=9.98 (s, 1H), 8.78 (t, J=5.6 Hz, 1H), 7.37-7.30 (m, 4H), 7.29-7.24 (m, 1H), 7.23-7.18 (m, 2H), 7.16-7.08 (m, 3H), 6.29 (dd, J=12.4, 2.4 Hz, 1H), 5.98 (dd, J=8.0, 2.4 Hz, 1H), 5.17 (t, J=4.4 Hz, 1H), 4.35 (dd, J=14.4, 6.0 Hz, 1H), 4.18 (dd, J=14.4, 5.2 Hz, 1H), 3.18 (d, J=12.0 Hz, 1H), 3.11-3.03 (m, 2H), 2.91-2.82 (m, 2H), 2.78 (dd, J=12.0, 2.8 Hz, 1H), 2.68 (d, J=4.4 Hz, 1H), 2.35-2.27 (m, 1H), 2.18-2.07 (m, 1H), 1.64-1.56 (m, 1H), 1.30 (s, 3H), 1.19 (t, J=7.2 Hz, 3H).

### Example 63A and 63B

### (3S,4S)-1-[(3R)-7-(ethylamino)-5-fluoro-3-methyl-2-oxo-indolin-3-yl]-4-phenyl-piperidine-3-carboxamide and (3S,4S)-1-[(3S)-7-(ethylamino)-5-fluoro-3-methyl-2-oxo-indolin-3-yl]-4-phenyl-piperidine-3-carboxamide

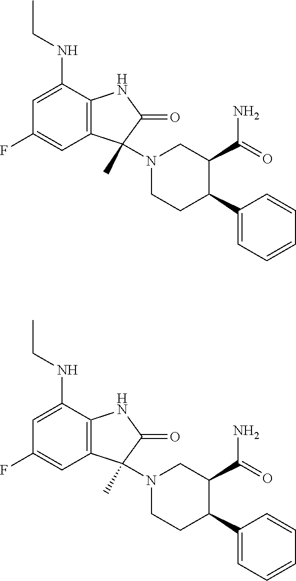 [SMILES: CCNc1cc(F)cc2c1NC(=O)[C@]2(C)N1CC[C@H](c2ccccc2)[C@H](C(N)=O)C1 ; CCNc1cc(F)cc2c1NC(=O)[C@@]2(C)N1CC[C@H](c2ccccc2)[C@H](C(N)=O)C1]

Example 63A and Example 63B were prepared in analogy to Example 59A and Example 59B, by replacing (3R)-1-tert-butoxycarbonylpiperidine-3-carboxylic acid (compound 59.1) with (3S)-1-tert-butoxycarbonylpiperidine-3-carboxylic acid in step (a).

Example 63A MS: calc'd 411 [(M+H)⁺], measured 411 [(M+H)⁺]. ¹H NMR (400 MHz, DMSO-d₆) δ=10.06 (s, 1H), 7.73 (s, 1H), 7.47-7.08 (m, 5H), 6.97 (s, 1H), 6.60-6.18 (m, 2H), 5.27 (s, 1H), 3.18-2.97 (m, 5H), 2.95-2.83 (m, 1H), 2.77-2.59 (m, 2H), 2.41-2.15 (m, 1H), 1.73-1.70 (m, 1H), 1.54-1.37 (m, 3H), 1.21 (t, J=7.2, 3H).

Example 63B MS: calc'd 411 [(M+H)⁺], measured 411 [(M+H)⁺]. ¹H NMR (400 MHz, DMSO-d₆) δ=10.05 (s, 1H), 7.71 (m, 1H), 7.33-7.06 (m, 5H), 6.88 (m, 1H), 6.58-6.22 (m, 2H), 5.26 (s, 1H), 3.24-3.00 (m, 5H), 2.95-2.86 (m, 1H), 2.77-2.68 (m, 1H), 2.65-2.56 (m, 1H), 2.36-2.18 (m, 1H), 1.83-1.62 (m, 1H), 1.57-1.36 (m, 3H), 1.21 (t, J=7.2 Hz, 3H).

### BIOLOGICAL EXAMPLES

### Example 64: 50% Inhibitory Concentration (IC₅₀) of In Vitro PDHK1 Activity

The inhibitory potency of compounds of PDHK1 activity was determined in an enzymatic assay. Pyruvate dehydrogenase complex (PDHc) is an enzyme that catalyzes the reaction of pyruvate and a lipoamide to give the acetylated dihydrolipoamide and carbon dioxide. PDHc has three subunits E1, E2 and E3. E1 has 3 serine phosphorylation sites S293, S232, S300. If E1 was phosphorylated, PDH will be inactivated. PDHK can phosphorylate PDH protein (also named PDHE1) in PDH complex (PDC) to inactivate it. In the PDHK1 enzymatic assays, the final reaction mixture contains 1 nM of PDHK1 (in house purification from Escherichia coli), 25 nM of PDHE1 (in house purification from Escherichia coli) and 20 μM of ATP, and the experiment was reacted at 30° C. for 45 minutes. After adding detection reagent AlphaScreen Histidine (Nickel Chelate, from Perkin Elmer), and anti-phosphorylation s-293 antibody (from Abcam), Anti-Rabbit IgG Alpha (from Perkin Elmer)), the plate was incubated at 30° C. for 60 min before measuring the light change using a luminometer (Envision)

TABLE 1
Enzymatic IC₅₀ values of the compounds
of this invention against PDHK1

| Example No. | IC₅₀ (μg/mL) |
|---|---|
| 1B | 0.007 |
| 2 | 0.015 |
| 3A | 0.036 |
| 4B | 0.054 |
| 5 | 0.046 |
| 6 | 0.143 |
| 7 | 0.154 |
| 8 | 0.022 |
| 9 | 0.015 |
| 10 | 0.059 |
| 11 | 0.020 |
| 12 | 0.062 |
| 13 | 0.004 |
| 14 | 0.007 |
| 15 | 0.060 |
| 16 | 0.047 |
| 17 | 0.012 |
| 18 | 0.059 |
| 19 | 0.016 |
| 20 | 0.038 |
| 21 | 0.037 |
| 22 | 0.024 |
| 23 | 0.049 |
| 24 | 0.016 |
| 25 | 0.142 |
| 26 | 0.067 |
| 27 | 0.086 |
| 28 | 0.032 |
| 29 | 0.035 |
| 30 | 0.065 |
| 31 | 0.197 |
| 32 | 0.135 |
| 33 | 0.068 |
| 34 | 0.032 |
| 35 | 0.030 |
| 36 | 0.047 |
| 37 | 0.039 |
| 38 | 0.026 |
| 39 | 0.007 |
| 40 | 0.017 |
| 41 | 0.032 |
| 42 | 0.020 |
| 43 | 0.016 |
| 44 | 0.022 |
| 45 | 0.011 |
| 46 | 0.012 |
| 47 | 0.019 |
| 48 | 0.025 |
| 49 | 0.007 |
| 50 | 0.014 |
| 51 | 0.017 |
| 52 | 0.020 |
| 53 | 0.015 |
| 54 | 0.009 |
| 55 | 0.037 |
| 56 | 0.005 |
| 57 | 0.019 |
| 58 | 0.03 |
| 59B | 0.007 |
| 60B | 0.009 |
| 61B | 0.023 |
| 62B | 0.038 |
| 63A | 0.009 |

1. A compound of formula (I), 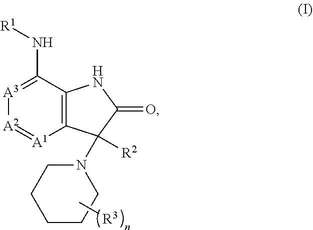
wherein
R¹ is C₁₋₆alkyl;
R² is C₁₋₆alkyl;
R³ is OR⁴, —(CH₂)ₙ—R⁵, NR⁶R⁷, COR⁸ or R⁹; wherein
R⁴ is phenyl substituted once or twice by substituents independently selected from carboxy, halogen, hydroxy, formyl, halosulfonyl and C₁₋₆alkyl;
R⁵ is phenyl substituted by carbamoyl, carboxy or cyano;
R⁶ is H, C₁₋₆alkyl, C₁₋₆alkylcarbonyl or carbamoyl;
R⁷ is C₂₋₆alkenylsulfonyl,
benzoyl substituted once, twice or three times by substituents independently selected from halogen, hydroxy and halosulfonyl,
COR^{a}, wherein R^{a} is 1,1-dioxothianyl, C₁₋₆alkyl, cyanoazaspiro[3.3]heptanyl, haloC₁₋₆alkylcarbonyl-azaspiro[3.3]heptanyl, C₂₋₆alkenylcarbonyl-azaspiro[3.3]heptanyl, cyanoazetidinyl or caynopiperidinyl,
phenyl substituted once or twice by substituents independently selected from cyano, hydroxy, C₁₋₆alkoxy, hydroxyC₁₋₆alkylcarbonyl, halosulfonyl, halosulfonyloxy and C₂₋₆alkenylsulfonyl,
pyrazinyl substituted by cyano,
pyridinyl substituted once or twice by substituents independently selected from halogen, C₁₋₆alkyl, carboxy, carbamoyl, cyano, hydroxy, hydroxyC₁₋₆alkyl, C₁₋₆alkylsulfonyl, halosulfonyl and halosulfonyloxy, or
pyrimidinyl substituted by cyano;
R⁸ is amino, C₁₋₆alkylamino, phenylamino or benzylamino;
R⁹ is C₁₋₆alkyl, halogen, hydroxy, phenyl, (carboxyphenoxy)phenyl, (carbamoylphenoxy)phenyl or (cyanophenoxy)phenyl;
A¹ is CH;
A² is CR^{b}, wherein R^{b} is halogen;
A³ is CH;
n is 1, 2, 3, 4, 5 or 6;
or a pharmaceutically acceptable salt thereof.

2. A compound of formula (II), 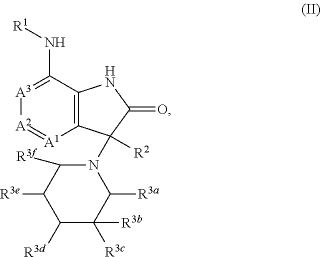
wherein
R¹ is C₁₋₆alkyl;
R² is C₁₋₆alkyl;
R^{3a} is H;
R^{3b} is OR⁴, —(CH₂)—R⁵, NR⁶R⁷ or COR⁸; wherein
R⁴ is phenyl substituted once or twice by substituents independently selected from carboxy, halogen, hydroxy, formyl, halosulfonyl and C₁₋₆alkyl;
R⁵ is phenyl substituted by carbamoyl, carboxy or cyano;
R⁶ is H, C₁₋₆alkyl, C₁₋₆alkylcarbonyl or carbamoyl;
R⁷ is C₂₋₆alkenylsulfonyl,
benzoyl substituted once, twice or three times by substituents independently selected from halogen, hydroxy and halosulfonyl,
COR^{a}, wherein R^{a} is 1,1-dioxothianyl, C₁₋₆alkyl, cyanoazaspiro[3.3]heptanyl, haloC₁₋₆alkylcarbonyl-azaspiro[3.3]heptanyl, C₂₋₆alkenylcarbonyl-azaspiro[3.3]heptanyl, cyanoazetidinyl or caynopiperidinyl,
phenyl substituted once or twice by substituents independently selected from cyano, hydroxy, C₁₋₆alkoxy, hydroxyC₁₋₆alkylcarbonyl, halosulfonyl, halosulfonyloxy and C₂₋₆alkenylsulfonyl,
pyrazinyl substituted by cyano,
pyridinyl substituted once or twice by substituents independently selected from halogen, C₁₋₆alkyl, carboxy, carbamoyl, cyano, hydroxy, hydroxyC₁₋₆alkyl, C₁₋₆alkylsulfonyl, halosulfonyl and halosulfonyloxy, or
pyrimidinyl substituted by cyano;
R⁸ is amino, C₁₋₆alkylamino, phenylamino or benzylamino;
R^{3c} is H or C₁₋₆alkyl;
R^{3d} is H or R⁹; wherein R⁹ is C₁₋₆alkyl, halogen, hydroxy, phenyl, (carboxyphenoxy)phenyl, (carbamoylphenoxy)phenyl or (cyanophenoxy)phenyl;
R^{3e} is H or halogen;
R^{3f} is H;
A¹ is CH;
A² is CR^{b}, wherein R^{b} is halogen;
A³ is CH;
n is 1, 2, 3, 4, 5 or 6;
or a pharmaceutically acceptable salt thereof.

3. A compound according to claim 1, wherein R⁴ is formyl(hydroxy)phenyl.

4. A compound according to claim 1, wherein R⁴ is 3-formyl-4-hydroxyphenyl.

5. A compound according to claim 1, wherein R⁶ is H.

6. A compound according to claim 1, wherein
R⁷ is COR^{a}, wherein R^{a} is C₁₋₆alkyl, cyanoazaspiro[3.3]heptanyl, haloC₁₋₆alkylcarbonyl-azaspiro[3.3]heptanyl or C₂₋₆alkenylcarbonyl-azaspiro[3.3]heptanyl,
phenyl substituted once or twice by substituents independently selected from C₁₋₆alkoxy, halosulfonyl and C₂₋₆alkenylsulfonyl, or
pyridinyl substituted by cyano or halosulfonyloxy.

7. A compound according to claim 6, wherein
R⁷ is COR^{a}, wherein R^{a} is acetyl, cyanoazaspiro[3.3]heptanyl, (chloroacetyl)azaspiro[3.3]heptanyl or (propenoyl)azaspiro[3.3]heptanyl,
phenyl substituted once or twice by substituents independently selected from methoxy, fluorosulfonyl and vinylsulfonyl, or
pyridinyl substituted by cyano or fluorosulfonyloxy.

8. A compound according to claim 1, wherein R⁹ is C₁₋₆alkyl, phenyl or (cyanophenoxy)phenyl.

9. A compound according to claim 8, wherein R⁹ is methyl, phenyl or (cyanophenoxy)phenyl.

10. A compound according to claim 1 wherein n is 1.

11. A compound according to claim 1, wherein
R¹ is C₁₋₆alkyl;
R² is C₁₋₆alkyl;
R³ is OR⁴, NR⁶R⁷ or R⁹; wherein
R⁴ is formyl(hydroxy)phenyl;
R⁶ is H;
R⁷ is COR^{a}, wherein R^{a} is C₁₋₆alkyl, cyanoazaspiro[3.3]heptanyl, haloC₁₋₆alkylcarbonyl-azaspiro[3.3]heptanyl or C₂₋₆alkenylcarbonyl-azaspiro[3.3]heptanyl,
phenyl substituted once or twice by substituents independently selected from C₁₋₆alkoxy, halosulfonyl and C₂₋₆alkenylsulfonyl, or
pyridinyl substituted by cyano or halosulfonyloxy;
R⁹ is C₁₋₆alkyl, phenyl or (cyanophenoxy)phenyl;
A¹ is CH;
A² is CR^{b}, wherein R^{b} is halogen;
A³ is CH;
or a pharmaceutically acceptable salt thereof.

12. A compound according to claim 11, wherein
R¹ is ethyl;
R² is methyl or ethyl;
R³ is OR⁴, NR⁶R⁷ or R⁹; wherein
R⁴ is formyl(hydroxy)phenyl;
R⁶ is H;
R⁷ is COR^{a}, wherein R^{a} is methyl, cyanoazaspiro[3.3]heptanyl, (chloroacetyl)azaspiro[3.3]heptanyl or (propenoyl)azaspiro[3.3]heptanyl,
phenyl substituted once or twice by substituents independently selected from methoxy, fluorosulfonyl and vinylsulfonyl, or
pyridinyl substituted by cyano or fluorosulfonyloxy;
R⁹ is methyl, phenyl or (cyanophenoxy)phenyl;
A¹ is CH;
A² is CR^{b}, wherein R^{b} is fluoro;
A³ is CH;
or a pharmaceutically acceptable salt thereof.

13. A compound according to claim 12, wherein
R¹ is ethyl;
R² is methyl or ethyl;
R³ is OR⁴, NR⁶R⁷ or R⁹; wherein
R⁴ is 3-formyl-4-hydroxyphenyl;
R⁶ is H;
R⁷ is acetyl, 2-cyano-2-azaspiro[3.3]heptane-6-carbonyl, 2-(2-chloroacetyl)-2-azaspiro[3.3]heptane-6-carbonyl or 2-prop-2-enoyl-2-azaspiro[3.3]heptane-6-carbonyl, 4-fluorosulfonyl-3-methoxyphenyl, 4-vinylsulfonylphenyl, 6-cyano-3-pyridinyl or 6-fluorosulfonyloxy-3-pyridinyl;
R⁹ is methyl, phenyl or 2-(4-cyanophenoxy)phenyl;
A¹ is CH;
A² is CR^{b}, wherein R^{b} is fluoro;
A³ is CH;
or a pharmaceutically acceptable salt thereof.

14. A compound according to claim 2, wherein
R¹ is C₁₋₆alkyl;
R² is C₁₋₆alkyl;
R^{3a} is H;
R^{3b} is OR⁴ or NR⁶R⁷; wherein
R⁴ is formyl(hydroxy)phenyl;
R⁶ is H;
R⁷ is COR^{a}, wherein R^{a} is C₁₋₆alkyl, cyanoazaspiro[3.3]heptanyl, haloC₁₋₆ alkylcarbonyl-azaspiro[3.3]heptanyl or C₂₋₆alkenylcarbonyl-azaspiro[3.3]heptanyl,
phenyl substituted once or twice by substituents independently selected from C₁₋₆alkoxy, halosulfonyl and C₂₋₆alkenylsulfonyl, or
pyridinyl substituted by cyano or halosulfonyloxy;
R^{3c} is H or C₁₋₆alkyl;
R^{3d} is H or R⁹; wherein R⁹ is C₁₋₆alkyl, phenyl or (cyanophenoxy)phenyl;
R^{3e} is H;
R^{3f} is H;
A¹ is CH;
A² is CR^{b}, wherein R^{b} is halogen;
A³ is CH;
or a pharmaceutically acceptable salt thereof.

15. A compound according to claim 14, wherein
R¹ is ethyl;
R² is methyl or ethyl;
R^{3a} is H;
R^{3b} is OR⁴ or NR⁶R⁷; wherein
R⁴ is formyl(hydroxy)phenyl;
R⁶ is H;
R⁷ is COR^{a}, wherein R^{a} is methyl, cyanoazaspiro[3.3]heptanyl, (chloroacetyl)azaspiro[3.3]heptanyl or (propenoyl)azaspiro[3.3]heptanyl,
phenyl substituted once or twice by substituents independently selected from methoxy, fluorosulfonyl and vinylsulfonyl, or
pyridinyl substituted by cyano or fluorosulfonyloxy;
R^{3c} is H or methyl;
R^{3d} is H or R⁹; wherein R⁹ is methyl, phenyl or (cyanophenoxy)phenyl;
R^{3e} is H;
R^{3f} is H;
A¹ is CH;
A² is CR^{b}, wherein R^{b} is fluoro;
A³ is CH;
or a pharmaceutically acceptable salt thereof.

16. A compound according to claim 15, wherein
R¹ is ethyl;
R² is methyl or ethyl;
R^{3a} is H;
R^{3b} is OR⁴ or NR⁶R⁷; wherein
R⁴ is formyl(hydroxy)phenyl;
R⁶ is H;
R⁷ is acetyl, 2-cyano-2-azaspiro[3.3]heptane-6-carbonyl, 2-(2-chloroacetyl)-2-azaspiro[3.3]heptane-6-carbonyl or 2-prop-2-enoyl-2-azaspiro[3.3]heptane-6-carbonyl, 4-fluorosulfonyl-3-methoxyphenyl, 4-vinylsulfonylphenyl, 6-cyano-3-pyridinyl or 6-fluorosulfonyloxy-3-pyridinyl;
R^{3c} is H or methyl;
R^{3d} is H or R⁹; wherein R⁹ is methyl, phenyl or 2-(4-cyanophenoxy)phenyl yl;
R^{3e} is H;
R^{3f} is H;
A¹ is CH;
A² is CR^{b}, wherein R^{b} is fluoro;
A³ is CH;
or a pharmaceutically acceptable salt thereof.

17. A compound selected from:
2-chloro-4-[[(3R)-1-[(3R)-7-(ethylamino)-5-fluoro-3-methyl-2-oxo-indolin-3-yl]-3-piperidyl]oxy]benzoic acid;
2-chloro-4-[[(3R)-1-[(3S)-7-(ethylamino)-5-fluoro-3-methyl-2-oxo-indolin-3-yl]-3-piperidyl]oxy]benzoic acid;
5-[[(3R)-1-[7-(ethylamino)-5-fluoro-3-methyl-2-oxo-indolin-3-yl]-3-piperidyl]oxy]-2-hydroxy-benzaldehyde;
4-[[(3R)-1-[(3R)-7-(ethylamino)-5-fluoro-3-methyl-2-oxo-indolin-3-yl]-3-piperidyl]oxy]benzenesulfonyl fluoride;
4-[[(3R)-1-[(3S)-7-(ethylamino)-5-fluoro-3-methyl-2-oxo-indolin-3-yl]-3-piperidyl]oxy]benzenesulfonyl fluoride;
4-[[(3R)-1-[(3R)-7-(ethylamino)-5-fluoro-3-methyl-2-oxo-indolin-3-yl]-3-piperidyl]oxy]-2-methoxy-benzenesulfonyl fluoride;
4-[[(3R)-1-[(3S)-7-(ethylamino)-5-fluoro-3-methyl-2-oxo-indolin-3-yl]-3-piperidyl]oxy]-2-methoxy-benzenesulfonyl fluoride;
4-[[(3S)-1-[7-(ethylamino)-5-fluoro-3-methyl-2-oxo-indolin-3-yl]-3-piperidyl]methyl]benzoic acid;
4-[[(3S)-1-[7-(ethylamino)-5-fluoro-3-methyl-2-oxo-indolin-3-yl]-3-piperidyl]methyl]benzamide;
4-[[(3S)-1-[7-(ethylamino)-5-fluoro-3-methyl-2-oxo-indolin-3-yl]-3-piperidyl]methyl]benzonitrile;
4-[[(3R)-1-[7-(ethylamino)-5-fluoro-3-methyl-2-oxo-indolin-3-yl]-3-piperidyl]amino]benzonitrile;
4-[[(3R)-1-[7-(ethylamino)-5-fluoro-3-methyl-2-oxo-indolin-3-yl]-3-piperidyl]amino]-2-hydroxy-benzonitrile;
5-[[(3S)-1-[7-(ethylamino)-5-fluoro-3-methyl-2-oxo-indolin-3-yl]-3-piperidyl]amino]pyridine-2-carboxylic acid;
5-[[(3R)-1-[7-(ethylamino)-5-fluoro-3-methyl-2-oxo-indolin-3-yl]-3-piperidyl]amino]pyridine-2-carboxylic acid;
5-[[(3R)-1-[7-(ethylamino)-5-fluoro-3-methyl-2-oxo-indolin-3-yl]-3-piperidyl]amino]pyridine-2-carboxamide;
5-[[(3R)-1-[7-(ethylamino)-5-fluoro-3-methyl-2-oxo-indolin-3-yl]-3-piperidyl]amino]pyridine-2-carbonitrile;
6-[[(3R)-1-[7-(ethylamino)-5-fluoro-3-methyl-2-oxo-indolin-3-yl]-3-piperidyl]amino]pyridine-3-carbonitrile;
5-[[(3R)-1-[7-(ethylamino)-5-fluoro-3-methyl-2-oxo-indolin-3-yl]-3-piperidyl]amino]pyrazine-2-carbonitrile;
6-[[(3R)-1-[7-(ethylamino)-5-fluoro-3-methyl-2-oxo-indolin-3-yl]-3-piperidyl]amino]pyridazine-3-carbonitrile;
5-[[(3R)-1-[7-(ethylamino)-5-fluoro-3-methyl-2-oxo-indolin-3-yl]-3-piperidyl]amino]pyrimidine-2-carbonitrile;
7-(ethylamino)-5-fluoro-3-[(3R)-3-[[6-(hydroxymethyl)-3-pyridyl]amino]-1-piperidyl]-3-methyl-indolin-2-one;
7-(ethylamino)-5-fluoro-3-[(3R)-3-[4-(2-hydroxyacetyl)anilino]-1-piperidyl]-3-methyl-indolin-2-one;
7-(ethylamino)-5-fluoro-3-methyl-3-[(3R)-3-[(6-methylsulfonyl-3-pyridyl)amino]-1-piperidyl]indolin-2-one;
7-(ethylamino)-5-fluoro-3-[(3R)-3-[(6-hydroxy-3-pyridyl)amino]-1-piperidyl]-3-methyl-indolin-2-one;
3-chloro-5-[[(3R)-1-[7-(ethylamino)-5-fluoro-3-methyl-2-oxo-indolin-3-yl]-3-piperidyl]amino]pyridine-2-carbonitrile;
5-[[(3R)-1-[7-(ethylamino)-5-fluoro-3-methyl-2-oxo-indolin-3-yl]-3-methyl-3-piperidyl]amino]pyridine-2-carbonitrile;
5-[[(3R)-1-[7-(ethylamino)-5-fluoro-3-methyl-2-oxo-indolin-3-yl]-3-piperidyl]-methyl-amino]pyridine-2-carboxamide;
N-(6-cyano-3-pyridyl)-N-[(3R)-1-[3-ethyl-7-(ethylamino)-5-fluoro-2-oxo-indolin-3-yl]-3-piperidyl]acetamide;
1-(6-cyano-3-pyridyl)-1-[(3R)-1-[3-ethyl-7-(ethylamino)-5-fluoro-2-oxo-indolin-3-yl]-3-piperidyl]urea;
3-[[(3R)-1-[7-(ethylamino)-5-fluoro-3-methyl-2-oxo-indolin-3-yl]-3-piperidyl]amino]benzenesulfonyl fluoride;
4-[[(3R)-1-[7-(ethylamino)-5-fluoro-3-methyl-2-oxo-indolin-3-yl]-3-piperidyl]amino]-2-methoxy-benzenesulfonyl fluoride;
6-[[(3R)-1-[7-(ethylamino)-5-fluoro-3-methyl-2-oxo-indolin-3-yl]-3-piperidyl]amino]pyridine-3-sulfonyl fluoride;
4-methyl-6-[[(3R)-1-[7-(ethylamino)-5-fluoro-3-methyl-2-oxo-indolin-3-yl]-3-piperidyl]amino]pyridine-3-sulfonyl fluoride;
7-(ethylamino)-5-fluoro-3-methyl-2-oxo-3-[(3R)-3-(3-fluorosulfonyloxyanilino)-1-piperidyl]indoline;
7-(ethylamino)-5-fluoro-3-methyl-2-oxo-3-[(3R)-3-(4-fluorosulfonyloxyanilino)-1-piperidyl]indoline;
7-(ethylamino)-5-fluoro-3-methyl-2-oxo-3-[(3R)-3-[(6-fluorosulfonyloxy-3-pyridyl)amino]-1-piperidyl]indoline;
7-(ethylamino)-5-fluoro-3-methyl-3-[(3R)-3-(4-vinylsulfonylanilino)-1-piperidyl]indolin-2-one;
N-[(3R)-1-[7-(ethylamino)-5-fluoro-3-methyl-2-oxo-indolin-3-yl]-3-piperidyl]-3,5-difluoro-4-hydroxy-benzamide;
N-[(3R)-1-[7-(ethylamino)-5-fluoro-3-methyl-2-oxo-indolin-3-yl]-3-piperidyl]-1,1-dioxo-thiane-4-carboxamide;
2-cyano-N-[(3R)-1-[7-(ethylamino)-5-fluoro-3-methyl-2-oxo-indolin-3-yl]-3-piperidyl]-2-azaspiro[3.3]heptane-6-carboxamide;
1-cyano-N-[(3R)-1-[7-(ethylamino)-5-fluoro-3-methyl-2-oxo-indolin-3-yl]-3-piperidyl]piperidine-4-carboxamide;
2-cyano-N-[(3R)-1-[3-ethyl-7-(ethylamino)-5-fluoro-2-oxo-indolin-3-yl]-3-piperidyl]-2-azaspiro[3.3]heptane-6-carboxamide;
2-(2-chloroacetyl)-N-[(3R)-1-[7-(ethylamino)-5-fluoro-3-methyl-2-oxo-indolin-3-yl]-3-piperidyl]-2-azaspiro[3.3]heptane-6-carboxamide;
N-[(3R)-1-[7-(ethylamino)-5-fluoro-3-methyl-2-oxo-indolin-3-yl]-3-piperidyl]-2-prop-2-enoyl-2-azaspiro[3.3]heptane-6-carboxamide;
3-[[(3R)-1-[7-(ethylamino)-5-fluoro-3-methyl-2-oxo-indolin-3-yl]-3-piperidyl]carbamoyl]benzenesulfonyl fluoride;
4-[[(3R)-1-[7-(ethylamino)-5-fluoro-3-methyl-2-oxo-indolin-3-yl]-3-piperidyl]carbamoyl]benzenesulfonyl fluoride;
7-(ethylamino)-5-fluoro-3-[(3R)-3-[(3-fluorosulfonyloxybenzoyl)amino]-1-piperidyl]-3-methyl-2-oxo-indoline;
5-[[(3R,5R)-1-[7-(ethylamino)-5-fluoro-3-methyl-2-oxo-indolin-3-yl]-5-fluoro-3-piperidyl]amino]pyridine-2-carbonitrile;
5-[[(3R,5S)-1-[7-(ethylamino)-5-fluoro-3-methyl-2-oxo-indolin-3-yl]-5-fluoro-3-piperidyl]amino]pyridine-2-carbonitrile;
5-[[(3R,5S)-1-[7-(ethylamino)-5-fluoro-3-methyl-2-oxo-indolin-3-yl]-5-methyl-3-piperidyl]amino]pyridine-2-carbonitrile;
5-[[(3S,4S)-1-[7-(ethylamino)-5-fluoro-3-methyl-2-oxo-indolin-3-yl]-4-fluoro-3-piperidyl]amino]pyridine-2-carbonitrile;
5-[[(3S,4S)-1-[3-ethyl-7-(ethylamino)-5-fluoro-2-oxo-indolin-3-yl]-4-fluoro-3-piperidyl]amino]pyridine-2-carbonitrile;
5-[[(3S,4S)-1-[7-(ethylamino)-5-fluoro-3-methyl-2-oxo-indolin-3-yl]-4-hydroxy-3-piperidyl]amino]pyridine-2-carbonitrile;
5-[[(3S,4R)-1-[7-(ethylamino)-5-fluoro-3-methyl-2-oxo-indolin-3-yl]-4-hydroxy-3-piperidyl]amino]pyridine-2-carbonitrile;
N-[(3R,4R)-1-[7-(ethylamino)-5-fluoro-3-methyl-2-oxo-indolin-3-yl]-4-phenyl-3-piperidyl]acetamide;
N-[(3R,4R)-1-[7-(ethylamino)-5-fluoro-3-methyl-2-oxo-indolin-3-yl]-4-phenyl-3-piperidyl]ethenesulfonamide;
1-cyano-N-[(3R,4R)-1-[7-(ethylamino)-5-fluoro-3-methyl-2-oxo-indolin-3-yl]-4-phenyl-3-piperidyl]azetidine-3-carboxamide;
5-[[(3R,4R)-1-[7-(ethylamino)-5-fluoro-3-methyl-2-oxo-indolin-3-yl]-4-phenyl-3-piperidyl]amino]pyridine-2-carbonitrile;
4-[2-[(3R,4R)-3-acetamido-1-[7-(ethylamino)-5-fluoro-3-methyl-2-oxo-indolin-3-yl]-4-piperidyl]phenoxy]benzoic acid;
4-[2-[(3R,4R)-3-acetamido-1-[7-(ethylamino)-5-fluoro-3-methyl-2-oxo-indolin-3-yl]-4-piperidyl]phenoxy]benzamide;
N-[(3R,4R)-4-[2-(4-cyanophenoxy)phenyl]-1-[7-(ethylamino)-5-fluoro-3-methyl-2-oxo-indolin-3-yl]-3-piperidyl]acetamide;
(3R,4R)-1-[(3R)-7-(ethylamino)-5-fluoro-3-methyl-2-oxo-indolin-3-yl]-4-phenyl-piperidine-3-carboxamide;
(3R,4R)-1-[(3S)-7-(ethylamino)-5-fluoro-3-methyl-2-oxo-indolin-3-yl]-4-phenyl-piperidine-3-carboxamide;
(3R,4R)-1-[(3R)-7-(ethylamino)-5-fluoro-3-methyl-2-oxo-indolin-3-yl]-N-methyl-4-phenyl-piperidine-3-carboxamide;
(3R,4R)-1-[(3S)-7-(ethylamino)-5-fluoro-3-methyl-2-oxo-indolin-3-yl]-N-methyl-4-phenyl-piperidine-3-carboxamide;
(3R,4R)-1-[(3R)-7-(ethylamino)-5-fluoro-3-methyl-2-oxo-indolin-3-yl]-N,4-diphenyl-piperidine-3-carboxamide;
(3R,4R)-1-[(3S)-7-(ethylamino)-5-fluoro-3-methyl-2-oxo-indolin-3-yl]-N,4-diphenyl-piperidine-3-carboxamide;
(3R,4R)-N-benzyl-1-[(3R)-7-(ethylamino)-5-fluoro-3-methyl-2-oxo-indolin-3-yl]-4-phenyl-piperidine-3-carboxamide;
(3R,4R)-N-benzyl-1-[(3S)-7-(ethylamino)-5-fluoro-3-methyl-2-oxo-indolin-3-yl]-4-phenyl-piperidine-3-carboxamide;
(3S,4S)-1-[(3R)-7-(ethylamino)-5-fluoro-3-methyl-2-oxo-indolin-3-yl]-4-phenyl-piperidine-3-carboxamide; and
(3S,4S)-1-[(3S)-7-(ethylamino)-5-fluoro-3-methyl-2-oxo-indolin-3-yl]-4-phenyl-piperidine-3-carboxamide;
or a pharmaceutically acceptable salt thereof.

18. A process for the preparation of a compound according to claim 2 comprising any of the following steps:
a) deprotection of compound of formula (XX), 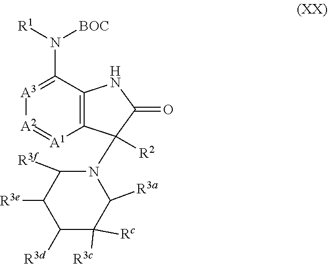
to form the compound of formula (IV), 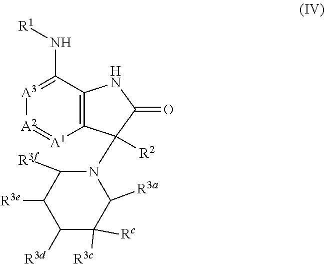
using a suitable acid;
b) reaction between compound of formula (XXIV), R⁷-Q, with compound of formula (XXIII), 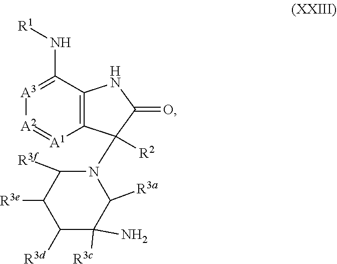
via aromatic substitution, acylation, sulfonation or coupling in the presence of a base, provide the final compound of formula (V), 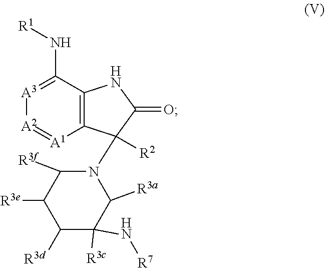
c) formation of compound of formula (VI), 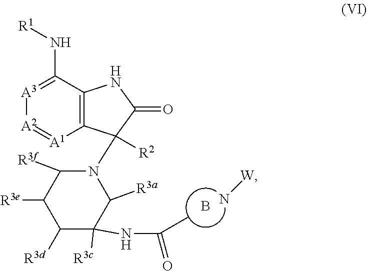
via reaction of compound of formula (XXX), 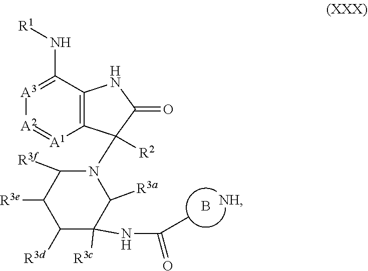
with BrCN, C₂₋₆alkenylcarbonyl chloride or C₂₋₆alkenylsulfonyl chloride in the presence of a base;
d) deprotection of compound of formula (XXXII), 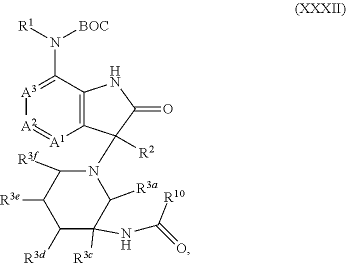
using trifluoroacetic acid, to afford compound of formula (VII), 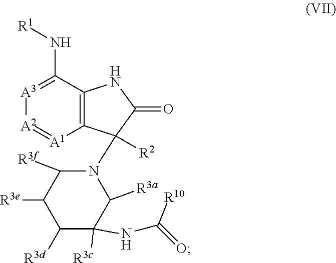
wherein R¹, R², R⁴, R⁵, R⁶, R⁷, R^{3a}, R^{3c}, R^{3d}, R^{3e}, R^{3f}, A¹, A², A³ and n are as defined in claim 2; ring B is unsubstituted or substituted heterocyclyl, particularly ring B is selected from azetidinyl, pyrrolidinyl, piperidinyl and azaspiro[3.3]heptanyl; W is cyano, haloC₁₋₆alkylcarbonyl, C₂₋₆alkenylcarbonyl or C₂₋₆alkenylsulfonyl; R^{c} is NR⁶R⁷, OR⁴ or —(CH₂)ₙ—R⁵; Q is F, Cl, or OH.

19. (canceled)

20. A pharmaceutical composition comprising a compound in accordance with claim 1 and a therapeutically inert carrier.

21. A method for the inhibition of PDHK1, the method comprises administering a therapeutically effective amount of a compound as defined in claim 1.

22-26. (canceled)

27. A method for the treatment or prophylaxis of autoimmune and inflammatory disease or cancer; wherein autoimmune and inflammatory disease is selected from inflammatory bowel disease (IBD), chronic obstructive pulmonary disease (COPD), idiopathic pulmonary fibrosis (IPF), psoriasis, systemic lupus erythematosus (SLE); wherein cancer is selected from oropharyngeal squamous cell carcinoma, liver cancer, lung cancer, stomach cancer, and colon cancer, which method comprises administering a therapeutically effective amount of a compound as defined in claim 1.

28. A method for the treatment or prophylaxis of inflammatory bowel disease (IBD), the method comprises administering a therapeutically effective amount of a compound as defined in claim 1.
